(19) United States
(12) Patent Application Publication
(10) Pub. No.: US 2025/0064927 A1
(43) Pub. Date: Feb. 27, 2025

(54) COMPOSITIONS COMPRISING CROSS-SPECIES-SPECIFIC ANTIBODIES AND USES THEREOF

(71) Applicant: Amgen Research (Munich) GMBH, Munich (DE)

(72) Inventors: Roman Kischel, Munich (DE); Tobias Raum, Munich (DE); Bernd Schlereth, Munich (DE); Doris Rau, Munich (DE); Ronny Cierpka, Munich (DE); Peter Kufer, Munich (DE)

(21) Appl. No.: 18/431,108

(22) Filed: Feb. 2, 2024

Related U.S. Application Data
(63) Continuation of application No. 16/116,293, filed on Aug. 29, 2018, now Pat. No. 11,925,684.
Application No. 16/116,293 is a continuation of application No. 13/567,672, filed on Aug. 6, 2012, now abandoned.
Application No. 13/567,672 is a continuation of application No. 12/083,351, filed on Jun. 3, 2008, now Pat. No. 8,236,308.
(60) Provisional application No. 60/724,781, filed on Oct. 11, 2005.

(51) Int. Cl. A61K 39/395; A61K 39/00; A61K 45/06; C07K 16/28; C07K 16/30; C07K 16/40; C07K 16/46

(52) U.S. Cl. CPC A61K 39/39558; A61K 45/06; C07K 16/2809; C07K 16/30; C07K 16/40; C07K 16/468; A61K 2039/505; C07K 2317/31; C07K 2317/34; C07K 2317/56; C07K 2317/622

(57) ABSTRACT

The present invention relates to uses of bispecific antibodies exhibiting cross-species specificity for evaluating the in vivo safety and/or activity and/or pharmacokinetic profile of the same in non-human species and humans. The present invention moreover relates to methods for evaluating the in vivo safety and/or activity and/or pharmacokinetic profile of said bispecific antibodies exhibiting cross-species specificity. The present invention also relates to methods of measuring the biological activity and/or efficacy of such bispecific antibodies exhibiting cross-species specificity. In addition, the present invention relates to pharmaceutical compositions comprising bispecific single chain antibodies exhibiting cross-species specificity and to methods for the preparation of pharmaceutical compositions comprising said bispecific single chain antibodies exhibiting cross-species specificity for the treatment of diseases.

This application contains, as a separate part of disclosure, a Sequence Listing in computer-readable form (Filename: 47403C_SeqListing.xml; Size: 348,097 bytes; Created: Feb. 1, 2024) which is incorporated by reference herein in its entiret

The present invention relates to uses of bispecific antibodies exhibiting cross-species specificity for evaluating the in vivo safety and/or activity and/or pharmacokinetic profile of the same in non-human species and humans. The present invention moreover relates to methods for evaluating the in vivo safety and/or activity and/or pharmacokinetic profile of said bispecific antibodies exhibiting cross-species specificity. The present invention also relates to methods of measuring the biological activity and/or efficacy of such bispecific antibodies exhibiting cross-species specificity. In addition, the present invention relates to pharmaceutical compositions comprising bispecific single chain antibodies exhibiting cross-species specificity and to methods for the preparation of pharmaceutical compositions comprising said bispecific single chain antibodies exhibiting cross-species specificity for the treatment of diseases.

In order to be marketed, any new candidate medication must pass through rigorous testing. Roughly, this testing can be subdivided into preclinical and clinical phases: Whereas the latter—further subdivided into the generally known clinical phases I, II and III—is performed in human patients, the former is performed in animals. Generally, the aim of pre-clinical testing is to prove that the drug candidate works and is efficacious and safe. Specifically, the purpose of these animal studies is to prove that the drug is not carcinogenic, mutagenic or teratogenic, as well as to understand the pharmacokinetic of the drug. Only when the safety in animals and possible effectiveness of the drug candidate has been established in preclinical testing will this drug candidate be approved for clinical testing in humans.

The behavior of a small molecule drug candidate, e.g. a new anthracycline-based antineoplastic agent, in animals will in many cases be indicative of the expected behavior of this drug candidate upon administration to humans. As a result, the data obtained from such preclinical testing will therefore generally have a high predictive power for the human case.

However, such compatibility is not to be expected with all types of drug candidates; certain molecular formats would be expected to behave one way in animals and another way in humans. In such cases, the predictive power of preclinical tests—and hence the likelihood of approval of the drug candidate for clinical testing—is greatly reduced.

One format of drug candidate which often acts differently in animals than in humans is an antibody. Generally, antibodies function by way of highly specific recognition of—usually proteinaceous-target molecules. Most antibody drug candidates are monoclonal antibodies; they recognize only a single site, or epitope, on their target molecule. However, while this discriminatory ability inherent to monoclonal antibodies and fragments thereof makes these compounds very interesting candidates for drug development, it also complicates their preclinical testing. This is because of species-dependent variations in the sequence of the target molecule bound by such antibodies. A monoclonal antibody or fragment thereof which specifically recognizes and binds to, say, molecule Y via epitope X in humans, will often fail to specifically recognize and bind to the corresponding molecule Y′ in a non-human species since the corresponding epitope X′ may be different from its human counterpart X. Thus, monoclonal antibodies (e.g. against human antigens) by design tend to have limited reactivity to phylogenetically distant species such as rodents, except in the very rare cases in which the antigen is highly conserved. Even among the group of monoclonal antibodies with reactivity to human and primate antigens, there are numerous examples of antibodies which react only with the human and chimpanzee antigen homologs. This has also been observed for anti-CD3 monoclonal antibodies. One of the most widely used and best characterized monoclonal antibodies specific for the CD3 complex is OKT-3 which reacts with chimpanzee CD3 but not with the CD3 homolog of other primates, such as macaques, or with dog CD3 (Sandusky et al., J. Med. Primatol. 15 (1986), 441-451). The anti-CD3 monoclonal antibody UCHT-1 is also reactive with CD3 from chimpanzee but not with CD3 from macaques (own data; see the following Examples). On the other hand, there are also examples of monoclonal antibodies which recognize macaque antigens, but not their human counterparts. One example of this group is monoclonal antibody FN-18 directed to CD3 from macaques (Uda et al., J. Med. Primatol. 30 (2001), 141-147). Interestingly, it has been found that peripheral lymphocytes from about 12% of cynomolgus monkeys lacked reactivity with anti-rhesus monkey CD3 monoclonal antibody (FN-18) due to a polymorphism of the CD3 antigen in macaques. Uda et al. described a substitution of two amino acids in the CD3 sequence of cynomolgus monkeys which are not reactive with FN-18 antibodies, as compared to CD3 derived from animals which are reactive with FN-18 antibodies (Uda et al., J Med Primatol. 32 (2003), 105-10; Uda et al., J Med Primatol. 33 (2004), 34-7).

Similar difficulties with the high specificity of monoclonal antibodies in preclinical animal testing are observed with bispecific antibodies, for example a recombinant bispecific single chain antibody of the general type disclosed in, for example, U.S. Pat. No. 5,260,203. This added difficulty is due to the fact that a bispecific antibody, for example a bispecific single chain antibody, comprises two distinct binding domains, either one of which—or both—may fail to recognize the non-human counterpart of its human target molecule. Effectively, the risk that e.g. a bispecific single chain antibody, will fail to recognize its intended respective target molecules in an animal is twice as high as with a monospecific antibody or fragment thereof.

There exist several known strategies for countering such problems.

One known approach is to perform preclinical testing of the (bispecific) antibody drug candidate or fragment thereof in a chimpanzee model. The chimpanzee is the closest genetic relative to human, identical to the latter in over 99% of its genome, so the chimpanzee variant of a molecule specifically bound by a (bispecific) antibody drug candidate or fragment thereof is very likely to be identical to the human variant of this molecule. The danger of non-recognition of this molecule by the (bispecific) antibody drug candidate or fragment thereof in chimpanzee is therefore minimized. However, testing in chimpanzees is very expensive and fraught with ethical problems. Furthermore, chimpanzees are endangered animals so that the number of animals which can be used in experimentation is very limited. For most developers of (bispecific) antibody therapeutics, such preclinical testing in chimpanzees is therefore precluded.

The above approach is described e.g. in Schlereth et al. (Cancer Immunol. Immunother. 20 (2005), 1-12). In this study, the biological activity of a clinical drug candidate, bispecific single chain antibody CD19×CD3, has been tested in chimpanzee. CD19×CD3 antibody, previously described in WO 99/54440 for therapeutic administration in humans, is a bispecific single chain antibody specifically binding to human B cell antigen CD19 and human T cell antigen CD3. The authors of this paper found that this bispecific single chain antibody bound to both human and chimpanzee variants of the CD3 and CD19 molecule. However, no reactivity of said bispecific single chain antibody to B and T cells from other species, i.e. mouse, beagle dog, and non-chimpanzee primates (cynomolgus, rhesus and baboon), could be found, again confirming the extreme species sensitivity of monoclonal antibodies.

Another approach adapts the molecule used in preclinical testing to the animal used for this testing. According to this approach, the requisite safety information is obtained in preclinical studies by constructing so-called “surrogate” antibodies for administration to test animals. Generally, such a surrogate antibody is an antibody which has been modified so as to specifically recognize and bind to the test animal counterpart of the target molecule bound by the non-surrogate antibody, i.e. the actual drug candidate in humans. Thus, in approaches using such “surrogate” antibodies, two different molecules have to be separately developed and investigated: the clinical drug candidate and a candidate for preclinical testing in an animal species corresponding to the target specificity of the clinical candidate. The major drawback of such surrogate approaches is that the surrogate antibody for preclinical testing has been modified vis-à-vis the actual drug candidate antibody. Therefore, the data obtained in preclinical testing using a surrogate antibody are often not directly applicable to the human case. As explained above, this reduced applicability ultimately reduces the predictive power of any preclinical study using these approaches.

While the above approach adapts the drug candidate to match the animal used for testing, other known approaches do exactly the converse; according to these other known approaches, the animal used for testing is adapted to the drug candidate intended for administration to humans.

One example of the adaptation of the test animal to the drug candidate intended for administration to humans, is the creation of a transgenic animal expressing the human molecule specifically bound by the (bispecific) antibody or fragment thereof instead of the non-human molecule which is endogenous to its own species. In this way, the (bispecific) antibody or fragment thereof administered in preclinical trials will encounter and bind to the human antigen in the transgenic test animal. For example, in a study designed by Bugelski et al. (Bugelski et al., Hum Exp Toxicol. 19 (2000), 230-243), preclinical safety assessment of monoclonal antibody Keliximab has been carried out in a human CD4 transgenic mouse in order to support chronic treatment of rheumatoid arthritis in human patients. Keliximab is a monoclonal antibody with specificity for human and chimpanzee CD4. The authors conclude that the use of transgenic mice expressing human proteins provides a useful alternative to studies in chimpanzees with biopharmaceutical agents having limited cross-species specificity (Bugelski et al., Hum Exp Toxicol. 19 (2000), 230-243). However, creation of transgenic animals for test purposes is very labor- and therefore cost-intensive.

In the same vein, an alternative approach often employed is to inject a non-transgenic test animal with human cells expressing the molecule to be specifically bound by the (bispecific) antibody or fragment thereof being tested. However, while avoiding the costs and time associated with constructing transgenic animal species, this approach presents other problems. For one, in approaches using e.g. immunocompetent mice, foreign cells introduced into the animal are often recognized by the immune system of the test animal and are systematically eliminated. Although immunodeficient mice allow the injection and growth of non-syngeneic cells, for instance in xenograft tumor models, the applicability of the data obtained for the drug candidate in such studies is limited due to the phylogenetic distance between rodents and humans. In addition, multiple blood extractions are problematic in lower animals, say a mouse. However, such multiple blood extractions are essential for the determination of pharmacokinetic parameters and the continuous testing of blood parameters for evaluating the biological effects of a drug candidate in preclinical animal testing.

In summary, there are two main approaches of obtaining preclinical data on safety and toxicity of a drug candidate for administration in humans. One way is the application of the clinical drug candidate to transgenic animal models, mostly mouse models. However, preclinical data are of limited explanatory power due to the fact that rodents are less related to humans compared to primates. Another way is the testing of surrogate molecules in a relevant animal species. These surrogate molecules are specific for the animals used and are therefore different from the clinical drug candidate developed for administration in humans. The problem is that the clinical drug candidate cannot directly be applied in an animal other than chimpanzees which is closely related to humans and has highly predictive power when used in preclinical testing. Existing methods for obtaining meaningful preclinical data regarding a (bispecific) antibody or fragment thereof undergoing testing as a drug candidate either match this antibody to the test animal, in which case the data obtained are often of only limited applicability for the drug candidate or, conversely, match the test animal to the antibody, in which case ethical and/or cost difficulty arise/s and, in the worst case, the applicability of the data obtained for the drug candidate may still be limited.

It is therefore an aim of the invention to provide a solution to the problems outlined above.

The solution to these problems is the provision of bispecific single chain antibodies exhibiting cross-species specificity which bind to human and non-chimpanzee primate target molecules and therefore can be used both for preclinical evaluation of safety, activity and/or pharmacokinetic profile of said bispecific antibody in primates and—in the identical form—as drugs in humans.

Accordingly, one aspect of the invention relates to the use of a bispecific single chain antibody comprising a first binding domain binding to a non-chimpanzee primate CD3, and a second binding domain binding to a cell surface antigen antigen, wherein said first binding domain binds to human and non-chimpanzee primate CD3,

for evaluating the (in vivo) safety and/or activity and/or pharmacokinetic profile of said bispecific single chain antibody in humans, comprising (i) administering said bispecific single chain antibody to a non-chimpanzee primate, (ii) measuring said (in vivo) safety and/or activity and/or pharmacokinetic profile of said bispecific single chain antibody in said non-chimpanzee primate, and (iii) evaluating the (in vivo) safety and/or activity and/or pharmacokinetic profile of said bispecific single chain antibody in humans.

In another aspect, the invention relates to a method for evaluating the biological activity/safety/toxicity of a bispecific single chain antibody as defined above, comprising
(i) administering said bispecific single chain antibody to a non-chimpanzee primate,
(ii) measuring the in vivo safety and/or activity and/or pharmacokinetic profile of said bispecific single chain antibody in said non-chimpanzee primate,
(iii) evaluating the in vivo safety and/or activity and/or pharmacokinetic profile of said bispecific single chain antibody in the non-chimpanzee primate, and
(iv) determining an effective and non-toxic dose of said bispecific single chain antibody and administering said dose to humans.

In particular, it is an aim of the invention to provide means and methods which improve the predictive value of data obtained in preclinical animal testing for the administration of the drug candidate to humans.

As used herein, a “bispecific single chain antibody” denotes a single polypeptide chain comprising two binding domains. Each binding domain comprises one variable region from an antibody heavy chain (“VH region”), wherein the VH region of the first binding domain specifically binds to said first molecule, i.e. the CD3 molecule, and the VH region of the second binding domain specifically binds to a cell surface antigen, as defined in more detail below. The two binding domains are optionally linked to one another by a short polypeptide spacer generally comprising on the order of 5 amino acids. Each binding domain may additionally comprise one variable region from an antibody light chain (“VL region”), the VH region and VL region within each of the first and second binding domains being linked to one another via a polypeptide linker, for example of the type disclosed and claimed in EP 623679 B1, but in any case long enough to allow the VH region and VL region of the first binding domain and the VH region and VL region of the second binding domain to pair with one another such that, together, they are able to specifically bind to the respective first and second molecules.

As used herein, the term “binds” or related expressions such as “binding” or “reactivity with/to” etc. refer to the ability of the first and/or second binding domains of the bispecific single chain antibody as defined herein to discriminate between the respective first and/or second molecule to such an extent that, from a pool of a plurality of different molecules as potential binding partners, only said respective first and/or second molecule is/are bound, or is/are significantly bound. Such binding measurements can be routinely performed e.g. on a Biacore apparatus.

More specifically, the first binding domain of the bispecific single chain antibody as defined herein binds to human CD3 and to non-chimpanzee primate CD3. The term “non-chimpanzee primate” is explained in more detail below. As evident to the person skilled in the art, it is not excluded from the scope of the invention that the first binding domain of the bispecific single chain antibodies exhibiting cross-species specificity as defined herein may also bind, e.g., to chimpanzee CD3. On the other hand, it is apparent that binding domains which only bind to human CD3, but not to non-chimpanzee primate CD3, are excluded from the scope of the invention. This applies mutatis mutandis to binding domains which only bind to non-chimpanzee primate CD3, but not to human CD3, such as e.g. those of monoclonal antibody FN-18.

The second binding domain of the bispecific single chain antibodies as defined herein binds to a cell surface antigen, preferably a tumor antigen, as set forth below. Preferably, both binding molecules of the bispecific single chain antibodies as defined herein are binding to their respective human and non-chimpanzee primate target molecules. The second binding domain, thus, binds to a human cell surface antigen and to the corresponding homolog of the cell surface antigen in a non-chimpanzee primate. The identification and determination of homologs of human cell surface antigens in non-chimpanzee primates is well known to the person skilled in the art and can be carried out e.g. by sequence alignments.

The term “cross-species specificity” or “interspecies specificity” as used herein means binding of at least one of the two binding domains, preferably of both binding domains, of the bispecific single chain antibody described herein to the same target molecule in humans and non-chimpanzee primates. Thus, “cross-species specificity” or “interspecies specificity” is to be understood as an interspecies reactivity to the same molecule X, but not to a molecule other than X. Cross-species specificity of a monoclonal antibody recognizing e.g. human CD3, to a non-chimpanzee primate CD3, e.g. macaque CD3, can be determined, for instance, by FACS analysis. The FACS analysis is carried out in a way that the respective monoclonal antibody is tested for binding to human and non-chimpanzee primate cells, e.g. macaque cells, expressing said human and non-chimpanzee primate CD3 antigens, respectively. An appropriate assay is shown in the following examples. For the generation of the first binding domain of the bispecific single chain antibodies as defined herein, e.g. monoclonal antibodies binding to both the human and non-chimpanzee CD3 (e.g. macaque CD3) can be used. Similarly, for the generation of the second binding domain of the bispecific single chain antibodies as defined herein, monoclonal antibodies binding to both of the respective human and non-chimpanzee primate cell surface antigens can be utilized. Appropriate binding domains for the bispecific single chain antibodies as defined herein can be derived from cross-species specific monoclonal antibodies by recombinant methods described in the art. A monoclonal antibody binding to a human cell surface antigen and to the homolog of said cell surface antigen in a non-chimpanzee primate can be tested by FACS assays as set forth above. It is evident to those skilled in the art that cross-species specific monoclonal antibodies can also be generated by hybridoma techniques described in the literature (Milstein and Köhler, Nature 256 (1975), 495-7). For example, mice may be alternately immunized with human and non-chimpanzee primate CD3. From these mice, cross-species specific antibody-producing hybridoma cells are isolated via hybridoma technology and analysed by FACS as set forth above. The generation and analysis of bispecific single chain antibodies exhibiting cross-species specificity as described herein is shown in the following examples. The advantages of the bispecific single chain antibodies exhibiting cross-species specificity include the points enumerated below.

As used herein, “human” and “man” refers to the species Homo sapiens. A “human” molecule is therefore the variant of that molecule as it is naturally expressed in Homo sapiens. As far as the medical uses of the constructs described herein are concerned, human patients are to be treated with the same.

As used herein, a “non-chimpanzee primate” or “non-chimp primate” or grammatical variants thereof refers to any primate other than chimpanzee, i.e. other than an animal of belonging to the genus Pan, and including the species Pan paniscus and Pan troglodytes, also known as Anthropopithecus troglodytes or Simia satyrus. A “primate”, “primate species”, “primates” or grammatical variants thereof denote/s an order of eutherian mammals divided into the two suborders of prosimians and anthropoids and comprising man, apes, monkeys and lemurs. Specifically, “primates” as used herein comprises the suborder Strepsirrhini (non-tarsier prosimians), including the infraorder Lemuriformes (itself including the superfamilies Cheirogaleoidea and Lemuroidea), the infraorder Chiromyiformes (itself including the family Daubentoniidae) and the infraorder Lorisiformes (itself including the families Lorisidae and Galagidae). “Primates” as used herein also comprises the suborder Haplorrhini, including the infraorder Tarsiiformes (itself including the family Tarsiidae), the infraorder Simiiformes (itself including the Platyrrhini, or New World monkeys, and the Catarrhini, including the Cercopithecidea, or Old-World Monkeys).

The non-chimpanzee primate species may be understood within the meaning of the invention to be a lemur, a tarsier, a gibbon, a marmoset (belonging to New World Monkeys of the family Cebidae) or an Old-World Monkey (belonging to the superfamily Cercopithecoidea).

As used herein, an “Old-World Monkey” comprises any monkey falling in the superfamily Cercopithecoidea, itself subdivided into the families: the Cercopithecinae, which are mainly African but include the diverse genus of macaques which are Asian and North African; and the Colobinae, which include most of the Asian genera but also the African colobus monkeys.

Specifically, within the subfamily Cercopithecinae, an advantageous non-chimpanzee primate may be from the Tribe Cercopithecini, within the genus Allenopithecus (Allen's Swamp Monkey, Allenopithecus nigroviridis); within the genus Miopithecus (Angolan Talapoin, Miopithecus talapoin; Gabon Talapoin, Miopithecus ogouensis); within the genus Erythrocebus (Patas Monkey, Erythrocebus patas); within the genus Chlorocebus (Green Monkey, Chlorocebus sabaceus; Grivet, Chlorocebus aethiops; Bale Mountains Vervet, Chlorocebus djamdjamensis; Tantalus Monkey, Chlorocebus tantalus; Vervet Monkey, Chlorocebus pygerythrus; Malbrouck, Chlorocebus cynosuros); or within the genus Cercopithecus (Dryas Monkey or Salongo Monkey, Cercopithecus dryas; Diana Monkey, Cercopithecus diana; Roloway Monkey, Cercopithecus roloway; Greater Spot-nosed Monkey, Cercopithecus nictitans; Blue Monkey, Cercopithecus mitis; Silver Monkey, Cercopithecus doggetti; Golden Monkey, Cercopithecus kandti; Sykes's Monkey, Cercopithecus albogularis; Mona Monkey, Cercopithecus mona; Campbell's Mona Monkey, Cercopithecus campbelli; Lowe's Mona Monkey, Cercopithecus lowei; Crested Mona Monkey, Cercopithecus pogonias; Wolf's Mona Monkey, Cercopithecus wolfi; Dent's Mona Monkey, Cercopithecus denti; Lesser Spot-nosed Monkey, Cercopithecus petaurista; White-throated Guenon, Cercopithecus erythrogaster; Sclater's Guenon, Cercopithecus sclateri; Red-cared Guenon, Cercopithecus erythrotis; Moustached Guenon, Cercopithecus cephus; Red-tailed Monkey, Cercopithecus ascanius; L′Hoest's Monkey, Cercopithecus lhoesti; Preuss's Monkey, Cercopithecus preussi; Sun-tailed Monkey, Cercopithecus solatus; Hamlyn's Monkey or Owl-faced Monkey, Cercopithecus hamlyni; De Brazza's Monkey, Cercopithecus neglectus).

Alternatively, an advantageous non-chimpanzee primate, also within the subfamily Cercopithecinae but within the Tribe Papionini, may be from within the genus Macaca (Barbary Macaque, Macaca sylvanus; Lion-tailed Macaque, Macaca silenus; Southern Pig-tailed Macaque or Beruk, Macaca nemestrina; Northern Pig-tailed Macaque, Macaca leonina; Pagai Island Macaque or Bokkoi, Macaca pagensis; Siberut Macaque, Macaca siberu; Moor Macaque, Macaca maura; Booted Macaque, Macaca ochreata; Tonkean Macaque, Macaca tonkeana; Heck's Macaque, Macaca hecki; Gorontalo Macaque, Macaca nigriscens; Celebes Crested Macaque or Black “Ape”, Macaca nigra; Cynomolgus monkey or Crab-eating Macaque or Long-tailed Macaque or Kera, Macaca fascicularis; Stump-tailed Macaque or Bear Macaque, Macaca arctoides; Rhesus Macaque, Macaca mulatta; Formosan Rock Macaque, Macaca cyclopis; Japanese Macaque, Macaca fuscata; Toque Macaque, Macaca sinica; Bonnet Macaque, Macaca radiata; Barbary Macaque, Macaca sylvanmus; Assam Macaque, Macaca assamensis; Tibetan Macaque or Milne-Edwards' Macaque, Macaca thibetana; Arunachal Macaque or Munzala, Macaca munzala); within the genus Lophocebus (Gray-checked Mangabey, Lophocebus albigena; Lophocebus albigena albigena; Lophocebus albigena osmani; Lophocebus albigena johnstoni; Black Crested Mangabey, Lophocebus aterrimus; Opdenbosch's Mangabey, Lophocebus opdenboschi; Highland Mangabey, Lophocebus kipunji); within the genus Papio (Hamadryas Baboon, Papio hamadryas; Guinea Baboon, Papio papio; Olive Baboon, Papio anubis; Yellow Baboon, Papio cynocephalus; Chacma Baboon, Papio ursinus); within the genus Theropithecus (Gelada, Theropithecus gelada); within the genus Cercocebus (Sooty Mangabey, Cercocebus atys; Cercocebus atys atys; Cercocebus atys lunulatus; Collared Mangabey, Cercocebus torquatus; Agile Mangabey, Cercocebus agilis; Golden-bellied Mangabey, Cercocebus chrysogaster; Tana River Mangabey, Cercocebus galeritus; Sanje Mangabey, Cercocebus sanjei); or within the genus Mandrillus (Mandrill, Mandrillus sphinx; Drill, Mandrillus leucophaeus).

Most preferred is Macaca fascicularis (also known as Cynomolgus monkey and, therefore, in the Examples named “Cynomolgus”) and Macaca mulatta (rhesus monkey, named “rhesus”).

Within the subfamily Colobinde, an advantageous non-chimpanzee primate may be from the African group, within the genus Colobus (Black Colobus, Colobus satanas; Angola Colobus, Colobus angolensis; King Colobus, Colobus polykomos; Ursine Colobus, Colobus vellerosus; Mantled Guereza, Colobus guereza); within the genus Piliocolobus (Western Red Colobus, Piliocolobus badius; Piliocolobus badius badius; Piliocolobus badius temminckii; Piliocolobus badius waldronae; Pennant's Colobus, Piliocolobus pennantii; Piliocolobus pennantii pennantii; Piliocolobus pennantii epieni; Piliocolobus pennantii bouvieri; Preuss's Red Colobus, Piliocolobus preussi; Thollon's Red Colobus, Piliocolobus tholloni; Central African Red Colobus, Piliocolobus foai; Piliocolobus foai foai; Piliocolobus foai ellioti; Piliocolobus foai oustaleti; Piliocolobus foai semlikiensis; Piliocolobus foai parmentierorum; Ugandan Red Colobus, Piliocolobus tephrosceles; Uzyngwa Red Colobus, Piliocolobus gordonorum; Zanzibar Red Colobus, Piliocolobus kirkii; Tana River Red Colobus, Piliocolobus rufomitratus); or within the genus Procolobus (Olive Colobus, Procolobus verus).

Within the subfamily Colobinae, an advantageous non-chimpanzee primate may alternatively be from the Langur (leaf monkey) group, within the genus Semnopithecus (Nepal Gray Langur, Semnopithecus schistaceus; Kashmir Gray Langur, Semnopithecus ajax; Tarai Gray Langur, Semnopithecus hector; Northern Plains Gray Langur, Semnopithecus entellus; Black-footed Gray Langur, Semnopithecus hypoleucos; Southern Plains Gray Langur, Semnopithecus dussumieri; Tufted Gray Langur, Semnopithecus priam); within the T. vetulus group or the genus Trachypithecus (Purple-faced Langur, Trachypithecus vetulus; Nilgiri Langur, Trachypithecus johnii); within the T. cristatus group of the genus Trachypithecus (Javan Lutung, Trachypithecus auratus; Silvery Leaf Monkey or Silvery Lutung, Trachypithecus cristatus; Indochinese Lutung, Trachypithecus germaini; Tenasserim Lutung, Trachypithecus barbei); within the T. obscurus group of the genus Trachypithecus (Dusky Leaf Monkey or Spectacled Leaf Monkey, Trachypithecus obscurus; Phayre's Leaf Monkey, Trachypithecus phayrei); within the T. pileatus group of the genus Trachypithecus (Capped Langur, Trachypithecus pileatus; Shortridge's Langur, Trachypithecus shortridgei; Gee's Golden Langur, Trachypithecus geei); within the T. francoisi group of the genus Trachypithecus (Francois' Langur, Trachypithecus francoisi; Hatinh Langur, Trachypithecus hatinhensis; White-headed Langur, Trachypithecus poliocephalus; Laotian Langur, Trachypithecus laotum; Delacour's Langur, Trachypithecus delacouri; Indochinese Black Langur, Trachypithecus ebenus); or within the genus Presbytis (Sumatran Surili, Presbytis melalophos; Banded Surili, Presbytis femoralis; Sarawak Surili, Presbytis chrysomelas; White-thighed Surili, Presbytis siamensis; White-fronted Surili, Presbytis frontata; Javan Surili, Presbytis comata; Thomas's Langur, Presbytis thomasi; Hose's Langur, Presbytis hosei; Maroon Leaf Monkey, Presbytis rubicunda; Mentawai Langur or Joja, Presbytis potenziani; Natuna Island Surili, Presbytis natunae).

Within the subfamily Colobinae, an advantageous non-chimpanzee primate may alternatively be from the Odd-Nosed group, within the genus Pygathrix (Red-shanked Douc, Pygathrix nemaeus; Black-shanked Douc, Pygathrix nigripes; Gray-shanked Douc, Pygathrix cinerea); within the genus Rhinopithecus (Golden Snub-nosed Monkey, Rhinopithecus roxellana; Black Snub-nosed Monkey, Rhinopithecus bieti; Gray Snub-nosed Monkey, Rhinopithecus brelichi; Tonkin Snub-nosed Langur, Rhinopithecus avunculus); within the genus Nasalis (Proboscis Monkey, Nasalis larvatus); or within the genus Simias (Pig-tailed Langur, Simias concolor).

As used herein, the term “marmoset” denotes any New World Monkeys of the genus Callithrix, for example belonging to the Atlantic marmosets of subgenus Callithrix (sic!) (Common Marmoset, Callithrix (Callithrix) jacchus; Black-tufted Marmoset, Callithrix (Callithrix) penicillata; Wied's Marmoset, Callithrix (Callithrix) kuhlii; White-headed Marmoset, Callithrix (Callithrix) geoffroyi; Buffy-headed Marmoset, Callithrix (Callithrix) flaviceps; Buffy-tufted Marmoset, Callithrix (Callithrix) aurita); belonging to the Amazonian marmosets of subgenus Mico (Rio Acari Marmoset, Callithrix (Mico) acariensis; Manicore Marmoset, Callithrix (Mico) manicorensis; Silvery Marmoset, Callithrix (Mico) argentata; White Marmoset, Callithrix (Mico) leucippe; Emilia's Marmoset, Callithrix (Mico) emiliae; Black-headed Marmoset, Callithrix (Mico) nigriceps; Marca's Marmoset, Callithrix (Mico) marcai; Black-tailed Marmoset, Callithrix (Mico) melanura; Santarem Marmoset, Callithrix (Mico) humeralifera; Maués Marmoset, Callithrix (Mico) mauesi; Gold-and-white Marmoset, Callithrix (Mico) chrysoleuca; Hershkovitz's Marmoset, Callithrix (Mico) intermedia; Satéré Marmoset, Callithrix (Mico) saterei); Roosmalens' Dwarf Marmoset belonging to the subgenus Callibella (Callithrix (Callibella) humilis); or the Pygmy Marmoset belonging to the subgenus Cebuella (Callithrix (Cebuella) pygmaea).

As used herein, CD3 denotes a molecule expressed as part of the T cell receptor and has the meaning as typically ascribed to it in the prior art. In human, it encompasses in individual or independently combined form all known CD3 subunits, for example CD3 epsilon, CD3 delta, CD3 gamma, CD3 zeta, CD3 alpha and CD3 beta. The non-chimpanzee primate CD3 antigens as referred to herein are, for example, Macaca fascicularis CD3 and Macaca mulatta CD3. In Macaca fascicularis, it encompasses CD3 epsilon FN-18 negative and CD3 epsilon FN-18 positive, CD3 gamma and CD3 delta. In Macaca mulatta, it encompasses CD3 epsilon, CD3 gamma and CD3 delta. Preferably, said CD3 as used herein is CD3 epsilon. The human CD3 epsilon is indicated in GenBank Accession No. NM_000733 and comprises SEQ ID NO. 134. The human CD3 gamma is indicated in GenBank Accession No. NM_000073 and comprises SEQ ID NO. 142. The human CD3 delta is indicated in GenBank Accession No. NM_000732 and comprises SEQ ID NO. 143.

The CD3 epsilon “FN-18 negative” of Macaca fascicularis (i.e. CD3 epsilon not recognized by monoclonal antibody FN-18 due to a polymorphism as set forth above) is indicated in GenBank Accession No. AB073994 and comprises SEQ ID NO. 136.

The CD3 epsilon “FN-18 positive” of Macaca fascicularis (i.e. CD3 epsilon recognized by monoclonal antibody FN-18) is indicated in GenBank Accession No. AB073993 and comprises SEQ ID NO. 135. The CD3 gamma of Macaca fascicularis is indicated in GenBank Accession No. AB073992 and comprises SEQ ID NO. 144. The CD3 delta of Macaca fascicularis is indicated in GenBank Accession No. AB073991 and comprises SEQ ID NO. 145.

The nucleic acid sequences and amino acid sequences of the respective CD3 epsilon, gamma and delta homologs of Macaca mulatta can be identified and isolated by recombinant techniques described in the art (Sambrook et al. Molecular Cloning: A Laboratory Manual; Cold Spring Harbor Laboratory Press, 3^{rd} edition 2001). This applies mutatis mutandis to the CD3 epsilon, gamma and delta homologs of other non-chimpanzee primates as defined herein.

As pointed out above and as disclosed herein, it is envisaged that the first binding domain of the bispecific single chain antibody comprised in the inventive pharmaceutical composition leads to an epitope of human and non-chimpanzee primate CD3 which comprises the amino acid sequence “phenylalanine (F)—serine (S)—glutamic acid (E)”. The person skilled in the art is readily in the position to deduce an epitope detected by a given antibody/binding molecule and/or (as in the present invention) a given “binding domain” of a single chain construct by methods known in the art, said methods are also illustrated in the appended examples and may comprise Western blot analysis, epitope mapping or pepspot analysis and the like.

The epitope to be detected by said first binding domain is preferably in the range of 15 amino acids+/−3 amino acids. Envisaged are (but not limiting) 18, 17, 16, 15, 14, 13, 12, 11, 10, 9, 8, 7, 6, 5, 4 or 3 amino acids in said epitope comprising the “F-S-E” stretch/“F-S-E” core epitope.

As shown in the following Examples, the minimum core epitope of human and non-chimpanzee primate CD3 bound by the first binding domain of the bispecific single chain antibody as defined herein is an epitope comprising the amino acid residues “FSE”. More specifically, the minimum epitope comprises the amino acid residues “FSEXE” (SEQ ID NOs. 202 and 204), wherein the substitution of methionine to leucine is a conserved amino acid substitution between two neutral, non-polar amino acid residues. The minimum epitope may be part of a discontinuous epitope. As used herein, the term “discontinuous epitope” is to be understood as at least two non-adjacent amino acid sequence stretches within a given polypeptide chain, here e.g. CD3 (preferably CD3 epsilon), which are simultaneously bound by an antibody. These amino acid stretches might be of different length and may also be involved in the interaction of antibody and antigen. Accordingly, in addition to the minimum (core) epitope as defined above, the bispecific single chain antibody may simultaneously bind to one, two or even more non-adjacent epitopes. This (these) non-adjacent epitope(s) in combination with the minimal (core) epitope could represent the contact site between antigen and antibody. According to this definition, such simultaneous binding may be of the polypeptide in linear form. Here, one may imagine the polypeptide forming an extended loop, in one region of which the two sequences for example are more or less in parallel and in proximity of one another. Non-adjacent epitopes in the linear sequence could form a three dimensional structure leading to a close proximity of these epitopes. In this state they are simultaneously bound by the bispecific single chain antibody as defined herein. According to this definition, simultaneous binding of the at least two sequence stretches of the polypeptide indicated above (including the minimum (core) epitope) may also take the form of antibody binding to a conformational epitope. Here, the mature polypeptide has already formed its tertiary conformation as it normally exists in vivo. In this tertiary conformation, the polypeptide chain is folded in such a manner as to bring the at least two sequence stretches indicated above into spatial proximity, for example, on the outer surface of a particular region of mature, folded polypeptide, where they are then recognized by virtue of their three-dimensional conformation in the context of the surrounding polypeptide sequences.

The term “cell surface antigen” as used herein denotes a molecule which is displayed on the surface of a cell. In most cases, this molecule will be located in or on the plasma membrane of the cell such that at least part of this molecule remains accessible from outside the cell in tertiary form. A non-limiting example of a cell surface molecule which is located in the plasma membrane is a transmembrane protein comprising, in its tertiary conformation, regions of hydrophilicity and hydrophobicity. Here, at least one hydrophobic region allows the cell surface molecule to be embedded, or inserted in the hydrophobic plasma membrane of the cell while the hydrophilic regions extend on either side of the plasma membrane into the cytoplasm and extracellular space, respectively. Non-limiting examples of a cell surface molecules which are located on the plasma membrane are proteins which have been modified at a cysteine residue to bear a palmitoyl group, proteins modified at a C-terminal cysteine residue to bear a farnesyl group or proteins which have been modified at the C-terminus to bear a glycosyl phosphatidyl inositol (“GPI”) anchor. These groups allow covalent attachment of proteins to the outer surface of the plasma membrane, where they remain accessible for recognition by extracellular molecules such as antibodies.

The “tumor antigen” as used herein may be understood as those antigens that are presented on tumor cells. These antigens can be presented on the cell surface with an extracellular part which is often combined with a transmembrane and cytoplasmic part of the molecule. These antigens can sometimes be presented only by tumor cells and never by the normal ones. Tumor antigens can be exclusively expressed on tumor cells or might represent a tumor specific mutation compared to normal cells. In this case, they are called tumor-specific antigens. More common are antigens that are presented by tumor cells and normal cells, and they are called tumor-associated antigens. These tumor-associated antigens can be overexpressed compared to normal cells or are accessible for antibody binding in tumor cells due to the less compact structure of the tumor tissue compared to normal tissue. Non-limiting examples of tumor antigens as used herein are EpCAM (Naundorf, Int. J. Cancer 100/1 (2002), 101-110), EGFR (Liu, Br. J. Cancer 82/12 (2000), 1991-1999; Bonner, Semin. Radiat. Oncol. 12 (2002), 11-20; Kiyota, Oncology 63/1 (2002), 92-98; Kuan, Brain Tumor Pathol. 17/2 (2000), 71-78), EGFRvIII (Kuan, Brain Tumor Pathol. 17/2 (2000), 71-78), or Carboanhydrase IX (MN/CA IX) (Uemura, Br. J. Cancer 81/4 (1999), 741-746; Longcaster, Cancer Res. 61/17 (2001), 6394-6399; Chia, J. Clin. Oncol. 19/16 (2001), 3660-3668; Beasley, Cancer Res. 61/13 (2001), 5262-5267).

The corresponding sequences of the human and non-chimpanzee primate nucleic acid and amino acid sequences can be found e.g. in NCBI databases.

A cross-species-specific monoclonal antibody binding to a human cell surface antigen (preferably a tumor antigen) and to the homolog of said cell surface antigen (preferably a tumor antigen) in a non-chimpanzee primate can be generated as set out above. “Homologs” as used herein refer to genes (encoding e.g. CD3, CD3 epsilon, cell surface antigens or tumor antigens) which encode gene products with similar or identical biological function in different species and which genes can be attributed to a common precursor gene. Cross-species specificity of said monoclonal antibody to the human and non-chimpanzee primate tumor antigen can be tested by FACS assays as set forth above. Alternatively, immunohistochemistry, radioimmunoassay, or ELISA assays may be used as known to the person skilled in the art. The second binding domain of the bispecific single chain antibody exhibiting cross-species specificity as described herein can for example be derived from such cross-species specific monoclonal antibodies by recombinant techniques described in the following examples.

The term “evaluating the in vivo safety and/or activity and/or pharmacokinetic profile” of the bispecific single chain antibody as used herein may be understood as set forth below. Before a new candidate medication can be marketed it must pass through rigorous testing, which may be roughly subdivided into preclinical testing in animals and clinical phases in human patients. The aim of preclinical testing in animals is to prove that the drug candidate is safe and efficacious (see e.g. the Preclinical safety evaluation of biotechnology-derived pharmaceuticals; ICH Harmonised Tripartite Guideline; ICH Steering Committee meeting on Jul. 16, 1997).

The term “drug”, “drug candidate” or “pharmaceutical composition” as used herein refers to bispecific single chain antibodies defined herein.

The biological activity of the bispecific single chain antibody as defined herein can be determined for instance by cytotoxicity assays, as described in the following examples, in WO 99/54440 or by Schlereth et al. (Cancer Immunol. Immunother. 20 (2005), 1-12). “Efficacy” or “in vivo efficacy” as used herein refers to the response to therapy by the bispecific single chain antibody as defined herein, using e.g. standardized NCI response criteria. The success or in vivo efficacy of the therapy using a bispecific single chain antibody as defined herein refers to the effectiveness of the bispecific single chain antibody as defined herein for its intended purpose, i.e. the ability of the bispecific antibody to cause its desired effect, i.e. depletion of pathologic cells, e.g. tumor cells. The in vivo efficacy may be monitored by established standard methods for the respective disease entities including, but not limited to white blood cell counts, differentials, Fluorescence Activated Cell Sorting, bone marrow aspiration. In addition, various disease specific clinical chemistry parameters and other established standard methods may be used. Furthermore, computer-aided tomography, X-ray, nuclear magnetic resonance tomography (e.g. for National Cancer Institute-criteria based response assessment [Cheson B D, Horning S J, Coiffier B, Shipp M A, Fisher R I, Connors J M, Lister T A, Vose J, Grillo-Lopez A, Hagenbeck A, Cabanillas F, Klippensten D, Hiddemann W, Castellino R, Harris N L, Armitage J O, Carter W, Hoppe R, Canellos G P. Report of an international workshop to standardize response criteria for non-Hodgkin's lymphomas. NCI Sponsored International Working Group. J Clin Oncol. 1999 April; 17 (4): 1244]), positron-emission tomography scanning, white blood cell counts, differentials, Fluorescence Activated Cell Sorting, bone marrow aspiration, lymph node biopsies/histologies, and various lymphoma specific clinical chemistry parameters (e.g. lactate dehydrogenase) and other established standard methods may be used.

Another major challenge in the development of drugs is the predictable modulation of pharmacokinetic properties. To this end, a pharmacokinetic profile of the drug candidate, i.e. a profile of the pharmacokinetic parameters that effect the ability of a particular drug to treat a given condition, is established. Pharmacokinetic parameters of the drug influencing the ability of a drug for treating a certain disease entity include, but are not limited to: half-life, volume of distribution, hepatic first-pass metabolism and the degree of blood serum binding. The efficacy of a given drug agent can be influenced by each of the parameters mentioned above. “Half-life” means the time where 50% of an administered drug are eliminated through biological processes, e.g. metabolism, excretion, etc.

By “hepatic first-pass metabolism” is meant the propensity of a drug to be metabolized upon first contact with the liver, i.e. during its first pass through the liver.

“Volume of distribution” means the degree of retention of a drug throughout the various compartments of the body, like e.g. intracellular and extracellular spaces, tissues and organs, etc. and the distribution of the drug within these compartments.

“Degree of blood serum binding” means the propensity of a drug to interact with and bind to blood serum proteins, such as albumin, leading to a reduction or loss of biological activity of the drug.

Pharmacokinetic parameters also include bioavailability, lag time (Tlag), Tmax, absorption rates, more onset and/or Cmax for a given amount of drug administered.

“Bioavailability” means the amount of a drug in the blood compartment.

“Lag time” means the time delay between the administration of the drug and its detection and measurability in blood or plasma.

“Tmax” is the time after which maximal blood concentration of the drug is reached, and “Cmax” is the blood concentration maximally obtained with a given drug. The time to reach a blood or tissue concentration of the drug which is required for its biological effect is influenced by all parameters. Pharmacokinetik parameters of the bispecific single chain antibodies exhibiting cross-species specificity which may be determined in preclinical animal testing in non-chimpanzee primates as outlined above are also set forth e.g. in the publication by Schlereth et al. (Cancer Immunol. Immunother. 20 (2005), 1-12).

The term “toxicity” as used herein refers to the toxic effects of a drug manifested in adverse events or severe adverse events. These side events might refer to a lack of tolerability of the drug in general and/or a lack of local tolerance after administration. Toxicity could also include teratogenic or carcinogenic effects caused by the drug.

The term “safety”, “in vivo safety” or “tolerability” as used herein defines the administration of a drug without inducing severe adverse events directly after administration (local tolerance) and during a longer period of application of the drug. “Safety”, “in vivo safety” or “tolerability” can be evaluated e.g. at regular intervals during the treatment and follow-up period. Measurements include clinical evaluation, e.g. organ manifestations, and screening of laboratory abnormalities. Clinical evaluation may be carried out and deviating to normal findings recorded/coded according to NCI-CTC and/or MedDRA standards. Organ manifestations may include criteria such as allergy/immunology, blood/bone marrow, cardiac arrhythmia, coagulation and the like, as set forth e.g. in the Common Terminology Criteria for adverse events v3.0 (CTCAE). Laboratory parameters which may be tested include for instance haematology, clinical chemistry, coagulation profile and urine analysis and examination of other body fluids such as serum, plasma, lymphoid or spinal fluid, liquor and the like. Safety can thus be assessed e.g. by physical examination, imaging techniques (i.e. ultrasound, x-ray, CT scans, Magnetic Resonance Imaging (MRI), other measures with technical devices (i.e. electrocardiogram), vital signs, by measuring laboratory parameters and recording adverse events. For example, adverse events in non-chimpanzee primates in the uses and methods according to the invention may be examined by histopathological and/or histochemical methods.

The term “effective and non-toxic dose” as used herein refers to a tolerable dose of the bispecific single chain antibody as defined herein which is high enough to cause depletion of pathologic cells, tumor elimination, tumor shrinkage or stabilisation of disease without or essentially without major toxic effects. Such effective and non-toxic doses may be determined e.g. by dose escalation studies described in the art and should be below the dose inducing severe adverse side events (dose limiting toxicity, DLT).

The above terms are also referred to e.g. in the Preclinical safety evaluation of biotechnology-derived pharmaceuticals S6; ICH Harmonised Tripartite Guideline; ICH Steering Committee meeting on Jul. 16, 1997.

It has been surprisingly found that it is possible to generate bispecific antibody-based therapeutics for humans wherein the identical molecule can also be used in preclinical animal testing. This is due to the unexpected identification of bispecific single chain antibodies which, in addition to binding to human antigens (and due to genetic similarity likely to chimpanzee counterparts), also bind to the homologs of said antigens of non-chimpanzee primates, such as macaques. Thus, the need to construct a surrogate bispecific single chain antibody for testing in a phylogenetic distant (from humans) species disappears. As a result, the very same bispecific single chain antibody can be used in animal preclinical testing as is intended to be administered to humans in clinical testing as well as following market approval. The ability to use the same molecule for preclinical animal testing as in later administration to humans virtually eliminates, or at least greatly reduces, the danger that the data obtained in preclinical animal testing are not applicable to the human case. In short, obtaining preclinical safety data in animals using the same molecule as will actually be administered to humans does much to ensure the applicability of the data to a human-relevant scenario. In contrast, in conventional approaches using surrogate molecules, said surrogate antibodies have to be molecularly adapted to the animal test system used for preclinical safety assessment. Thus, the surrogate antibody to be used in human therapy in fact differs in sequence and also likely in structure from the one used in preclinical testing in pharmacokinetic parameters and/or biological activity, with the consequence that data obtained in preclinical animal testing have limited applicability/transferability to the human case. The use of surrogate molecules requires the construction, production, purification and characterization of a completely new antibody construct. This leads to additional development costs and time necessary to obtain that molecule. In sum, surrogates have to be developed separately in addition to the actual drug to be used in human therapy, so that two lines of development for two bispecific single chain antibody molecules have to be carried out. Therefore, a major advantage of the bispecific antibody-based constructs exhibiting cross-species specificity described herein is that the identical molecule can be used for therapeutics in humans and in preclinical animal testing.

On the other hand, it is also no longer necessary to adapt the test animal to the bispecific antibody-drug candidate intended for administration to humans, such as e.g. the creation of transgenic animals producing the human molecules bound by the bispecific antibody. The bispecific single chain antibodies exhibiting cross-species specificity according to the uses and methods of invention can be directly used for preclinical testing in non-chimpanzee primates, without any genetic manipulation of the animals. As well known to those skilled in the art, approaches in which the test animal is adapted to the drug candidate always bear the risk that the results obtained in the preclinical safety testing are less representative and predictive for humans due to the modification of the animal. For example, in transgenic animals, the proteins encoded by the transgenes are often highly over-expressed. Thus, data obtained for the biological activity of an antibody against this protein antigen may be limited in their predictive value for humans in which the protein is expressed at much lower, more physiological levels.

A further advantage of the uses of the bispecific single chain antibody exhibiting cross-species specificity of the invention lies in the avoidance of chimpanzee as a species for animal testing. Chimpanzees are the closest relatives to humans and were recently grouped into the family of hominids based on the genome sequencing data (Wildman et al., PNAS 100 (2003), 7181). Therefore, data obtained with chimpanzee is generally considered to be highly predictive for humans. However, due to their status as endangered species, the number of chimpanzees which can be used for medical experiments is highly restricted. As stated above, maintenance of chimpanzees for animal testing is therefore both costly and ethically problematic. The uses of the bispecific single chain antibody of the invention avoids both financial burden and ethical objection during preclinical testing without prejudicing the quality, i.e. applicability, of the animal testing data obtained. In light of this, the uses of bispecific single chain antibodies exhibiting cross-species specificity and methods according to the invention for preclinical animal testing in non-chimpanzee primates provides for a reasonable alternative for studies in chimpanzees.

A further advantage of the bispecific single chain antibody of the invention is the ability of extracting multiple blood samples when using it as part of animal preclinical testing, for example in the course of pharmacokinetic animal studies. Multiple blood extractions can be much more readily obtained with a non-chimpanzee primate than with lower animals, say a mouse. The extraction of multiple blood samples allows continuous testing of blood parameters for the determination of the biological effects induced by the bispecific single chain antibody of the invention. Furthermore, the extraction of multiple blood samples enables the researcher to evaluate the pharmacokinetic profile of the bispecific single chain antibody as defined herein. In addition, potential side effects which may be induced by said bispecific single chain antibody reflected in blood parameters can be measured in different blood samples extracted during the course of the administration of said antibody. This allows the determination of the potential toxicity profile of the bispecific single chain antibody as defined herein.

The advantages of the pharmaceutical compositions comprising bispecific single chain antibodies exhibiting cross-species specificity, uses of said bispecific antibodies and methods according to the invention may be briefly summarized as follows:

First, the bispecific single chain antibody exhibiting cross-species specificity used in preclinical testing is the same as the one used in human therapy. Thus, it is no longer necessary to develop two independent molecules which may differ in their pharmacokinetic properties and biological activity. This is highly advantageous in that e.g. the pharmacokinetic results are more directly transferable and applicable to the human setting than e.g. in conventional surrogate approaches.

Second, the uses of the bispecific antibody exhibiting cross-species specificity and methods according to the invention for the preparation of therapeutics in human is less cost- and labor-intensive than surrogate approaches.

Third, chimpanzee as a species for animal testing is avoided.

Fourth, multiple blood samples can be extracted for extensive pharmacokinetic studies.

A further aspect of the invention relates to a method of determining the biological activity and/or efficacy of a bispecific single chain antibody as defined above, wherein said bispecific single chain antibody is administered to a non-chimpanzee primate and the in vivo activity is measured.

Preferably, said in vivo activity is T cell activation, tumor target cell depletion, cytotoxicity, toxicity, occurrence of adverse side effects, and/or cytokine release. Methods for the determination of said in vivo activity are set forth e.g. in WO 99/54440.

The present invention in another aspect also provides for a pharmaceutical composition for the treatment of a human patient, comprising a bispecific single chain antibody which comprises
(i) a first binding domain binding to a non-chimpanzee primate CD3, and
(ii) a second binding domain binding to a cell surface antigen,
wherein said first binding domain binds to human and non-chimpanzee primate CD3.

In accordance with this invention, the term “pharmaceutical composition” relates to a composition for administration to a patient, preferably a human patient. Preferably, the pharmaceutical composition comprises suitable formulations of carriers, stabilizers and/or excipients. In a preferred embodiment, the pharmaceutical composition comprises a composition for parenteral, transdermal, intraluminal, intraarterial, intrathecal and/or intranasal administration or by direct injection into tissue. It is in particular envisaged that said composition is administered to a patient via infusion or injection. Administration of the suitable compositions may be effected by different ways, e.g., by intravenous, intraperitoneal, subcutaneous, intramuscular, topical or intradermal administration. The composition of the present invention may further comprise a pharmaceutically acceptable carrier. Examples of suitable pharmaceutical carriers are well known in the art and include phosphate buffered saline solutions, water, emulsions, such as oil/water emulsions, various types of wetting agents, sterile solutions, liposomes, etc., Compositions comprising such carriers can be formulated by well known conventional methods. These compositions can be administered to the subject at a suitable dose which can be determined e.g. by dose escalating studies by administration of increasing doses of the bispecific single chain antibody exhibiting cross-species specificity described herein to non-chimpanzee primates, for instance macaques. As set forth above, the bispecific single chain antibody exhibiting cross-species specificity described herein can be advantageously used in identical form in preclinical testing in non-chimpanzee primates and as drug in humans. These compositions can also be administered in combination with other proteinaccous and non-proteinaceous drugs. These drugs may be administered simultaneously with the composition comprising the bispecific single chain antibody as defined herein or separately before or after administration of said bispecific antibody in timely defined intervals and doses. The dosage regimen will be determined by the attending physician and clinical factors. As is well known in the medical arts, dosages for any one patient depend upon many factors, including the patient's size, body surface area, age, the particular compound to be administered, sex, time and route of administration, general health, and other drugs being administered concurrently. Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, inert gases and the like. In addition, the composition of the present invention might comprise proteinaceous carriers, like, e.g., serum albumin or immunoglobulin, preferably of human origin. It is envisaged that the composition of the invention might comprise, in addition to the bispecific single chain antibody as defined herein, further biologically active agents, depending on the intended use of the composition. Such agents might be drugs acting on the gastro-intestinal system, drugs acting as cytostatica, drugs preventing hyperurikemia, drugs inhibiting immunoreactions (e.g. corticosteroids), drugs modulating the inflammatory response, drugs acting on the circulatory system and/or agents such as cytokines known in the art.

According to a preferred embodiment of the pharmaceutical composition of the invention, the first binding domain of the bispecific single chain antibody as defined herein binds to an epitope of human and non-chimpanzee primate CD3 comprising the amino acid sequence “FSE”. The minimum core epitope comprising the amino acid residues “FSE”, the minimum epitope comprising the amino acid sequence “FSEXE” (SEQ ID NOs. 202 and 204; wherein “X” corresponds to a leucine (L) or to a methionine (M)) or non-adjacent epitopes as defined herein are preferably 18, 17, 16, 15, 14, 13, 12, 11, 10, 9, 8, 7, 6, 5, 4 or 3 amino acid residues in length. Preferably, said epitopes are 13 amino acid residues in length. Even more preferred, the epitope with the “FSEXE” (SEQ ID NOs. 202 and 204; wherein “X” corresponds to a leucine (L) or to a methionine (M))—motif comprises the amino acid sequence “EFSELEQSGYYVC” (SEQ ID NO. 195) of human CD3 epsilon. In cynomolgus CD3 epsilon, the corresponding epitope reads “EFSEMEQSGYYVC” (SEQ ID NO. 201). The substitution of methionine to leucine is a conserved amino acid substitution between two neutral, non-polar amino acid residues. The corresponding sequence of the preferred epitope “EFSEXEQSGYYVC” wherein X represents L (Leucine) or M (Methionine) is depicted in SEQ ID NO. 207. As shown in the following Examples, the bispecific single chain antibody as defined herein not only binds to this epitope, but also to amino-acid stretches non-adjacent to said minimal epitope. For example, the bispecific single chain antibody as defined herein in addition to the minimum core epitope may simultaneously bind to (an) epitope(s) of human CD3 epsilon contained in said CD3 epsilon chain. Accordingly, said epitope may additionally comprise the amino acid sequence “QYPGSEILWQHND” (SEQ ID NO. 203). Also additional or (further) epitopes of cynomolgus CD3 epsilon contained in said chain may be detected by the binding molecule or molecule comprising the binding domains as defined therein. These additional or further sequences may comprise the amino acid sequence “QDGNEEMGSITQT” (SEQ ID NO. 199) and “YYVSYPRGSNPED” (SEQ ID NO. 200). Thus, the minimal epitope is most likely part of a discontinuous epitope or a conformational epitope. As evident to a person skilled in the art, the scope of the present invention includes bispecific single chain antibodies not only binding to this minimum (core) epitope, but also to one, two or even more non-adjacent amino acid sequence stretches within CD3 (preferably CD3 epsilon). Based on the results shown in the following Examples it is concluded that cross-species specific anti-CD3 antibodies contact CD3 epsilon in the area of amino acid residues 57-61 of both cynomolgus and human CD3 epsilon comprising the amino acid stretches FSEME (SEQ ID NO. 206) and FSELE (SEQ ID NO. 205) of cynomolgus and human CD3 epsilon, respectively, with the motif FSE forming the epitope core. This result—although plausible because of the accessibility of the E-F-loop (amino acids 56-62) of human CD3 epsilon (Kjer-Nielsen et al., PNAS 101 (2004), p. 7675-80) comprising the amino acids FSELE (SEQ ID NO. 205) or FSEME (SEQ ID NO. 206)—is surprising since there is no overlap of this newly defined epitope with the known epitope on the CD3 epsilon-chain of anti-CD3 antibodies OKT-3 and UCHT-1 (Kjer-Nielsen et al., loc.cit; Arnett et al., PNAS 101 (2004), p. 16268-73) which have so far been regarded as representative of all anti-CD3 antibodies thought to form a single family with the same or a very similar epitope. In summary, the epitopes “FSE” and “FSEXE” (SEQ ID NO. 204) are distinct from the epitopes recognized by UCHT-1 or OKT-3 (Kjer-Nielsen et al., PNAS 101 (2004), p. 7675-80; Arnett et al., PNAS 101 (2004), p. 16268-73) and are unique for cross-species specific anti-CD3 antibodies binding to human and macaque CD3. Preferably, the minimum epitope comprises the amino acid sequence “FSEXE” (SEQ ID NO. 204), wherein X represents L (Leucine) or M (Methionine) and stands for a substitution of non-polar, neutral amino acid residues.

It is envisaged that in the pharmaceutical composition of the invention, said first binding domain of the bispecific single chain antibody of the pharmaceutical composition of the invention is located C-terminally to the second binding domain. However, also part of this invention is a bispecific construct, wherein the “first binding domain to a non-chimpanzee primate CD3” is located N-terminally to the herein defined “second binding domain to a cell surface antigen”.

As shown in the following examples, the advantages as described hereinabove are realizable not only when the first binding domain (binding to CD3) is located C-terminally to the second binding domain, i.e. closer to the C-terminus of the bispecific single chain antibody than the second binding domain, but also when the first binding domain (binding to CD3) is located N-terminally to the second binding domain, i.e. closer to the N-terminus of the bispecific single chain antibody than the second binding domain. The arrangement of the binding domains in the bispecific single chain antibody defined herein may therefore be one in which the first binding domain is located C-terminally to the second binding domain. The arrangement of the V chains may be VH (cell surface antigen)-VL (cell surface antigen)-VL (CD3)-VH(CD3), VH (cell surface antigen)-VL (cell surface antigen)-VH(CD3)-VL (CD3), VL (cell surface antigen)-VH (cell surface antigen)-VL (CD3)-VH(CD3) or VL (cell surface antigen)-VH (cell surface antigen)-VH(CD3)-VL (CD3). For an arrangement, in which the first binding domain is located N-terminally to the second binding domain, the following orders are possible: VH(CD3)-VL (CD3)-VL (cell surface antigen)-VH (cell surface antigen), VH(CD3)-VL (CD3)-VH (cell surface antigen)-VL (cell surface antigen), VL (CD3)-VH(CD3)-VL (cell surface antigen)-VH (cell surface antigen) or VL (CD3)-VH(CD3)-VH (cell surface antigen)-VL (cell surface antigen). As used herein, “N-terminally to” or “C-terminally to” and grammatical variants thereof denote relative location within the primary amino acid sequence rather than placement at the absolute N- or C-terminus of the bispecific single chain antibody. Hence, as a non-limiting example, a first binding domain which is “located C-terminally to the second binding domain” simply denotes that the first binding domain is located to the carboxyl side of the second binding domain within the bispecific single chain antibody, and does not exclude the possibility that an additional sequence, for example a His-tag, or another proteinaceous or non-proteinaceous compound such as a radioisotope, is located at the ultimate C-terminus of the bispecific single chain antibody.

In another preferred embodiment of the pharmaceutical composition, the second binding domain binds to a cell surface antigen and to the non-chimpanzee primate homolog of said cell surface antigen.

According to this embodiment of the invention, both the first and second binding domains of the bispecific single chain antibody described herein specifically bind to both human and non-chimpanzee primate variants of said first and second molecules, respectively. In light of the above statements, this is particularly advantageous since sufficient (cross-species) specificity exists on both sides of the bispecific single chain antibody, thus ensuring interspecies compatibility with respect to both first and second molecules and hence optimal extrapolability of the data obtained in preclinical animal studies to the case of administration in humans.

Preferably, said cell surface antigen is a tumor antigen. Even more preferred, said tumor antigen is EpCAM (Naundorf, Int. J. Cancer 100/1 (2002), 101-110), EGFR (Liu, Br. J. Cancer 82/12 (2000), 1991-1999; Bonner, Semin. Radiat. Oncol. 12 (2002), 11-20; Kiyota, Oncology 63/1 (2002), 92-98; Kuan, Brain Tumor Pathol. 17/2 (2000), 71-78), EGFRvIII (Kuan, Brain Tumor Pathol. 17/2 (2000), 71-78), or Carboanhydrase IX (MN/CA IX) (Uemura, Br. J. Cancer 81/4 (1999), 741-746; Longcaster, Cancer Res. 61/17 (2001), 6394-6399; Chia, J. Clin. Oncol. 19/16 (2001), 3660-3668; Beasley, Cancer Res. 61/13 (2001), 5262-5267).

Particularly preferred as cell surface antigen and/or tumor antigen is EpCAM. As shown in the following Examples, the present application for the first time provides for the nucleic acid and amino acid sequences of the extracellular domain of cynomolgus EpCAM shown in SEQ ID NOs. 47 and 48, respectively. Said sequences are essential tools for the generation and characterization of the bispecific single chain antibodies as defined herein exhibiting cross-species specificity to human and cynomolgus EpCAM.

In a further preferred embodiment of the pharmaceutical composition of the invention, the first binding domain comprises a VH region having an amino acid sequence as shown in any of SEQ ID NOs. 2, 110 or 6. It is envisaged and preferred that the VH region of the first binding domain comprises at least a third CDR (CDR-H3) comprising an amino acid sequence as set out in SEQ ID NO. 112 or CDR-H3* comprising an amino acid sequence as set out in SEQ ID NO. 113. The first binding domain may additionally comprise a second CDR (CDR-H2) comprising an amino acid sequence as set out in SEQ ID NO. 114. Further, the first binding domain may in addition comprise a first CDR (CDR-H1) comprising an amino acid sequence as set out in SEQ ID NO. 115. The VH region of the first binding domain thus may comprise one, two or all three of the mentioned CDRs. The mentioned CDRs are included for example in the VH regions shown in SEQ ID NOs. 2 and 110.

Alternatively, it is envisaged that the VH region of the first binding domain comprises a third CDR (CDR-H3) comprising an amino acid sequence as set out in SEQ ID NO. 119. Preferably, the first binding domain additionally comprises a second CDR (CDR-H2) comprising an amino acid sequence as set out in SEQ ID NO. 120. Especially preferred, the first binding domain additionally comprises a first CDR (CDR-H1) comprising an amino acid sequence as set out in SEQ ID NO. 121. The VH region of the first binding domain thus may comprise one, two or all three of the mentioned CDRs. The above-indicated CDRs are included for example in the VH region shown in SEQ ID NO. 6.

In another preferred embodiment of the pharmaceutical composition, the first binding domain comprises a VL region having an amino acid sequence as shown in any of SEQ ID NOs. 4, 148, 168 or 8. It is envisaged and preferred that the VL region of the first binding domain comprises at least a third CDR (CDR-L3) comprising an amino acid sequence as set out in SEQ ID NO. 116. The VL region may further comprise a second CDR (CDR-L2) comprising an amino acid sequence as set out in SEQ ID NO. 117. The VL region may in addition comprise a first CDR (CDR-L1) comprising an amino acid sequence as set out in SEQ ID NO. 118. The VL region of the first binding domain thus may comprise one, two or all three of the mentioned CDRs. The above-indicated CDRs are included for example in the VL regions shown in SEQ ID NOs. 4, 148 and 168.

Alternatively, it is envisaged that the VL region of the first binding domain comprises a third CDR (CDR-L3) comprising an amino acid sequence as set out in SEQ ID NO. 164. Preferably, the first binding domain additionally comprises a second CDR (CDR-L2) comprising an amino acid sequence as set out in SEQ ID NO. 165. Especially preferred, the first binding domain additionally comprises a first CDR (CDR-L1) comprising an amino acid sequence as set out in SEQ ID NO. 166. The VL region of the first binding domain thus may comprise one, two or all three of the mentioned CDRs. The above-indicated CDRs are included for example in the VL region shown in SEQ ID NO. 8.

Preferably, the first binding domain comprises CDR-L1 (SEQ ID NO. 118), CDR-L2 (SEQ ID NO. 117), and CDR-L3 (SEQ ID NO. 116) and CDR-H1 (SEQ ID NO. 115), CDR-H2 (SEQ ID NO. 114) and CDR-H3 (SEQ ID NO. 112) or CDR-H3* comprising the amino acid sequence “VSWFAY” (SEQ ID NO. 113).

Alternatively, the first binding domain comprises CDR-L1 (SEQ ID NO. 166), CDR-L2 (SEQ ID NO. 165), and CDR-L3 (SEQ ID NO. 164) and CDR-H1 (SEQ ID NO. 121), CDR-H2 (SEQ ID NO. 120) and CDR H3 (SEQ ID NO. 119).

Even more preferred, the VH region of the first binding domain comprises or consists of the amino acid sequence shown in SEQ ID NO. 2 and the VL region of the first binding domain comprises or consists of the amino acid sequence shown SEQ ID NO. 4; or the VH region of the first binding domain comprises or consists of the amino acid sequence shown in SEQ ID NO. 110 and the VL region of the first binding domain comprises or consists of the amino acid sequence shown in SEQ ID NO. 148; or the VH region of the first binding domain comprises or consists of the amino acid sequence shown in SEQ ID NO. 110 and the VL region of the first binding domain comprises or consists of the amino acid sequence shown in SEQ ID NO. 168, or the VH region of the first binding domain comprises or consists of the amino acid sequence shown in SEQ ID NO. 6 and the VL region of the first binding domain comprises or consists of the amino acid sequence shown in SEQ ID NO. 8. Or the VH region of the first binding domain comprises or consists of the amino acid sequence shown in SEQ ID NO. 2 and the VL region of the first binding domain comprises or consists of the amino acid sequence shown in SEQ ID NO. 148. Or the VH region of the first binding domain comprises or consists of the amino acid sequence shown in SEQ ID NO. 110 and the VL region of the first binding domain comprises or consists of the amino acid sequence shown in SEQ ID NO. 4. Or the VH region of the first binding domain comprises or consists of the amino acid sequence shown in SEQ ID NO. 2 and the VL region of the first binding domain comprises or consists of the amino acid sequence shown in SEQ ID NO. 168.

As set forth above, the order of the variable regions of the first binding domain may be VH-VL or VL-VH. Both arrangements are within the scope of the invention. For a first binding domain comprising the VH of SEQ ID NO. 2 and the VL of SEQ ID NO. 4, the VH-VL arrangement is shown in SEQ ID NOs. 9 and 10, whereas the VL-VH arrangement is depicted in SEQ ID NOs. 11 and 12.

For a first binding domain comprising the VH of SEQ ID NO. 110 and the VL of SEQ ID NO. 148, the VH-VL arrangement is shown in SEQ ID NOs. 146 and 147. For a first binding domain comprising the VH of SEQ ID NO. 110 and the VL of SEQ ID NO. 168, the VH-VL arrangement is shown in SEQ ID NOs. 169 and 170, whereas the VL-VH arrangement is depicted in SEQ ID NOs. 193 and 194. For a first binding domain comprising the VH of SEQ ID NO. 6 and the VL of SEQ ID NO. 8, the VH-VL arrangement is shown in SEQ ID NOs. 13 and 14, whereas the VL-VH arrangement is depicted in SEQ ID NOs. 15 and 16.

Similarly, the order of the variable regions of the second binding domain may be VH-VL or VL-VH. Both arrangements are within the scope of the invention. For example, the VH-VL arrangement of a second binding domain exhibiting cross-species specificity to human and cynomolgus EpCAM is shown in SEQ ID NOs. 53 and 54, whereas the VL-VH arrangement is depicted in SEQ ID NOs. 55 and 56.

In a particularly preferred embodiment of the pharmaceutical composition of the invention, the bispecific single chain antibody as defined herein comprises an amino acid sequence selected from the group consisting of:
(a) an amino acid sequence as depicted in any of SEQ ID NOs. 38, 40, 124, 42 or 44;
(b) an amino acid sequence encoded by a nucleic acid sequence as shown in SEQ ID NOs. 37, 39, 125, 41 or 43;
(c) an amino acid sequence encoded by a nucleic acid sequence hybridizing under stringent conditions to the complementary nucleic acid sequence of (b);
(d) an amino acid sequence encoded by a nucleic acid sequence which is degenerate as a result of the genetic code to a nucleotide sequence of (b); and
(e) an amino acid sequence at least 85% identical, more preferred at least 90% identical, most preferred at least 95% identical to the amino acid sequence of (a) or (b).

In the above-indicated preferred embodiment, only the first binding domain (binding to CD3) exhibits cross-species specificity.

Most preferably, the bispecific single chain antibody as defined herein comprises an amino acid sequence selected from the group consisting of:
(a) an amino acid sequence as depicted in any of SEQ ID NOs. 66, 68, 74, 76, 122, 70, 72, 78, 80, 172, 174, 176, 178, 180, 182, 184, 186, 188, 190, or 192;
(b) an amino acid sequence encoded by a nucleic acid sequence as shown in SEQ ID NOs. 65, 67, 73, 75, 123, 69, 71, 77, 79, 171, 173, 175, 177, 179, 181, 183, 185, 187, 189, or 191;
(c) an amino acid sequence encoded by a nucleic acid sequence hybridizing under stringent conditions to the complementary nucleic acid sequence of (b);
(f) an amino acid sequence encoded by a nucleic acid sequence which is degenerate as a result of the genetic code to a nucleotide sequence of (b); and
(g) an amino acid sequence at least 85% identical, more preferred at least 90% identical, most preferred at least 95% identical to the amino acid sequence of (a) or (b).

In this above-indicated embodiment, both the first and second binding domains exhibit cross-species specificity.

In another preferred embodiment of the pharmaceutical composition, the non-chimpanzee primate is a baboon, marmoset or an old world monkey.

In an even more preferred embodiment of the pharmaceutical composition, the old world monkey is a monkey of the macaque genus.

Most preferably, the monkey of the macaque genus is Assamese macaque (Macaca assamensis), Barbary macaque (Macaca sylvanus), Bonnet macaque (Macaca radiata), Booted or Sulawesi-Booted macaque (Macaca ochreata), Sulawesi-crested macaque (Macaca nigra), Formosan rock macaque (Macaca cyclopsis), Japanese snow macaque or Japanese macaque (Macaca fuscata), Cynomologus monkey or crab-eating macaque or long-tailed macaque or Java macaque (Macaca fascicularis), Lion-tailed macaque (Macaca silenus), Pigtailed macaque (Macaca nemestrina), Rhesus macaque (Macaca mulatta), Tibetan macaque (Macaca thibetana), Tonkean macaque (Macaca tonkeana), Toque macaque (Macaca sinica), Stump-tailed macaque or Red-faced macaque or Bear monkey (Macaca arctoides), or Moor macaque (Macaca maurus).

Preferably, the non-chimpanzee primate CD3 comprises or consists of an amino acid sequence shown in SEQ ID NOs. 135, 136, 144, or 145.

According to a further embodiment of the pharmaceutical composition of the invention, at least one of said first or second binding domains is human, humanized, CDR-grafted and/or deimmunized.

The term “human” antibody as used herein is to be understood as meaning that the bispecific single chain antibody as defined herein, comprises (an) amino acid sequence(s) contained in the human germline antibody repertoire. For the purposes of definition herein, said bispecific single chain antibody may therefore be considered human if it consists of such (a) human germline amino acid sequence(s), i.e. if the amino acid sequence(s) of the bispecific single chain antibody in question is (are) identical to (an) expressed human germline amino acid sequence(s). A bispecific single chain antibody as defined herein may also be regarded as human if it consists of (a) sequence(s) that deviate(s) from its (their) closest human germline sequence(s) by no more than would be expected due to the imprint of somatic hypermutation. Additionally, the antibodies of many non-human mammals, for example rodents such as mice and rats, comprise VH CDR3 amino acid sequences which one may expect to exist in the expressed human antibody repertoire as well. Any such sequence(s) of human or non-human origin which may be expected to exist in the expressed human repertoire would also be considered “human” for the purposes of the present invention.

As used herein, the term “humanized”, “humanization”, “human-like” or grammatically related variants thereof are used interchangeably to refer to a bispecific single chain antibody comprising in at least one of its binding domains at least one complementarity determining region (“CDR”) from a non-human antibody or fragment thereof. Humanization approaches are described for example in WO 91/09968 and U.S. Pat. No. 6,407,213. As non-limiting examples, the term encompasses the case in which a variable region of at least one binding domain comprises a single CDR region, for example the third CDR region of the VH, from another non-human animal, for example a rodent, as well as the case in which a or both variable region/s comprise at each of their respective first, second and third CDRs the CDRs from said non-human animal. In the event that all CDRs of a binding domain of the bispecific single chain antibody have been replaced by their corresponding equivalents from, for example, a rodent, one typically speaks of “CDR-grafting”, and this term is to be understood as being encompassed by the term “humanized” or grammatically related variants thereof as used herein. The term “humanized” or grammatically related variants thereof also encompasses cases in which, in addition to replacement of one or more CDR regions within a VH and/or VL of the first and/or second binding domain further mutation/s (e.g. substitutions) of at least one single amino acid residue/s within the framework (“FR”) regions between the CDRs has/have been effected such that the amino acids at that/those positions correspond/s to the amino acid/s at that/those position/s in the animal from which the CDR regions used for replacement is/are derived. As is known in the art, such individual mutations are often made in the framework regions following CDR-grafting in order to restore the original binding affinity of the non-human antibody used as a CDR-donor for its target molecule. The term “humanized” may further encompass (an) amino acid substitution(s) in the CDR regions from a non-human animal to the amino acid(s) of a corresponding CDR region from a human antibody, in addition to the amino acid substitutions in the framework regions as described above.

As used herein, the term “deimmunized,” “deimmunization” or grammatically related variants thereof denotes modification of the first and/or second binding domain vis-à-vis an original wild type construct by rendering said wild type construct non-immunogenic or less immunogenic in humans. Deimmunization approaches are shown e.g. in WO 00/34317, WO 98/52976, WO 02/079415 or WO 92/10755. The term “deimmunized” also relates to constructs, which show reduced propensity to generate T cell epitopes. In accordance with this invention, the term “reduced propensity to generate T cell epitopes” relates to the removal of T-cell epitopes leading to specific T-cell activation. Furthermore, “reduced propensity to generate T cell epitopes” means substitution of amino acids contributing to the formation of T cell epitopes, i.e. substitution of amino acids, which are essential for formation of a T cell epitope. In other words, “reduced propensity to generate T cell epitopes” relates to reduced immunogenicity or reduced capacity to induce antigen independent T cell proliferation. The term “T cell epitope” relates to short peptide sequences which can be released during the degradation of peptides, polypeptides or proteins within cells and subsequently be presented by molecules of the major histocompatibility complex (MHC) in order to trigger the activation of T cells; see inter alia WO 02/066514. For peptides presented by MHC class II such activation of T cells can then give rise to an antibody response by direct stimulation of T cells to produce said antibodies. “Reduced propensity to generate T-cell epitopes” and/or “deimmunization” may be measured by techniques known in the art. Preferably, deimmunization of proteins may be tested in vitro by T cell proliferation assay. In this assay PBMCs from donors representing >80% of HLA-DR alleles in the world are screened for proliferation in response to either wild type or de-immunized peptides. Ideally cell proliferation is only detected upon loading of the antigen-presenting cells with wild type peptides. Alternatively, one may test deimmunization by expressing HLA-DR tetramers representing all haplotypes. These tetramers may be tested for peptide binding or loaded with peptides substitute for antigen-presenting cells in proliferation assays. In order to test whether deimmunized peptides are presented on HLA-DR haplotypes, binding of e.g. fluorescence-labeled peptides on PBMCs can be measured. Furthermore, deimmunization can be proven by determining whether antibodies against the deimmunized molecules have been formed after administration in patients. Preferably, antibody derived molecules are deimmunized in the framework regions and most of the CDR regions are not modified in order to generate reduced propensity to induce T cell epitope so that the binding affinity of the CDR regions is not affected. Even elimination of one T cell epitope results in reduced immunogenicity.

The invention also provides for a pharmaceutical composition comprising a nucleic acid sequence encoding a bispecific single chain antibody as defined herein.

The invention further relates to a pharmaceutical composition comprising a vector which comprises a nucleic acid sequence as defined above. Preferably said vector further comprises a regulatory sequence which is operably linked to said nucleic acid sequence defined above. More preferably, said vector is an expression vector.

In a further aspect, the invention relates to a pharmaceutical composition comprising a host transformed or transfected with a vector defined above.

A further aspect of the invention relates to a pharmaceutical composition as defined hereinabove, further comprising a proteinaceous compound capable of providing an activation signal for immune effector cells.

Preferably, the pharmaceutical composition further comprises suitable formulations of carriers, stabilizers and/or excipients.

In another aspect, the invention relates to a process for the production of a pharmaceutical composition as defined above, said process comprising culturing a host as defined above under conditions allowing the expression of the bispecific single chain antibody as defined hereinabove and recovering the produced bispecific single chain antibody from the culture.

A further aspect of the invention relates to a use of a bispecific single chain antibody as defined hereinabove or as produced by the process as defined hereinabove, a nucleic acid molecule as defined hereinabove, a vector as defined hereinabove or a host as defined hereinabove for the preparation of a pharmaceutical composition for the prevention, treatment or amelioration of a disease. Another aspect of the invention relates to a method for the prevention, treatment or amelioration of a disease in a subject in the need thereof, said method comprising the step of administration of an effective amount of a pharmaceutical composition of the invention or as produced according by the process set forth above.

Preferably, said disease is a proliferative disease, a tumorous disease, or an immunological disorder. Even more preferred, said tumorous disease is a malignant disease, preferably cancer. Cross-species specific bispecific single chain antibodies as defined herein with specificity for EpCAM, EGFR or EGFRvIII can be used for the therapy of epithelial cancers and tumors. Cross-species specific bispecific single chain antibody constructs as defined herein with specificity for CAIX can be used for the treatment of tumors with hypoxical regions or areas. Moreover, said CAIX constructs may be used for the treatment of renal or cervical carcinomas. In another preferred embodiment of the uses or methods of the invention, said pharmaceutical composition as defined hereinabove is suitable to be administered in combination with an additional drug, i.e. as part of a co-therapy. In said co-therapy, an active agent may be optionally included in the same pharmaceutical composition as the bispecific single chain antibody, or may be included in a separate pharmaceutical composition. In this latter case, said separate pharmaceutical composition is suitable for administration prior to, simultaneously as or following administration of said pharmaceutical composition comprising the bispecific single chain antibody. The additional drug or pharmaceutical composition may be a non-proteinaccous compound or a proteinaccous compound. In the case that the additional drug is a proteinaccous compound, it is advantageous that the proteinaccous compound be capable of providing an activation signal for immune effector cells.

Preferably, said proteinaccous compound or non-proteinaccous compound may be administered simultaneously or non-simultaneously with a bispecific single chain antibody as defined hereinabove, a nucleic acid molecule as defined hereinabove, a vector as defined as defined hereinabove, or a host as defined as defined hereinabove. Preferably, said subject to be treated is a human.

In a further aspect, the invention relates to a kit comprising a bispecific single chain antibody as defined hereinabove, a nucleic acid molecule as defined hereinabove, a vector as defined hereinabove, or a host as defined hereinabove.

These and other embodiments are disclosed and encompassed by the description and Examples of the present invention. Recombinant techniques and methods in immunology are described e.g. in Sambrook et al. Molecular Cloning: A Laboratory Manual; Cold Spring Harbor Laboratory Press, 3^{rd} edition 2001; Lefkovits; Immunology Methods Manual; The Comprehensive Sourcebook of Techniques; Academic Press, 1997; Golemis; Protein-Protein Interactions: A Molecular Cloning Manual; Cold Spring Laboratory Press, 2002. Further literature concerning any one of the antibodies, methods, uses and compounds to be employed in accordance with the present invention may be retrieved from public libraries and databases, using for example electronic devices. For example, the public database “Medline”, available on the Internet, may be utilized, for example under http://www.ncbi.nlm. nih.gov/PubMed/medline.html. Further databases and addresses, such as http://www.ncbi. nim.nih.qov/, http://www.infobioacn.fr/, http://www.fmi.ch/bioloqy/research tools.html, http://www.tiqr.orQ/. are known to the person skilled in the art and can also be obtained using, e.g., http://www.lvcos.com

### THE FIGURES SHOW

FIG. 1: Identification of cross-species specific antibodies to macaque CD3: Cross-species specificity of an anti-CD3 antibody shown in SEQ ID NO. 162 described in WO 99/54440, OKT-3, an Ig comprising SEQ ID NOs. 6 and 8, an Ig comprising SEQ ID NOs. 2 and 4 and UCHT-1 to macaque (cynomolgus) CD3 were tested with Flow Cytometry as described in Example 1. An immunoglobulin (Ig) comprising SEQ ID NOs. 6 and 8 and an Ig comprising SEQ ID NOs. 2 and 4 show cross-species specificity to macaque CD3. In contrast, the anti-CD3 antibody shown in SEQ ID NO. 162, OKT-3 and UCHT-1 fail to bind to macaque CD3.

FIG. 2: FACS assay for binding of an Ig comprising SEQ ID NOs. 2 and 4, an Ig comprising SEQ ID NOs. 6 and 8 and monoclonal antibody (mAb) FN-18 to HPB-ALL cells and PBMC of Macaca fascicularis (cynomolgus). HPB-ALL cells express the human CD3 complex. Cells stained with the respective antibodies are shown in comparison to unstained cells. Strong antigen binding on human as well as on cynomolgus cells was detected for the Ig comprising SEQ ID NOs. 2 and 4. For the Ig comprising SEQ ID NOs. 6 and 8, strong binding to human cells but weaker binding to cynomolgus cells was observed. For FN-18, strong binding to cynomolgus cells could be observed, whereas no binding to human cells could be detected.

FIG. 3: FACS assay for binding of 5-10LHxSEQ ID NO. 12, 5-10LHxSEQ ID NO. 10, 5-10LHxSEQ ID NO. 16 and 5-10LHxSEQ ID NO. 14 to human Kato III cells expressing EpCAM or human EpCAM transfected CHO cells and to HPB-ALL cells. Cells bound by the respective constructs (depicted as non-filled curves) are shown in comparison to cells incubated only with the detection antibodies (depicted as filled curves). Antigen binding of all bispecific constructs was clearly detectable for the anti human EpCAM specificity as well as for the anti CD3 specificities on the HPB-ALL cell line positive for human CD3.

FIG. 4: Cytotoxicity assay for 5-10LHxSEQ ID NO. 12, 5-10LHxSEQ ID NO. 10 and 5-10LHxSEQ ID NO. 14 with human Kato III cells as target cells and human PBMC as effector cells. All constructs showed cytotoxic activity.

FIG. 5: Cytotoxicity assay for 5-10LHxSEQ ID NO. 12, 5-10LHxSEQ ID NO. 10, and 5-10LHxSEQ ID NO. 14 with Kato III cells as target cells and cynomolgus PBMC as effector cells. 5-10LHxSEQ ID NO. 14, 5-10LHxSEQ ID NO. 12 and 5-10LHxSEQ ID NO. 10 showed cytotoxic activity. 5-10LHxdi-anti CD3 (deimmunised anti-CD3 antibody shown in SEQ ID NO. 163) which fails to bind to cynomolgus CD3 was used as a negative control.

FIG. 6: Amino acid sequence alignment of the extracellular portion of the cynomolgus EpCAM antigen (also shown in SEQ ID NO. 48) and the human EpCAM antigen.

FIG. 7: FACS assay for the detection of the cynomolgus EpCAM antigen on transfected CHO cells. Supernatants of three different anti human EpCAM hybridomas (M79, 3B10, 2G8) were tested for binding. Transfectants (depicted as non-filled curves) as compared to untransfected cells (depicted as filled curves) showed binding only with the supernatant of the 2G8 hybridoma which is therefore recognized as antibody cross-species specific for human and cynomolgus EpCAM.

FIG. 8: FACS assay for binding of 2G8LHxSEQ ID NO. 12, 2G8LHxSEQ ID NO. 10, 2G8LHxSEQ ID NO. 16, 2G8LHxSEQ ID NO. 14, 2G8HLxSEQ ID NO. 12, 2G8HLxSEQ ID NO. 10, 2G8HLxSEQ ID NO. 16 and 2G8HLxSEQ ID NO. 14 on Kato III (FIG. 8A) cells or cynomolgus EpCAM transfected CHO cells (FIG. 8B) and HPB-ALL cells. Antigen binding was clearly detectable for the anti EpCAM specificities as well as for the anti CD3 specificities. As a negative control for binding to cynomolgus EpCAM, the 5-10LHxSEQ ID NO. 10 construct was included which shows binding to human CD3 (on HPB-ALL cells) but no binding to cynomolgus EpCAM (cynomolgus EpCAM transfected CHO cells).

FIG. 9: Cytotoxicity assay for 2G8LHxSEQ ID NO. 10 and 2G8HLxSEQ ID NO. 12 with cynomolgus EpCAM transfected CHO cells as target cells and human PBMC as effector cells. 2G8LHxSEQ ID NO. 10 and 2G8HLxSEQ ID NO. 12 showed cytotoxic activity. 5-10LHxdi-anti CD3 (deimmunised anti-CD3 antibody shown in SEQ ID NO. 163) was included as negative control. 5-10LH fails to bind to cynomolgus EpCAM.

FIG. 10: Cytotoxicity assay for 2G8LHxSEQ ID NO. 10 and 2G8HLxSEQ ID NO. 12 with cynomolgus EpCAM transfected CHO cells as target cells and cynomolgus PBMC as effector cells. 2G8LHxSEQ ID NO. 10 and 2G8HLxSEQ ID NO. 12 showed cytotoxic activity. 5-10LHxdi-anti CD3 (deimmunised anti-CD3 antibody shown in SEQ ID NO. 163) was included as negative control. This construct fails to bind to cynomolgus CD3 and cynomolgus EpCAM.

FIG. 11: Amino acid comparison of SEQ ID NO. 2 and human VH segment (hu) 3-73.

FIG. 12: Amino acid and nucleotide sequences of a cross-species specific human-like VH region (also shown in SEQ ID NOs. 110 and 111, respectively).

FIG. 13: FACS analysis of a scFv comprising the human-like VH chain shown in SEQ ID NO. 110 and the VL chain shown in SEQ ID NO: 148. The complete scFv amino acid sequence is shown in SEQ ID NO. 146. The control scFv of SEQ ID NO. 10 shows a clear shift on human CD3 positive HPB-All cells and thus binds to human CD3. The scFv depicted in SEQ ID NO. 146 also shows clear binding to said CD3 positive human cells.

FIG. 14: Binding analysis of the scFv of SEQ ID NO. 146. The control scFv of SEQ ID NO. 10 shows a clear shift on cynomolgus CD3 positive T cells and thus binds to cynomolgus CD3 positive cells. Also the scFv of SEQ ID NO: 146 shows clear binding to cynomolgus CD3 positive cells.

FIG. 15: Alignment of amino acid sequences of human and cynomolgus CD3 epsilon.

FIG. 16: Amino acid sequences of the 13mer peptides derived from cynomolgus CD3 epsilon (43 peptide-spots).

FIG. 17: Amino acid sequences of the 13mer peptides derived from human CD3 epsilon (47 peptide-spots).

FIG. 18: Pepspots developed by enhanced chemiluminescence (A) Control pepspot with horseradish-peroxidase conjugated goat-anti-mouse IgG (B) Pepspot with cross-species specific anti-CD3 antibody I corresponding to an immunoglobulin (Ig) comprising the VH chain shown in SEQ ID NO. 2 and the VL chain shown in SEQ ID NO. 4.

FIG. 19: Pepspot with cross-species specific anti-CD3 antibody II corresponding to an immunoglobulin (Ig) comprising the VH chain shown in SEQ ID NO. 6 and the VL chain shown in SEQ ID NO. 8.

FIG. 20: Contact residues of OKT-3 and UCHT-1 and E-F-loop epitope of cross-species specific anti-CD3 antibodies I and II referred to in FIGS. 18 and 19, respectively, on cynomolgus and human CD3 epsilon.

FIG. 21: Amino acid sequence comparison of the murine VL shown in SEQ ID NO. 4 to the human germline lambda 7a segment.

FIG. 22: Binding of the murine scFv shown in SEQ ID NO. 10 and the human-like scFv shown in SEQ ID NO. 170 to human CD3-positive HPB-ALL cells.

FIG. 23: Upper Panel: Equal binding of the murine scFv shown in SEQ ID NO. 10 and the human-like scFv shown in SEQ ID NO. 170 to human and cynomolgus T cells in PBMCs. Lower Panel: When preincubated with 10 μg/ml of the murine IgG antibody mAb I described in Example 1 having the same binding specificity as the scFvs (i.e. for CD3 epsilon), the shifts of cells stained with the above-mentioned murine scFv or the human-like scFv decrease significantly, underlining the similar binding region of the scFvs and the original murine antibody mAb I.

FIG. 24: Pepspots developed by the alkaline phosphatase detection system (A) Control pepspot with alkaline phosphatase conjugated goat-anti-mouse IgG (B) Pepspot with cross-species specific anti-CD3 antibody comprising the human-like VH shown in SEQ ID NO. 110 and the human-like VL shown in SEQ ID NO. 168 as described in Example 18.

FIG. 25: Dot Blot Assay with the cross-species specific anti-CD3 antibody comprising the human-like VH of SEQ ID NO. 110 and the human-like VL of SEQ ID NO. 168 as described in Example 19 in (A) and the anti-CD3 murine IgG1 antibody UCHT1 (B) binding to the blotted peptides “biotin-linker-EFSELEQSGYYVC” (1) and “EFSELEQSGYYVC-biotin” (2) derived from human CD3 epsilon

FIG. 26: FACS binding analysis of cross-species specific bispecific single chain construct CAIX HL×SEQ ID NO. 194 to HPB-ALL (human CD3+), cynomolgus PBMC (cynomolgus CD3+), A549 (human CAIX+) and CYNOM-K1 (cynomolgus CAIX+) cells, respectively. The FACS staining was performed as described in Example 23. The thick line represents cells incubated with 1 μg/ml purified monomeric protein that were subsequently incubated with the anti-his antibody and the PE labeled detection antibody. The thin histogram line reflects the negative control: cells only incubated with the anti-his antibody and the detection antibody.

FIG. 27: FACS binding analysis of cross-species specific bispecific single chain construct CAIX HL×SEQ ID NO. 170 to HPB-ALL (human CD3+), cynomolgus PBMC (cynomolgus CD3+), A549 (human CAIX+) and 4MBr-5 (macaque CAIX+) cells, respectively. The FACS staining was performed as described in Example 23. The thick line represents cells incubated with 1 μg/ml purified monomeric protein that were subsequently incubated with the anti-his antibody and the PE labeled detection antibody. The thin histogram line reflects the negative control: cells only incubated with the anti-his antibody and the detection antibody.

FIG. 28: FACS binding analysis of cross-species specific bispecific single chain construct CAIX LH×SEQ ID NO. 170 to HPB-ALL (human CD3+), cynomolgus PBMC (cynomolgus CD3+), A549 (human CAIX+) and 4MBr-5 (macaque CAIX+) cells, respectively. The FACS staining was performed as described in Example 23. The thick line represents cells incubated with 1 μg/ml purified monomeric protein that were subsequently incubated with the anti-his antibody and the PE labeled detection antibody. The thin histogram line reflects the negative control: cells only incubated with the anti-his antibody and the detection antibody.

FIG. 29: FACS binding analysis of cross-species specific bispecific single chain construct EGFR HL×SEQ ID NO. 170 to HPB-ALL (human CD3+), cynomolgus PBMC (cynomolgus CD3+), A431 (human EGFR+) and CHO cells transfected with cynomolgus EGFR (cynomolgus EGFR+), respectively. The FACS staining was performed as described in Example 23. The thick line represents cells incubated with 1 μg/ml purified monomeric protein that were subsequently incubated with the anti-his antibody and the PE labeled detection antibody. The thin histogram line reflects the negative control: cells only incubated with the anti-his antibody and the detection antibody.

FIG. 30: FACS binding analysis of cross-species specific bispecific single chain construct EGFR LH×SEQ ID NO. 170 to HPB-ALL (human CD3+), cynomolgus PBMC (cynomolgus CD3+), A431 (human EGFR+) and CHO cells transfected with cynomolgus EGFR (cynomolgus EGFR+), respectively. The FACS staining was performed as described in Example 23. The thick line represents cells incubated with 1 μg/ml purified monomeric protein that were subsequently incubated with the anti-his antibody and the PE labeled detection antibody. The thin histogram line reflects the negative control: cells only incubated with the anti-his antibody and the detection antibody.

FIG. 31: FACS binding analysis of cross-species specific bispecific single chain construct EGFR HL×SEQ ID NO. 194 to HPB-ALL (human CD3+), cynomolgus PBMC (cynomolgus CD3+), A431 (human EGFR+) and CHO cells transfected with cynomolgus EGFR (cynomolgus EGFR+), respectively. The FACS staining was performed as described in Example 23. The thick line represents cells incubated with 1 μg/ml purified monomeric protein that were subsequently incubated with the anti-his antibody and the PE labeled detection antibody. The thin histogram line reflects the negative control: cells only incubated with the anti-his antibody and the detection antibody.

FIG. 32: FACS binding analysis of cross-species specific bispecific single chain construct EGFR LH×SEQ ID NO. 194 to HPB-ALL (human CD3+), cynomolgus PBMC (cynomolgus CD3+), A431 (human EGFR+) and CHO cells transfected with cynomolgus EGFR (cynomolgus EGFR+), respectively. The FACS staining was performed as described in Example 23. The thick line represents cells incubated with 1 μg/ml purified monomeric protein that were subsequently incubated with the anti-his antibody and the PE labeled detection antibody. The thin histogram line reflects the negative control: cells only incubated with the anti-his antibody and the detection antibody.

FIG. 33: FACS binding analysis of cross-species specific bispecific single chain construct SEQ ID NO. 170×EGFR HL to HPB-ALL (human CD3+), cynomolgus PBMC (cynomolgus CD3+), A431 (human EGFR+) and CHO cells transfected with cynomolgus EGFR (cynomolgus EGFR+), respectively. The FACS staining was performed as described in Example 23. The thick line represents cells incubated with 1 μg/ml purified monomeric protein that were subsequently incubated with the anti-his antibody and the PE labeled detection antibody. The thin histogram line reflects the negative control: cells only incubated with the anti-his antibody and the detection antibody.

FIG. 34: FACS binding analysis of cross-species specific bispecific single chain construct SEQ ID NO. 170×EGFR LH to HPB-ALL (human CD3+), cynomolgus PBMC (cynomolgus CD3+), A431 (human EGFR+) and CHO cells transfected with cynomolgus EGFR (cynomolgus EGFR+), respectively. The FACS staining was performed as described in Example 23. The thick line represents cells incubated with 1 μg/ml purified monomeric protein that were subsequently incubated with the anti-his antibody and the PE labeled detection antibody. The thin histogram line reflects the negative control: cells only incubated with the anti-his antibody and the detection antibody.

FIG. 35: FACS binding analysis of cross-species specific bispecific single chain construct SEQ ID NO. 194×EGFR HL to HPB-ALL (human CD3+), cynomolgus PBMC (cynomolgus CD3+), A431 (human EGFR+) and CHO cells transfected with cynomolgus EGFR (cynomolgus EGFR+), respectively. The FACS staining was performed as described in Example 23. The thick line represents cells incubated with 1 μg/ml purified monomeric protein that were subsequently incubated with the anti-his antibody and the PE labeled detection antibody. The thin histogram line reflects the negative control: cells only incubated with the anti-his antibody and the detection antibody.

FIG. 36: FACS binding analysis of cross-species specific bispecific single chain construct SEQ ID NO. 194×EGFR LH to HPB-ALL (human CD3+), cynomolgus PBMC (cynomolgus CD3+), A431 (human EGFR+) and CHO cells transfected with cynomolgus EGFR (cynomolgus EGFR+), respectively. The FACS staining was performed as described in Example 23. The thick line represents cells incubated with 1 μg/ml purified monomeric protein that were subsequently incubated with the anti-his antibody and the PE labeled detection antibody. The thin histogram line reflects the negative control: cells only incubated with the anti-his antibody and the detection antibody.

FIG. 37: Cytotoxic activity induced by CAIX and CD3 cross-species specific bispecific single chain antibody constructs redirected to indicated target cell lines. Stimulated CD8 positive T cells from human and cynomolgus origin were used as effector cells, respectively. The assay was performed as described in Examples 24 and 25. In the left panel of FIG. 37, a bispecific single chain antibody with a variable domain reactive with CAIX and a de-immunized human CD3-specific variable domain has been used as a positive control. In the right panel, the same construct has been used as a negative control.

FIG. 38: Cytotoxic activity induced by the CAIX and CD3 cross-species specific bispecific single chain antibody construct CAIX HL×SEQ ID NO. 194 redirected to target cell line A549. Stimulated CD8 positive T cells from human and cynomolgus origin were used as effector cells, respectively. The assay was performed as described in Examples 24 and 25.

FIG. 39: Cytotoxic activity induced by EGFR and CD3 cross-species specific bispecific single chain antibody constructs redirected to CHO cells transfected with cynomolgus EGFR as target cell line. Stimulated CD8 positive T cells from cynomolgus origin were used as effector cells. The measurements shown in this figure were performed in a single assay. The assay was performed as described in Example 24. A bispecific single chain antibody with a variable domain reactive with EGFR and a de-immunized human CD3-specific variable domain (EGFR LH×di-anti CD3) has been used as a negative control.

FIG. 40: Cytotoxic activity induced by EGFR and CD3 cross-species specific bispecific single chain antibody constructs redirected to human A431 as target cell line. Stimulated CD8 positive T cells from human origin were used as effector cells. The measurements shown in this figure were performed in a single assay. The assay was performed as described in Example 24. A bispecific single chain antibody with a variable domain reactive with EGFR and a de-immunized human CD3-specific variable domain (EGFR LH×di-anti CD3) has been used as a positive control. As a negative control, an irrelevant bispecific single chain antibody has been used.

### THE FOLLOWING EXAMPLES ILLUSTRATE THE INVENTION

### Example 1: Flow Cytometric Analysis of Cross-Species Specific Antibodies

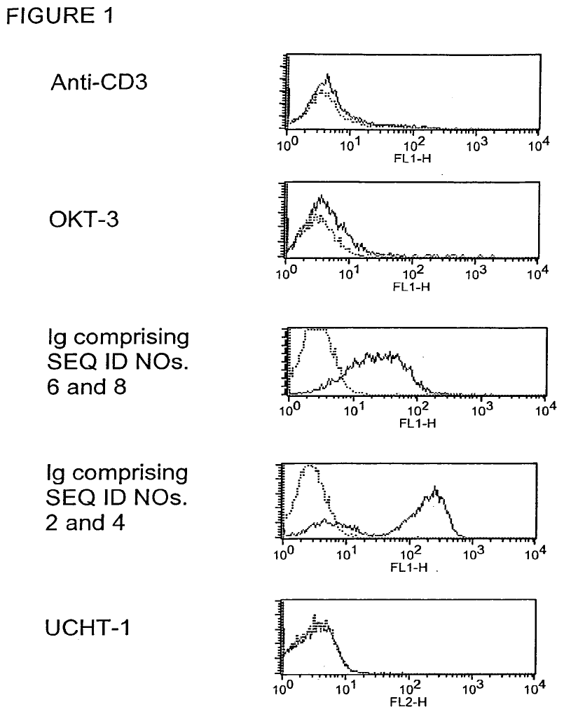

Cross-species specificity of anti-human CD3 antibodies to macaque CD3 (CD3 of Macaca fascicularis, in the following also named “Cynomolgus”) was tested by flow cytometric analysis. Antibodies tested were an anti-CD3 antibody as described in WO 99/54440 (as shown in SEQ ID NO. 162 of the present application), monoclonal antibody (mAb) OKT-3 (Jansen-Cilag), UCHT-1-PE (BD PharMingen, San Diego, California), an immunoglobulin (Ig) comprising the VH and VL chains shown in SEQ ID NOs. 2 and 4, respectively, and an Ig comprising the VH and VL chains shown in SEQ ID NOs. 6 and 8, respectively. 2×10⁵ cells (macaque T cell lines of Macaca fascicularis and Macaca mulatta, respectively, as kindly provided by H. Fickenscher, Heidelberg, Germany) per sample were stained for 30 minutes at 4° C. in 25 μl of PBS/1% FCS/0.05% NaN₃ containing working dilutions of monoclonal antibodies (as determined individually by titration). Cells were washed two times in PBS/1% FCS/0.05% NaN₃ and a secondary antibody was added where necessary. After the addition of the secondary antibody, cells were washed again two times in the same solution and 10.000 living cells were acquired. A FACS Calibur flow cytometer and the CellQuest software from Becton Dickinson were used to collect and analyze the data. Non viable cells were excluded using forward and side scatter electronic gating. Isotype control or secondary antibody only were used as a negative control. As can be seen from FIG. 1, only the Ig comprising the VH and VL chains shown in SEQ ID NOs. 2 and 4, respectively, and the Ig comprising the VH and VL chains shown in SEQ ID NOs. 6 and 8, respectively, showed cross-species specificity for a non-chimpanzee primate CD3, i.e. macaque CD3.

### Example 2: FACS Assay for Binding of an Ig Comprising SEQ ID NOs. 2 and 4, an Ig Comprising SEQ ID NOs. 6 and 8 and mAb FN18 to HPB-ALL Cells and Cynomolgus PBMC

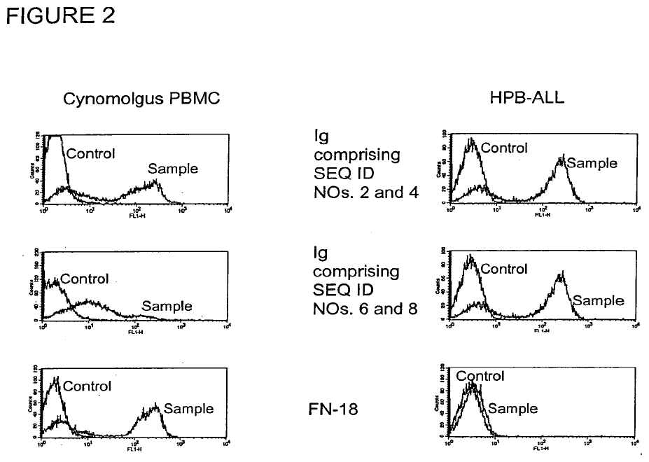

Binding of an Ig comprising SEQ ID NOs. 2 and 4, an Ig comprising SEQ ID NOs. 6 and 8 and mAb FN18 to the cynomolgus CD3 antigen on cynomolgus PBMC and to the human CD3 antigen on HPB-ALL cells (DSMZ No. ACC 483) was tested using an FACS assay. For that purpose, 2,5×10⁵ cells were incubated with the FITC-conjugated Ig comprising SEQ ID NOs. 6 and 8 and the FITC-conjugated Ig comprising SEQ ID NOs. 2 and 4 diluted 1:25 in 50 μl PBS with 2% FCS, respectively. The incubation with the FITC-conjugated mAb FN18 antibody (Biosource International) was performed in 50 μl of undiluted antibody. The samples were measured on a FACSscan (BD biosciences, Heidelberg, FRG). The results for the assay are shown in FIG. 2. Strong antigen binding on human as well as on cynomolgus cells was detected for the Ig comprising SEQ ID NOs. 2 and 4. For the Ig comprising SEQ ID NOs. 6 and 8, strong binding to human cells but weaker binding to cynomolgus cells was observed. For FN18, strong binding to cynomolgus cells could be observed whereas no binding to human cells could be detected.

### Example 3: Sequence Determination of the Variable Regions of Two Anti-Human CD3 Antibodies Exhibiting Species Specificity for Non-Human Primates

For the sequence determination of the variable regions of the cross-species specific anti-CD3 Igs of Examples 1 and 2, PCR (denaturation at 93° C. for 5 min, annealing at 58° C. for 1 min, elongation at 72° C. for 1 min for the first cycle; denaturation at 93° C. for 1 min, annealing at 58° C. for 1 min, elongation at 72° C. for 1 min for 30 cycles; terminal extension at 72° C. for 5 min) was used to amplify the coding sequences of the variable regions of the antibodies. As the sequence of the 5′ region of the variable regions is not known, instead of a single primer a set of 5′ primers was used in combination with a constant 3′ primer whereby the 3′ primer was chosen according to the isotype of the respective antibody and there were two different sets of primers for the 5′ region, one for the light chain variable region and the other for the heavy chain variable region. The primer combinations used in the PCR reactions are given below.

Heavy chain variable region:
5′ primer:
(SEQ ID NO. 81)
5′-SAGGTGCAGCTCGAGGAGTCAGGACCT-3′
(SEQ ID NO. 82)
5′-GAGGTCCAGCTCGAGCAGTCTGGACCT-3′
(SEQ ID NO. 83)
5′-CAGGTCCAACTCGAGCAGCCTGGGGCT-3′
(SEQ ID NO. 84)
5′-GAGGTTCAGCTCGAGCAGTCTGGGGCA-3′
(SEQ ID NO. 85)
5′-GARGTGAAGCTCGAGGAGTCTGGAGGA-3′
(SEQ ID NO. 86)
5′-GAGGTGAAGCTTCTCGAGTCTGGAGGT-3′
(SEQ ID NO. 87)
5′-GAAGTGAAGCTCGAGGAGTCTGGGGGA-3′
(SEQ ID NO. 88)
5′-GAGGTTCAGCTCGAGCAGTCTGGAGCT-3′
(SEQ ID NO. 89)
5′-GGGCTCGAGCACCATGGRATGSAGCTGKGTMATSCTCTT-3′
(SEQ ID NO. 90)
5′-GGGCTCGAGCACCATGRACTTCGGGYTGAGCTKGGTTTT-3′
(SEQ ID NO. 91)
5′-GGGCTCGAGCACCATGGCTGTCTTGGGGCTGCTCTTCT-3′
3′ primer:
(SEQ ID NO. 92)
5′-GAGGAATTCGAACTGGACAGGGATCCAGAGTTCC-3′
(SEQ ID NO. 93)
5′-CGGAATTCGAATGACATGGACATCTGGGTCATCC-3′
Light chain variable region:
5′ primer:
(SEQ ID NO. 94)
5′-CCAGTTCCGAGCTCGTTGTGACTCAGGAATCT-3′
(SEQ ID NO. 95)
5′-CCAGTTCCGAGCTCGTGTTGACGCAGCCGCCC-3′
(SEQ ID NO. 96)
5′-CCAGTTCCGAGCTCGTGCTCACCCAGTCTCCA-3′
(SEQ ID NO. 97)
5′-CCAGTTCCGAGCTCCAGATGACCCAGTCTCCA-3′
(SEQ ID NO. 98)
5′-CCAGATGTGAGCTCGTGATGACCCAGACTCCA-3′
(SEQ ID NO. 99)
5′-CCAGATGTGAGCTCGTCATGACCCAGTCTCCA-3′
(SEQ ID NO. 100)
5′-CCAGTTCCGAGCTCGTGATGACACAGTCTCCA-3′
(SEQ ID NO. 101)
5′-GGGGAGCTCCACCATGGAGACAGACACACTCCTGCTAT-3′
(SEQ ID NO. 102)
5′-GGGGAGCTCCACCATGGATTTTCAAGTGCAGATTTTCAG-3′
(SEQ ID NO. 103)
5′-GGGGAGCTCCACCATGGAGWCACAKWCTCAGGTCTTTRTA-3′
(SEQ ID NO. 104)
5′-GGGGAGCTCCACCATGKCCCCWRCTCAGYTYCTKGT-3′
3′ primer:
(SEQ ID NO. 105)
5′-GAGGAATTCGAACTGCTCACTGGATGGTGGG-3′
(SEQ ID NO. 106)
5′-CGGAATTCGAACAAACTCTTCTCCACAGTGTGACC-3′

All PCR products with a length between 350 and 700 base pairs were isolated, purified and sequenced with the respective 3′ primer according to standard protocols (Sambrook, Molecular Cloning; A Laboratory Manual, Cold Spring Harbor Laboratory Press, Cold Spring Harbor, New York (1989) (2001)).

The obtained sequences were examined for functional variable region coding sequences and for the heavy chain and the light chain of each antibody a sequence coding for the variable region was obtained. The nucleotide and amino acid sequences of the heavy and light chain variable regions of the cross-species specific anti-CD3 antibodies are described in SEQ ID NOs. 1 through 8 in the sequence listing included in the description, respectively.

### Example 4: Cloning of Anti Human EpCAM and CD3 Cross-Species Specific Bispecific Single Chain Antibodies

To generate bispecific single chain antibodies comprising the aforementioned CD3 cross-species specificities, the amplified variable regions had to be modified by PCR to obtain the corresponding single chain Fv antibody fragments. To determine suitable arrangements of the light and heavy chain variable regions in the single chain Fv antibody, two different single chain Fv antibodies were generated for each antibody. To this end, a two-step fusion PCR was used to amplify the sequence coding for the variable regions. A set of appropriate primers was designed to perform the PCR-based cloning steps, finally resulting in a single chain antibody connecting the two variable domains with a 15 amino acid linker ([Gly₄Ser]₃) in the order VH-Linker-VL and VL-Linker-VH. The corresponding nucleotide and amino acid sequences are described in SEQ ID NO. 9 through 12 and in SEQ ID NO. 13 through 16 of the sequence listing included in the description.

In short the following primer combinations were used:

For VL-VH scFv antibody shown in SEQ ID NOs. 11 and 12: SEQ ID NOs. 17 to 20.

For VH-VL scFv antibody shown in SEQ ID NOs. 9 and 10: SEQ ID NOs. 21 to 24.

For VL-VH scFv antibody shown in SEQ ID NOs. 15 and 16: SEQ ID NOs. 25 to 28.

For VH-VL scFv antibody shown in SEQ ID NOs. 13 and 14: SEQ ID NOs. 29 to 32.

To generate the single chain antibody, two PCRs with the respective primer combinations were performed. During this PCR overlapping complementary sequences were introduced into the PCR-products stemming from the respective linker primers that combined to form the coding sequence of the 15 amino acid linker during the subsequent fusion PCR. The amplified VH and VL domains were fused in a next PCR in which only the outer primers and both PCR-products were required. The resulting scFv antibody is flanked at the 5′ end with a small Ser(Gly₄)Ser linker preceded by the restriction enzyme recognition site for BspEI and at the 3′end with a 6 histidine affinity tag followed by a stop codon and by the restriction enzyme recognition site for SalI. The second single chain Fv antibody was an anti human EpCAM specificity designated “5-10” which is described in SEQ ID NO. 33 and 34 of the sequence listing included in the description. To accomplish the fusion of the single chain Fv antibodies and to allow for eukaryotic expression, the coding sequence of the single chain Fv antibodies was then cloned via BspEI (5′ to the Ser (Gly₄) Ser linker) and SalI into the pEFDHFR expression vector (pEFDHFR was described in Mack et al. Proc. Natl. Acad. Sci. USA 92 (1995) 7021-7025) containing the coding sequence for the human EpCAM specific single chain Fv antibody 5-10 and the restriction enzyme recognition site for BspEI. The coding sequence of an murine immunoglobulin leader peptide is described in SEQ ID NO. 35 and 36 of the sequence listing included in the description, preceded by a Kozak translation initiation consensus sequence and the restriction enzyme recognition site for EcoRI. Single clones of the constructs were isolated and sequenced with primers complementary to flanking regions in the vector according to standard protocols (Sambrook, Molecular Cloning; A Laboratory Manual, Cold Spring Harbor Laboratory Press, Cold Spring Harbor, New York (1989) (2001)). For further experiments a clone of each construct was selected. The nucleotide and amino acid sequences are described for 5-10LHxSEQ ID NO. 12 in SEQ ID NOs. 37 and 38, for 5-10LHxSEQ ID NO. 10 in SEQ ID NOs. 39 and 40, for 5-10LHxSEQ ID NO. 16 in SEQ ID NOs. 41 and 42 and for 5-10LHxSEQ ID NOs. 14 in SEQ ID NO. 43 and 44 of the sequence listing included in the description.

### Example 5: Expression of the 5-10LHxSEQ ID NO. 12, 5-10LHxSEQ ID NO. 10, 5-10LHxSEQ ID NO. 16 and 5-10LHxSEQ ID NO. 14 Bispecific Single Chain Antibodies in CHO Cells

The plasmids with the sequences coding for the bispecific single chain antibodies were transfected into DHFR deficient CHO cells for eukaryotic expression of the construct as described in Kaufmann R. J. (1990) Methods Enzymol. 185, 537-566). Gene amplification of the construct was induced by increasing concentrations of Methotrexat (MTX) to a final concentration of up to 500 nM MTX. The transfected cells were then expanded and 1 liter of supernatant produced. The construct was finally purified out of the culture supernatant as described in Kufer et al. Cancer immunity Vol. 1, p. 10 (2001).

### Example 6: FACS Assay for Binding of 5-10LHxSEQ ID NO. 12, 5-10LHxSEQ ID NO. 10, 5-10LHxSEQ ID NO. 16 and 5-10LHxSEQ ID NO. 14 to Kato III Cells or Human EpCAM Transfected CHO Cells and to HPB-ALL Cells

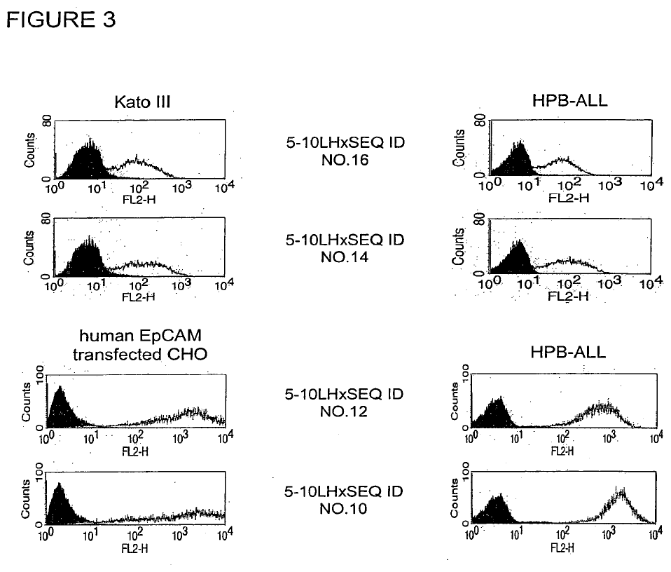

Binding of the bifunctional constructs to the EpCAM antigen on human Kato III cells expressing EpCAM (ATCC No. HTB-103) or on human EpCAM transfected CHO cells and to the human CD3 antigen on HPB-ALL cells was tested using an FACS assay. For that purpose 2.5×10⁵ cells were incubated with 50 μl of cell culture supernatant containing the construct. The binding of the construct was detected with an anti-His antibody (Penta-His Antibody, BSA free, obtained from Qiagen GmbH, Hilden, FRG) at 2 μg/ml in 50 μl PBS with 2% FCS. As a second step reagent a R-Phycoerythrin-conjugated affinity purified F(ab′)2 fragment, goat anti-mouse IgG, Fc-gamma fragment specific antibody, diluted 1:100 in 50 μl PBS with 2% FCS (obtained from Dianova, Hamburg, FRG) was used. The samples were measured on a FACSscan (BD biosciences, Heidelberg, FRG). Antigen binding was clearly detectable for the anti human EpCAM specificity as well as for the anti CD3 specificities on the cell line positive for human CD3 (see FIG. 3).

### Example 7: Cytotoxicity Assay for 5-10LHxSEQ ID NO. 12, 5-10LHxSEQ ID NO. 10, and 5-10LHxSEQ ID NO. 14 with Kato III Cells as Target Cells and Human PBMC as Effector Cells

Bioactivity of 5-10LHxSEQ ID NO. 12, 5-10LHxSEQ ID NO. 10, and 5LH-10×SEQ ID NO. 14 was analyzed by FACS-based in vitro cytotoxicity assays using the human EpCAM positive Kato III cells as target cells and human PBMCs as effector cells. Target cells were washed twice with PBS and labeled with PKH26 dye (Sigma-Aldrich, Germany) according to the manufacturer's instructions. Labeled target cells were washed twice with RPMI/10% FCS and mixed with freshly isolated effector cells at an E:T ratio of 10:1. Two times 10⁴ target and 2×10⁵ effector cells in a volume of 50 μl RPMI/10% FCS were added per well in a 96-well round bottom plate. Ten-fold serial dilutions of different bispecific single chain constructs were prepared in RPMI/10% FCS to obtain a starting concentration of 1000 ng/ml in the final reaction volume. 50 μl of the different solutions were added in triplicates to the corresponding wells. Individual cytotoxicity mixtures were incubated for 24 to 48 hours at 37° C., 5% CO₂.

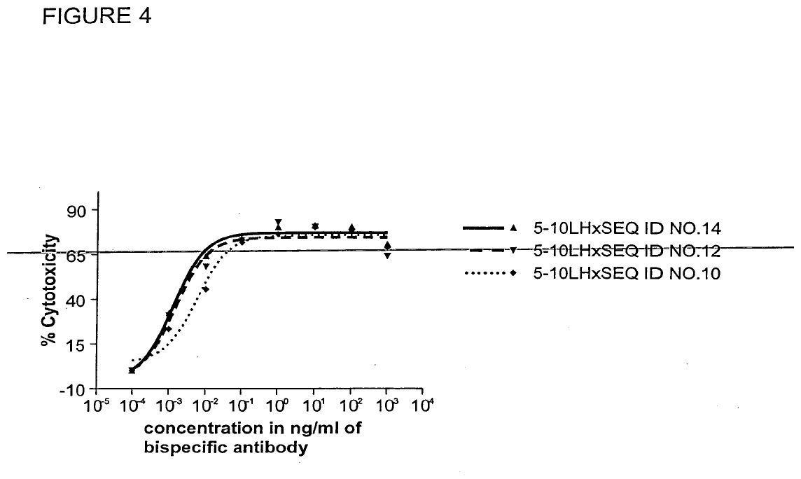

Subsequently the measurement of cytotoxic activity was performed. To this end, Propidium iodide (PI) was added to a final concentration of 1 μg/ml per well and plates were incubated for 10 minutes at room temperature. The number of PKH and PI positive and negative target cells was determined by FACS. Cytotoxicity was measured as the ratio of PKH-positive and PI negative (living target cells) over the mean of living target cells (PKH-positive and PI negative) in the control containing no construct according to the formula: cytotoxicity (%)=[(PI-negative cells/mean of PI-negative cells in control)×100]. Sigmoidal dose response killing curves were analyzed by Prism Software (GraphPad Software Inc., San Diego, USA) and the BiTE concentration calculated that induced half maximal killing (EC50 value). The results of this assay are shown below in FIG. 4. All constructs showed cytotoxic activity. The resulting EC50 values for 5-10LHxSEQ ID NO. 14, 5-10LHxSEQ ID NO. 12 and 5-10LHxSEQ ID NO. 10 were 1.3 pg/ml, 1.5 pg/ml and 5.8 pg/ml respectively.

### Example 8: Cytotoxicity Assay for 5-10LHxSEQ ID NO. 12, 5-10LHxSEQ ID NO. 10, and 5-10LHxSEQ ID NO. 14 with Kato III Cells as Target Cells and Cynomolgus PBMC as Effector Cells

Bioactivity of 5-10LHxSEQ ID NO. 12, 5-10LHxSEQ ID NO. 10, and 5-10LHxSEQ ID NO. 14 was analyzed by FACS-based in vitro cytotoxicity assays using the human EpCAM positive Kato III cells as target cells and cynomolgus PBMCs as effector cells.

Target cells were washed twice with PBS and labeled with PKH26 dye (Sigma-Aldrich, Germany) according to the manufacturer's instructions. Labeled target cells were washed twice with RPMI/10% FCS and mixed with freshly isolated effector cells at an E:T ratio of 10:1. Two times 10⁴ target and 2×10⁵ effector cells in a volume of 50 μl RPMI/10% FCS were added per well in a 96-well round bottom plate. Ten-fold serial dilutions of different Bispecific single chain antibodies were prepared in RPMI/10% FCS to obtain a starting concentration of 1000 ng/ml in the final reaction volume. 50 μl of the different solutions were added in triplicates to the corresponding wells. Individual cytotoxicity mixtures were incubated for 24 to 48 hours at 37° C., 5% CO₂.

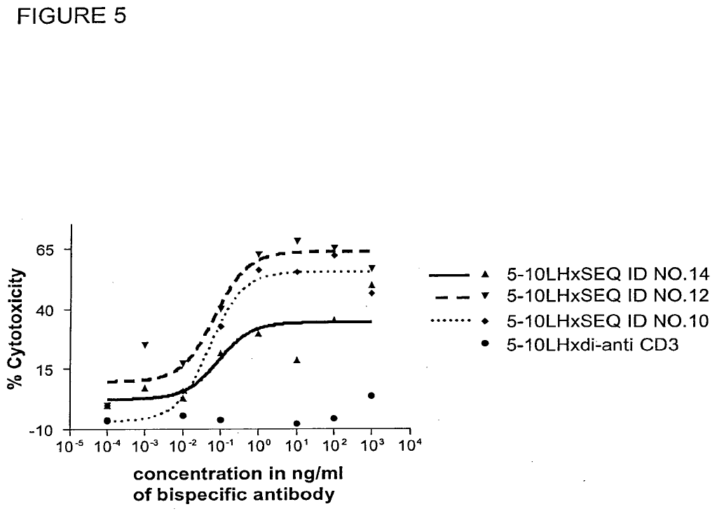

Subsequently the measurement of cytotoxic activity was performed. To this end, propidium iodide (PI) was added to a final concentration of 1 μg/ml per well and plates were incubated for 10 minutes at room temperature. The number of PKH and PI positive and negative target cells was determined by FACS. Cytotoxicity was measured as the ratio of PKH-positive and PI negative (living target cells) over the mean of living target cells (PKH-positive and PI negative) in the control containing no construct according to the formula: cytotoxicity (%)=[(PI-negative cells/mean of PI-negative cells in control)×100]. Sigmoidal dose response killing curves were analyzed by Prism Software (GraphPad Software Inc., San Diego, USA) and the bispecific single chain antibody concentration calculated that induced half maximal killing (EC50 value). The results of this assay are shown below in FIG. 5. 5-10LHxSEQ ID NO. 14, 5-10LHxSEQ ID NO. 12 and 5-10LHxSEQ ID NO. 10 showed cytotoxic activity. The resulting EC50 values for 5-10LHxSEQ ID NO. 14, 5-10LHxSEQ ID NO. 12 and 5-10LHxSEQ ID NO. 10 were 87 μg/ml, 69 μg/ml and 52 μg/ml respectively. 5-10LHxdi-anti CD3 (deimmunised anti-CD3 antibody as shown in SEQ ID NO. 163) showed no activity. This is due to the fact that di-anti CD3 antibody only binds to human CD3, but not to cynomolgus CD3.

### Example 9: Sequence Determination of the Cynomolgus EpCAM Antigen and Generation of Cynomolgus EpCAM Transfected CHO Cells

To obtain the cynomolgus EpCAM antigen for testing of cross-species specificity of anti human EpCAM antibodies, first the coding sequence of the cynomolgus EpCAM antigen had to be determined. To this end, colon tissue samples of 3 animals were used in parallel for the isolation of total RNA and cDNA synthesis by random-primed reverse transcription, which were performed according to standard protocols (Sambrook, Molecular Cloning; A Laboratory Manual, Cold Spring Harbor Laboratory Press, Cold Spring Harbor, New York (1989) (2001)). A PCR (denaturation at 93° C. for 5 min, annealing at 58° C. for 1 min, elongation at 72° C. for 1 min for the first cycle; denaturation at 93° C. for 1 min, annealing at 58° C. for 1 min, elongation at 72° C. for 1 min for 35 cycles; terminal extension at 72° C. for 5 min) was used to amplify the coding sequence of the EpCAM antigen. As the coding sequence of the cynomolgus EpCAM antigen was not known, appropriate primers (5′ primer described in SEQ ID NO. 45, 3′ primer described in SEQ ID NO. 46) for the PCR reaction were designed according to the known coding sequence of the human EpCAM antigen (Szala S. et al., Proc Natl Acad Sci USA. 87 (1990); p. 3542-6). Primers were also designed as to allow for expression of the coding sequence of the entire antigen. For the 3 samples, PCR of 960 base pairs were isolated, purified and subcloned via XbaI and SalI, into pEFDHFR. Multiple clones for each sample were sequenced according to standard protocols (Sambrook, Molecular Cloning; A Laboratory Manual, Cold Spring Harbor Laboratory Press, Cold Spring Harbor, New York (1989) (2001)) using appropriate sequencing primers complementary to flanking sequences in the vector.

The novel nucleotide and amino acid sequences of the cynomolgus EpCAM antigen are described in SEQ ID NOs. 47 and 48 in the sequence listing included in the description, respectively.

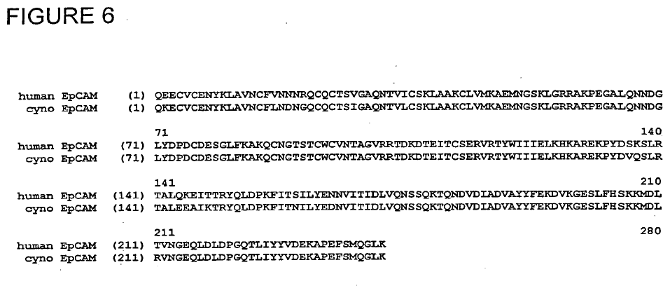

The obtained sequences were examined by comparison with the coding sequence of the human EpCAM antigen. As shown in FIG. 6, there is a high degree of sequence homology between the coding sequence of the human EpCAM antigen and the sequences obtained from the colon samples of the 3 cynomolgus monkeys.

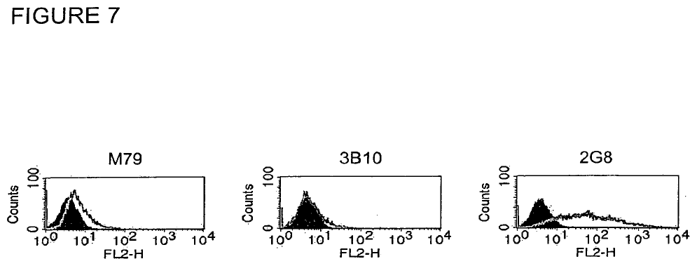

To generate a cell line positive for cynomolgus EpCAM, a clone of the aforementioned coding sequence of the cynomolgus EpCAM antigen subcloned into pEFDHFR with a verified nucleotide sequence was transfected into DHFR deficient CHO cells for eukaryotic expression of the construct as described in Kaufmann R. J. (1990) Methods Enzymol. 185, 537-566). Gene amplification of the construct was induced by increasing concentrations of MTX to a final concentration of up to 500 nM MTX. The transfected cells were then tested for expression of cynomolgus EpCAM using an FACS assay. For that purpose, a number of 2,5×10⁵ cells was incubated with 50 μl supernatant three different mouse anti human EpCAM hybridomas (M79-Fogler et al., Cancer Res. 48 (1988); p. 6303-8; 3B10-Passlick et al. Int. J. Cancer 87 (2000), p. 548-552; 2G8-Balzar et al., J. Mol. Med. 77 (1999), p. 699-712). The binding of the antibodies was detected with a R-Phycoerythrin-conjugated affinity purified F(ab′)2 fragment, goat anti-mouse IgG, Fc-gamma fragment specific antibody, diluted 1:100 in 50 μl PBS with 2% FCS (obtained from Dianova, Hamburg, FRG) was used. The samples were measured on a FACSscan (BD biosciences, Heidelberg, FRG). The anti EpCAM antibody 2G8 was recognized as cross-species specific and the expression of cynomolgus EpCAM was confirmed (see FIG. 7). Transfectants (depicted as non-filled curves) as compared to untransfected cells (depicted as filled curves) showed binding only with the supernatant of the 2G8 hybridoma which is therefore recognized as antibody species specific for human and cynomolgus EpCAM.

### Example 10: Sequence Determination of the Variable Regions of an Anti Human EpCAM Antibody Cross-Species Specific for Non-Human Primates

For the sequence determination of the variable regions of the anti-EpCAM antibody 2G8, the respective hybridoma cell line was used for isolation of total RNA and cDNA synthesis by random-primed reverse transcription, which were performed according to standard protocols (Sambrook, Molecular Cloning; A Laboratory Manual, Cold Spring Harbour Laboratory Press, Cold Spring Harbour, New York (1989) (2001)). A PCR (denaturation at 93° C. for 5 min, annealing at 58° C. for 1 min, elongation at 72° C. for 1 min for the first cycle; denaturation at 93° C. for 1 min, annealing at 58° C. for 1 min, elongation at 72° C. for 1 min for 30 cycles; terminal extension at 72° C. for 5 min) was used to amplify the coding sequences of the variable regions of the antibody. As the sequence of the 5′ region of the variable regions is not known the aforementioned set of 5′ primers was used in combination with a constant 3′ primer whereby the 3′ primer was chosen according to the isotype of the antibody.

Heavy chain variable region:
3′ primer:
(SEQ ID NO. 107)
5′-TATGCAACTAGTACAACCACAATCCCTGGG-3′
Light chain variable region:
3′ primer:
(SEQ ID NO. 108)
5′-GCGCCGTCTAGAATTAACACTCATTCCTGTTGAA-3′

All PCR products with a length between 350 and 700 base pairs were isolated, purified and sequenced with the respective 3′ primer according to standard protocols (Molecular Cloning; A Laboratory Manual, Cold Spring Harbor Laboratory Press, Cold Spring Harbor, New York (1989) (2001)).

The obtained sequences were examined for functional variable region coding sequences and for the heavy chain and the light chain of the antibody a sequence coding for the variable region was isolated. The nucleotide and amino acid sequences of the variable regions are described in SEQ ID NOs. 49 through 52 in the sequence listing included in the description, respectively.

### Example 11: Cloning of EpCAM and CD3 Cross-Species Specific Bispecific Single Chain Antibodies

To generate bispecific single chain antibody molecules comprising the aforementioned CD3 cross-species specificity and the aforementioned EpCAM cross-species specificity, the amplified variable regions of the 2G8 antibody had to be modified by PCR to obtain the corresponding single chain Fv antibody fragments. Two single chain Fv antibodies with different arrangements of the light and heavy chain variable regions were generated. To this end, a two-step fusion PCR was used to amplify the sequence coding for the variable regions. A set of appropriate primers was designed to perform the PCR-based cloning steps, finally resulting in a 2G8 single chain antibody connecting the two variable domains with a 15 amino acid linker ([Gly₄Ser]₃) in the order VH-Linker-VL and VL-Linker-VH. The nucleotide and amino acid sequences are described in SEQ ID NOs. 53 through 56 of the sequence listing included in the description, respectively.

In short the following primer combinations were used:

For 2G8 VL-VH scFv antibody (hereafter designated as 2G8LH shown in SEQ ID NOs. 55 and 56): SEQ ID NOs. 57 to 60.

For 2G8 VH-VL scFv antibody (hereafter designated as 2G8HL shown in SEQ ID NOs. 53 and 54): SEQ ID NOs. 61 to 64.

To generate the single chain antibody, two PCRs with the respective primer combinations were performed. During this PCR, overlapping complementary sequences were introduced into the PCR-products (stemming from the respective linker primers that combined to form the coding sequence of the 15 amino acid linker during the subsequent fusion PCR). The amplified VH and VL domains were fused in this fusion PCR in which only the outer primers and both PCR-products were required. The resulting scFv antibody is flanked at the 5′end with the restriction enzyme recognition site for BsrGI and at the 3′end with the restriction enzyme recognition site for BspEI. The coding sequence of the EpCAM specific single chain Fv antibodies was then cloned via BsrGI and BspEI into the pEFDHFR expression vectors described above replacing the 5-10LH scFv. Single clones of the constructs were isolated and sequenced with primers complementary to flanking regions in the vector according to standard protocols (Sambrook, Molecular Cloning; A Laboratory Manual, Cold Spring Harbor Laboratory Press, Cold Spring Harbor, New York (1989) (2001)). For further experiments a clone of each construct was selected. The nucleotide and amino acid sequences are described for 2G8LHxSEQ ID NO. 12 in SEQ ID NOs. 65 and 66, for 2G8LHxSEQ ID NO. 10 in SEQ ID NOs. 67 and 68, for 2G8LHxSEQ ID NO. 16 in SEQ ID NOs. 69 and 70, for 2G8LHxSEQ ID NO. 14 in SEQ ID NOs. 71 and 72, for 2G8HLxSEQ ID NO. 12 in SEQ ID NOs. 73 and 74, for 2G8HLxSEQ ID NO. 10 in SEQ ID NOs. 75 and 76, for 2G8HLxSEQ ID NO. 16 in SEQ ID NOs. 77 and 78, and for 2G8HLxSEQ ID NO. 14 in SEQ ID NOs. 79 and 80 of the sequence listing included in the description.

### Example 12: Expression of the 2G8LHxSEQ ID NO. 12, 2G8LHxSEQ ID NO. 10, 2G8LHxSEQ ID NO. 16, 2G8LHxSEQ ID NO. 14, 2G8HLxSEQ ID NO. 12, 2G8HLxSEQ ID NO. 10, 2G8HLxSEQ ID NO. 16 and 2G8HLxSEQ ID NO. 14 Bispecific Single Chain Antibodies in CHO Cells

The plasmids with the sequences coding for the bispecific single chain antibodies were transfected into DHFR deficient CHO cells for eukaryotic expression of the construct as described in Kaufmann R. J. (1990) Methods Enzymol. 185, 537-566). Gene amplification of the construct was induced by increasing concentrations of MTX to a final concentration of up to 500 nM MTX. The transfected cells were then expanded and 1 liter of supernatant produced. The construct was finally purified out of the culture supernatant as described in Kufer et al. Cancer Immunity Vol. 1, p. 10 (2001).

### Example 13: FACS Assay for Binding of 2G8LHxSEQ ID NO. 12, 2G8LHxSEQ ID NO. 10, 2G8LHxSEQ ID NO. 16, 2G8LHxSEQ ID NO. 14, 2G8HLxSEQ ID NO. 12, 2G8HLxSEQ ID NO. 10, 2G8HLxSEQ ID NO. 16 and 2G8HLxSEQ ID NO. 14 on Kato III Cells or Cynomolgus EpCAM Transfected CHO Cells and HPB-ALL Cells

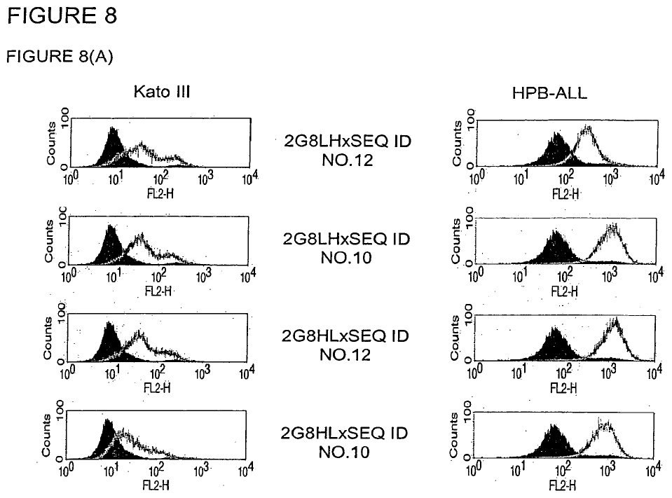

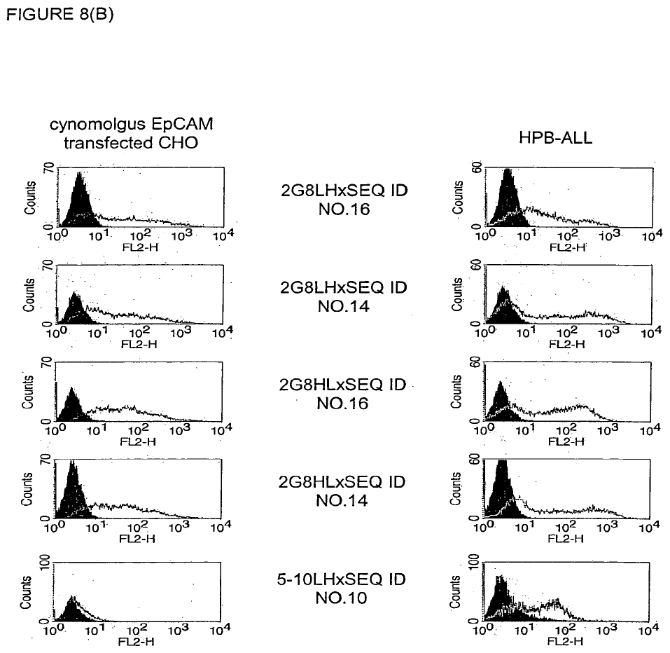

Binding of the bifunctional constructs from cell culture supernatants or binding of purified bifunctional constructs to the human EpCAM antigen on Kato III cells or cynomolgus EpCAM transfected CHO cells and to the CD3 antigen on HPB-ALL cells was tested using an FACS assay. For that purpose 2,5×10⁵ cells were incubated with 50 μl supernatant or with 5 μg/ml of the purified constructs in 50 μl PBS with 2% FCS. The binding of the constructs was detected with an anti-His antibody (Penta-His Antibody, BSA free, obtained from Qiagen GmbH, Hilden, FRG) at 2 μg/ml in 50 μl PBS with 2% FCS. As a second step reagent a R-Phycoerythrin-conjugated affinity purified F(ab′)2 fragment, goat anti-mouse IgG, Fc-gamma fragment specific antibody, diluted 1:100 in 50 μl PBS with 2% FCS (obtained from Dianova, Hamburg, FRG) was used. The samples were measured on a FACSscan (BD biosciences, Heidelberg, FRG). Antigen binding was clearly detectable for the anti EpCAM specificities as well as for the anti CD3 specificities (see FIG. 8). As a negative control for binding to cynomolgus EpCAM, the 5-10LHxSEQ ID NO. 10 construct was included which shows binding on human CD3 (HPB-ALL cells) but no binding to cynomolgus EpCAM (cynomolgus EpCAM transfected CHO cells). The 5-10LH part only binds to human EpCAM.

### Example 14: Cytotoxicity Assay for 2G8LHxSEQ ID NO. 10 and 2G8HLxSEQ ID NO. 12 with Cynomolgus EpCAM Transfected CHO Cells as Target Cells and Human PBMC as Effector Cells

Bioactivity of selected bispecific single chain antibodies was analyzed by FACS-based in vitro cytotoxicity assays using the cynomolgus EpCAM transfected CHO cells as target cells and human PBMCs as effector cells.

Target cells were washed twice with PBS and labeled with PKH26 dye (Sigma-Aldrich, Germany) according to the manufacturer's instructions. Labeled target cells were washed twice with RPMI/10% FCS and mixed with freshly isolated effector cells at an E:T ratio of 10:1. 2×10⁴ target and 2×10⁵ effector cells in a volume of 50 μl RPMI/10% FCS were added per well in a 96-well round bottom plate. Ten-fold serial dilutions of different bispecific single chain antibodies were prepared in RPMI/10% FCS to obtain a starting concentration of 5000 ng/ml in the final reaction volume. 50 μl of the different solutions were added in triplicates to the corresponding wells and incubated for 24 to 48 hours at 37° C., 5% CO₂.

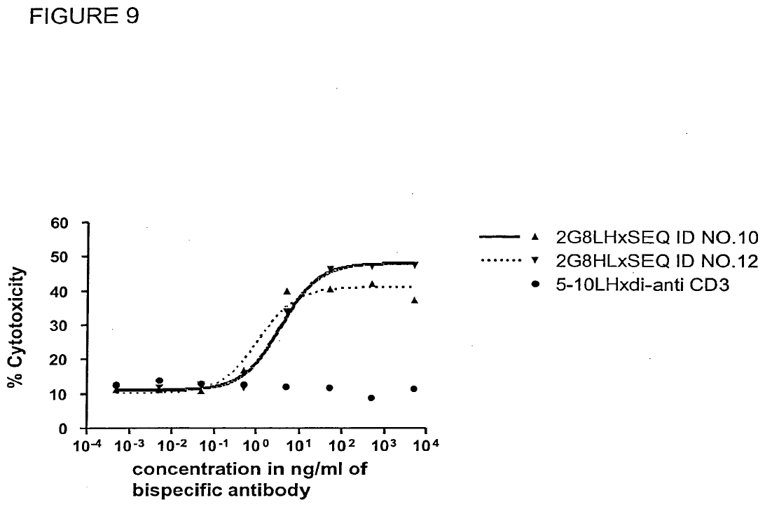

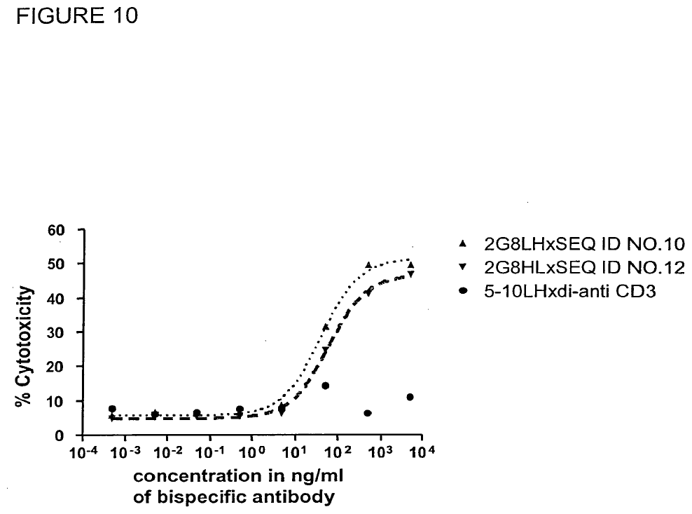

Subsequently, the measurement of cytotoxic activity was performed. To this end propidium iodide (PI) was added to a final concentration of 1 μg/ml per well and plates were incubated for 10 minutes at room temperature. The number of PKH and PI positive target cells was determined by FACS. Cytotoxicity was measured as the ratio of PI positive (dead cells) over total number of target cells (PKH-positive) according to the formula: cytotoxicity (%)=[(PI-positive cells/PKH-positive cells)×100]. Sigmoidal dose response killing curves were analyzed by Prism Software (GraphPad Software Inc., San Diego, USA) and the bispecific single chain antibody concentration calculated that induced half maximal killing (EC50 value). The results of this assay are shown below in FIG. 9. The resulting EC50 values for 2G8LHxSEQ ID NO. 10 and 2G8HLxSEQ ID NO. 12 were 1103 μg/ml and 3638 μg/ml, respectively. 5-10LHxdi-anti CD3 (deimmunised version of the anti-CD3 antibody as shown in SEQ ID NO. 163 binding to human CD3, but not to cynomolgus CD3) was included as negative control and showed no activity. This is due to the fact that 5-10LH only binds to human EpCAM but lacks cross-species specificity to cynomolgus EpCAM.

### Example 15: Cytotoxicity Assay for 2G8LHxSEQ ID NO. 10 and 2G8HLxSEQ ID NO. 12 with Cynomolgus EpCAM Transfected CHO Cells as Target Cells and Cynomolgus PBMC as Effector Cells

Bioactivity of selected bispecific single chain antibodies was analyzed by FACS-based in vitro cytotoxicity assays using the cynomolgus EpCAM transfected CHO cells as target cells and cynomolgus PBMCs as effector cells.

Target cells were washed twice with PBS and labeled with PKH26 dye (Sigma-Aldrich, Germany) according to the manufacturer's instructions. Labeled target cells were washed twice with RPMI/10% FCS and mixed with freshly isolated effector cells at an E:T ratio of 10:1. 2×10⁴ target and 2×10⁵ effector cells in a volume of 50 μl RPMI/10% FCS were added per well in a 96-well round bottom plate. Ten-fold serial dilutions of different bispecific single chain antibodies were prepared in RPMI/10% FCS to obtain a starting concentration of 5000 ng/ml in the final reaction volume. 50 μl of the different solutions were added in triplicates to the corresponding wells. Individual cytotoxicity mixtures were incubated for 24 to 48 hours at 37° C., 5% CO₂.

Subsequently the measurement of cytotoxic activity was performed as described in Example 14. The resulting EC50 values for 2G8LHxSEQ ID NO. 10 and 2G8HLxSEQ ID NO. 12 were 39810 μg/ml and 60350 μg/ml respectively. 5-10LHxdi-anti CD3 (deimmunised version of the anti-CD3 antibody as shown in SEQ ID NO. 163) was included as negative control and showed no activity. Di-anti CD3 only binds to human CD3, but fails to bind to macaque/cynomolgus CD3. 5-10LH only binds to human EpCAM but lacks cross-species specificity to cynomolgus EpCAM.

### Example 16: Generation of a Human-Like CD3 Antibody Fragment that Binds to Human and Cynomolgus CD3

### 1. Determination of a Correlating Human VH

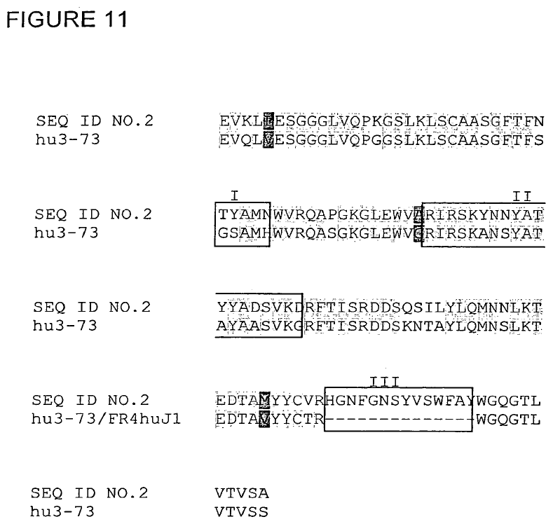

The amino acid sequence of the murine VH chain shown in SEQ ID NO. 2 was aligned to the repertoire of human VH germline sequences (http://vbase.mrc-cpe.cam.ac.uk) using the Vector NTI DNA analysis software. On the basis of this analysis, the human VH segment 3-73 was chosen as a template sequence (see FIG. 11). Definitions of CDRs and frameworks are according to the Kabat numbering scheme.

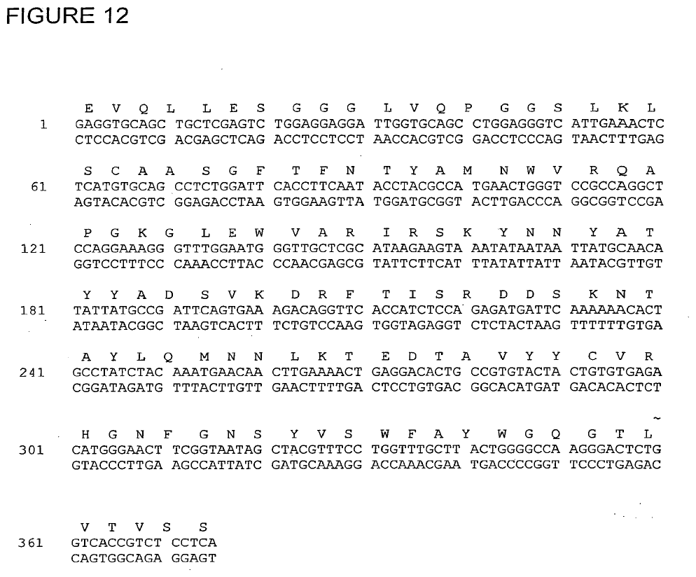

The corresponding amino acid residues that differ between the VH chain shown in SEQ ID NO. 2 and the human VH segment 3-73 within the framework regions were mutated on the DNA level towards the human residues. However, the construct retained potentially crucial framework residues of the original murine VH sequence (according to the Kabat numbering scheme): H-30, H-41, H49, H82b, H-93 (see FIG. 12). In this way, an amino acid sequence was designed that was identical to the murine VH chain shown in SEQ ID NO. 2 sequence within its CDRs. The corresponding amino acid sequence is shown in SEQ ID NO. 110, whereas the corresponding nucleic acid sequence is shown in SEQ ID NO. 111; see also FIG. 12. The N-terminal VH sequence was changed to “EVQLLE” to generate a suitable N-terminal cloning site (see FIG. 12).

### 2. Gene Synthesis and Cloning of the Human-Like VH Region

The afore-mentioned human-like VH region was gene synthesized (Entelechon, Germany) and subcloned via the restriction sites XhoI and BstEII into a suitable bacterial expression vector. This vector already contained the sequence coding for a VL chain (amino acid sequence shown in SEQ ID NO. 148; N-terminus in comparison to the original VL shown in SEQ ID NO. 4 slightly changed for cloning reasons) pairing with the human like VH region followed by a Flag and a His-6 Tag and preceded by a leader sequence that directs the functional scFv into the periplasma of E. coli. The functional domain arrangement after cloning was Leader sequence-VH-(G4S) 3-VL-Flag-His6.

3. Functional Analysis of scFv Constructs Having the Original Murine VH Shown in SEQ ID NO. 2/VL Shown in SEQ ID NO. 4 in Comparison to the Human-Like VH Shown in SEQ ID NO. 110/VL Shown in SEQ ID NO. 148

Plasmid DNA encoding a) for the original murine VH (SEQ ID NO. 2) and VL (SEQ ID NO. 4) and b) for the human-like VH (SEQ ID NO. 110) combined with the VL (SEQ ID NO. 148) was each transformed into E. coli TG1 according to standard protocols. The nucleotide and amino acid sequences of the VH-VL scFv comprising the original murine VH (SEQ ID NO. 2) and VL (SEQ ID NO. 4) are shown in SEQ ID NOs. 9 and 10, respectively. The nucleotide and amino acid sequences of the VH-VL scFv comprising the human-like VH (SEQ ID NO. 110) and the VL (SEQ ID NO. 148) are shown in SEQ ID NOs. 147 and 146, respectively.

Expression of different clones was performed in E. coli TG-1 in 96-well format. 100 μl LB/0.1% glucose were inoculated with 10 μl of an overnight culture of single clones and grown for 4 h at 37° C. After addition of IPTG to a final concentration of 1 mM, the culture was grown at 30° C. for another 18-20 h. Per well, 40 μl of BEL-buffer (400 mM boric acid, 320 mM NaCl, 4 mM EDTA pH 8.0+2.5 mg/ml lysozyme) were added and shaken at room temperature for 1 h. Cellular debris was eliminated by centrifugation and supernatants were tested in flow cytometric experiments.

The human T cell line HPB-All and T cells in cynomolgus peripheric blood mononuclear cells (PBMC) were used as human CD3 and cynomolgus CD3 positive cells, respectively. Typically 100,000 cells were incubated with 50 μl of the scFv containing bacterial supernatants and incubated for 30 min on ice.

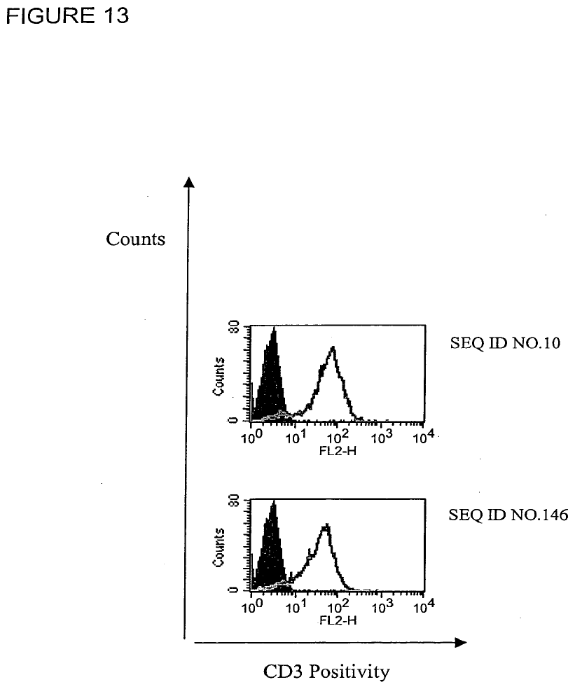

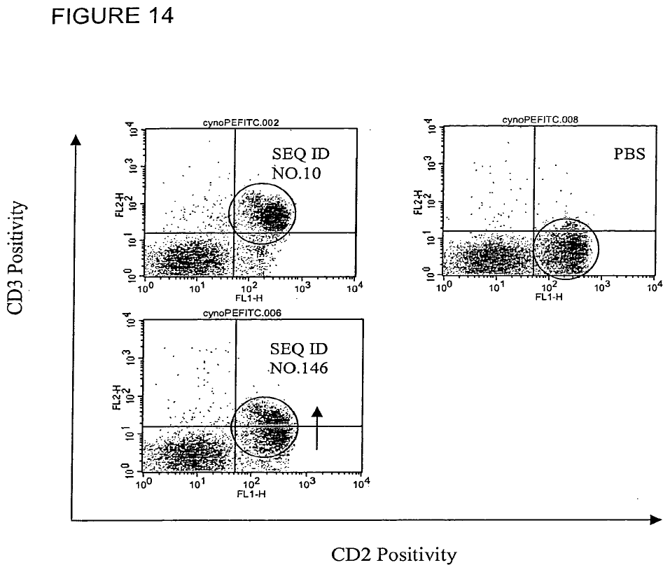

Afterwards the cells were washed three times with PBS and subsequently resuspended in 50 μl PBS containing anti-His antibody (Pentahis, Roche) and further incubated on ice for 30 min. Then the cells were washed three times with PBS and incubated with a PE labeled anti mouse IgG antibody for 30 more min. on ice (in this step cynomolgus PBMCs were coincubated with anti-CD2 FITC to identify the T cells in the PBMC mixure). After washing the cells for one time the cells were resuspended in a suitable buffer and positivity of cell bound antibody construct determined in a flow cytometer (FACScalibur) and analyzed. The control scFv of SEQ ID NO. 10 shows a clear shift on human CD3 positive cells as well as on cynomolgus CD3 positive cells indicative of binding to both human and cynomolgus CD3. The scFv shown in SEQ ID NO. 146 containing the human-like VH also shows clear binding to CD3 positive human (see FIG. 13) and cynomolgus cells (see FIG. 14).

### 4. Determination of a Correlating Human VL

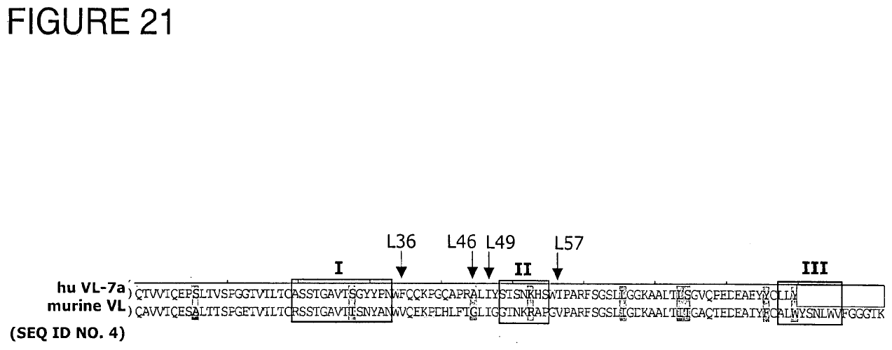

The amino acid sequence of the murine VH chain shown in SEQ ID NO. 2 was aligned to the repertoire of human VL germline sequences (http://vbasc.mrc-cpc.cam.ac.uk) using the Vector NTI DNA analysis software. On the basis of this analysis, the human Vlambda segment 7a was chosen as a template sequence (see FIG. 21). Definitions of CDRs and frameworks are according to the Kabat numbering scheme.

The corresponding amino acid residues that differ between the murine VL chain shown in SEQ ID NO. 4 and the human Vlambda segment 7a within the framework regions were mutated on the DNA level towards the human residues. However, the construct retained potentially crucial framework residues of the original murine Vlambda sequence (according to the Kabat numbering scheme): L 36, L 46, L 49, L 57 (see FIG. 21). In this way, an amino acid sequence was designed that was identical to the murine VL chain shown in SEQ ID NO. 4 sequence within its CDRs. The corresponding amino acid sequence of the generated human-like VL is shown in SEQ ID NO. 168, whereas the corresponding nucleic acid sequence is shown in SEQ ID NO. 167. The N-terminal VL sequence was changed to “EL” to generate a suitable N-terminal cloning site.

### 5. Gene Synthesis and Cloning of the Human-Like VL Region

The above-mentioned human-like VL region was gene synthesized (Entelechon, Germany) and subcloned via the restriction sites SacI and BsiWI into a suitable bacterial expression vector. This vector already contained the sequence coding for the above-mentioned human-like VH chain (amino acid sequence shown in SEQ ID NO. 110) pairing with the human-like VL region (amino acid sequence shown in SEQ ID NO. 168) followed by a Flag and a His-6 Tag and preceded by a leader sequence that directs the functional scFv into the periplasma of E. coli. The functional domain arrangement after cloning was Leader sequence-VH-(G4S) 3 linker-VL-Flag tag-His6 tag.

6. Functional Analysis of scFv Constructs Having the Human-Like VH Shown in SEQ ID NO. 110 Combined with the Human-Like VL Shown in SEQ ID NO. 168

Plasmid DNA encoding a) for the original murine VH (SEQ ID NO. 2) and VL (SEQ ID NO. 4) and b) for the human-like VH (SEQ ID NO. 110) combined with the human-like VL (SEQ ID NO. 168) was each transformed into E. coli TG1 according to standard protocols. The nucleotide and amino acid sequences of the VH-VL scFv comprising the original murine VH (SEQ ID NO. 2) and VL (SEQ ID NO. 4) are shown in SEQ ID NOs. 9 and 10, respectively. The nucleotide and amino acid sequences of the VH-VL scFv comprising the human-like VH (SEQ ID NO. 110) and the human-like VL (SEQ ID NO. 168) are shown in SEQ ID NOs. 169 and 170, respectively. The nucleotide and amino acid sequences of the VL-VH scFv comprising the human-like VL (SEQ ID NO. 168) and the human-like VH (SEQ ID NO. 110) are shown in SEQ ID NOs. 193 and 194, respectively. Due to different cloning strategies, the amino acid sequence of the VL-VH scFv of SEQ ID NO. 194 shows three amino acid exchanges in comparison to the one of the VH-VL scFv of SEQ ID NO. 170, however, without affecting the binding capacity and specificity of said scFv.

Expression of different clones was performed in E. coli TG-1 in 96-well format. 100 μl LB/0.1% glucose were inoculated with 10 μl of an overnight culture of single clones and grown for 4 h at 37° C. After addition of IPTG to a final concentration of 1 mM, the culture was grown at 30° C. for another 18-20 h. Per well, 40 μl of BEL-buffer (400 mM boric acid, 320 mM NaCl, 4 mM EDTA pH 8.0+2.5 mg/ml lysozyme) were added and shaken at room temperature for 1 h. Cellular debris was eliminated by centrifugation and supernatants were tested in flow cytometric experiments.

The human T cell line HPB-ALL and human and cynomolgus T cells in peripheric blood mononuclear cells (PBMCs) were used as human CD3 and cynomolgus CD3 positive cells, respectively.

Typically 100,000 cells were incubated with 50 μl of the scFv containing bacterial supernatants and incubated for 30 min on ice.

a) HPB-ALL cells were washed three times with PBS and subsequently resuspended in 50 μl PBS containing anti-His antibody (Pentahis, Roche) and further incubated on ice for 30 min. Then the cells were washed three times with PBS and incubated with a PE labeled anti mouse IgG antibody for 30 more min. on ice. After washing the cells for one time the cells were resuspended in a suitable buffer and positivity of cell bound antibody construct determined in a flow cytometer (FACScalibur) and analyzed.

b) Human and cynomolgus PBMCs (containing T cells) were washed three times with PBS and subsequently resuspended in 50 μl PBS containing biotinylated anti-His antibody (biotinylated Pentahis, Roche) and further incubated on ice for 30 min. Then the cells were washed three times with PBS and incubated with PE labeled Streptavidin for 30 more min. on ice. In this step, PBMCs were coincubated with anti-CD2 FITC to identify the T cells in the PBMC mixure.

After washing the cells from a) or b) for one time the cells were resuspended in a suitable buffer and positivity of cell bound antibody construct determined in a flow cytometer (FACScalibur) and analyzed.

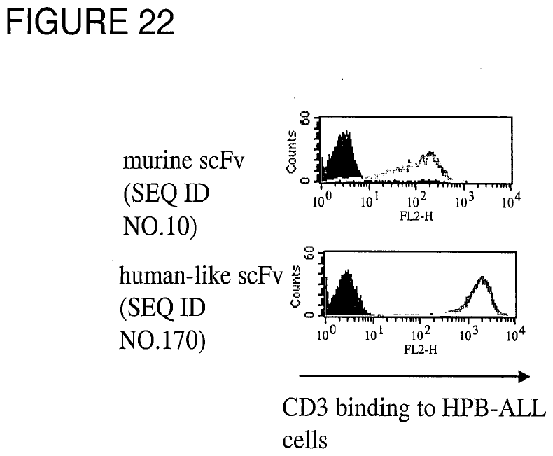

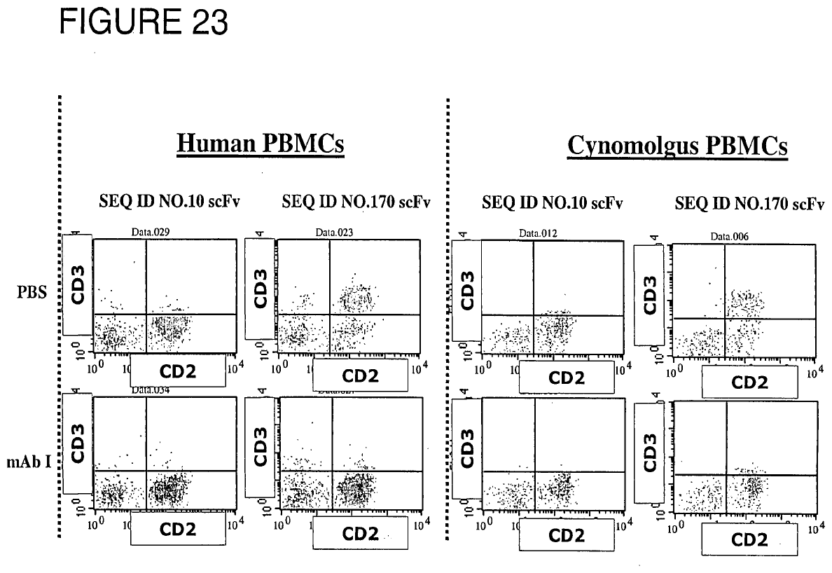

The control scFv of SEQ ID NO. 10 (murine VH of SEQ ID NO. 4-murine VL of SEQ ID NO. 2) shows a clear shift on human CD3 positive cells as depicted in FIG. 22. The shift on human and cynomolgus T cells is less pronounced, most probably due to the less sensitive detection system (FIG. 23).

The human-like scFv of SEQ ID NO. 170 (human-like VH of SEQ ID NO. 110-human-like VL of SEQ ID NO. 168) shows a positive shift on HPB-ALL cells (FIG. 22) and clear shifts on human as well as cynomolgus T cells (FIG. 23, upper panel).

When preincubated with 10 μg/ml of the murine IgG antibody mAb I described in Example 1 having the same specificity as the scFvs (i.e. for CD3 epsilon), the shifts of cells stained with the above-mentioned murine scFv or the human-like scFv decrease significantly, underlining the similar binding region of the scFvs and the original murine antibody; see FIG. 23 lower panel.

### Example 17: Determination of an Epitope for Cross-Species Specific Anti-CD3 Antibodies Binding Both Human and Cynomolgus CD3 Epsilon

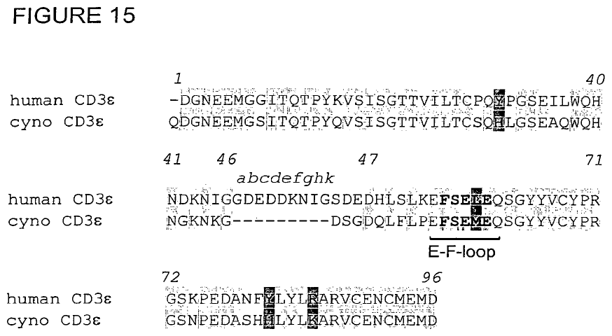

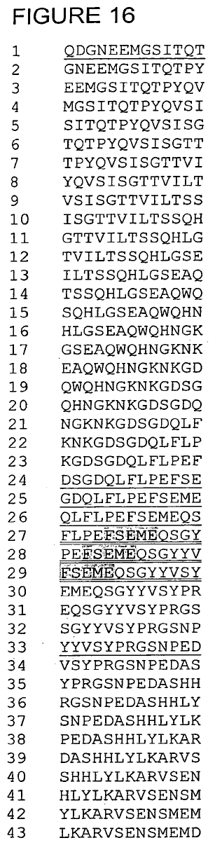

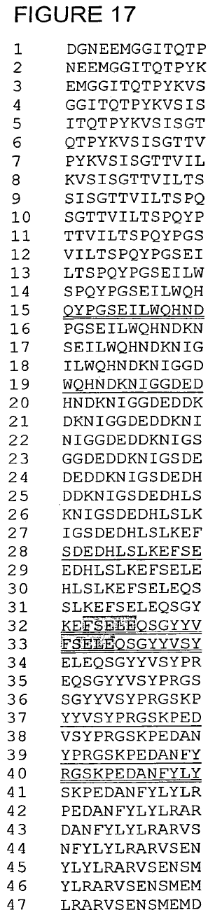

In order to determine the epitope of human and cynomolgus CD3 epsilon bound by cross-species specific anti-CD3 antibodies, epitope mapping was carried out with antibody I (Ig comprising the VH chain shown in SEQ ID NO. 2 and the VL chain shown in SEQ ID NO. 4) and antibody II (Ig comprising the VH chain shown in SEQ ID NO. 6 and the VL chain shown in SEQ ID NO. 8), both binding to human and cynomolgus CD3 epsilon; see also FIG. 1. For the peptide-spotting (“pepspot”) analysis, overlapping 13mer peptides derived from the amino acid sequences of human and cynomolgus CD3 epsilon (see FIG. 15) were covalently linked to a Whatman 50 cellulose-ß-alanine-membrane via the C-terminus while the acetylated N-terminus remained free. In the peptides, the amino acid cystein—wherever occurring in the corresponding CD3 epsilon sequence—was exchanged by the amino acid serin. The individual 13mer peptides generated (by JPT Peptide Technologies GmbH) are shown in FIGS. 16 and 17. For cynomolgus CD3 epsilon, 43 spots have been tested, whereas for the human CD3 epsilon 47 spots have been tested. The length of the overlapping sequence of two adjacent peptides was set to be 11 amino acids.

The pepspot experiments were performed as follows. According to the manufacturer's protocol, the membrane was rinsed with methanol for 1 min, washed with 1×TBS and blocked with 1×TBS/1% (w/v) blocking reagent (BM Chemiluminescence Blotting Substrate (POD) of Roche Diagnostics GmbH) for 3 h. All incubation and washing steps were performed on an orbital shaker at room temperature, except for the overnight incubation of the primary antibody. Directly after discarding the blocking solution, the membranes were incubated overnight with 5 or 3 μg/ml of cross-species specific anti-CD3 antibodies as set forth above in 1×TBS/0.5% (w/v) blocking reagent at 4° C. on an orbital shaker. After washing 4 times with 1×TBS/0.05% Tween for 15 min, detection of bound anti-CD3 antibody was accomplished by incubation for 2 h with a commercially available horseradish-peroxidase-conjugated anti-IgG (F (ab) 2 specific) antibody or an alkaline phosphatase-labeled anti-IgG antibody (diluted according to the manufacturer's recommendation in 1×TBS/0.5% blocking reagent, respectively). Subsequently, the membranes were washed 6 times with 1×TBS/0.05% Tween for 15 min. Horseradish-peroxidase was visualized by enhanced chemiluminescence (luminescence substrate solution A and starting solution B mixed 100:1; BM Chemiluminescence Blotting Substrate (POD) of Roche Diagnostics GmbH) and a BioMax Film (Kodak). Alkaline phosphatase was visualized using 5-bromo-4-chloro-indolyl phosphate/nitro blue tetrazolium liquid substrate system (Sigma).

To exclude unspecific binding of horseradish-peroxidase-conjugated secondary antibody, the membrane was incubated with secondary antibody only. All other steps were performed as in the experiment above.

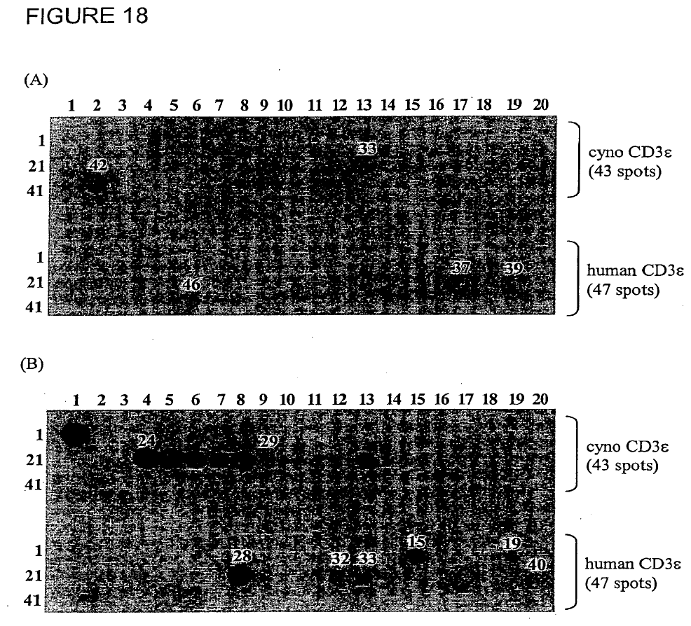

The control pepspot assay (see FIG. 18(A)) showed signals on spots 33 and 42 of cynomolgus CD3 epsilon and on spots 37, 39 and 46 of human CD3 epsilon. These signals are regarded as unspecific and will not be mentioned further.

### 1. Anti-CD3 Antibody I (Ig Comprising the VH Chain Shown in SEQ ID NO. 2 and the VL Chain Shown in SEQ ID NO. 4)

### (i) Binding on Cynomolgus CD3 Epsilon

Strong binding signals of cross-species specific anti-CD3 antibody I (Ig comprising the VH chain shown in SEQ ID NO. 2 and the VL chain shown in SEQ ID NO. 4) to peptides derived from cynomolgus CD3 epsilon were detected on spot 1 as well as on the stretch of peptide-spots 24-29 (FIG. 18(B)). The latter corresponds to amino acid residues 47-69 of cynomolgus CD3 epsilon (see FIG. 15). All 13mer peptides covering this region contain one minimal amino acid motif 56-59 (EFSE). Spot 1 corresponds to amino acid residues 1-13 (QDGNEEMGSITQT) of cynomolgus CD3 epsilon.

### (ii) Binding on Human CD3 Epsilon

Cross-species specific anti-CD3 antibody I bound to peptide-spots 15, 28, 32, 33 and 40 derived from human CD3 epsilon (see FIG. 18(B)). The stretch of peptide-spots 28 to 33 corresponds to the amino acid residues 47-69 of human CD3 epsilon and comprises the minimal amino acid motif 57-59 (FSE). Spots 15 and 40 correspond to amino acid residues 30-42 (QYPGSEILWQHND) and 71-83 (RGSKPEDANFYLY), respectively.

### 2. Anti-CD3 Antibody II (Ig Comprising the VH Chain Shown in SEQ ID NO. 6 and the VL Chain Shown in SEQ ID NO. 8)

### (i) Binding on Cynomolgus CD3 Epsilon

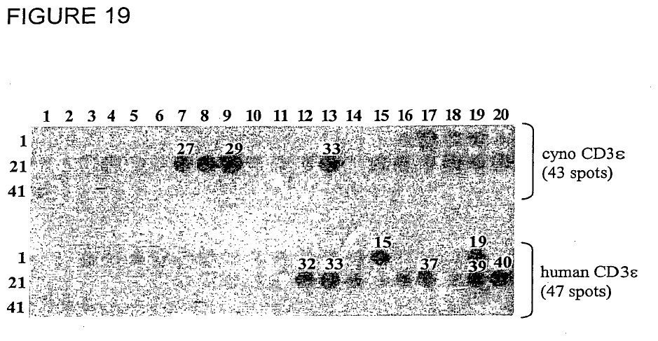

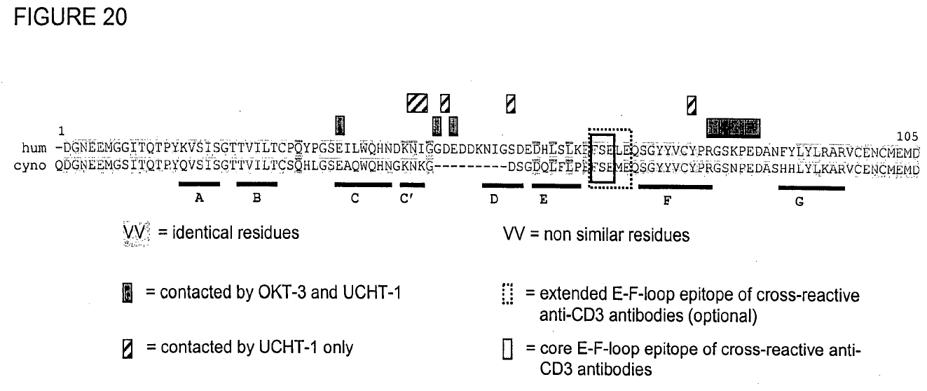

The pepspot analysis with cross-species specific anti-CD3 antibody II (Ig comprising the VH chain shown in SEQ ID NO. 6 and the VL chain shown in SEQ ID NO. 8) showed strong signals to cynomolgus CD3 epsilon on the stretch of peptide-spots 27-29 as well as on spot 33 (see FIG. 19). The stretch spanning spots 27 and 29 corresponds to the amino acid residues 53-69 of cynomolgus CD3 epsilon (see FIG. 15), wherein the 13mer peptides have the minimal stretch of amino acids 57-61 (FSEME) in common. Spot 33 correlates with amino acid residues 65-77 (YYVSYPRGSNPED).

### (ii) Binding on Human CD3 Epsilon

Cross-reactive anti-CD3 antibody II bound the peptide-spots 15, 19, 32 and 33, 37, 39 and 40 of human CD3 epsilon (see FIG. 19). Spot 19 corresponds to amino acid residues 38-46d (WQHNDKNIGGDED) of human CD3 epsilon (see FIG. 15). The small stretch of spots 32 to 33 corresponds to amino acid residues 55-69 containing the minimal peptide FSELE (amino acids 57-61). The spots 37 and 39 match amino acid residues 65-77 (YYVSYPRGSKPED) and 69-81 (YPRGSKPEDANFY) of human CD3 epsilon, respectively. The correlations of spots 15 and 40 are already mentioned above.

In summary, both cross-species specific anti-CD3 antibodies recognize discontinous epitopes on human and cynomolgus CD3 epsilon. Regarding cynomolgus CD3 epsilon both cross-species specific anti-CD3 antibodies recognized a clear overlapping stretch of peptide-spots 27-29 (see FIG. 16). All 13mer peptides covering this region contain one minimal peptide FSEME (amino acid residues 57-61 of cynomolgus CD3 epsilon). The peptide-intersection on human CD3 epsilon bound by both antibodies can be determined for spots 32 and 33 (see FIG. 17). This section contains the minimal peptide FSELE corresponding to residues 57-61 of human CD3 epsilon.

Based on these results it is concluded that cross-species specific CD3 antibody fragments contact CD3 epsilon in the area of amino acid residues 57-61 of both cynomolgus and human CD3 epsilon comprising the amino acid stretches FSEME and FSELE of cynomolgus and human CD3 epsilon, respectively, with the motif FSE forming the epitope core. This result—although plausible because of the accessibility of the E-F-loop (amino acids 56-62; see FIG. 15) of human CD3 epsilon (Kjer-Nielsen et al., PNAS 101 (2004), p. 7675-80) comprising the amino acids FSELE or FSEME—is nevertheless surprising since there is no overlap of this newly defined epitope with the known epitope on the CD3 epsilon-chain of anti-CD3 antibodies OKT3 and UCHT1 (see FIG. 17; Kjer-Nielsen et al., loc.cit; Arnett et al., PNAS 101 (2004), p. 16268-73) which have so far been regarded as representative of all anti-CD3 antibodies thought to form a single family with the same or a very similar epitope.

### Example 18: Determination of the Epitope for the Human-Like Cross-Species Specific Anti-CD3 Antibody Binding Both to Human and Cynomolgus CD3 Epsilon

The epitope mapping of the human-like cross-species specific anti-CD3 antibody fragment described in Example 16 (SEQ ID NO. 170) was carried out by peptide-spotting (“pepspot”) analysis as described in Example 17. For this purpose, said single chain Fv fragment shown in SEQ ID NO. 170 was converted into a full IgG antibody with a murine gamma1 heavy chain comprising the VH region as shown in SEQ ID NO. 110 and a kappa light chain comprising the VL region as shown in SEQ ID NO. 168. The procedure of the pepspot experiment was identical to the protocol used in Example 17.

The pepspot membrane was incubated with 4 μg/ml of the mentioned IgG1 antibody, and an alkaline phosphatase-labeled goat-anti-mouse IgG antibody detecting bound CD3 antibody. A second membrane was incubated with alkaline phosphatase-labeled goat-anti-mouse IgG antibody alone to reveal unspecific binding of the detection antibody.

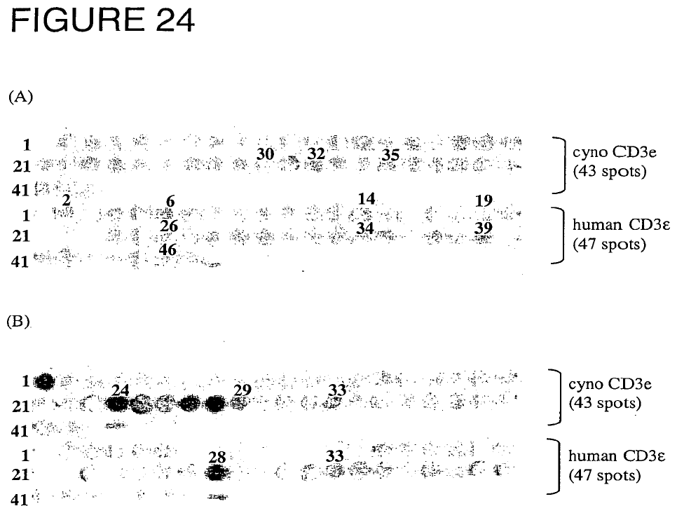

The following signals detected in the control pepspot assay (see FIG. 24(A)) have been regarded as unspecific and will not be mentioned further: the stained spot-stretches 10-13, 15-19, 30-32, 35-41 of cynomolgus CD3 epsilon and 2-6, 14-19, 26, 34-39 and 46 of human CD3 epsilon.

### (i) Binding on Cynomolgus CD3 Epsilon

The cross-species specific anti-CD3 antibody (murine IgG1 comprising the VH chain shown in SEQ ID NO. 110 and the VL chain shown in SEQ ID NO. 168) bound to the peptide-spots 1 and 33 as well as to the amino acid stretch of peptide-spots 24-29 (FIG. 24(B)) derived from cynomolgus CD3 epsilon. The stretch spanning spots 24 and 29 corresponds to the amino acid residues 47-69 of cynomolgus CD3 epsilon (see FIGS. 15 and 16), wherein the 13mer peptides have the minimal stretch of amino acids 56-59 (EFSE) in common. Spot 1 and spot 33 correspond to amino acid residues 1-13 (“QDGNEEMGSITQT”; SEQ ID NO. 199) and 65-77 (“YYVSYPRGSNPED”; SEQ ID NO. 200) of cynomolgus CD3 epsilon, respectively.

### (ii) Binding on Human CD3 Epsilon

Binding signals of the mentioned cross-species specific anti-CD3 IgG1 antibody to peptides derived from human CD3 epsilon (see FIG. 24(B)) were found on spots 28 and 33, which correspond to the amino acid residues 47-59 and 57-69 of human CD3 epsilon (see FIG. 17), respectively. The two stained spots comprise the minimal amino acid motif 57-59 (FSE).

The human-like cross-species specific anti-CD3 antibody recognizes the same discontinuous epitopes on human and cynomolgus CD3 epsilon as antibody I and II described in Examples 1 and 17. Binding signals of said human-like antibody on the peptide membrane reveal the peptide-intersections corresponding to the amino acid sequence “FSEME” (amino acid residues 57-61) of cynomolgus CD3 epsilon and those corresponding to amino acid sequence “FSELE” (amino acid residues 57-61) of human CD3 epsilon as core region. This is in line with the epitope determined for the cross-species specific anti-CD3 antibodies I and II on both cynomolgus and human CD3 epsilon (see Example 17).

### Example 19: Verification of the Identified Epitope on Human CD3 Epsilon for the Human-Like Cross-Species Specific Anti-CD3 Antibody

To verify the epitope of the human-like cross-species specific anti-CD3 antibody fragment described in Example 16 on human CD3 epsilon, the identified binding region as determined in Experiment 18 was further analyzed by a dot-blotting assay using a 13mer peptide covering the defined binding area of amino acid residues “FSELE” on human CD3 epsilon. This peptide comprises the amino acid sequence “EFSELEQSGYYVC” (SEQ ID NO. 195) of human CD3 epsilon. The peptide exists in two forms and is either biotinylated N- or C-terminally. In case of the N-terminal labelling, a short linker connects the peptide with the biotin. As described in Example 18, the antibody fragment was converted to a murine IgG format with a murine gamma1 heavy chain comprising the VH region as shown in SEQ ID NO. 110 and a kappa light chain comprising the VL region as shown in SEQ ID NO. 168. The dot blotting was performed as follows. The Minifold I Spot Blot System from Schleicher & Schuell was used for immobilizing the peptides on a nitrocellulose membrane (Protran BA 85, 0.45 μm). 75 μg of each peptide in 100 μl TBS were filtered through the membrane using vacuum. After the filtration step the membrane was blocked with 1×TBS/1% (w/v) blocking reagent (BM Chemiluminescence Blotting Substrate (POD) of Roche Diagnostics GmbH) for 2 h. All incubation and washing steps were performed on an orbital shaker at room temperature, except for the overnight incubation of the primary antibody. Directly after discarding the blocking solution, the membrane was incubated overnight with 3 μg/ml of the above-mentioned anti-CD3 antibody in 1×TBS/0.5% (w/v) blocking reagent at 4° C. on an orbital shaker. As a control, the anti-CD3 murine IgG1 antibody UCHT1 (BD Biosciences) binding to human CD3 epsilon was applied to a second membrane blotted with the same amounts of the two peptides. After washing three times with 1×TBS/0.05% Tween for 10 min, detection of bound anti-CD3 antibody was accomplished by incubation for 2 h with a commercially available alkaline phosphatase-conjugated anti-IgG antibody (diluted according to the manufacturer's recommendation in 1×TBS/0.5% blocking reagent). Subsequently, the membranes were washed three times with 1×TBS/0.05% Tween for 10 min. Alkaline phosphatase was visualized using 5-bromo-4-chloro-indolyl phosphate/nitro blue tetrazolium liquid substrate system (Sigma).

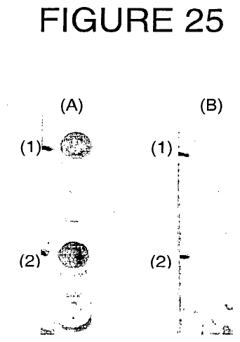

The mentioned CD3 specific antibody comprising the VH region shown in SEQ ID NO. 110 and the VL region shown in SEQ ID NO. 168 bound to both forms of the peptide “EFSELEQSGYYVC” (SEQ ID NO. 195) blotted to the membrane (see FIGS. 25(A)(1) and (2)), whereas no binding could be obtained for the anti-CD3 murine IgG antibody UCHT1 (see FIG. 25(B) (1) und (2)). The epitope recognized by anti-CD3 antibody UCHT1 is described e.g. in Kjer-Nielsen et al., loc.cit; Arnett et al., PNAS (2204), p.16268-73. These results support the identification of the newly defined epitope of the herein-described anti-CD3 antibody (with the VH region shown in SEQ ID NO. 110 and the VL region shown in SEQ ID NO. 168). Said epitope corresponds to the amino acid residues “EFSELEQSGYYVC” (SEQ ID NO. 195) on the human CD3 epsilon chain and comprises the amino acid stretch “FSELE”.

### Example 20: Generation of CHO Cells Transfected with Cynomolgus EGFR

A shock frozen piece of EGFR positive cynomolgus colon was used to obtain the total RNA that was isolated according to the instructions of the kit manual (Qiagen, RNeasy Mini Kit). The obtained RNA was used for cDNA synthesis by random-primed reverse transcription. For cloning of the full length sequence of the EGFR antigen the following oligonucleotides were used:

5′ EGFR AG XbaI
(SEQ ID NO. 197)
5′-GGTCTAGAGCATGCGACCCTCCGGGACGGCCGGG-3′
3′ EGFR AG SalI
(SEQ ID NO. 198)
5′-TTTTAAGTCGACTCATGCTCCAATAAATTCACTGCT-3′.

A PCR (denaturation at 93° C. for 5 min, annealing at 58° C. for 1 min, elongation at 72° C. for 2 min for the first cycle; denaturation at 93° C. for 1 min, annealing at 58° C. for 1 min, elongation at 72° C. for 2 min for 30 cycles; terminal extension at 72° C. for 5 min) was used to amplify the coding sequence. The PCR product was subsequently digested with XbaI and SalI, ligated into the appropriately digested expression vector pEF-DHFR, and transformed into E. coli. The afore-mentioned procedures were carried out according to standard protocols (Sambrook, Molecular Cloning; A Laboratory Manual, 3rd edition, Cold Spring Harbour Laboratory Press, Cold Spring Harbour, New York (2001)). A clone with sequence-verified nucleotide sequence was transfected into DHFR deficient CHO cells for eukaryotic expression of the construct. Eukaryotic protein expression in DHFR deficient CHO cells was performed as described in Kaufmann R. J. (1990) Methods Enzymol. 185, 537-566. Gene amplification of the construct was induced by increasing concentrations of MTX to a final concentration of up to 20 nM MTX.

### Example 21: Generation of EGFR and CD3 Cross-Species Specific Bispecific Single Chain Antibodies

Generally, bispecific single chain antibody molecules, each comprising a domain with a binding specificity for the human and the cynomolgus CD3 antigen as well as a domain with a binding specificity for the human and the cynomolgus EGFR antigen, were designed as set out in the following Table 1:

TABLE 1
Formats of anti-CD3 and anti-EGFR cross-species specific
bispecific single chain antibody molecules

| SEQ ID | Formats of protein constructs |
|---|---|
| (nucl/prot) | (N → C) |
| 171/172 | EGFR HL × SEQ ID NO. 170 |
| 173/174 | EGFR LH × SEQ ID NO. 170 |
| 175/176 | EGFR HL × SEQ ID NO. 194 |
| 177/178 | EGFR LH × SEQ ID NO. 194 |
| 179/180 | SEQ ID NO. 170 × EGFR HL |
| 181/182 | SEQ ID NO. 194 × EGFR HL |
| 183/184 | SEQ ID NO. 170 × EGFR LH |
| 185/186 | SEQ ID NO. 194 × EGFR LH |

The afore-mentioned constructs containing the variable light-chain (L) and variable heavy-chain (H) domains reactive with the human and cynomolgus EGFR derived from murine hybridomas were obtained by gene synthesis and subsequent cloning into an expression vector comprising the CD3 specific VH and VL combinations reactive with the human and cynomolgus CD3. Herein, SEQ ID NO. 170 corresponds to amino acid sequence of the anti-CD3 VH-VL scFv comprising the human-like VH (SEQ ID NO. 110) and the human-like VL (SEQ ID NO. 168). SEQ ID NO. 194 corresponds to the amino acid sequence of the anti-CD3 VL-VH scFv comprising the human-like VL (SEQ ID NO. 168) and the human-like VH (SEQ ID NO. 110). The constructs were then transfected into DHFR-deficient CHO-cells by electroporation.

### Example 22: Expression and Purification of the EGFR and CD3 Cross-Species Specific Bispecific Single Chain Antibodies

The bispecific single chain antibodies were expressed in chinese hamster ovary cells (CHO). Eukaryotic protein expression in DHFR deficient CHO cells was performed as described in Kaufmann R. J. (1990) Methods Enzymol. 185, 537-566. Gene amplification of the constructs were induced by increasing concentrations of MTX to a final concentration of up to 20 nM MTX. After two passages of stationary culture the cells were grown in roller bottles with CHO modified MEM medium for 7 days before harvest. The cells were removed by centrifugation and the supernatant containing the expressed protein was stored at −20° C.

Äkta® FPLC System (Pharmacia) and Unicorn® Software were used for chromatography. All chemicals were of research grade and purchased from Sigma (Deisenhofen) or Merck (Darmstadt). Immobilized metal affinity chromatography (“IMAC”) was performed using a Fractogel® column (Merck) which was loaded with ZnCl₂ according to the protocol provided by the manufacturer. The column was equilibrated with buffer A2 (20 mM sodium phosphate buffer pH 7.5, 0.4 M NaCl) and the cell culture supernatant (500 ml) was applied to the column (10 ml) at a flow rate of 3 ml/min. The column was washed with buffer A2 to remove unbound sample. Bound protein was eluted using a two step gradient of buffer B2 (20 mM sodium phosphate buffer pH 7.5, 0.4 M NaCl, 0.5 M Imidazol) according to the following:
Step 1: 20% buffer B2 in 6 column volumes;
Step 2: 100% buffer B2 in 6 column volumes.

Eluted protein fractions from step 2 were pooled for further purification.

Gel filtration chromatography was performed on a Sephadex S200 HiPrep column (Pharmacia) equilibrated with PBS (Gibco). Eluted protein samples (flow rate 1 ml/min) were subjected to standard SDS-PAGE and Western Blot for detection. Prior to purification, the column was calibrated for molecular weight determination (molecular weight marker kit, Sigma MW GF-200). Protein concentrations were determined using protein assay dye (MicroBCA, Pierce) and IgG (Biorad) as standard protein.

The bispecific single chain antibodies were isolated in a two step purification process of IMAC and gel filtration. The main product had a molecular weight of about 52 kDa under native conditions as determined by gel filtration in PBS. This molecular weight corresponds to the bispecific single chain antibody. All constructs were purified according to this method.

Purified bispecific single chain antibody protein was analyzed in SDS PAGE under reducing conditions performed with pre-cast 4-12% Bis Tris gels (Invitrogen). Sample preparation and application were performed according to the protocol provided by the manufacturer. The molecular weight was determined with MultiMark protein standard (Invitrogen). The gel was stained with colloidal Coomassie (Invitrogen protocol). The purity of the isolated protein was >95% as determined by SDS-PAGE.

Western Blot was performed using an Optitran® BA-S83 membrane and the Invitrogen Blot Module according to the protocol provided by the manufacturer. The antibodies used were directed against the His Tag (Penta His, Qiagen) and Goat-anti-mouse Ig labeled with alkaline phosphatase (AP) (Sigma), and BCIP/NBT (Sigma) as substrate. The bispecific single chain antibody could be specifically detected by Western Blot. A single band was detected at 52 kD corresponding to the purified bispecific molecule.

### Example 23: Flow Cytometric Binding Analysis of the EGFR and CD3 Cross-Species Specific Bispecific Antibodies

In order to test the functionality of the cross-species specific bispecific antibody constructs with regard to binding capability to human and cynomolgus EGFR and CD3, respectively, a FACS analysis was performed. For this purpose the EGFR positive epidermoid carcinoma A431 cells (ATCC, CRL-1555) and CD3 positive human T cell leukemia cell line HPB-ALL (DSMZ, Braunschweig, ACC483) were used to check the binding to human antigens. The binding reactivity to cynomolgus antigens was tested by using the generated cynomolgus EGFR transfectants described in Example 20 and cynomolgus PBMCs which were obtained by Ficoll density gradient centrifugation. 200,000 cells of the respective cell population were incubated for 30 min on ice with 50 μl of the purified protein of the cross-species specific bispecific antibody constructs (1 μg/ml). The cells were washed twice in PBS and binding of the construct was detected with an unlabeled murine Penta His antibody (diluted 1:20 in 50 μl PBS with 2% FCS; Qiagen; Order No. 34660). After washing, bound anti His antibodies were detected with an Fc gamma-specific antibody (Dianova) conjugated to phycoerythrin, diluted 1:100 in 50 μl PBS with 2% FCS. Fresh culture medium was used as a negative control.

Cells were analyzed by flow cytometry on a FACS-Calibur apparatus (Becton Dickinson, Heidelberg). FACS staining and measuring of the fluorescence intensity were performed as described in Current Protocols in Immunology (Coligan, Kruisbeek, Margulies, Shevach and Strober, Wiley-Interscience, 2002).

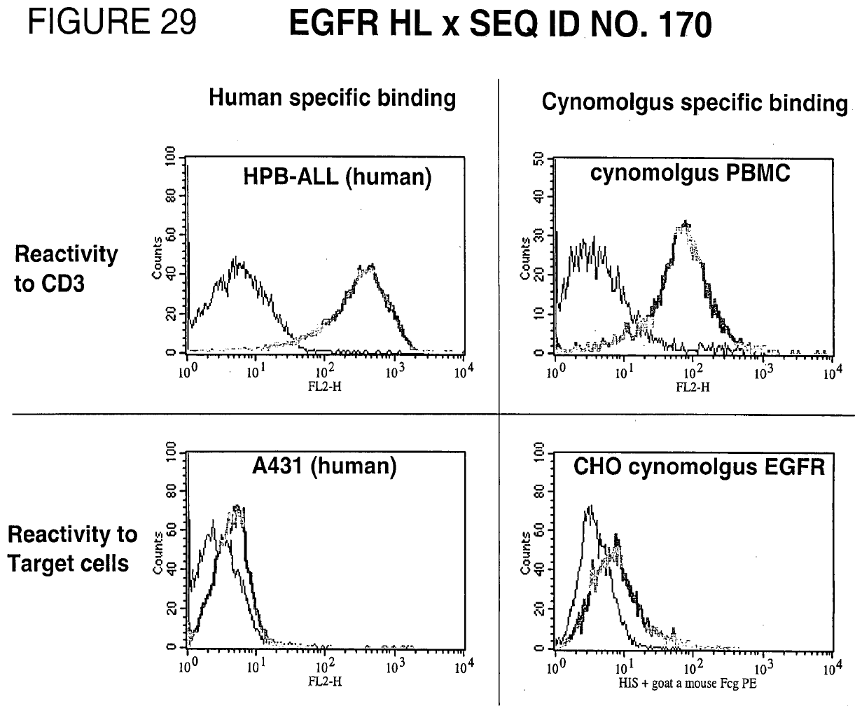

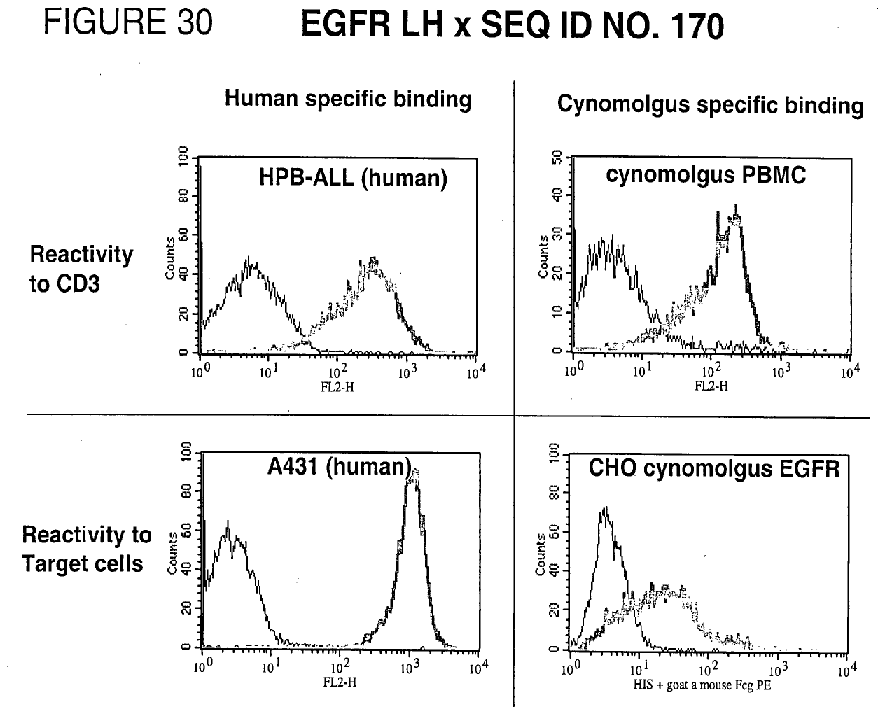

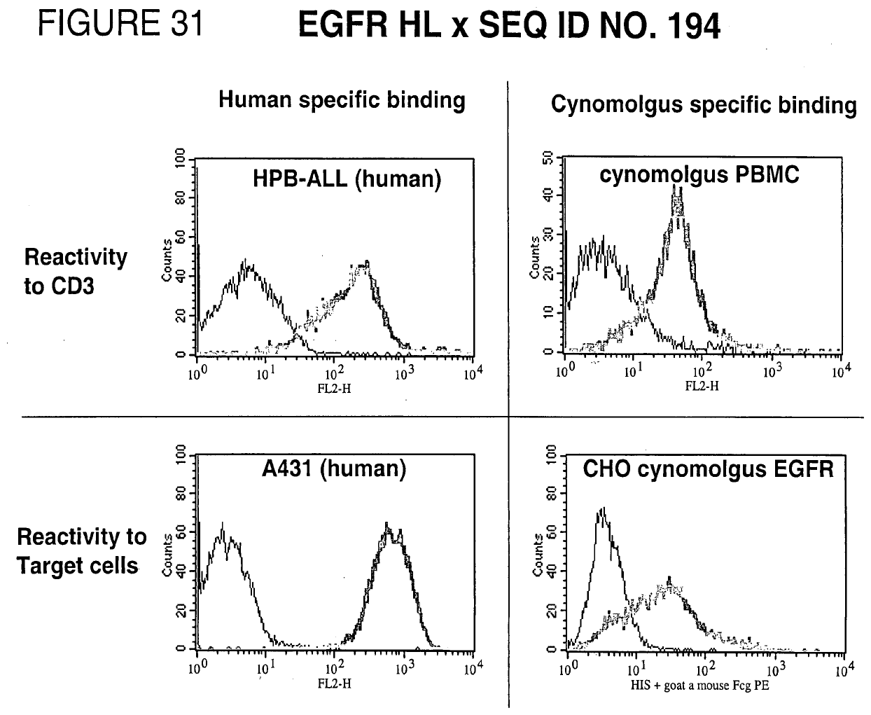

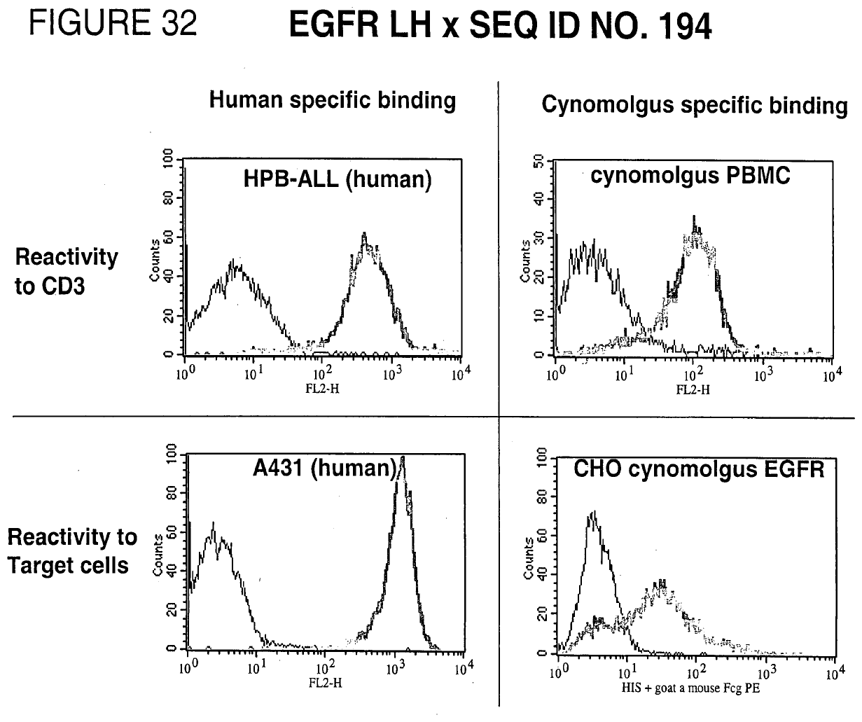

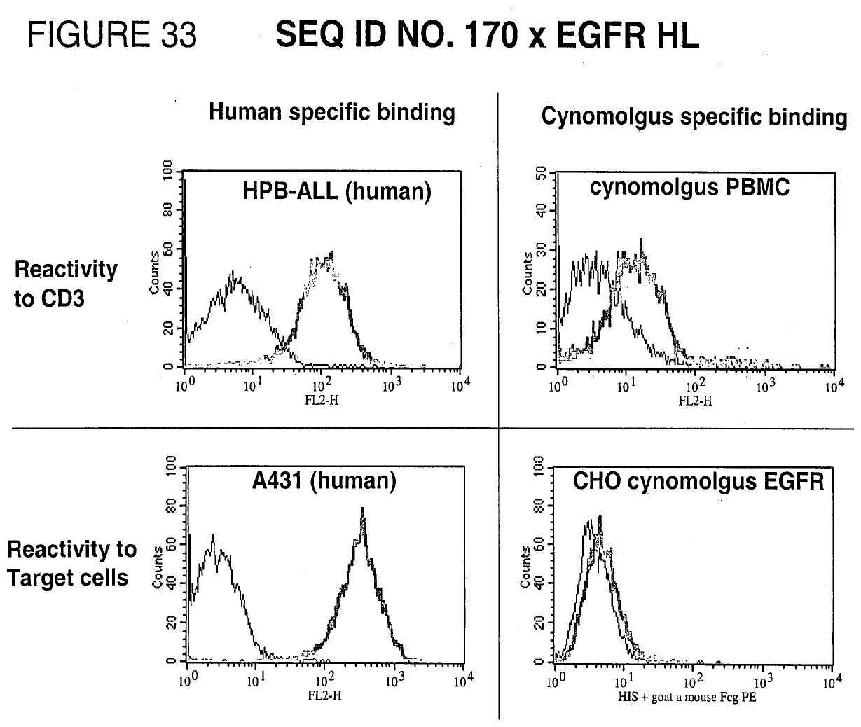

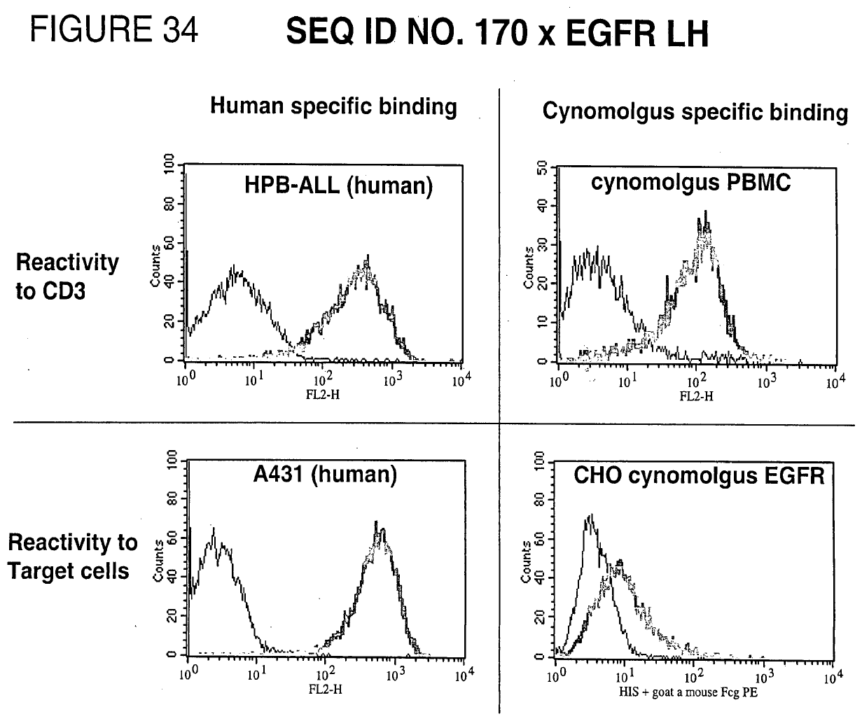

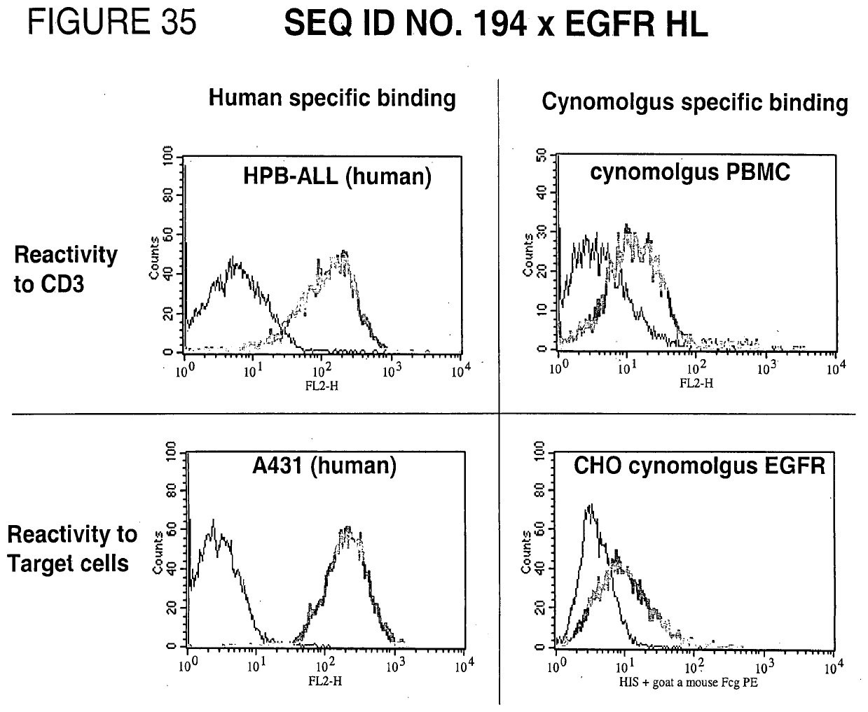

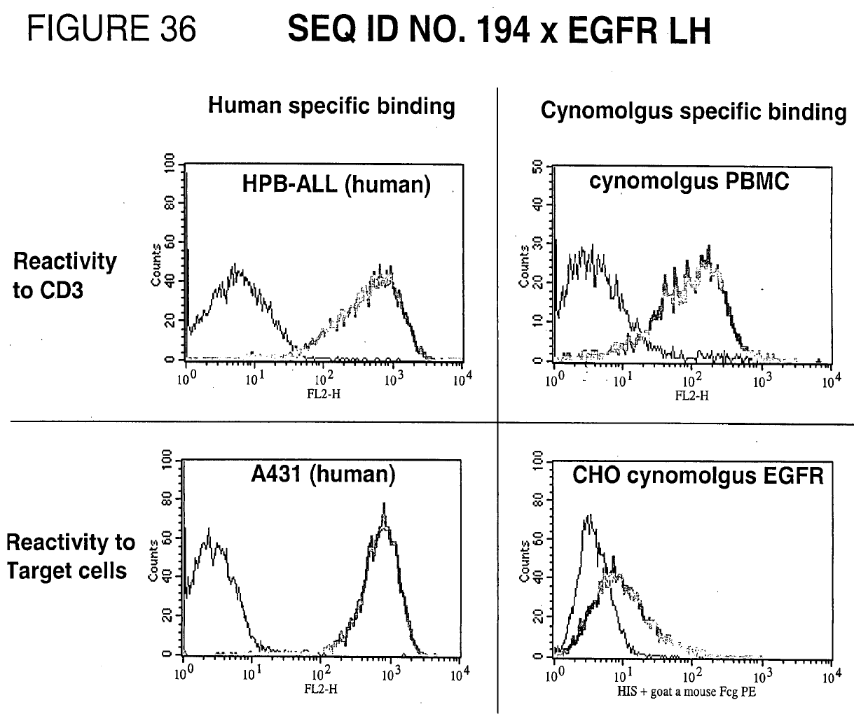

The binding ability of several domain arrangements were clearly detectable as shown in FIGS. 29 to 36. In FACS analysis, all constructs with different arrangement of VH and VL domains specific for EGFR and CD3 showed binding to CD3 and EGFR compared to the negative control using culture medium and 1, and 2. detection antibody. In summary, the cross-species specificity of the bispecific antibody to human and cynomolgus CD3 and EGFR antigens could clearly be demonstrated.

### Example 24: Bioactivity of EGFR and CD3 Cross-Species Specific Bispecific Single Chain Antibodies

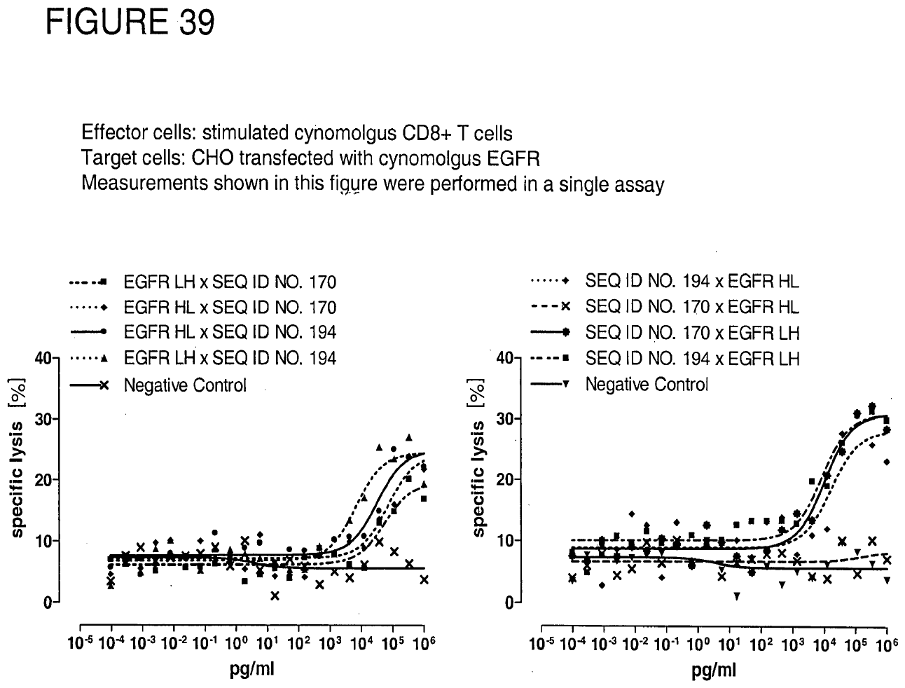

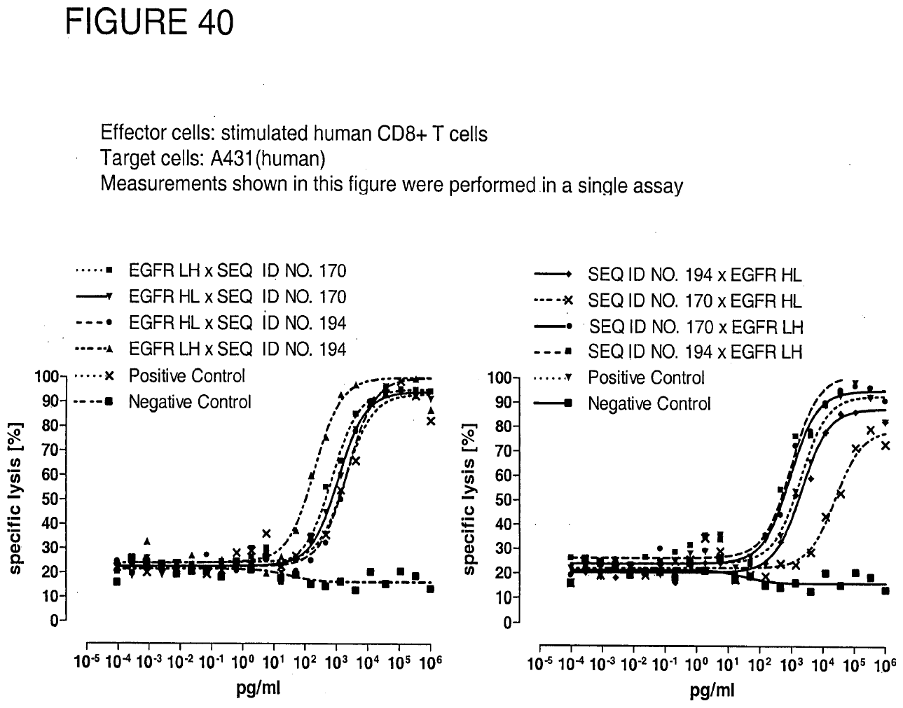

Bioactivity of the generated bispecific single chain antibodies was analyzed by chromium 51 release in vitro cytotoxicity assays using the EGFR positive cell lines described in Example 23; see also FIGS. 39 and 40. As effector cells stimulated human CD8 positive T cells or stimulated cynomolgus PBMCs were used, respectively.

The generation of the stimulated CD8+ T cells was performed as follows:

A Petri dish (85 mm diameter, Nunc) was pre-coated with a commercially available anti-CD3 specific antibody in a final concentration of 1 μg/ml for 1 hour at 37° C. Unbound protein was removed by one washing step with PBS. The fresh PBMC's were isolated from peripheral blood (30-50 ml human blood or 10 ml cynomolgus blood) by Ficoll gradient centrifugation according to standard protocols. 3-5×10⁷ PBMCs were added to the precoated petri dish in 50 ml of RPMI 1640/10% FCS/IL-2 20 U/ml (Proleukin, Chiron) and stimulated for 2 days. At the third day the cells were collected, washed once with RPMI 1640. IL-2 was added to a final concentration of 20 U/ml and cultivated again for one day. The CD8+ CTLs were isolated by depleting CD4+ T cells an CD56+NK cells.

Target cells were washed twice with PBS and labeled with 11,1 MBq⁵¹Cr in a final volume of 100 μl RPMI with 50% FCS for 45 minutes at 37° C. Subsequently the labeled target cells were washed 3 times with 5 ml RPMI and then used in the cytotoxicity assay. The assay was performed in a 96 well plate in a total volume of 250 μl supplemented RPMI (as above) with an ET ratio of 10:1 corresponding to 1000 target cells and 10000 effector cells per well. 1 μg/ml of the cross-species specific bispecific single chain antibody molecules and 20 threefold dilutions thereof were applied. The assay time was 18 hours and cytotoxicity was measured as relative values of released chromium in the supernatant related to the difference of maximum lysis (addition of Triton-X) and spontaneous lysis (without effector cells). All measurements were done in quadruplicates. Measurement of chromium activity in the supernatants was performed with a Wizard 3 gammacounter (Perkin Elmer Life Sciences GmbH, Köln, Germany). Analysis of the experimental data was performed with Prism 4 for Windows (version 4.02, GraphPad Software Inc., San Diego, California, USA). Sigmoidal dose response curves typically had R² values >0.90 as determined by the software. EC50 values calculated by the analysis program were used for comparison of bioactivity.

As shown in FIGS. 39 and 40, all of the generated cross-species specific bispecific single chain antibody constructs revealed cytotoxic activity against human EGFR positive target cells elicited by human CD8+ cells and cynomolgus EGFR positive target cells elicited by cynomolgus CD8+ cells. In FIG. 39, a bispecific single chain antibody with a variable domain reactive with EGFR and a de-immunized human CD3-specific variable domain (EGFR LH×di-anti CD3) has been used as a negative control. In FIG. 40, the same construct (EGFR LH×di-anti CD3) has been used as a positive control. As a negative control, an irrelevant bispecific single chain antibody has been used.

### Example 25: Generation and Characterization of Carboanhydrase IX (CAIX) and CD3 Cross-Species Specific Bispecific Single Chain Antibodies

TABLE 2
Formats of CAIX and CD3 cross-species specific bispecific single
chain antibodies

| SEQ ID | Formats of protein constructs |
|---|---|
| (nucl/prot) | (N → C) |
| 189/190 | CAIX HL × SEQ ID NO. 170 |
| 191/192 | CAIX LH × SEQ ID NO. 170 |
| 187/188 | CAIX HL × SEQ ID NO. 194 |

In analogy to the afore-mentioned Examples, Carboanhydrase IX (CAIX/MN) and CD3 cross-species specific bispecific single chain antibodies containing the variable light-chain (L) and variable heavy-chain (H) domains reactive with the human and cynomolgus CAIX antigen were created and subsequently cloned into an expression vector comprising the CD3 specific VH and VL combinations reactive with the human and cynomolgus CD3. The experiments were carried out in essence as described in Examples 20 to 24, with the following exceptions:

The FACS binding experiments were performed with the CAIX positive human lung carcinoma cell line A549 (ATCC, CCL-185) to assess the binding capability to the human CAIX antigen. The cross-species specificity to cynomolgus tissue was tested by deploying the cynomolgus skin cell line CYNOM-K1 (National Institute for Cancer Research (IST) of Genova, Italy, ECACC 90071809) or the rhesus monkey epithelial cell line 4MBr-5 (ATCC, CCL-208). The same changes in cell lines apply to the cytotoxicity assays performed with the CAIX and CD3 cross-species specific bispecific single chain antibodies.

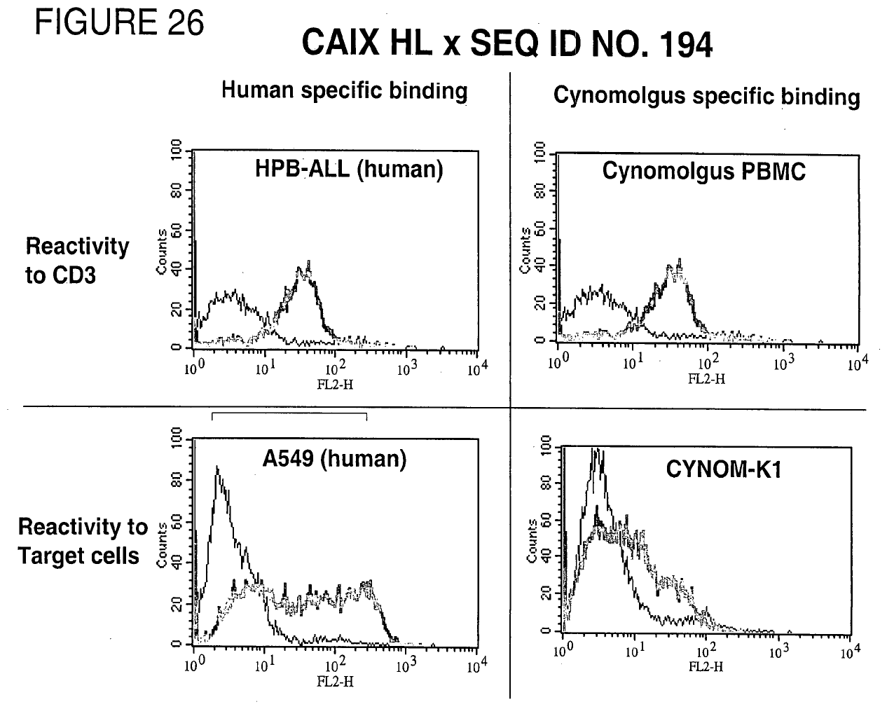

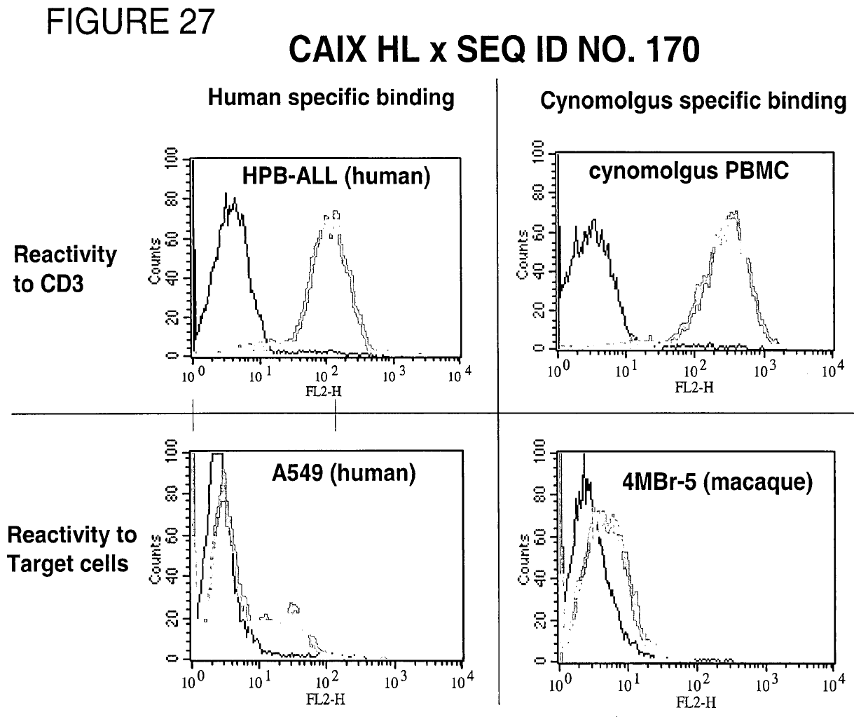

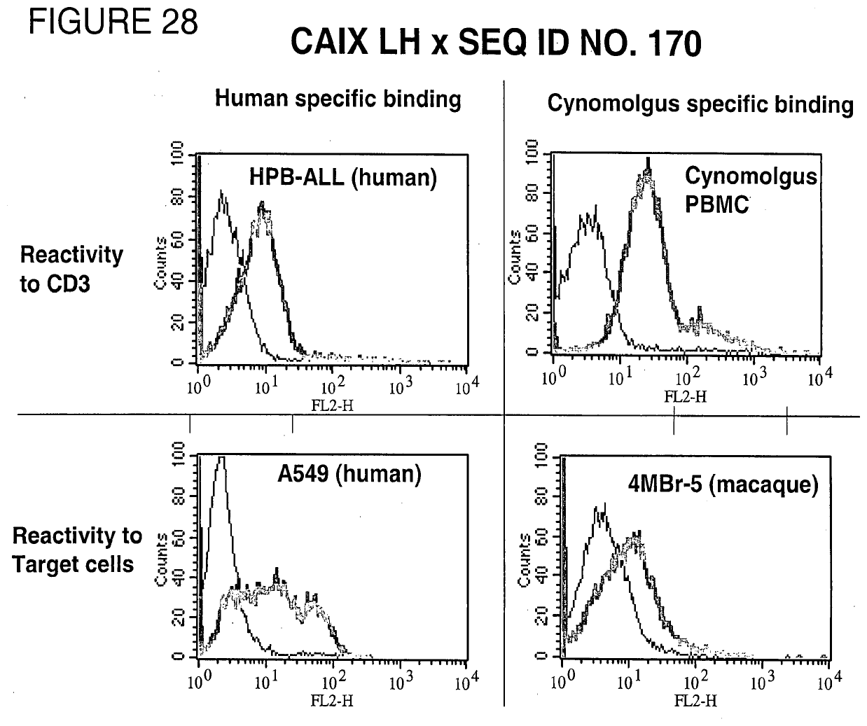

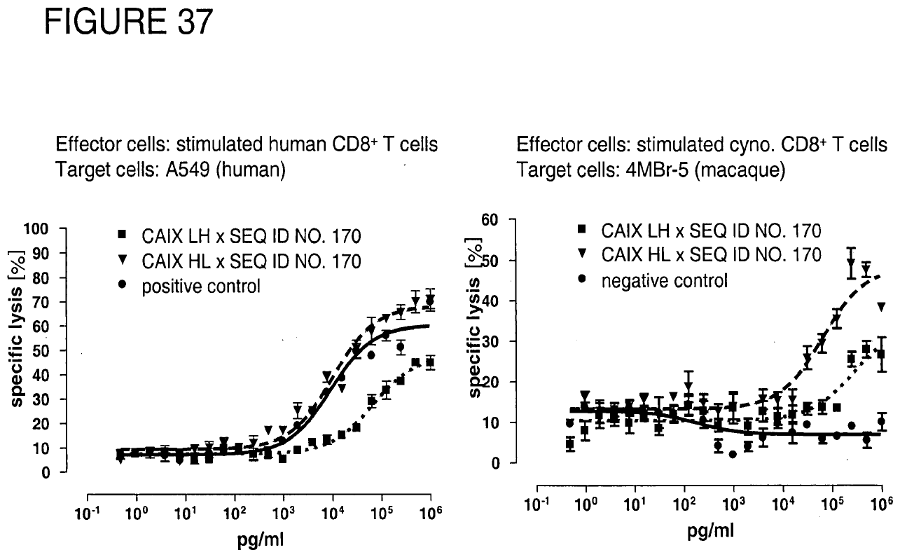

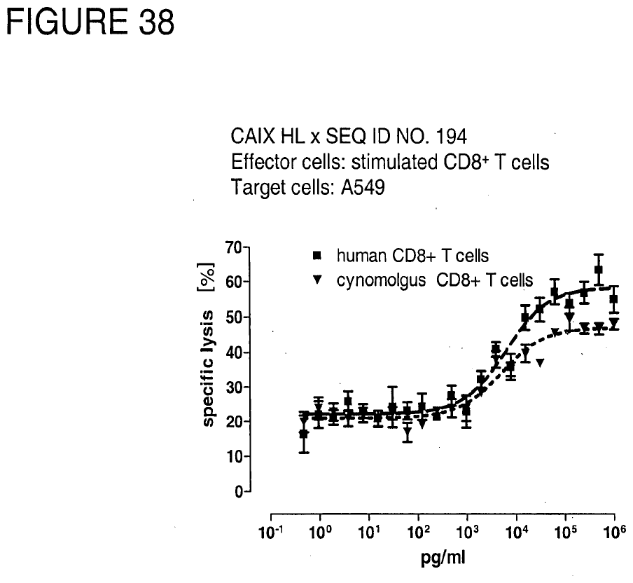

As depicted in FIGS. 26 to 28, the generated CAIX and CD3 cross-species specific bispecific single chain antibodies demonstrated binding to both the human and cynomolgus antigens and proved to be fully cross-species specific. The cytolytic bioactivity of the analysed constructs is shown in FIGS. 37 and 38. In the left panel of FIG. 37, a bispecific single chain antibody with a variable domain reactive with CAIX and a de-immunized human CD3-specific variable domain has been used as a positive control. In the right panel, the same construct has been used as a negative control.

APPENDIX

| SEQ | | | | |
|---|---|---|---|---|
| ID | DESIG- | | | |
| NO. | NATION | SOURCE | TYPE | SEQUENCE |
| 1 | VH | Murine | NA | gaggtgaagcttctcgagtctg |
| | | | | gaggaggattggtgcagcctaa |
| | | | | agggtcattgaaactctcatgt |
| | | | | gcagcctctggattcaccttca |
| | | | | atacctacgccatgaactgggt |
| | | | | ccgccaggctccaggaaagggt |
| | | | | ttggaatgggttgctcgcataa |
| | | | | gaagtaaatataataattatgc |
| | | | | aacatattatgccgattcagtg |
| | | | | aaagacaggttcaccatctcca |
| | | | | gagatgattcacaaagcattct |
| | | | | ctatctacaaatgaacaacttg |
| | | | | aaaactgaggacacagccatgt |
| | | | | actactgtgtgagacatgggaa |
| | | | | cttcggtaatagctacgtttcc |
| | | | | tggtttgcttactggggccaag |
| | | | | ggactctggtcactgtctctgc |
| | | | | a |
| 2 | VH | Murine | AA | EVKLLESGGGLVQPKGSLKLSC |
| | | | | AASGFTFNTYAMNWVRQAPGKG |
| | | | | LEWVARIRSKYNNYATYYADSV |
| | | | | KDRFTISRDDSQSILYLQMNNL |
| | | | | KTEDTAMYYCVRHGNFGNSYVS |
| | | | | WFAYWGQGTLVTVSA |
| 3 | VL | Murine | NA | caggctgttgtgactcaggaat |
| | | | | ctgcactcaccacatcacctgg |
| | | | | tgaaacagtcacactcacttgt |
| | | | | cgctcaagtactggggctgtta |
| | | | | caactagtaactatgccaactg |
| | | | | ggtccaagaaaaaccagatcat |
| | | | | ttattcactggtctaataggtg |
| | | | | gtaccaacaagcgagctccagg |
| | | | | tgtgcctgccagattctcaggc |
| | | | | tccctgattggagacaaggctg |
| | | | | ccctcaccatcacaggggcaca |
| | | | | gactgaggatgaggcaatatat |
| | | | | ttctgtgctctatggtacagca |
| | | | | acctctgggtgttcggtggagg |
| | | | | aaccaaactgactgtccta |
| 4 | VL | Murine | AA | QAVVTQESALTTSPGETVTLTC |
| | | | | RSSTGAVTTSNYANWVQEKPDH |
| | | | | LFTGLIGGTNKRAPGVPARFSG |
| | | | | SLIGDKAALTITGAQTEDEAIY |
| | | | | FCALWYSNLWVFGGGTKLTVL |
| 5 | VH | Murine | NA | caggtccagctgcagcagtctg |
| | | | | gggctgaactggcaagacctgg |
| | | | | ggcctcagtgaagatgtcctgc |
| | | | | aaggcttctggctacaccttta |
| | | | | ctagatctacgatgcactgggt |
| | | | | aaaacagaggcctggacagggt |
| | | | | ctggaatggattggatacatta |
| | | | | atcctagcagtgcttatactaa |
| | | | | ttacaatcagaaattcaaggac |
| | | | | aaggccacattgactgcagaca |
| | | | | aatcctccagtacagcctacat |
| | | | | gcaactgagtagcctgacatct |
| | | | | gaggactctgcagtctattact |
| | | | | gtgcaagtccgcaagtccacta |
| | | | | tgattacaacgggtttccttac |
| | | | | tggggccaagggactctggtca |
| | | | | ctgtctctgca |
| 6 | VH | Murine | AA | QVQLQQSGAELARPGASVKMSC |
| | | | | KASGYTFTRSTMHWVKQRPGQG |
| | | | | LEWIGYINPSSAYTNYNQKFKD |
| | | | | KATLTADKSSSTAYMQLSSLTS |
| | | | | EDSAVYYCASPQVHYDYNGFPY |
| | | | | WGQGTLVTVSA |
| 7 | VL | Murine | NA | caagttgttctcacccagtctc |
| | | | | cagcaatcatgtctgcatttcc |
| | | | | aggggagaaggtcaccatgacc |
| | | | | tgcagtgccagctcaagtgtaa |
| | | | | gttacatgaactggtaccagca |
| | | | | gaagtcaggcacctcccccaaa |
| | | | | agatggatttatgactcatcca |
| | | | | aactggcttctggagtccctgc |
| | | | | tcgcttcagtggcagtgggtct |
| | | | | gggacctcttattctctcacaa |
| | | | | tcagcagcatggagactgaaga |
| | | | | tgctgccacttattactgccag |
| | | | | cagtggagtcgtaacccaccca |
| | | | | cgttcggaggggggaccaagct |
| | | | | acaaattaca |
| 8 | VL | Murine | AA | QVVLTQSPAIMSAFPGEKVTMT |
| | | | | CSASSSVSYMNWYQQKSGTSPK |
| | | | | RWIYDSSKLASGVPARFSGSGS |
| | | | | GTSYSLTISSMETEDAATYYCQ |
| | | | | QWSRNPPTFGGGTKLQIT |
| 9 | VH-VL | arti- | NA | gaggtgaagcttctcgagtctg |
| | scFv | ficial | | gaggaggattggtgcagcctaa |
| | | | | agggtcattgaaactctcatgt |
| | | | | gcagcctctggattcaccttca |
| | | | | atacctacgccatgaactgggt |
| | | | | ccgccaggctccaggaaagggt |
| | | | | ttggaatgggttgctcgcataa |
| | | | | gaagtaaatataataattatgc |
| | | | | aacatattatgccgattcagtg |
| | | | | aaagacaggttcaccatctcca |
| | | | | gagatgattcacaaagcattct |
| | | | | ctatctacaaatgaacaacttg |
| | | | | aaaactgaggacacagccatgt |
| | | | | actactgtgtgagacatgggaa |
| | | | | cttcggtaatagctacgtttcc |
| | | | | tggtttgcttactggggccaag |
| | | | | ggactctggtcactgtctctgc |
| | | | | aggtggtggtggttctggcggc |
| | | | | ggcggctccggtggtggtggtt |
| | | | | ctcaggctgttgtgactcagga |
| | | | | atctgcactcaccacatcacct |
| | | | | ggtgaaacagtcacactcactt |
| | | | | gtcgctcaagtactggggctgt |
| | | | | tacaactagtaactatgccaac |
| | | | | tgggtccaagaaaaaccagatc |
| | | | | atttattcactggtctaatagg |
| | | | | tggtaccaacaagcgagctcca |
| | | | | ggtgtgcctgccagattctcag |
| | | | | gctccctgattggagacaaggc |
| | | | | tgccctcaccatcacaggggca |
| | | | | cagactgaggatgaggcaatat |
| | | | | atttctgtgctctatggtacag |
| | | | | caacctctgggtgttcggtgga |
| | | | | ggaaccaaactgactgtccta |
| 10 | VH-VL | arti- | AA | EVKLLESGGGLVQPKGSLKLSC |
| | scFv | ficial | | RSKYNNYATYYADSVKDRFTIS |
| | | | | RDDSQSILYLQMNNLKTEDTAM |
| | | | | YYCVRHGNFGNSYVSWFAYWGQ |
| | | | | GTLVTVSAGGGGSGGGGSGGGG |
| | | | | SQAVVTQESALTTSPGETVTLT |
| | | | | CRSSTGAVTTSNYANWVQEKPD |
| | | | | HLFTGLIGGTNKRAPGVPARFS |
| | | | | GSLIGDKAALTITGAQTEDEAI |
| | | | | YFCALWYSNLWVFGGGTKLTVL |
| 11 | VL-VH | arti- | NA | caggctgttgtgactcaggaat |
| | scFv | ficial | | ctgcactcaccacatcacctgg |
| | | | | tgaaacagtcacactcacttgt |
| | | | | cgctcaagtactggggctgtta |
| | | | | caactagtaactatgccaactg |
| | | | | ggtccaagaaaaaccagatcat |
| | | | | ttattcactggtctaataggtg |
| | | | | gtaccaacaagcgagctccagg |
| | | | | tgtgcctgccagattctcaggc |
| | | | | tccctgattggagacaaggctg |
| | | | | ccctcaccatcacaggggcaca |
| | | | | gactgaggatgaggcaatatat |
| | | | | ttctgtgctctatggtacagca |
| | | | | acctctgggtgttcggtggagg |
| | | | | aaccaaactgactgtcctaggt |
| | | | | ggtggtggttctggcggcggcg |
| | | | | gctccggtggtggtggttctga |
| | | | | ggtgaagcttctcgagtctgga |
| | | | | ggaggattggtgcagcctaaag |
| | | | | ggtcattgaaactctcatgtgc |
| | | | | agcctctggattcaccttcaat |
| | | | | acctacgccatgaactgggtcc |
| | | | | gccaggctccaggaaagggttt |
| | | | | ggaatgggttgctcgcataaga |
| | | | | agtaaatataataattatgcaa |
| | | | | catattatgccgattcagtgaa |
| | | | | agacaggttcaccatctccaga |
| | | | | gatgattcacaaagcattctct |
| | | | | atctacaaatgaacaacttgaa |
| | | | | aactgaggacacagccatgtac |
| | | | | tactgtgtgagacatgggaact |
| | | | | tcggtaatagctacgtttcctg |
| | | | | gtttgcttactggggccaaggg |
| | | | | actctggtcactgtctctgca |
| 12 | VL-VH | arti- | AA | QAVVTQESALTTSPGETVTLTC |
| | scFv | ficial | | RSSTGAVTTSNYANWVQEKPDH |
| | | | | LFTGLIGGTNKRAPGVPARFSG |
| | | | | SLIGDKAALTITGAQTEDEAIY |
| | | | | FCALWYSNLWVFGGGTKLTVLG |
| | | | | GGGSGGGGSGGGGSEVKLLESG |
| | | | | GGLVQPKGSLKLSCAASGFTFN |
| | | | | TYAMNWVRQAPGKGLEWVARIR |
| | | | | SKYNNYATYYADSVKDRFTISR |
| | | | | DDSQSILYLQMNNLKTEDTAMY |
| | | | | YCVRHGNFGNSYVSWFAYWGQG |
| | | | | TLVTVSA |
| 13 | VH-VL | arti- | NA | caggtccagctgcagcagtctg |
| | scFv | ficial | | gggctgaactggcaagacctgg |
| | | | | ggcctcagtgaagatgtcctgc |
| | | | | aaggcttctggctacaccttta |
| | | | | ctagatctacgatgcactgggt |
| | | | | aaaacagaggcctggacagggt |
| | | | | ctggaatggattggatacatta |
| | | | | atcctagcagtgcttatactaa |
| | | | | ttacaatcagaaattcaaggac |
| | | | | aaggccacattgactgcagaca |
| | | | | aatcctccagtacagcctacat |
| | | | | gcaactgagtagcctgacatct |
| | | | | gaggactctgcagtctattact |
| | | | | gtgcaagtccgcaagtccacta |
| | | | | tgattacaacgggtttccttac |
| | | | | tggggccaagggactctggtca |
| | | | | ctgtctctgcaggtggtggtgg |
| | | | | ttctggcggcggcggctccggt |
| | | | | ggtggtggttctcaagttgttc |
| | | | | tcacccagtctccagcaatcat |
| | | | | gtctgcatttccaggggagaag |
| | | | | gtcaccatgacctgcagtgcca |
| | | | | gctcaagtgtaagttacatgaa |
| | | | | ctggtaccagcagaagtcaggc |
| | | | | acctcccccaaaagatggattt |
| | | | | atgactcatccaaactggcttc |
| | | | | tggagtccctgctcgcttcagt |
| | | | | ggcagtgggtctgggacctctt |
| | | | | attctctcacaatcagcagcat |
| | | | | ggagactgaagatgctgccact |
| | | | | tattactgccagcagtggagtc |
| | | | | gtaacccacccacgttcggagg |
| | | | | ggggaccaagctacaaattaca |
| 14 | VH-VL | arti- | AA | QVQLQQSGAELARPGASVKMSC |
| | scFv | ficial | | KASGYTFTRSTMHWVKQRPGQG |
| | | | | LEWIGYINPSSAYTNYNQKFKD |
| | | | | KATLTADKSSSTAYMQLSSLTS |
| | | | | EDSAVYYCASPQVHYDYNGFPY |
| | | | | WGQGTLVTVSAGGGGSGGGGSG |
| | | | | GGGSQVVLTQSPAIMSAFPGEK |
| | | | | VTMTCSASSSVSYMNWYQQKSG |
| | | | | TSPKRWIYDSSKLASGVPARFS |
| | | | | GSGSGTSYSLTISSMETEDAAT |
| | | | | YYCQQWSRNPPTFGGGTKLQIT |
| 15 | VL-VH | arti- | NA | caagttgttctcacccagtctc |
| | scFv | ficial | | cagcaatcatgtctgcatttcc |
| | | | | aggggagaaggtcaccatgacc |
| | | | | tgcagtgccagctcaagtgtaa |
| | | | | gttacatgaactggtaccagca |
| | | | | gaagtcaggcacctcccccaaa |
| | | | | agatggatttatgactcatcca |
| | | | | aactggcttctggagtccctgc |
| | | | | tcgcttcagtggcagtgggtct |
| | | | | gggacctcttattctctcacaa |
| | | | | tcagcagcatggagactgaaga |
| | | | | tgctgccacttattactgccag |
| | | | | cagtggagtcgtaacccaccca |
| | | | | cgttcggaggggggaccaagct |
| | | | | acaaattacaggtggtggtggt |
| | | | | tctggcggcggcggctccggtg |
| | | | | gtggtggttctcaggtccagct |
| | | | | gcagcagtctggggctgaactg |
| | | | | gcaagacctggggcctcagtga |
| | | | | agatgtcctgcaaggcttctgg |
| | | | | ctacacctttactagatctacg |
| | | | | atgcactgggtaaaacagaggc |
| | | | | ctggacagggtctggaatggat |
| | | | | tggatacattaatcctagcagt |
| | | | | gcttatactaattacaatcaga |
| | | | | aattcaaggacaaggccacatt |
| | | | | gactgcagacaaatcctccagt |
| | | | | acagcctacatgcaactgagta |
| | | | | gcctgacatctgaggactctgc |
| | | | | agtctattactgtgcaagtccg |
| | | | | caagtccactatgattacaacg |
| | | | | ggtttccttactggggccaagg |
| | | | | gactctggtcactgtctctgca |
| 16 | VL-VH | arti- | AA | QVVLTQSPAIMSAFPGEKVTMT |
| | scFv | ficial | | CSASSSVSYMNWYQQKSGTSPK |
| | | | | RWIYDSSKLASGVPARFSGSGS |
| | | | | GTSYSLTISSMETEDAATYYCQ |
| | | | | QWSRNPPTFGGGTKLQITGGGG |
| | | | | SGGGGSGGGGSQVQLQQSGAEL |
| | | | | ARPGASVKMSCKASGYTFTRST |
| | | | | MHWVKQRPGQGLEWIGYINPSS |
| | | | | AYTNYNQKFKDKATLTADKSSS |
| | | | | TAYMQLSSLTSEDSAVYYCASP |
| | | | | QVHYDYNGFPYWGQGTLVTVSA |
| 17 | 5′ LH | arti- | NA | acatccggaggtggtggatccc |
| | | ficial | | aggctgttgtgactcaggaatc |
| | | | | tgc |
| 18 | 3′ VL | arti- | NA | ggagccgccgccgccagaacca |
| | Linker | ficial | | ccaccacctaggacagtcagtt |
| | | | | tggttcc |
| 19 | 5′ VH | arti- | NA | tctggcggcggcggctccggtg |
| | Linker | ficial | | gtggtggttctgaggtgaagct |
| | | | | tctcgagtctggaggaggattg |
| | | | | gtgc |
| 20 | 3′ LH | arti- | NA | agtgggtcgacctaatgatgat |
| | | ficial | | ggtgatgatgtgcagagacagt |
| | | | | gaccagagtccc |
| 21 | 5′ HL | arti- | NA | acatccggaggtggtggatccg |
| | | ficial | | aggtgaagcttctcgagtctgg |
| | | | | aggaggattggtgc |
| 22 | 3′ VH | arti- | NA | ggagccgccgccgccagaacca |
| | Linker | ficial | | ccaccacctgcagagacagtga |
| | | | | ccagagtccc |
| 23 | 5′ VL | arti- | NA | tctggcggcggcggctccggtg |
| | Linker | ficial | | gtggtggttctcaggctgttgt |
| | | | | gactcaggaatctgc |
| 24 | 3′ HL | arti- | NA | agtgggtcgacctaatgatgat |
| | | ficial | | ggtgatgatgtaggacagtcag |
| | | | | tttggttcctcc |
| 25 | 5′ LH | arti- | NA | acatccggaggtggtggatccc |
| | | ficial | | aagttgttctcacccagtctcc |
| 26 | 3′ VL | arti- | NA | ggagccgccgccgccagaacca |
| | Linker | ficial | | ccaccacctgtaatttgtagct |
| | | | | tggtccccc |
| 27 | 5′ VH | arti- | NA | tctggcggcggcggctccggtg |
| | Linker | ficial | | gtggtggttctcaggtccagct |
| | | | | gcagcagtctgg |
| 28 | 3′ LH | arti- | NA | agtgggtcgacctaatgatgat |
| | | ficial | | ggtgatgatgtgcagagacagt |
| | | | | gaccagagtcc |
| 29 | 5′ HL | arti- | NA | acatccggaggtggtggatccc |
| | | ficial | | aggtccagctgcagcagtctgg |
| 30 | 3′ VH | arti- | NA | ggagccgccgccgccagaacca |
| | Linker | ficial | | ccaccacctgcagagacagtga |
| | | | | ccagagtcc |
| 31 | 5′ VL | arti- | NA | tctggcggcggcggctccggtg |
| | Linker | ficial | | gtggtggttctcaagttgttct |
| | | | | cacccagtctcc |
| 32 | 3′ HL | arti- | NA | agtgggtcgacctaatgatgat |
| | | ficial | | ggtgatgatgtgtaatttgtag |
| | | | | cttggtccccc |
| 33 | 5-10 LH | arti- | NA | gagctcgtgatgacacagtctc |
| | scFv | ficial | | catcctccctgactgtgacagc |
| | | | | aggagagaaggtcactatgagc |
| | | | | tgcaagtccagtcagagtctgt |
| | | | | taaacagtggaaatcaaaagaa |
| | | | | ctacttgacctggtaccagcag |
| | | | | aaaccagggcagcctcctaaac |
| | | | | tgttgatctactgggcatccac |
| | | | | tagggaatctggggtccctgat |
| | | | | cgcttcacaggcagtggatctg |
| | | | | gaacagatttcactctcaccat |
| | | | | cagcagtgtgcaggctgaagac |
| | | | | ctggcagtttattactgtcaga |
| | | | | atgattatagttatccgctcac |
| | | | | gttcggtgctgggaccaagctt |
| | | | | gagatcaaaggtggtggtggtt |
| | | | | ctggcggcggcggctccggtgg |
| | | | | tggtggttctgaggtgcagctg |
| | | | | ctcgagcagtctggagctgagc |
| | | | | tggtaaggcctgggacttcagt |
| | | | | gaagatatcctgcaaggcttct |
| | | | | ggatacgccttcactaactact |
| | | | | ggctaggttgggtaaagcagag |
| | | | | gcctggacatggacttgagtgg |
| | | | | attggagatattttccctggaa |
| | | | | gtggtaatatccactacaatga |
| | | | | gaagttcaagggcaaagccaca |
| | | | | ctgactgcagacaaatcttcga |
| | | | | gcacagcctatatgcagctcag |
| | | | | tagcctgacatttgaggactct |
| | | | | gctgtctatttctgtgcaagac |
| | | | | tgaggaactgggacgagcctat |
| | | | | ggactactggggccaagggacc |
| | | | | acggtcaccgtctcctcc |
| 34 | 5-10 LH | arti- | AA | ELVMTQSPSSLTVTAGEKVTMS |
| | scFv | ficial | | CKSSQSLLNSGNQKNYLTWYQQ |
| | | | | KPGQPPKLLIYWASTRESGVPD |
| | | | | RFTGSGSGTDFTLTISSVQAED |
| | | | | LAVYYCQNDYSYPLTFGAGTKL |
| | | | | EIKGGGGSGGGGSGGGGSEVQL |
| | | | | LEQSGAELVRPGTSVKISCKAS |
| | | | | GYAFTNYWLGWVKQRPGHGLEW |
| | | | | IGDIFPGSGNIHYNEKFKGKAT |
| | | | | LTADKSSSTAYMQLSSLTFEDS |
| | | | | AVYFCARLRNWDEPMDYWGQGT |
| | | | | TVTVSS |
| 35 | Leader | mouse | NA | atgggatggagctgtatcatcc |
| | peptide | cDNA | | tcttcttggtagcaacagctac |
| | | | | aggtgtacactcc |
| 36 | Leader | mouse | AA | MGWSCIILFLVATATGVHS |
| | peptide | cDNA | | |
| 37 | 5-10 LHx | arti- | NA | gagctcgtgatgacacagtctc |
| | SEQ ID | ficial | | catcctccctgactgtgacagc |
| | NO. 12 | | | aggagagaaggtcactatgagc |
| | | | | tgcaagtccagtcagagtctgt |
| | | | | taaacagtggaaatcaaaagaa |
| | | | | ctacttgacctggtaccagcag |
| | | | | aaaccagggcagcctcctaaac |
| | | | | tgttgatctactgggcatccac |
| | | | | tagggaatctggggtccctgat |
| | | | | cgcttcacaggcagtggatctg |
| | | | | gaacagatttcactctcaccat |
| | | | | cagcagtgtgcaggctgaagac |
| | | | | ctggcagtttattactgtcaga |
| | | | | atgattatagttatccgctcac |
| | | | | gttcggtgctgggaccaagctt |
| | | | | gagatcaaaggtggtggtggtt |
| | | | | ctggcggcggcggctccggtgg |
| | | | | tggtggttctgaggtgcagctg |
| | | | | ctcgagcagtctggagctgagc |
| | | | | tggtaaggcctgggacttcagt |
| | | | | gaagatatcctgcaaggcttct |
| | | | | ggatacgccttcactaactact |
| | | | | ggctaggttgggtaaagcagag |
| | | | | gcctggacatggacttgagtgg |
| | | | | attggagatattttccctggaa |
| | | | | gtggtaatatccactacaatga |
| | | | | gaagttcaagggcaaagccaca |
| | | | | ctgactgcagacaaatcttcga |
| | | | | gcacagcctatatgcagctcag |
| | | | | tagcctgacatttgaggactct |
| | | | | gctgtctatttctgtgcaagac |
| | | | | tgaggaactgggacgagcctat |
| | | | | ggactactggggccaagggacc |
| | | | | acggtcaccgtctcctccggag |
| | | | | gtggtggatcccaggctgttgt |
| | | | | gactcaggaatctgcactcacc |
| | | | | acatcacctggtgaaacagtca |
| | | | | cactcacttgtcgctcaagtac |
| | | | | tggggctgttacaactagtaac |
| | | | | tatgccaactgggtccaagaaa |
| | | | | aaccagatcatttattcactgg |
| | | | | tctaataggtggtaccaacaag |
| | | | | cgagctccaggtgtgcctgcca |
| | | | | gattctcaggctccctgattgg |
| | | | | agacaaggctgccctcaccatc |
| | | | | acaggggcacagactgaggatg |
| | | | | aggcaatatatttctgtgctct |
| | | | | atggtacagcaacctctgggtg |
| | | | | ttcggtggaggaaccaaactga |
| | | | | ctgtcctaggtggtggtggttc |
| | | | | tggcggcggcggctccggtggt |
| | | | | ggtggttctgaggtgaagcttc |
| | | | | tcgagtctggaggaggattggt |
| | | | | gcagcctaaagggtcattgaaa |
| | | | | ctctcatgtgcagcctctggat |
| | | | | tcaccttcaatacctacgccat |
| | | | | gaactgggtccgccaggctcca |
| | | | | ggaaagggtttggaatgggttg |
| | | | | ctcgcataagaagtaaatataa |
| | | | | taattatgcaacatattatgcc |
| | | | | gattcagtgaaagacaggttca |
| | | | | ccatctccagagatgattcaca |
| | | | | aagcattctctatctacaaatg |
| | | | | aacaacttgaaaactgaggaca |
| | | | | cagccatgtactactgtgtgag |
| | | | | acatgggaacttcggtaatagc |
| | | | | tacgtttcctggtttgcttact |
| | | | | ggggccaagggactctggtcac |
| | | | | tgtctctgca |
| 38 | 5-10 LHx | arti- | AA | ELVMTQSPSSLTVTAGEKVTMS |
| | SEQ ID | ficial | | CKSSQSLLNSGNQKNYLTWYQQ |
| | NO. 12 | | | KPGQPPKLLIYWASTRESGVPD |
| | | | | RFTGSGSGTDFTLTISSVQAED |
| | | | | LAVYYCQNDYSYPLTFGAGTKL |
| | | | | EIKGGGGSGGGGSGGGGSEVQL |
| | | | | LEQSGAELVRPGTSVKISCKAS |
| | | | | GYAFTNYWLGWVKQRPGHGLEW |
| | | | | IGDIFPGSGNIHYNEKFKGKAT |
| | | | | LTADKSSSTAYMQLSSLTFEDS |
| | | | | AVYFCARLRNWDEPMDYWGQGT |
| | | | | TVTVSSGGGGSQAVVTQESALT |
| | | | | TSPGETVTLTCRSSTGAVTTSN |
| | | | | YANWVQEKPDHLFTGLIGGTNK |
| | | | | RAPGVPARFSGSLIGDKAALTI |
| | | | | TGAQTEDEAIYFCALWYSNLWV |
| | | | | FGGGTKLTVLGGGGSGGGGSGG |
| | | | | GGSEVKLLESGGGLVQPKGSLK |
| | | | | LSCAASGFTFNTYAMNWVRQAP |
| | | | | GKGLEWVARIRSKYNNYATYYA |
| | | | | DSVKDRFTISRDDSQSILYLQM |
| | | | | NNLKTEDTAMYYCVRHGNFGNS |
| | | | | YVSWFAYWGQGTLVTVSA |
| 39 | 5-10 LHx | arti- | NA | gagctcgtgatgacacagtctc |
| | SEQ ID | ficial | | catcctccctgactgtgacagc |
| | NO. 10 | | | aggagagaaggtcactatgagc |
| | | | | tgcaagtccagtcagagtctgt |
| | | | | taaacagtggaaatcaaaagaa |
| | | | | ctacttgacctggtaccagcag |
| | | | | aaaccagggcagcctcctaaac |
| | | | | tgttgatctactgggcatccac |
| | | | | tagggaatctggggtccctgat |
| | | | | cgcttcacaggcagtggatctg |
| | | | | gaacagatttcactctcaccat |
| | | | | cagcagtgtgcaggctgaagac |
| | | | | ctggcagtttattactgtcaga |
| | | | | atgattatagttatccgctcac |
| | | | | gttcggtgctgggaccaagctt |
| | | | | gagatcaaaggtggtggtggtt |
| | | | | ctggcggcggcggctccggtgg |
| | | | | tggtggttctgaggtgcagctg |
| | | | | ctcgagcagtctggagctgagc |
| | | | | tggtaaggcctgggacttcagt |
| | | | | gaagatatcctgcaaggcttct |
| | | | | ggatacgccttcactaactact |
| | | | | ggctaggttgggtaaagcagag |
| | | | | gcctggacatggacttgagtgg |
| | | | | attggagatattttccctggaa |
| | | | | gtggtaatatccactacaatga |
| | | | | gaagttcaagggcaaagccaca |
| | | | | ctgactgcagacaaatcttcga |
| | | | | gcacagcctatatgcagctcag |
| | | | | tagcctgacatttgaggactct |
| | | | | gctgtctatttctgtgcaagac |
| | | | | tgaggaactgggacgagcctat |
| | | | | ggactactggggccaagggacc |
| | | | | acggtcaccgtctcctccggag |
| | | | | gtggtggatccgaggtgaagct |
| | | | | tctcgagtctggaggaggattg |
| | | | | gtgcagcctaaagggtcattga |
| | | | | aactctcatgtgcagcctctgg |
| | | | | attcaccttcaatacctacgcc |
| | | | | atgaactgggtccgccaggctc |
| | | | | caggaaagggtttggaatgggt |
| | | | | tgctcgcataagaagtaaatat |
| | | | | aataattatgcaacatattatg |
| | | | | ccgattcagtgaaagacaggtt |
| | | | | caccatctccagagatgattca |
| | | | | caaagcattctctatctacaaa |
| | | | | tgaacaacttgaaaactgagga |
| | | | | cacagccatgtactactgtgtg |
| | | | | agacatgggaacttcggtaata |
| | | | | gctacgtttcctggtttgctta |
| | | | | ctggggccaagggactctggtc |
| | | | | actgtctctgcaggtggtggtg |
| | | | | gttctggcggcggcggctccgg |
| | | | | tggtggtggttctcaggctgtt |
| | | | | gtgactcaggaatctgcactca |
| | | | | ccacatcacctggtgaaacagt |
| | | | | cacactcacttgtcgctcaagt |
| | | | | actggggctgttacaactagta |
| | | | | actatgccaactgggtccaaga |
| | | | | aaaaccagatcatttattcact |
| | | | | ggtctaataggtggtaccaaca |
| | | | | agcgagctccaggtgtgcctgc |
| | | | | cagattctcaggctccctgatt |
| | | | | ggagacaaggctgccctcacca |
| | | | | tcacaggggcacagactgagga |
| | | | | tgaggcaatatatttctgtgct |
| | | | | ctatggtacagcaacctctggg |
| | | | | tgttcggtggaggaaccaaact |
| | | | | gactgtccta |
| 40 | 5-10 LHx | arti- | AA | ELVMTQSPSSLTVTAGEKVTMS |
| | SEQ ID | ficial | | CKSSQSLLNSGNQKNYLTWYQQ |
| | NO. 10 | | | KPGQPPKLLIYWASTRESGVPD |
| | | | | RFTGSGSGTDFTLTISSVQAED |
| | | | | LAVYYCQNDYSYPLTFGAGTKL |
| | | | | EIKGGGGSGGGGSGGGGSEVQL |
| | | | | LEQSGAELVRPGTSVKISCKAS |
| | | | | GYAFTNYWLGWVKQRPGHGLEW |
| | | | | IGDIFPGSGNIHYNEKFKGKAT |
| | | | | LTADKSSSTAYMQLSSLTFEDS |
| | | | | AVYFCARLRNWDEPMDYWGQGT |
| | | | | TVTVSSGGGGSEVKLLESGGGL |
| | | | | VQPKGSLKLSCAASGFTFNTYA |
| | | | | MNWVRQAPGKGLEWVARIRSKY |
| | | | | NNYATYYADSVKDRFTISRDDS |
| | | | | QSILYLQMNNLKTEDTAMYYCV |
| | | | | RHGNFGNSYVSWFAYWGQGTLV |
| | | | | TVSAGGGGSGGGGSGGGGSQAV |
| | | | | VTQESALTTSPGETVTLTCRSS |
| | | | | TGAVTTSNYANWVQEKPDHLFT |
| | | | | GLIGGTNKRAPGVPARFSGSLI |
| | | | | GDKAALTITGAQTEDEAIYFCA |
| | | | | LWYSNLWVFGGGTKLTVL |
| 41 | 5-10 LHx | arti- | NA | gagctcgtgatgacacagtctc |
| | SEQ ID | ficial | | catcctccctgactgtgacagc |
| | NO. 16 | | | aggagagaaggtcactatgagc |
| | | | | tgcaagtccagtcagagtctgt |
| | | | | taaacagtggaaatcaaaagaa |
| | | | | ctacttgacctggtaccagcag |
| | | | | aaaccagggcagcctcctaaac |
| | | | | tgttgatctactgggcatccac |
| | | | | tagggaatctggggtccctgat |
| | | | | cgcttcacaggcagtggatctg |
| | | | | gaacagatttcactctcaccat |
| | | | | cagcagtgtgcaggctgaagac |
| | | | | ctggcagtttattactgtcaga |
| | | | | atgattatagttatccgctcac |
| | | | | gttcggtgctgggaccaagctt |
| | | | | gagatcaaaggtggtggtggtt |
| | | | | ctggcggcggcggctccggtgg |
| | | | | tggtggttctgaggtgcagctg |
| | | | | ctcgagcagtctggagctgagc |
| | | | | tggtaaggcctgggacttcagt |
| | | | | gaagatatcctgcaaggcttct |
| | | | | ggatacgccttcactaactact |
| | | | | ggctaggttgggtaaagcagag |
| | | | | gcctggacatggacttgagtgg |
| | | | | attggagatattttccctggaa |
| | | | | gtggtaatatccactacaatga |
| | | | | gaagttcaagggcaaagccaca |
| | | | | ctgactgcagacaaatcttcga |
| | | | | gcacagcctatatgcagctcag |
| | | | | tagcctgacatttgaggactct |
| | | | | gctgtctatttctgtgcaagac |
| | | | | tgaggaactgggacgagcctat |
| | | | | ggactactggggccaagggacc |
| | | | | acggtcaccgtctcctccggag |
| | | | | gtggtggatcccaagttgttct |
| | | | | cacccagtctccagcaatcatg |
| | | | | tctgcatttccaggggagaagg |
| | | | | tcaccatgacctgcagtgccag |
| | | | | ctcaagtgtaagttacatgaac |
| | | | | tggtaccagcagaagtcaggca |
| | | | | cctcccccaaaagatggattta |
| | | | | tgactcatccaaactggcttct |
| | | | | ggagtccctgctcgcttcagtg |
| | | | | gcagtgggtctgggacctctta |
| | | | | ttctctcacaatcagcagcatg |
| | | | | gagactgaagatgctgccactt |
| | | | | attactgccagcagtggagtcg |
| | | | | taacccacccacgttcggaggg |
| | | | | gggaccaagctacaaattacag |
| | | | | gtggtggtggttctggcggcgg |
| | | | | cggctccggtggtggtggttct |
| | | | | caggtccagctgcagcagtctg |
| | | | | gggctgaactggcaagacctgg |
| | | | | ggcctcagtgaagatgtcctgc |
| | | | | aaggcttctggctacaccttta |
| | | | | ctagatctacgatgcactgggt |
| | | | | aaaacagaggcctggacagggt |
| | | | | ctggaatggattggatacatta |
| | | | | atcctagcagtgcttatactaa |
| | | | | ttacaatcagaaattcaaggac |
| | | | | aaggccacattgactgcagaca |
| | | | | aatcctccagtacagcctacat |
| | | | | gcaactgagtagcctgacatct |
| | | | | gaggactctgcagtctattact |
| | | | | gtgcaagtccgcaagtccacta |
| | | | | tgattacaacgggtttccttac |
| | | | | tggggccaagggactctggtca |
| | | | | ctgtctctgca |
| 42 | 5-10 LHx | arti- | AA | ELVMTQSPSSLTVTAGEKVTMS |
| | SEQ ID | ficial | | CKSSQSLLNSGNQKNYLTWYQQ |
| | NO. 16 | | | KPGQPPKLLIYWASTRESGVPD |
| | | | | RFTGSGSGTDFTLTISSVQAED |
| | | | | LAVYYCQNDYSYPLTFGAGTKL |
| | | | | EIKGGGGSGGGGSGGGGSEVQL |
| | | | | LEQSGAELVRPGTSVKISCKAS |
| | | | | GYAFTNYWLGWVKQRPGHGLEW |
| | | | | IGDIFPGSGNIHYNEKFKGKAT |
| | | | | LTADKSSSTAYMQLSSLTFEDS |
| | | | | AVYFCARLRNWDEPMDYWGQGT |
| | | | | TVTVSSGGGGSQVVLTQSPAIM |
| | | | | SAFPGEKVTMTCSASSSVSYMN |
| | | | | WYQQKSGTSPKRWIYDSSKLAS |
| | | | | GVPARFSGSGSGTSYSLTISSM |
| | | | | ETEDAATYYCQQWSRNPPTFGG |
| | | | | GTKLQITGGGGSGGGGSGGGGS |
| | | | | QVQLQQSGAELARPGASVKMSC |
| | | | | KASGYTFTRSTMHWVKQRPGQG |
| | | | | LEWIGYINPSSAYTNYNQKFKD |
| | | | | KATLTADKSSSTAYMQLSSLTS |
| | | | | EDSAVYYCASPQVHYDYNGFPY |
| | | | | WGQGTLVTVSA |
| 43 | 5-10 LHx | arti- | NA | gagctcgtgatgacacagtctc |
| | SEQ ID | ficial | | catcctccctgactgtgacagc |
| | NO. 14 | | | aggagagaaggtcactatgagc |
| | | | | tgcaagtccagtcagagtctgt |
| | | | | taaacagtggaaatcaaaagaa |
| | | | | ctacttgacctggtaccagcag |
| | | | | aaaccagggcagcctcctaaac |
| | | | | tgttgatctactgggcatccac |
| | | | | tagggaatctggggtccctgat |
| | | | | cgcttcacaggcagtggatctg |
| | | | | gaacagatttcactctcaccat |
| | | | | cagcagtgtgcaggctgaagac |
| | | | | ctggcagtttattactgtcaga |
| | | | | atgattatagttatccgctcac |
| | | | | gttcggtgctgggaccaagctt |
| | | | | gagatcaaaggtggtggtggtt |
| | | | | ctggcggcggcggctccggtgg |
| | | | | tggtggttctgaggtgcagctg |
| | | | | ctcgagcagtctggagctgagc |
| | | | | tggtaaggcctgggacttcagt |
| | | | | gaagatatcctgcaaggcttct |
| | | | | ggatacgccttcactaactact |
| | | | | ggctaggttgggtaaagcagag |
| | | | | gcctggacatggacttgagtgg |
| | | | | attggagatattttccctggaa |
| | | | | gtggtaatatccactacaatga |
| | | | | gaagttcaagggcaaagccaca |
| | | | | ctgactgcagacaaatcttcga |
| | | | | gcacagcctatatgcagctcag |
| | | | | tagcctgacatttgaggactct |
| | | | | gctgtctatttctgtgcaagac |
| | | | | tgaggaactgggacgagcctat |
| | | | | ggactactggggccaagggacc |
| | | | | acggtcaccgtctcctccggag |
| | | | | gtggtggatcccaggtccagct |
| | | | | gcagcagtctggggctgaactg |
| | | | | gcaagacctggggcctcagtga |
| | | | | agatgtcctgcaaggcttctgg |
| | | | | ctacacctttactagatctacg |
| | | | | atgcactgggtaaaacagaggc |
| | | | | ctggacagggtctggaatggat |
| | | | | tggatacattaatcctagcagt |
| | | | | gcttatactaattacaatcaga |
| | | | | aattcaaggacaaggccacatt |
| | | | | gactgcagacaaatcctccagt |
| | | | | acagcctacatgcaactgagta |
| | | | | gcctgacatctgaggactctgc |
| | | | | agtctattactgtgcaagtccg |
| | | | | caagtccactatgattacaacg |
| | | | | ggtttccttactggggccaagg |
| | | | | gactctggtcactgtctctgca |
| | | | | ggtggtggtggttctggcggcg |
| | | | | gcggctccggtggtggtggttc |
| | | | | tcaagttgttctcacccagtct |
| | | | | ccagcaatcatgtctgcatttc |
| | | | | caggggagaaggtcaccatgac |
| | | | | ctgcagtgccagctcaagtgta |
| | | | | agttacatgaactggtaccagc |
| | | | | agaagtcaggcacctcccccaa |
| | | | | aagatggatttatgactcatcc |
| | | | | aaactggcttctggagtccctg |
| | | | | ctcgcttcagtggcagtgggtc |
| | | | | tgggacctcttattctctcaca |
| | | | | atcagcagcatggagactgaag |
| | | | | atgctgccacttattactgcca |
| | | | | gcagtggagtcgtaacccaccc |
| | | | | acgttcggaggggggaccaagc |
| | | | | tacaaattaca |
| 44 | 5-10 LHx | arti- | AA | ELVMTQSPSSLTVTAGEKVTMS |
| | SEQ ID | ficial | | CKSSQSLLNSGNQKNYLTWYQQ |
| | NO. 14 | | | KPGQPPKLLIYWASTRESGVPD |
| | | | | RFTGSGSGTDFTLTISSVQAED |
| | | | | LAVYYCQNDYSYPLTFGAGTKL |
| | | | | EIKGGGGSGGGGSGGGGSEVQL |
| | | | | LEQSGAELVRPGTSVKISCKAS |
| | | | | GYAFTNYWLGWVKQRPGHGLEW |
| | | | | IGDIFPGSGNIHYNEKFKGKAT |
| | | | | LTADKSSSTAYMQLSSLTFEDS |
| | | | | AVYFCARLRNWDEPMDYWGQGT |
| | | | | TVTVSSGGGGSQVQLQQSGAEL |
| | | | | ARPGASVKMSCKASGYTFTRST |
| | | | | MHWVKQRPGQGLEWIGYINPSS |
| | | | | AYTNYNQKFKDKATLTADKSSS |
| | | | | TAYMQLSSLTSEDSAVYYCASP |
| | | | | QVHYDYNGFPYWGQGTLVTVSA |
| | | | | GGGGSGGGGSGGGGSQVVLTQS |
| | | | | PAIMSAFPGEKVTMTCSASSSV |
| | | | | SYMNWYQQKSGTSPKRWIYDSS |
| | | | | KLASGVPARFSGSGSGTSYSLT |
| | | | | ISSMETEDAATYYCQQWSRNPP |
| | | | | TFGGGTKLQIT |
| 45 | 5′EpCAM | arti- | NA | ggttctagaccaccatggcgcc |
| | | ficial | | cccgcaggtcctcgcgttcgg |
| 46 | 3′EpCAM | arti- | NA | agtgggtcgacttatgcattga |
| | | ficial | | gttccctatgcatctcaccc |
| 47 | cyno- | Cyno- | NA | cagaaagaatgtgtctgtgaaa |
| | molgus | molgus | | actacaagctggccgtaaactg |
| | EpCAM | cDNA | | ctttttgaatgacaatggtcaa |
| | extra- | | | tgccagtgtacttcgattggtg |
| | cellular | | | cacaaaatactgtcctttgctc |
| | portion | | | aaagctggctgccaaatgtttg |
| | | | | gtgatgaaggcagaaatgaacg |
| | | | | gctcaaaacttgggagaagagc |
| | | | | gaaacctgaaggggctctccag |
| | | | | aacaatgatggcctttacgatc |
| | | | | ctgactgcgatgagagcgggct |
| | | | | ctttaaggccaagcagtgcaac |
| | | | | ggcacctccacgtgctggtgtg |
| | | | | tgaacactgctggggtcagaag |
| | | | | aactgacaaggacactgaaata |
| | | | | acctgctctgagcgagtgagaa |
| | | | | cctactggatcatcattgaatt |
| | | | | aaaacacaaagcaagagaaaaa |
| | | | | ccttatgatgttcaaagtttgc |
| | | | | ggactgcacttgaggaggcgat |
| | | | | caaaacgcgttatcaactggat |
| | | | | ccaaaatttatcacaaatattt |
| | | | | tgtatgaggataatgttatcac |
| | | | | tattgatctggttcaaaattct |
| | | | | tctcagaaaactcagaatgatg |
| | | | | tggacatagctgatgtggctta |
| | | | | ttattttgaaaaagatgttaaa |
| | | | | ggtgaatccttgtttcattcta |
| | | | | agaaaatggacctgagagtaaa |
| | | | | tggggaacaactggatctggat |
| | | | | cctggtcaaactttaatttatt |
| | | | | atgtcgatgaaaaagcacctga |
| | | | | attctcaatgcagggtctaaaa |
| 48 | cyno- | Cyno- | AA | QKECVCENYKLAVNCFLNDNGQ |
| | molgus | molgus | | CQCTSIGAQNTVLCSKLAAKCL |
| | EpCAM | cDNA | | VMKAEMNGSKLGRRAKPEGALQ |
| | extra- | | | NNDGLYDPDCDESGLFKAKQCN |
| | cellular | | | GTSTCWCVNTAGVRRTDKDTEI |
| | portion | | | TCSERVRTYWIIIELKHKAREK |
| | | | | PYDVQSLRTALEEAIKTRYQLD |
| | | | | PKFITNILYEDNVITIDLVQNS |
| | | | | SQKTQNDVDIADVAYYFEKDVK |
| | | | | GESLFHSKKMDLRVNGEQLDLD |
| | | | | PGQTLIYYVDEKAPEFSMQGLK |
| 49 | 2G8 VH | Hybri- | NA | gaggttcagctgcagcagtctg |
| | | doma | | gggcagagcttgtgaggtcagg |
| | | | | ggcctcagtcaagttgtcctgc |
| | | | | acagcttctggcttcaacatta |
| | | | | aagactactatttgcactgggt |
| | | | | gaagcagaggcctgaacagggc |
| | | | | ctggagtggattgcctggattg |
| | | | | atcttgagaatggtgatattaa |
| | | | | atatgccccgaagtttcagggc |
| | | | | aaggccactataactgcagaca |
| | | | | catcctccaacacagcctacct |
| | | | | gcagctcagcagcctgacatct |
| | | | | gaggacactgccgtctattact |
| | | | | gtaatccctattactacggtag |
| | | | | taactacgactatgctatggac |
| | | | | tactggggtcaaggaacctcag |
| | | | | tcaccgtctcctca |
| 50 | 2G8 VH | Hybri- | AA | EVQLQQSGAELVRSGASVKLSC |
| | | doma | | TASGFNIKDYYLHWVKQRPEQG |
| | | | | LEWIAWIDLENGDIKYAPKFQG |
| | | | | KATITADTSSNTAYLQLSSLTS |
| | | | | EDTAVYYCNPYYYGSNYDYAMD |
| | | | | YWGQGTSVTVSS |
| 51 | 2G8 VL | Hybri- | NA | gatgttgtgatgacccagactc |
| | | doma | | cactcactttgtcggttaccat |
| | | | | tggacaaccagcctctatctct |
| | | | | tgcaagtcaagtcagagcctct |
| | | | | tatatagtaatggaaaaaccta |
| | | | | tttgaactggatattacagagg |
| | | | | ccaggccagtctccaaagcgcc |
| | | | | taatctatctggtgtctaaact |
| | | | | ggactctggagtccctgacagg |
| | | | | ttcactggcagtggatcaggaa |
| | | | | cagattttacgctgaaaatcag |
| | | | | cagagtggaggctgaggatttg |
| | | | | ggagtttattactgcgtgcaag |
| | | | | gtacacattttcctctcacgtt |
| | | | | cggtgctgggaccaagctggag |
| | | | | ctgaaa |
| 52 | 2G8 VL | Hybri- | AA | DVVMTQTPLTLSVTIGQPASIS |
| | | doma | | CKSSQSLLYSNGKTYLNWILQR |
| | | | | PGQSPKRLIYLVSKLDSGVPDR |
| | | | | FTGSGSGTDFTLKISRVEAEDL |
| | | | | GVYYCVQGTHFPLTFGAGTKLE |
| | | | | LK |
| 53 | 2G8VH-VL | arti- | NA | gaggttcagctgcagcagtctg |
| | scFv | ficial | | gggcagagcttgtgaggtcagg |
| | | | | ggcctcagtcaagttgtcctgc |
| | | | | acagcttctggcttcaacatta |
| | | | | aagactactatttgcactgggt |
| | | | | gaagcagaggcctgaacagggc |
| | | | | ctggagtggattgcctggattg |
| | | | | atcttgagaatggtgatattaa |
| | | | | atatgccccgaagtttcagggc |
| | | | | aaggccactataactgcagaca |
| | | | | catcctccaacacagcctacct |
| | | | | gcagctcagcagcctgacatct |
| | | | | gaggacactgccgtctattact |
| | | | | gtaatccctattactacggtag |
| | | | | taactacgactatgctatggac |
| | | | | tactggggtcaaggaacctcag |
| | | | | tcaccgtctcctcaggtggtgg |
| | | | | tggttctggcggcggcggctcc |
| | | | | ggtggtggtggttctgatgttg |
| | | | | tgatgacccagactccactcac |
| | | | | tttgtcggttaccattggacaa |
| | | | | ccagcctctatctcttgcaagt |
| | | | | caagtcagagcctcttatatag |
| | | | | taatggaaaaacctatttgaac |
| | | | | tggatattacagaggccaggcc |
| | | | | agtctccaaagcgcctaatcta |
| | | | | tctggtgtctaaactggactct |
| | | | | ggagtccctgacaggttcactg |
| | | | | gcagtggatcaggaacagattt |
| | | | | tacgctgaaaatcagcagagtg |
| | | | | gaggctgaggatttgggagttt |
| | | | | attactgcgtgcaaggtacaca |
| | | | | ttttcctctcacgttcggtgct |
| | | | | gggaccaagctggagctgaaa |
| 54 | 2G8VH-VL | arti- | AA | EVQLQQSGAELVRSGASVKLSC |
| | scFv | ficial | | TASGFNIKDYYLHWVKQRPEQG |
| | | | | LEWIAWIDLENGDIKYAPKFQG |
| | | | | KATITADTSSNTAYLQLSSLTS |
| | | | | EDTAVYYCNPYYYGSNYDYAMD |
| | | | | YWGQGTSVTVSSGGGGSGGGGS |
| | | | | GGGGSDVVMTQTPLTLSVTIGQ |
| | | | | PASISCKSSQSLLYSNGKTYLN |
| | | | | WILQRPGQSPKRLIYLVSKLDS |
| | | | | GVPDRFTGSGSGTDFTLKISRV |
| | | | | EAEDLGVYYCVQGTHFPLTFGA |
| | | | | GTKLELK |
| 55 | 2G8VL-VH | arti- | NA | gatgttgtgatgacccagactc |
| | scFv | ficial | | cactcactttgtcggttaccat |
| | | | | tggacaaccagcctctatctct |
| | | | | tgcaagtcaagtcagagcctct |
| | | | | tatatagtaatggaaaaaccta |
| | | | | tttgaactggatattacagagg |
| | | | | ccaggccagtctccaaagcgcc |
| | | | | taatctatctggtgtctaaact |
| | | | | ggactctggagtccctgacagg |
| | | | | ttcactggcagtggatcaggaa |
| | | | | cagattttacgctgaaaatcag |
| | | | | cagagtggaggctgaggatttg |
| | | | | ggagtttattactgcgtgcaag |
| | | | | gtacacattttcctctcacgtt |
| | | | | cggtgctgggaccaagctggag |
| | | | | ctgaaaggtggtggtggttctg |
| | | | | gcggcggcggctccggtggtgg |
| | | | | tggttctgaggttcagctgcag |
| | | | | cagtctggggcagagcttgtga |
| | | | | ggtcaggggcctcagtcaagtt |
| | | | | gtcctgcacagcttctggcttc |
| | | | | aacattaaagactactatttgc |
| | | | | actgggtgaagcagaggcctga |
| | | | | acagggcctggagtggattgcc |
| | | | | tggattgatcttgagaatggtg |
| | | | | atattaaatatgccccgaagtt |
| | | | | tcagggcaaggccactataact |
| | | | | gcagacacatcctccaacacag |
| | | | | cctacctgcagctcagcagcct |
| | | | | gacatctgaggacactgccgtc |
| | | | | tattactgtaatccctattact |
| | | | | acggtagtaactacgactatgc |
| | | | | tatggactactggggtcaagga |
| | | | | acctcagtcaccgtctcctcc |
| 56 | 2G8VL-VH | arti- | AA | DVVMTQTPLTLSVTIGQPASIS |
| | scFv | ficial | | CKSSQSLLYSNGKTYLNWILQR |
| | | | | PGQSPKRLIYLVSKLDSGVPDR |
| | | | | FTGSGSGTDFTLKISRVEAEDL |
| | | | | GVYYCVQGTHFPLTFGAGTKLE |
| | | | | LKGGGGSGGGGSGGGGSEVQLQ |
| | | | | QSGAELVRSGASVKLSCTASGF |
| | | | | NIKDYYLHWVKQRPEQGLEWIA |
| | | | | WIDLENGDIKYAPKFQGKATIT |
| | | | | ADTSSNTAYLQLSSLTSEDTAV |
| | | | | YYCNPYYYGSNYDYAMDYWGQG |
| | | | | TSVTVSS |
| 57 | 5′2G8 LH | arti- | NA | aggtgtacactccgatgttgtg |
| | | ficial | | atgacccagactccactcactt |
| | | | | tgtcg |
| 58 | 3′2G8 VL | arti- | NA | ggagccgccgccgccagaacca |
| | Linker | ficial | | ccaccacctttcagctccagct |
| | | | | tggtcccagc |
| 59 | 5′2G8 VH | arti- | NA | tctggcggcggcggctccggtg |
| | Linker | ficial | | gtggtggttctgaggttcagct |
| | | | | gcagcagtctgg |
| 60 | 3′2G8 LH | arti- | NA | acatccggaggagacggtgact |
| | | ficial | | gaggttcc |
| 61 | 5′2G8 HL | arti- | NA | aggtgtacactccgaggttcag |
| | | ficial | | ctgcagcagtctggg |
| 62 | 3′2G8 VH | arti- | NA | ggagccgccgccgccagaacca |
| | Linker | ficial | | ccaccacctgaggagacggtga |
| | | | | ctgaggttcc |
| 63 | 5′2G8 VL | arti- | NA | tctggcggcggcggctccggtg |
| | Linker | ficial | | gtggtggttctgatgttgtgat |
| | | | | gacccagactccactcactttg |
| | | | | tcg |
| 64 | 3′2G8 HL | arti- | NA | acatccggatttcagctccagc |
| | | ficial | | ttggtcccagc |
| 65 | 2G8LHx | arti- | NA | gatgttgtgatgacccagactc |
| | SEQ ID | ficial | | cactcactttgtcggttaccat |
| | NO. 12 | | | tggacaaccagcctctatctct |
| | | | | tgcaagtcaagtcagagcctct |
| | | | | tatatagtaatggaaaaaccta |
| | | | | tttgaactggatattacagagg |
| | | | | ccaggccagtctccaaagcgcc |
| | | | | taatctatctggtgtctaaact |
| | | | | ggactctggagtccctgacagg |
| | | | | ttcactggcagtggatcaggaa |
| | | | | cagattttacgctgaaaatcag |
| | | | | cagagtggaggctgaggatttg |
| | | | | ggagtttattactgcgtgcaag |
| | | | | gtacacattttcctctcacgtt |
| | | | | cggtgctgggaccaagctggag |
| | | | | ctgaaaggtggtggtggttctg |
| | | | | gcggcggcggctccggtggtgg |
| | | | | tggttctgaggttcagctgcag |
| | | | | cagtctggggcagagcttgtga |
| | | | | ggtcaggggcctcagtcaagtt |
| | | | | gtcctgcacagcttctggcttc |
| | | | | aacattaaagactactatttgc |
| | | | | actgggtgaagcagaggcctga |
| | | | | acagggcctggagtggattgcc |
| | | | | tggattgatcttgagaatggtg |
| | | | | atattaaatatgccccgaagtt |
| | | | | tcagggcaaggccactataact |
| | | | | gcagacacatcctccaacacag |
| | | | | cctacctgcagctcagcagcct |
| | | | | gacatctgaggacactgccgtc |
| | | | | tattactgtaatccctattact |
| | | | | acggtagtaactacgactatgc |
| | | | | tatggactactggggtcaagga |
| | | | | acctcagtcaccgtctcctccg |
| | | | | gaggtggtggatcccaggctgt |
| | | | | tgtgactcaggaatctgcactc |
| | | | | accacatcacctggtgaaacag |
| | | | | tcacactcacttgtcgctcaag |
| | | | | tactggggctgttacaactagt |
| | | | | aactatgccaactgggtccaag |
| | | | | aaaaaccagatcatttattcac |
| | | | | tggtctaataggtggtaccaac |
| | | | | aagcgagctccaggtgtgcctg |
| | | | | ccagattctcaggctccctgat |
| | | | | tggagacaaggctgccctcacc |
| | | | | atcacaggggcacagactgagg |
| | | | | atgaggcaatatatttctgtgc |
| | | | | tctatggtacagcaacctctgg |
| | | | | gtgttcggtggaggaaccaaac |
| | | | | tgactgtcctaggtggtggtgg |
| | | | | ttctggcggcggcggctccggt |
| | | | | ggtggtggttctgaggtgaagc |
| | | | | ttctcgagtctggaggaggatt |
| | | | | ggtgcagcctaaagggtcattg |
| | | | | aaactctcatgtgcagcctctg |
| | | | | gattcaccttcaatacctacgc |
| | | | | catgaactgggtccgccaggct |
| | | | | ccaggaaagggtttggaatggg |
| | | | | ttgctcgcataagaagtaaata |
| | | | | taataattatgcaacatattat |
| | | | | gccgattcagtgaaagacaggt |
| | | | | tcaccatctccagagatgattc |
| | | | | acaaagcattctctatctacaa |
| | | | | atgaacaacttgaaaactgagg |
| | | | | acacagccatgtactactgtgt |
| | | | | gagacatgggaacttcggtaat |
| | | | | agctacgtttcctggtttgctt |
| | | | | actggggccaagggactctggt |
| | | | | cactgtctctgca |
| 66 | 2G8LHx | arti- | AA | DVVMTQTPLTLSVTIGQPASIS |
| | SEQ ID | ficial | | CKSSQSLLYSNGKTYLNWILQR |
| | NO. 12 | | | PGQSPKRLIYLVSKLDSGVPDR |
| | | | | FTGSGSGTDFTLKISRVEAEDL |
| | | | | GVYYCVQGTHFPLTFGAGTKLE |
| | | | | LKGGGGSGGGGSGGGGSEVQLQ |
| | | | | QSGAELVRSGASVKLSCTASGF |
| | | | | NIKDYYLHWVKQRPEQGLEWIA |
| | | | | WIDLENGDIKYAPKFQGKATIT |
| | | | | ADTSSNTAYLQLSSLTSEDTAV |
| | | | | YYCNPYYYGSNYDYAMDYWGQG |
| | | | | TSVTVSSGGGGSQAVVTQESAL |
| | | | | TTSPGETVTLTCRSSTGAVTTS |
| | | | | NYANWVQEKPDHLFTGLIGGTN |
| | | | | KRAPGVPARFSGSLIGDKAALT |
| | | | | ITGAQTEDEAIYFCALWYSNLW |
| | | | | VFGGGTKLTVLGGGGSGGGGSG |
| | | | | GGGSEVKLLESGGGLVQPKGSL |
| | | | | KLSCAASGFTFNTYAMNWVRQA |
| | | | | PGKGLEWVARIRSKYNNYATYY |
| | | | | ADSVKDRFTISRDDSQSILYLQ |
| | | | | MNNLKTEDTAMYYCVRHGNFGN |
| | | | | SYVSWFAYWGQGTLVTVSA |
| 67 | 2G8LHx | arti- | NA | gatgttgtgatgacccagactc |
| | SEQ ID | ficial | | cactcactttgtcggttaccat |
| | NO. 10 | | | tggacaaccagcctctatctct |
| | | | | tgcaagtcaagtcagagcctct |
| | | | | tatatagtaatggaaaaaccta |
| | | | | tttgaactggatattacagagg |
| | | | | ccaggccagtctccaaagcgcc |
| | | | | taatctatctggtgtctaaact |
| | | | | ggactctggagtccctgacagg |
| | | | | ttcactggcagtggatcaggaa |
| | | | | cagattttacgctgaaaatcag |
| | | | | cagagtggaggctgaggatttg |
| | | | | ggagtttattactgcgtgcaag |
| | | | | gtacacattttcctctcacgtt |
| | | | | cggtgctgggaccaagctggag |
| | | | | ctgaaaggtggtggtggttctg |
| | | | | gcggcggcggctccggtggtgg |
| | | | | tggttctgaggttcagctgcag |
| | | | | cagtctggggcagagcttgtga |
| | | | | ggtcaggggcctcagtcaagtt |
| | | | | gtcctgcacagcttctggcttc |
| | | | | aacattaaagactactatttgc |
| | | | | actgggtgaagcagaggcctga |
| | | | | acagggcctggagtggattgcc |
| | | | | tggattgatcttgagaatggtg |
| | | | | atattaaatatgccccgaagtt |
| | | | | tcagggcaaggccactataact |
| | | | | gcagacacatcctccaacacag |
| | | | | cctacctgcagctcagcagcct |
| | | | | gacatctgaggacactgccgtc |
| | | | | tattactgtaatccctattact |
| | | | | acggtagtaactacgactatgc |
| | | | | tatggactactggggtcaagga |
| | | | | acctcagtcaccgtctcctccg |
| | | | | gaggtggtggatccgaggtgaa |
| | | | | gcttctcgagtctggaggagga |
| | | | | ttggtgcagcctaaagggtcat |
| | | | | tgaaactctcatgtgcagcctc |
| | | | | tggattcaccttcaatacctac |
| | | | | gccatgaactgggtccgccagg |
| | | | | ctccaggaaagggtttggaatg |
| | | | | ggttgctcgcataagaagtaaa |
| | | | | tataataattatgcaacatatt |
| | | | | atgccgattcagtgaaagacag |
| | | | | gttcaccatctccagagatgat |
| | | | | tcacaaagcattctctatctac |
| | | | | aaatgaacaacttgaaaactga |
| | | | | ggacacagccatgtactactgt |
| | | | | gtgagacatgggaacttcggta |
| | | | | atagctacgtttcctggtttgc |
| | | | | ttactggggccaagggactctg |
| | | | | gtcactgtctctgcaggtggtg |
| | | | | gtggttctggcggcggcggctc |
| | | | | cggtggtggtggttctcaggct |
| | | | | gttgtgactcaggaatctgcac |
| | | | | tcaccacatcacctggtgaaac |
| | | | | agtcacactcacttgtcgctca |
| | | | | agtactggggctgttacaacta |
| | | | | gtaactatgccaactgggtcca |
| | | | | agaaaaaccagatcatttattc |
| | | | | actggtctaataggtggtacca |
| | | | | acaagcgagctccaggtgtgcc |
| | | | | tgccagattctcaggctccctg |
| | | | | attggagacaaggctgccctca |
| | | | | ccatcacaggggcacagactga |
| | | | | ggatgaggcaatatatttctgt |
| | | | | gctctatggtacagcaacctct |
| | | | | gggtgttcggtggaggaaccaa |
| | | | | actgactgtccta |
| 68 | 2G8LHx | arti- | AA | DVVMTQTPLTLSVTIGQPASIS |
| | SEQ ID | ficial | | CKSSQSLLYSNGKTYLNWILQR |
| | NO. 10 | | | PGQSPKRLIYLVSKLDSGVPDR |
| | | | | FTGSGSGTDFTLKISRVEAEDL |
| | | | | GVYYCVQGTHFPLTFGAGTKLE |
| | | | | LKGGGGSGGGGSGGGGSEVQLQ |
| | | | | QSGAELVRSGASVKLSCTASGF |
| | | | | NIKDYYLHWVKQRPEQGLEWIA |
| | | | | WIDLENGDIKYAPKFQGKATIT |
| | | | | ADTSSNTAYLQLSSLTSEDTAV |
| | | | | YYCNPYYYGSNYDYAMDYWGQG |
| | | | | TSVTVSSGGGGSEVKLLESGGG |
| | | | | LVQPKGSLKLSCAASGFTFNTY |
| | | | | AMNWVRQAPGKGLEWVARIRSK |
| | | | | YNNYATYYADSVKDRFTISRDD |
| | | | | SQSILYLQMNNLKTEDTAMYYC |
| | | | | VRHGNFGNSYVSWFAYWGQGTL |
| | | | | VTVSAGGGGSGGGGSGGGGSQA |
| | | | | VVTQESALTTSPGETVTLTCRS |
| | | | | STGAVTTSNYANWVQEKPDHLF |
| | | | | TGLIGGTNKRAPGVPARFSGSL |
| | | | | IGDKAALTITGAQTEDEAIYFC |
| | | | | ALWYSNLWVFGGGTKLTVL |
| 69 | 2G8LHx | arti- | NA | gatgttgtgatgacccagactc |
| | SEQ ID | ficial | | cactcactttgtcggttaccat |
| | NO. 16 | | | tggacaaccagcctctatctct |
| | | | | tgcaagtcaagtcagagcctct |
| | | | | tatatagtaatggaaaaaccta |
| | | | | tttgaactggatattacagagg |
| | | | | ccaggccagtctccaaagcgcc |
| | | | | taatctatctggtgtctaaact |
| | | | | ggactctggagtccctgacagg |
| | | | | ttcactggcagtggatcaggaa |
| | | | | cagattttacgctgaaaatcag |
| | | | | cagagtggaggctgaggatttg |
| | | | | ggagtttattactgcgtgcaag |
| | | | | gtacacattttcctctcacgtt |
| | | | | cggtgctgggaccaagctggag |
| | | | | ctgaaaggtggtggtggttctg |
| | | | | gcggcggcggctccggtggtgg |
| | | | | tggttctgaggttcagctgcag |
| | | | | cagtctggggcagagcttgtga |
| | | | | ggtcaggggcctcagtcaagtt |
| | | | | gtcctgcacagcttctggcttc |
| | | | | aacattaaagactactatttgc |
| | | | | actgggtgaagcagaggcctga |
| | | | | acagggcctggagtggattgcc |
| | | | | tggattgatcttgagaatggtg |
| | | | | atattaaatatgccccgaagtt |
| | | | | tcagggcaaggccactataact |
| | | | | gcagacacatcctccaacacag |
| | | | | cctacctgcagctcagcagcct |
| | | | | gacatctgaggacactgccgtc |
| | | | | tattactgtaatccctattact |
| | | | | acggtagtaactacgactatgc |
| | | | | tatggactactggggtcaagga |
| | | | | acctcagtcaccgtctcctccg |
| | | | | gaggtggtggatcccaagttgt |
| | | | | tctcacccagtctccagcaatc |
| | | | | atgtctgcatttccaggggaga |
| | | | | aggtcaccatgacctgcagtgc |
| | | | | cagctcaagtgtaagttacatg |
| | | | | aactggtaccagcagaagtcag |
| | | | | gcacctcccccaaaagatggat |
| | | | | ttatgactcatccaaactggct |
| | | | | tctggagtccctgctcgcttca |
| | | | | gtggcagtgggtctgggacctc |
| | | | | ttattctctcacaatcagcagc |
| | | | | atggagactgaagatgctgcca |
| | | | | cttattactgccagcagtggag |
| | | | | tcgtaacccacccacgttcgga |
| | | | | ggggggaccaagctacaaatta |
| | | | | caggtggtggtggttctggcgg |
| | | | | cggcggctccggtggtggtggt |
| | | | | tctcaggtccagctgcagcagt |
| | | | | ctggggctgaactggcaagacc |
| | | | | tggggcctcagtgaagatgtcc |
| | | | | tgcaaggcttctggctacacct |
| | | | | ttactagatctacgatgcactg |
| | | | | ggtaaaacagaggcctggacag |
| | | | | ggtctggaatggattggataca |
| | | | | ttaatcctagcagtgcttatac |
| | | | | taattacaatcagaaattcaag |
| | | | | gacaaggccacattgactgcag |
| | | | | acaaatcctccagtacagccta |
| | | | | catgcaactgagtagcctgaca |
| | | | | tctgaggactctgcagtctatt |
| | | | | actgtgcaagtccgcaagtcca |
| | | | | ctatgattacaacgggtttcct |
| | | | | tactggggccaagggactctgg |
| | | | | tcactgtctctgca |
| 70 | 2G8LHx | arti- | AA | DVVMTQTPLTLSVTIGQPASIS |
| | SEQ ID | ficial | | CKSSQSLLYSNGKTYLNWILQR |
| | NO. 16 | | | PGQSPKRLIYLVSKLDSGVPDR |
| | | | | FTGSGSGTDFTLKISRVEAEDL |
| | | | | GVYYCVQGTHFPLTFGAGTKLE |
| | | | | LKGGGGSGGGGSGGGGSEVQLQ |
| | | | | QSGAELVRSGASVKLSCTASGF |
| | | | | NIKDYYLHWVKQRPEQGLEWIA |
| | | | | WIDLENGDIKYAPKFQGKATIT |
| | | | | ADTSSNTAYLQLSSLTSEDTAV |
| | | | | YYCNPYYYGSNYDYAMDYWGQG |
| | | | | TSVTVSSGGGGSQVVLTQSPAI |
| | | | | MSAFPGEKVTMTCSASSSVSYM |
| | | | | NWYQQKSGTSPKRWIYDSSKLA |
| | | | | SGVPARFSGSGSGTSYSLTISS |
| | | | | METEDAATYYCQQWSRNPPTFG |
| | | | | GGTKLQITGGGGSGGGGSGGGG |
| | | | | SQVQLQQSGAELARPGASVKMS |
| | | | | CKASGYTFTRSTMHWVKQRPGQ |
| | | | | GLEWIGYINPSSAYTNYNQKFK |
| | | | | DKATLTADKSSSTAYMQLSSLT |
| | | | | SEDSAVYYCASPQVHYDYNGFP |
| | | | | YWGQGTLVTVSA |
| 71 | 2G8LHx | arti- | NA | gatgttgtgatgacccagactc |
| | SEQ ID | ficial | | cactcactttgtcggttaccat |
| | NO. 14 | | | tggacaaccagcctctatctct |
| | | | | tgcaagtcaagtcagagcctct |
| | | | | tatatagtaatggaaaaaccta |
| | | | | tttgaactggatattacagagg |
| | | | | ccaggccagtctccaaagcgcc |
| | | | | taatctatctggtgtctaaact |
| | | | | ggactctggagtccctgacagg |
| | | | | ttcactggcagtggatcaggaa |
| | | | | cagattttacgctgaaaatcag |
| | | | | cagagtggaggctgaggatttg |
| | | | | ggagtttattactgcgtgcaag |
| | | | | gtacacattttcctctcacgtt |
| | | | | cggtgctgggaccaagctggag |
| | | | | ctgaaaggtggtggtggttctg |
| | | | | gcggcggcggctccggtggtgg |
| | | | | tggttctgaggttcagctgcag |
| | | | | cagtctggggcagagcttgtga |
| | | | | ggtcaggggcctcagtcaagtt |
| | | | | gtcctgcacagcttctggcttc |
| | | | | aacattaaagactactatttgc |
| | | | | actgggtgaagcagaggcctga |
| | | | | acagggcctggagtggattgcc |
| | | | | tggattgatcttgagaatggtg |
| | | | | atattaaatatgccccgaagtt |
| | | | | tcagggcaaggccactataact |
| | | | | gcagacacatcctccaacacag |
| | | | | cctacctgcagctcagcagcct |
| | | | | gacatctgaggacactgccgtc |
| | | | | tattactgtaatccctattact |
| | | | | acggtagtaactacgactatgc |
| | | | | tatggactactggggtcaagga |
| | | | | acctcagtcaccgtctcctccg |
| | | | | gaggtggtggatcccaggtcca |
| | | | | gctgcagcagtctggggctgaa |
| | | | | ctggcaagacctggggcctcag |
| | | | | tgaagatgtcctgcaaggcttc |
| | | | | tggctacacctttactagatct |
| | | | | acgatgcactgggtaaaacaga |
| | | | | ggcctggacagggtctggaatg |
| | | | | gattggatacattaatcctagc |
| | | | | agtgcttatactaattacaatc |
| | | | | agaaattcaaggacaaggccac |
| | | | | attgactgcagacaaatcctcc |
| | | | | agtacagcctacatgcaactga |
| | | | | gtagcctgacatctgaggactc |
| | | | | tgcagtctattactgtgcaagt |
| | | | | ccgcaagtccactatgattaca |
| | | | | acgggtttccttactggggcca |
| | | | | agggactctggtcactgtctct |
| | | | | gcaggtggtggtggttctggcg |
| | | | | gcggcggctccggtggtggtgg |
| | | | | ttctcaagttgttctcacccag |
| | | | | tctccagcaatcatgtctgcat |
| | | | | ttccaggggagaaggtcaccat |
| | | | | gacctgcagtgccagctcaagt |
| | | | | gtaagttacatgaactggtacc |
| | | | | agcagaagtcaggcacctcccc |
| | | | | caaaagatggatttatgactca |
| | | | | tccaaactggcttctggagtcc |
| | | | | ctgctcgcttcagtggcagtgg |
| | | | | gtctgggacctcttattctctc |
| | | | | acaatcagcagcatggagactg |
| | | | | aagatgctgccacttattactg |
| | | | | ccagcagtggagtcgtaaccca |
| | | | | cccacgttcggaggggggacca |
| | | | | agctacaaattaca |
| 72 | 2G8LHx | arti- | AA | DVVMTQTPLTLSVTIGQPASIS |
| | SEQ ID | ficial | | CKSSQSLLYSNGKTYLNWILQR |
| | NO. 14 | | | PGQSPKRLIYLVSKLDSGVPDR |
| | | | | FTGSGSGTDFTLKISRVEAEDL |
| | | | | GVYYCVQGTHFPLTFGAGTKLE |
| | | | | LKGGGGSGGGGSGGGGSEVQLQ |
| | | | | QSGAELVRSGASVKLSCTASGF |
| | | | | NIKDYYLHWVKQRPEQGLEWIA |
| | | | | WIDLENGDIKYAPKFQGKATIT |
| | | | | ADTSSNTAYLQLSSLTSEDTAV |
| | | | | YYCNPYYYGSNYDYAMDYWGQG |
| | | | | TSVTVSSGGGGSQVQLQQSGAE |
| | | | | LARPGASVKMSCKASGYTFTRS |
| | | | | TMHWVKQRPGQGLEWIGYINPS |
| | | | | SAYTNYNQKFKDKATLTADKSS |
| | | | | STAYMQLSSLTSEDSAVYYCAS |
| | | | | PQVHYDYNGFPYWGQGTLVTVS |
| | | | | AGGGGSGGGGSGGGGSQVVLTQ |
| | | | | SPAIMSAFPGEKVTMTCSASSS |
| | | | | VSYMNWYQQKSGTSPKRWIYDS |
| | | | | SKLASGVPARFSGSGSGTSYSL |
| | | | | TISSMETEDAATYYCQQWSRNP |
| | | | | PTFGGGTKLQIT |
| 73 | 2G8HLx | arti- | NA | gaggttcagctgcagcagtctg |
| | SEQ ID | ficial | | gggcagagcttgtgaggtcagg |
| | NO. 12 | | | ggcctcagtcaagttgtcctgc |
| | | | | acagcttctggcttcaacatta |
| | | | | aagactactatttgcactgggt |
| | | | | gaagcagaggcctgaacagggc |
| | | | | ctggagtggattgcctggattg |
| | | | | atcttgagaatggtgatattaa |
| | | | | atatgccccgaagtttcagggc |
| | | | | aaggccactataactgcagaca |
| | | | | catcctccaacacagcctacct |
| | | | | gcagctcagcagcctgacatct |
| | | | | gaggacactgccgtctattact |
| | | | | gtaatccctattactacggtag |
| | | | | taactacgactatgctatggac |
| | | | | tactggggtcaaggaacctcag |
| | | | | tcaccgtctcctcaggtggtgg |
| | | | | tggttctggcggcggcggctcc |
| | | | | ggtggtggtggttctgatgttg |
| | | | | tgatgacccagactccactcac |
| | | | | tttgtcggttaccattggacaa |
| | | | | ccagcctctatctcttgcaagt |
| | | | | caagtcagagcctcttatatag |
| | | | | taatggaaaaacctatttgaac |
| | | | | tggatattacagaggccaggcc |
| | | | | agtctccaaagcgcctaatcta |
| | | | | tctggtgtctaaactggactct |
| | | | | ggagtccctgacaggttcactg |
| | | | | gcagtggatcaggaacagattt |
| | | | | tacgctgaaaatcagcagagtg |
| | | | | gaggctgaggatttgggagttt |
| | | | | attactgcgtgcaaggtacaca |
| | | | | ttttcctctcacgttcggtgct |
| | | | | gggaccaagctggagctgaaat |
| | | | | ccggaggtggtggatcccaggc |
| | | | | tgttgtgactcaggaatctgca |
| | | | | ctcaccacatcacctggtgaaa |
| | | | | cagtcacactcacttgtcgctc |
| | | | | aagtactggggctgttacaact |
| | | | | agtaactatgccaactgggtcc |
| | | | | aagaaaaaccagatcatttatt |
| | | | | cactggtctaataggtggtacc |
| | | | | aacaagcgagctccaggtgtgc |
| | | | | ctgccagattctcaggctccct |
| | | | | gattggagacaaggctgccctc |
| | | | | accatcacaggggcacagactg |
| | | | | aggatgaggcaatatatttctg |
| | | | | tgctctatggtacagcaacctc |
| | | | | tgggtgttcggtggaggaacca |
| | | | | aactgactgtcctaggtggtgg |
| | | | | tggttctggcggcggcggctcc |
| | | | | ggtggtggtggttctgaggtga |
| | | | | agcttctcgagtctggaggagg |
| | | | | attggtgcagcctaaagggtca |
| | | | | ttgaaactctcatgtgcagcct |
| | | | | ctggattcaccttcaataccta |
| | | | | cgccatgaactgggtccgccag |
| | | | | gctccaggaaagggtttggaat |
| | | | | gggttgctcgcataagaagtaa |
| | | | | atataataattatgcaacatat |
| | | | | tatgccgattcagtgaaagaca |
| | | | | ggttcaccatctccagagatga |
| | | | | ttcacaaagcattctctatcta |
| | | | | caaatgaacaacttgaaaactg |
| | | | | aggacacagccatgtactactg |
| | | | | tgtgagacatgggaacttcggt |
| | | | | aatagctacgtttcctggtttg |
| | | | | cttactggggccaagggactct |
| | | | | ggtcactgtctctgca |
| 74 | 2G8HLx | arti- | AA | EVQLQQSGAELVRSGASVKLSC |
| | SEQ ID | ficial | | TASGFNIKDYYLHWVKQRPEQG |
| | NO. 12 | | | LEWIAWIDLENGDIKYAPKFQG |
| | | | | KATITADTSSNTAYLQLSSLTS |
| | | | | EDTAVYYCNPYYYGSNYDYAMD |
| | | | | YWGQGTSVTVSSGGGGSGGGGS |
| | | | | GGGGSDVVMTQTPLTLSVTIGQ |
| | | | | PASISCKSSQSLLYSNGKTYLN |
| | | | | WILQRPGQSPKRLIYLVSKLDS |
| | | | | GVPDRFTGSGSGTDFTLKISRV |
| | | | | EAEDLGVYYCVQGTHFPLTFGA |
| | | | | GTKLELKSGGGGSQAVVTQESA |
| | | | | LTTSPGETVTLTCRSSTGAVTT |
| | | | | SNYANWVQEKPDHLFTGLIGGT |
| | | | | NKRAPGVPARFSGSLIGDKAAL |
| | | | | TITGAQTEDEAIYFCALWYSNL |
| | | | | WVFGGGTKLTVLGGGGSGGGGS |
| | | | | GGGGSEVKLLESGGGLVQPKGS |
| | | | | LKLSCAASGFTFNTYAMNWVRQ |
| | | | | APGKGLEWVARIRSKYNNYATY |
| | | | | YADSVKDRFTISRDDSQSILYL |
| | | | | QMNNLKTEDTAMYYCVRHGNFG |
| | | | | NSYVSWFAYWGQGTLVTVSA |
| 75 | 2G8HLx | arti- | NA | gaggttcagctgcagcagtctg |
| | SEQ ID | ficial | | gggcagagcttgtgaggtcagg |
| | NO. 10 | | | ggcctcagtcaagttgtcctgc |
| | | | | acagcttctggcttcaacatta |
| | | | | aagactactatttgcactgggt |
| | | | | gaagcagaggcctgaacagggc |
| | | | | ctggagtggattgcctggattg |
| | | | | atcttgagaatggtgatattaa |
| | | | | atatgccccgaagtttcagggc |
| | | | | aaggccactataactgcagaca |
| | | | | catcctccaacacagcctacct |
| | | | | gcagctcagcagcctgacatct |
| | | | | gaggacactgccgtctattact |
| | | | | gtaatccctattactacggtag |
| | | | | taactacgactatgctatggac |
| | | | | tactggggtcaaggaacctcag |
| | | | | tcaccgtctcctcaggtggtgg |
| | | | | tggttctggcggcggcggctcc |
| | | | | ggtggtggtggttctgatgttg |
| | | | | tgatgacccagactccactcac |
| | | | | tttgtcggttaccattggacaa |
| | | | | ccagcctctatctcttgcaagt |
| | | | | caagtcagagcctcttatatag |
| | | | | taatggaaaaacctatttgaac |
| | | | | tggatattacagaggccaggcc |
| | | | | agtctccaaagcgcctaatcta |
| | | | | tctggtgtctaaactggactct |
| | | | | ggagtccctgacaggttcactg |
| | | | | gcagtggatcaggaacagattt |
| | | | | tacgctgaaaatcagcagagtg |
| | | | | gaggctgaggatttgggagttt |
| | | | | attactgcgtgcaaggtacaca |
| | | | | ttttcctctcacgttcggtgct |
| | | | | gggaccaagctggagctgaaat |
| | | | | ccggaggtggtggatccgaggt |
| | | | | gaagcttctcgagtctggagga |
| | | | | ggattggtgcagcctaaagggt |
| | | | | cattgaaactctcatgtgcagc |
| | | | | ctctggattcaccttcaatacc |
| | | | | tacgccatgaactgggtccgcc |
| | | | | aggctccaggaaagggtttgga |
| | | | | atgggttgctcgcataagaagt |
| | | | | aaatataataattatgcaacat |
| | | | | attatgccgattcagtgaaaga |
| | | | | caggttcaccatctccagagat |
| | | | | gattcacaaagcattctctatc |
| | | | | tacaaatgaacaacttgaaaac |
| | | | | tgaggacacagccatgtactac |
| | | | | tgtgtgagacatgggaacttcg |
| | | | | gtaatagctacgtttcctggtt |
| | | | | tgcttactggggccaagggact |
| | | | | ctggtcactgtctctgcaggtg |
| | | | | gtggtggttctggcggcggcgg |
| | | | | ctccggtggtggtggttctcag |
| | | | | gctgttgtgactcaggaatctg |
| | | | | cactcaccacatcacctggtga |
| | | | | aacagtcacactcacttgtcgc |
| | | | | tcaagtactggggctgttacaa |
| | | | | ctagtaactatgccaactgggt |
| | | | | ccaagaaaaaccagatcattta |
| | | | | ttcactggtctaataggtggta |
| | | | | ccaacaagcgagctccaggtgt |
| | | | | gcctgccagattctcaggctcc |
| | | | | ctgattggagacaaggctgccc |
| | | | | tcaccatcacaggggcacagac |
| | | | | tgaggatgaggcaatatatttc |
| | | | | tgtgctctatggtacagcaacc |
| | | | | tctgggtgttcggtggaggaac |
| | | | | caaactgactgtccta |
| 76 | 2G8HLx | arti- | AA | EVQLQQSGAELVRSGASVKLSC |
| | SEQ ID | ficial | | TASGFNIKDYYLHWVKQRPEQG |
| | NO. 10 | | | LEWIAWIDLENGDIKYAPKFQG |
| | | | | KATITADTSSNTAYLQLSSLTS |
| | | | | EDTAVYYCNPYYYGSNYDYAMD |
| | | | | YWGQGTSVTVSSGGGGSGGGGS |
| | | | | GGGGSDVVMTQTPLTLSVTIGQ |
| | | | | PASISCKSSQSLLYSNGKTYLN |
| | | | | WILQRPGQSPKRLIYLVSKLDS |
| | | | | GVPDRFTGSGSGTDFTLKISRV |
| | | | | EAEDLGVYYCVQGTHFPLTFGA |
| | | | | GTKLELKSGGGGSEVKLLESGG |
| | | | | GLVQPKGSLKLSCAASGFTFNT |
| | | | | YAMNWVRQAPGKGLEWVARIRS |
| | | | | KYNNYATYYADSVKDRFTISRD |
| | | | | DSQSILYLQMNNLKTEDTAMYY |
| | | | | CVRHGNFGNSYVSWFAYWGQGT |
| | | | | LVTVSAGGGGSGGGGSGGGGSQ |
| | | | | AVVTQESALTTSPGETVTLTCR |
| | | | | SSTGAVTTSNYANWVQEKPDHL |
| | | | | FTGLIGGTNKRAPGVPARFSGS |
| | | | | LIGDKAALTITGAQTEDEAIYF |
| | | | | CALWYSNLWVFGGGTKLTVL |
| 77 | 2G8HLx | arti- | NA | gaggttcagctgcagcagtctg |
| | SEQ ID | ficial | | gggcagagcttgtgaggtcagg |
| | NO. 16 | | | ggcctcagtcaagttgtcctgc |
| | | | | acagcttctggcttcaacatta |
| | | | | aagactactatttgcactgggt |
| | | | | gaagcagaggcctgaacagggc |
| | | | | ctggagtggattgcctggattg |
| | | | | atcttgagaatggtgatattaa |
| | | | | atatgccccgaagtttcagggc |
| | | | | aaggccactataactgcagaca |
| | | | | catcctccaacacagcctacct |
| | | | | gcagctcagcagcctgacatct |
| | | | | gaggacactgccgtctattact |
| | | | | gtaatccctattactacggtag |
| | | | | taactacgactatgctatggac |
| | | | | tactggggtcaaggaacctcag |
| | | | | tcaccgtctcctcaggtggtgg |
| | | | | tggttctggcggcggcggctcc |
| | | | | ggtggtggtggttctgatgttg |
| | | | | tgatgacccagactccactcac |
| | | | | tttgtcggttaccattggacaa |
| | | | | ccagcctctatctcttgcaagt |
| | | | | caagtcagagcctcttatatag |
| | | | | taatggaaaaacctatttgaac |
| | | | | tggatattacagaggccaggcc |
| | | | | agtctccaaagcgcctaatcta |
| | | | | tctggtgtctaaactggactct |
| | | | | ggagtccctgacaggttcactg |
| | | | | gcagtggatcaggaacagattt |
| | | | | tacgctgaaaatcagcagagtg |
| | | | | gaggctgaggatttgggagttt |
| | | | | attactgcgtgcaaggtacaca |
| | | | | ttttcctctcacgttcggtgct |
| | | | | gggaccaagctggagctgaaat |
| | | | | ccggaggtggtggatcccaagt |
| | | | | tgttctcacccagtctccagca |
| | | | | atcatgtctgcatttccagggg |
| | | | | agaaggtcaccatgacctgcag |
| | | | | tgccagctcaagtgtaagttac |
| | | | | atgaactggtaccagcagaagt |
| | | | | caggcacctcccccaaaagatg |
| | | | | gatttatgactcatccaaactg |
| | | | | gcttctggagtccctgctcgct |
| | | | | tcagtggcagtgggtctgggac |
| | | | | ctcttattctctcacaatcagc |
| | | | | agcatggagactgaagatgctg |
| | | | | ccacttattactgccagcagtg |
| | | | | gagtcgtaacccacccacgttc |
| | | | | ggaggggggaccaagctacaaa |
| | | | | ttacaggtggtggtggttctgg |
| | | | | cggcggcggctccggtggtggt |
| | | | | ggttctcaggtccagctgcagc |
| | | | | agtctggggctgaactggcaag |
| | | | | acctggggcctcagtgaagatg |
| | | | | tcctgcaaggcttctggctaca |
| | | | | cctttactagatctacgatgca |
| | | | | ctgggtaaaacagaggcctgga |
| | | | | cagggtctggaatggattggat |
| | | | | acattaatcctagcagtgctta |
| | | | | tactaattacaatcagaaattc |
| | | | | aaggacaaggccacattgactg |
| | | | | cagacaaatcctccagtacagc |
| | | | | ctacatgcaactgagtagcctg |
| | | | | acatctgaggactctgcagtct |
| | | | | attactgtgcaagtccgcaagt |
| | | | | ccactatgattacaacgggttt |
| | | | | ccttactggggccaagggactc |
| | | | | tggtcactgtctctgca |
| 78 | 2G8HLx | arti- | AA | EVQLQQSGAELVRSGASVKLSC |
| | SEQ ID | ficial | | TASGFNIKDYYLHWVKQRPEQG |
| | NO. 16 | | | LEWIAWIDLENGDIKYAPKFQG |
| | | | | KATITADTSSNTAYLQLSSLTS |
| | | | | EDTAVYYCNPYYYGSNYDYAMD |
| | | | | YWGQGTSVTVSSGGGGSGGGGS |
| | | | | GGGGSDVVMTQTPLTLSVTIGQ |
| | | | | PASISCKSSQSLLYSNGKTYLN |
| | | | | WILQRPGQSPKRLIYLVSKLDS |
| | | | | GVPDRFTGSGSGTDFTLKISRV |
| | | | | EAEDLGVYYCVQGTHFPLTFGA |
| | | | | GTKLELKSGGGGSQVVLTQSPA |
| | | | | IMSAFPGEKVTMTCSASSSVSY |
| | | | | MNWYQQKSGTSPKRWIYDSSKL |
| | | | | ASGVPARFSGSGSGTSYSLTIS |
| | | | | SMETEDAATYYCQQWSRNPPTF |
| | | | | GGGTKLQITGGGGSGGGGSGGG |
| | | | | GSQVQLQQSGAELARPGASVKM |
| | | | | SCKASGYTFTRSTMHWVKQRPG |
| | | | | QGLEWIGYINPSSAYTNYNQKF |
| | | | | KDKATLTADKSSSTAYMQLSSL |
| | | | | TSEDSAVYYCASPQVHYDYNGF |
| | | | | PYWGQGTLVTVSA |
| 79 | 2G8HLx | arti- | NA | gaggttcagctgcagcagtctg |
| | SEQ ID | ficial | | gggcagagcttgtgaggtcagg |
| | NO. 14 | | | ggcctcagtcaagttgtcctgc |
| | | | | acagcttctggcttcaacatta |
| | | | | aagactactatttgcactgggt |
| | | | | gaagcagaggcctgaacagggc |
| | | | | ctggagtggattgcctggattg |
| | | | | atcttgagaatggtgatattaa |
| | | | | atatgccccgaagtttcagggc |
| | | | | aaggccactataactgcagaca |
| | | | | catcctccaacacagcctacct |
| | | | | gcagctcagcagcctgacatct |
| | | | | gaggacactgccgtctattact |
| | | | | gtaatccctattactacggtag |
| | | | | taactacgactatgctatggac |
| | | | | tactggggtcaaggaacctcag |
| | | | | tcaccgtctcctcaggtggtgg |
| | | | | tggttctggcggcggcggctcc |
| | | | | ggtggtggtggttctgatgttg |
| | | | | tgatgacccagactccactcac |
| | | | | tttgtcggttaccattggacaa |
| | | | | ccagcctctatctcttgcaagt |
| | | | | caagtcagagcctcttatatag |
| | | | | taatggaaaaacctatttgaac |
| | | | | tggatattacagaggccaggcc |
| | | | | agtctccaaagcgcctaatcta |
| | | | | tctggtgtctaaactggactct |
| | | | | ggagtccctgacaggttcactg |
| | | | | gcagtggatcaggaacagattt |
| | | | | tacgctgaaaatcagcagagtg |
| | | | | gaggctgaggatttgggagttt |
| | | | | attactgcgtgcaaggtacaca |
| | | | | ttttcctctcacgttcggtgct |
| | | | | gggaccaagctggagctgaaat |
| | | | | ccggaggtggtggatcccaggt |
| | | | | ccagctgcagcagtctggggct |
| | | | | gaactggcaagacctggggcct |
| | | | | cagtgaagatgtcctgcaaggc |
| | | | | ttctggctacacctttactaga |
| | | | | tctacgatgcactgggtaaaac |
| | | | | agaggcctggacagggtctgga |
| | | | | atggattggatacattaatcct |
| | | | | agcagtgcttatactaattaca |
| | | | | atcagaaattcaaggacaaggc |
| | | | | cacattgactgcagacaaatcc |
| | | | | tccagtacagcctacatgcaac |
| | | | | tgagtagcctgacatctgagga |
| | | | | ctctgcagtctattactgtgca |
| | | | | agtccgcaagtccactatgatt |
| | | | | acaacgggtttccttactgggg |
| | | | | ccaagggactctggtcactgtc |
| | | | | tctgcaggtggtggtggttctg |
| | | | | gcggcggcggctccggtggtgg |
| | | | | tggttctcaagttgttctcacc |
| | | | | cagtctccagcaatcatgtctg |
| | | | | catttccaggggagaaggtcac |
| | | | | catgacctgcagtgccagctca |
| | | | | agtgtaagttacatgaactggt |
| | | | | accagcagaagtcaggcacctc |
| | | | | ccccaaaagatggatttatgac |
| | | | | tcatccaaactggcttctggag |
| | | | | tccctgctcgcttcagtggcag |
| | | | | tgggtctgggacctcttattct |
| | | | | ctcacaatcagcagcatggaga |
| | | | | ctgaagatgctgccacttatta |
| | | | | ctgccagcagtggagtcgtaac |
| | | | | ccacccacgttcggagggggga |
| | | | | ccaagctacaaattaca |
| 80 | 2G8HLx | arti- | AA | EVQLQQSGAELVRSGASVKLSC |
| | SEQ ID | ficial | | TASGFNIKDYYLHWVKQRPEQG |
| | NO. 14 | | | LEWIAWIDLENGDIKYAPKFQG |
| | | | | KATITADTSSNTAYLQLSSLTS |
| | | | | EDTAVYYCNPYYYGSNYDYAMD |
| | | | | YWGQGTSVTVSSGGGGSGGGGS |
| | | | | GGGGSDVVMTQTPLTLSVTIGQ |
| | | | | PASISCKSSQSLLYSNGKTYLN |
| | | | | WILQRPGQSPKRLIYLVSKLDS |
| | | | | GVPDRFTGSGSGTDFTLKISRV |
| | | | | EAEDLGVYYCVQGTHFPLTFGA |
| | | | | GTKLELKSGGGGSQVQLQQSGA |
| | | | | ELARPGASVKMSCKASGYTFTR |
| | | | | STMHWVKQRPGQGLEWIGYINP |
| | | | | SSAYTNYNQKFKDKATLTADKS |
| | | | | SSTAYMQLSSLTSEDSAVYYCA |
| | | | | SPQVHYDYNGFPYWGQGTLVTV |
| | | | | SAGGGGSGGGGSGGGGSQVVLT |
| | | | | QSPAIMSAFPGEKVTMTCSASS |
| | | | | SVSYMNWYQQKSGTSPKRWIYD |
| | | | | SSKLASGVPARFSGSGSGTSYS |
| | | | | LTISSMETEDAATYYCQQWSRN |
| | | | | PPTFGGGTKLQIT |
| 81 | 5′ primer | arti- | NA | 5′-SAGGTGCAGCTCGAGGAGT |
| | VH | ficial | | CAGGACCT-3′ |
| 82 | 5′ primer | arti- | NA | 5′-GAGGTCCAGCTCGAGCAGT |
| | VH | ficial | | CTGGACCT-3′ |
| 83 | 5′ primer | arti- | NA | 5′-CAGGTCCAACTCGAGCAGC |
| | VH | ficial | | CTGGGGCT-3′ |
| 84 | 5′ primer | arti- | NA | 5′-GAGGTTCAGCTCGAGCAGT |
| | VH | ficial | | CTGGGGCA-3′ |
| 85 | 5′ primer | arti- | NA | 5′-GARGTGAAGCTCGAGGAGT |
| | VH | ficial | | CTGGAGGA-3′ |
| 86 | 5′ primer | arti- | NA | 5′-GAGGTGAAGCTTCTCGAGT |
| | VH | ficial | | CTGGAGGT-3′ |
| 87 | 5′ primer | arti- | NA | 5′-GAAGTGAAGCTCGAGGAGT |
| | VH | ficial | | CTGGGGGA-3′ |
| 88 | 5′ primer | arti- | NA | 5′-GAGGTTCAGCTCGAGCAGT |
| | VH | ficial | | CTGGAGCT-3′ |
| 89 | 5′ primer | arti- | NA | 5′-GGGCTCGAGCACCATGGRA |
| | VH | ficial | | TGSAGCTGKGTMATSCTCT |
| 90 | 5′ primer | arti- | NA | 5′-GGGCTCGAGCACCATGRAC |
| | VH | ficial | | TTCGGGYTGAGCTKGGTTT |
| | | | | T-3′ |
| 91 | 5′ primer | arti- | NA | 5′-GGGCTCGAGCACCATGGCT |
| | VH | ficial | | GTCTTGGGGCTGCTCTTCT-3′ |
| 92 | 3′ primer | arti- | NA | 5′-GAGGAATTCGAACTGGACA |
| | VH | ficial | | GGGATCCAGAGTTCC-3′ |
| 93 | 3′ primer | arti- | NA | 5′-CGGAATTCGAATGACATGG |
| | VH | ficial | | ACATCTGGGTCATCC-3′ |
| 94 | 5′ primer | arti- | NA | 5′-CCAGTTCCGAGCTCGTTGT |
| | VL | ficial | | GACTCAGGAATCT-3′ |
| 95 | 5′ primer | arti- | NA | 5′-CCAGTTCCGAGCTCGTGTT |
| | VL | ficial | | GACGCAGCCGCCC-3′ |
| 96 | 5′ primer | arti- | NA | 5′-CCAGTTCCGAGCTCGTGCT |
| | VL | ficial | | CACCCAGTCTCCA-3′ |
| 97 | 5′ primer | arti- | NA | 5′-CCAGTTCCGAGCTCCAGAT |
| | VL | ficial | | GACCCAGTCTCCA-3′ |
| 98 | 5′ primer | arti- | NA | 5′-CCAGATGTGAGCTCGTGAT |
| | VL | ficial | | GACCCAGACTCCA-3′ |
| 99 | 5′ primer | arti- | NA | 5′-CCAGATGTGAGCTCGTCAT |
| | VL | ficial | | GACCCAGTCTCCA-3′ |
| 100 | 5′ primer | arti- | NA | 5′-CCAGTTCCGAGCTCGTGAT |
| | VL | ficial | | GACACAGTCTCCA-3′ |
| 101 | 5′ primer | arti- | NA | 5′-GGGGAGCTCCACCATGGAG |
| | VL | ficial | | ACAGACACACTCCTGCTAT-3′ |
| 102 | 5′ primer | arti- | NA | 5′-GGGGAGCTCCACCATGGAT |
| | VL | ficial | | TTTCAAGTGCAGATTTTCA |
| | | | | G-3′ |
| 103 | 5′ primer | arti- | NA | 5′-GGGGAGCTCCACCATGGAG |
| | VL | ficial | | WCACAKWCTCAGGTCTTTRT |
| | | | | A-3′ |
| 104 | 5′ primer | arti- | NA | 5′-GGGGAGCTCCACCATGKCC |
| | VL | ficial | | CCWRCTCAGYTYCTKGT-3′ |
| 105 | 3′ primer | arti- | NA | 5′-GAGGAATTCGAACTGCTCA |
| | VL | ficial | | CTGGATGGTGGG-3′ |
| 106 | 3′ primer | arti- | NA | 5′-CGGAATTCGAACAAACTCT |
| | VL | ficial | | TCTCCACAGTGTGACC-3′ |
| 107 | 3′ primer | arti- | NA | 5′-TATGCAACTAGTACAACCA |
| | VH | ficial | | CAATCCCTGGG-3′ |
| 108 | 3′ primer | arti- | NA | 5′-GCGCCGTCTAGAATTAACA |
| | VL | ficial | | CTCATTCCTGTTGAA-3′ |
| 109 | 5-10 LH x | arti- | AA | ELVMTQSPSSLTVTAGEKVTMS |
| | deimmu- | ficial | | CKSSQSLLNSGNQKNYLTWYQQ |
| | nised | | | KPGQPPKLLIYWASTRESGVPD |
| | (di) | | | RFTGSGSGTDFTLTISSVQAED |
| | anti-CD3 | | | LAVYYCQNDYSYPLTFGAGTKL |
| | | | | EIKGGGGSGGGGSGGGGSEVQL |
| | | | | LEQSGAELVRPGTSVKISCKAS |
| | | | | GYAFTNYWLGWVKQRPGHGLEW |
| | | | | IGDIFPGSGNIHYNEKFKGKAT |
| | | | | LTADKSSSTAYMQLSSLTFEDS |
| | | | | AVYFCARLRNWDEPMDYWGQGT |
| | | | | TVTVSSGGGGSDVQLVQSGAEV |
| | | | | KKPGASVKVSCKASGYTFTRYT |
| | | | | MHWVRQAPGQGLEWIGYINPSR |
| | | | | GYTNYADSVKGRFTITTDKSTS |
| | | | | TAYMELSSLRSEDTATYYCARY |
| | | | | YDDHYCLDYWGQGTTVTVSSGE |
| | | | | GTSTGSGGSGGSGGADDIVLTQ |
| | | | | SPATLSLSPGERATLSCRASQS |
| | | | | VSYMNWYQQKPGKAPKRWIYDT |
| | | | | SKVASGVPARFSGSGSGTDYSL |
| | | | | TINSLEAEDAATYYCQQWSSNP |
| | | | | LTFGGGTKVEIK |
| 110 | Human- | arti- | AA | EVQLLESGGGLVQPGGSLKLSC |
| | like VH | ficial | | AASGFTFNTYAMNWVRQAPGKG |
| | | | | LEWVARIRSKYNNYATYYADSV |
| | | | | KDRFTISRDDSKNTAYLQMNNL |
| | | | | KTEDTAVYYCVRHGNFGNSYVS |
| | | | | WFAYWGQGTLVTVSS |
| 111 | Human- | arti- | NA | gaggtgcagctgctcgagtctg |
| | like VH | ficial | | gaggaggattggtgcagcctgg |
| | | | | agggtcattgaaactctcatgt |
| | | | | gcagcctctggattcaccttca |
| | | | | atacctacgccatgaactgggt |
| | | | | ccgccaggctccaggaaagggt |
| | | | | ttggaatgggttgctcgcataa |
| | | | | gaagtaaatataataattatgc |
| | | | | aacatattatgccgattcagtg |
| | | | | aaagacaggttcaccatctcca |
| | | | | gagatgattcaaaaaacactgc |
| | | | | ctatctacaaatgaacaacttg |
| | | | | aaaactgaggacactgccgtgt |
| | | | | actactgtgtgagacatgggaa |
| | | | | cttcggtaatagctacgtttcc |
| | | | | tggtttgcttactggggccaag |
| | | | | ggactctggtcaccgtctcctc |
| | | | | a |
| 112 | VH: CDR3 | murine | AA | HGNFGNSYVSWFAY |
| 113 | VH: short | murine | AA | VSWFAY |
| | CDR3 | | | |
| 114 | VH: CDR2 | murine | AA | RIRSKYNNYATYYADSVKD |
| 115 | VH: CDR1 | murine | AA | TYAMN |
| 116 | VL: CDR3 | murine | AA | ALWYSNLWV |
| 117 | VL: CDR2 | murine | AA | GTNKRAP |
| 118 | VL: CDR1 | murine | AA | RSSTGAVTTSNYAN |
| 119 | VH: CDR3 | murine | AA | PQVHYDYNGFPY |
| 120 | VH: CDR2 | murine | AA | YINPSSAYTNYNQKFKD |
| 121 | VH: CDR1 | murine | AA | GYTFTRSTMH |
| 122 | 2G8 LHx | arti- | AA | DVVMTQTPLTLSVTIGQPASIS |
| | SEQ ID | ficial | | CKSSQSLLYSNGKTYLNWILQR |
| | NO. 146 | | | PGQSPKRLIYLVSKLDSGVPDR |
| | | | | FTGSGSGTDFTLKISRVEAEDL |
| | | | | GVYYCVQGTHFPLTFGAGTKLE |
| | | | | LKGGGGSGGGGSGGGGSEVQLQ |
| | | | | QSGAELVRSGASVKLSCTASGF |
| | | | | NIKDYYLHWVKQRPEQGLEWIA |
| | | | | WIDLENGDIKYAPKIFQGKATI |
| | | | | TADTSSNTAYLQLSSLTSEDTA |
| | | | | VYYCNPYYYGSNYDYAMDYWGQ |
| | | | | GTSVTVSSGGGGSEVKLLESGG |
| | | | | GLVQPGGSLKLSCAASGFTFNT |
| | | | | YAMNWVRQAPGKGLEWVARIRS |
| | | | | KYNNYATYYADSVKDRFTISRD |
| | | | | DSKNTAYLQMNNLKTEDTAVYY |
| | | | | CVRHGNFGNSYVSWFAYWGQGT |
| | | | | LVTVSSGGGGSGGGGSGGGGSE |
| | | | | LVVTQESALTTSPGETVTLTCR |
| | | | | SSTGAVTTSNYANWVQEKPDHL |
| | | | | FTGLIGGTNKRAPGVPARFSGS |
| | | | | LIGDKAALTITGAQTEDEAIYF |
| | | | | CALWYSNLWVFGGGTKLTVLHH |
| | | | | HHHH |
| 123 | 2G8 LHx | arti- | NA | gatgttgtgatgacccagactc |
| | SEQ ID | ficial | | cactcactttgtcggttaccat |
| | NO. 146 | | | tggacaaccagcctctatctct |
| | | | | tgcaagtcaagtcagagcctct |
| | | | | tatatagtaatggaaaaaccta |
| | | | | tttgaactggatattacagagg |
| | | | | ccaggccagtctccaaagcgcc |
| | | | | taatctatctggtgtctaaact |
| | | | | ggactctggagtccctgacagg |
| | | | | ttcactggcagtggatcaggaa |
| | | | | cagattttacgctgaaaatcag |
| | | | | cagagtggaggctgaggatttg |
| | | | | ggagtttattactgcgtgcaag |
| | | | | gtacacattttcctctcacgtt |
| | | | | cggtgctgggaccaagctggag |
| | | | | ctgaaaggtggtggtggttctg |
| | | | | gcggcggcggctccggtggtgg |
| | | | | tggttctgaggttcagctgcag |
| | | | | cagtctggggcagagcttgtga |
| | | | | ggtcaggggcctcagtcaagtt |
| | | | | gtcctgcacagcttctggcttc |
| | | | | aacattaaagactactatttgc |
| | | | | actgggtgaagcagaggcctga |
| | | | | acagggcctggagtggattgcc |
| | | | | tggattgatcttgagaatggtg |
| | | | | atattaaatatgccccgaagtt |
| | | | | tcagggcaaggccactataact |
| | | | | gcagacacatcctccaacacag |
| | | | | cctacctgcagctcagcagcct |
| | | | | gacatctgaggacactgccgtc |
| | | | | tattactgtaatccctattact |
| | | | | acggtagtaactacgactatgc |
| | | | | tatggactactggggtcaagga |
| | | | | acctcagtcaccgtctcctccg |
| | | | | gaggtggtggatccgaggtgaa |
| | | | | gcttctcgagtctggaggagga |
| | | | | ttggtgcagcctggagggtcat |
| | | | | tgaaactctcatgtgcagcctc |
| | | | | tggattcaccttcaatacctac |
| | | | | gccatgaactgggtccgccagg |
| | | | | ctccaggaaagggtttggaatg |
| | | | | ggttgctcgcataagaagtaaa |
| | | | | tataataattatgcaacatatt |
| | | | | atgccgattcagtgaaagacag |
| | | | | gttcaccatctccagagatgat |
| | | | | tcaaaaaacactgcctatctac |
| | | | | aaatgaacaacttgaaaactga |
| | | | | ggacactgccgtgtactactgt |
| | | | | gtgagacatgggaacttcggta |
| | | | | atagctacgtttcctggtttgc |
| | | | | ttactggggccaagggactctg |
| | | | | gtcaccgtctcctcaggtggtg |
| | | | | gtggttctggcggcggcggctc |
| | | | | cggtggtggtggttctgagctc |
| | | | | gttgtgactcaggaatctgcac |
| | | | | tcaccacatcacctggtgaaac |
| | | | | agtcacactcacttgtcgctca |
| | | | | agtactggggctgttacaacta |
| | | | | gtaactatgccaactgggtcca |
| | | | | agaaaaaccagatcatttattc |
| | | | | actggtctaataggtggtacca |
| | | | | acaagcgagcaccaggtgtgcc |
| | | | | tgccagattctcaggctccctg |
| | | | | attggagacaaggctgccctca |
| | | | | ccatcacaggggcacagactga |
| | | | | ggatgaggcaatatatttctgt |
| | | | | gctctatggtacagcaacctct |
| | | | | gggtgttcggtggaggaaccaa |
| | | | | actgactgtcctacatcatcac |
| | | | | catcatcat |
| 124 | 5-10 LHx | arti- | AA | ELVMTQSPSSLTVTAGEKVTMS |
| | SEQ ID | ficial | | CKSSQSLLNSGNQKNYLTWYQQ |
| | NO. 146 | | | KPGQPPKLLIYWASTRESGVPD |
| | | | | RFTGSGSGTDFTLTISSVQAED |
| | | | | LAVYYCQNDYSYPLTFGAGTKL |
| | | | | EIKGGGGSGGGGSGGGGSEVQL |
| | | | | LEQSGAELVRPGTSVKISCKAS |
| | | | | GYAFTNYWLGWVKQRPGHGLEW |
| | | | | IGDIFPGSGNIHYNEKFKGKAT |
| | | | | LTADKSSSTAYMQLSSLTFEDS |
| | | | | AVYFCARLRNWDEPMDYWGQGT |
| | | | | TVTVSSGGGGSEVKLLESGGGL |
| | | | | VQPGGSLKLSCAASGFTFNTYA |
| | | | | MNWVRQAPGKGLEWVARIRSKY |
| | | | | NNYATYYADSVKDRFTISRDDS |
| | | | | KNTAYLQMNNLKTEDTAVYYCV |
| | | | | RHGNFGNSYVSWFAYWGQGTLV |
| | | | | TVSSGGGGSGGGGSGGGGSELV |
| | | | | VTQESALTTSPGETVTLTCRSS |
| | | | | TGAVTTSNYANWVQEKPDHLFT |
| | | | | GLIGGTNKRAPGVPARFSGSLI |
| | | | | GDKAALTITGAQTEDEAIYFCA |
| | | | | LWYSNLWVFGGGTKLTVLHHHH |
| | | | | HH |
| 125 | 5-10 LHx | arti- | NA | gagctcgtgatgacacagtctc |
| | SEQ ID | ficial | | catcctccctgactgtgacagc |
| | NO. 146 | | | aggagagaaggtcactatgagc |
| | | | | tgcaagtccagtcagagtctgt |
| | | | | taaacagtggaaatcaaaagaa |
| | | | | ctacttgacctggtaccagcag |
| | | | | aaaccagggcagcctcctaaac |
| | | | | tgttgatctactgggcatccac |
| | | | | tagggaatctggggtccctgat |
| | | | | cgcttcacaggcagtggatctg |
| | | | | gaacagatttcactctcaccat |
| | | | | cagcagtgtgcaggctgaagac |
| | | | | ctggcagtttattactgtcaga |
| | | | | atgattatagttatccgctcac |
| | | | | gttcggtgctgggaccaagctt |
| | | | | gagatcaaaggtggtggtggtt |
| | | | | ctggcggcggcggctccggtgg |
| | | | | tggtggttctgaggtgcagctg |
| | | | | ctcgagcagtctggagctgagc |
| | | | | tggtaaggcctgggacttcagt |
| | | | | gaagatatcctgcaaggcttct |
| | | | | ggatacgccttcactaactact |
| | | | | ggctaggttgggtaaagcagag |
| | | | | gcctggacatggacttgagtgg |
| | | | | attggagatattttccctggaa |
| | | | | gtggtaatatccactacaatga |
| | | | | gaagttcaagggcaaagccaca |
| | | | | ctgactgcagacaaatcttcga |
| | | | | gcacagcctatatgcagctcag |
| | | | | tagcctgacatttgaggactct |
| | | | | gctgtctatttctgtgcaagac |
| | | | | tgaggaactgggacgagcctat |
| | | | | ggactactggggccaagggacc |
| | | | | acggtcaccgtctcctccggag |
| | | | | gtggtggatccgaggtgaagct |
| | | | | tctcgagtctggaggaggattg |
| | | | | gtgcagcctggagggtcattga |
| | | | | aactctcatgtgcagcctctgg |
| | | | | attcaccttcaatacctacgcc |
| | | | | atgaactgggtccgccaggctc |
| | | | | caggaaagggtttggaatgggt |
| | | | | tgctcgcataagaagtaaatat |
| | | | | aataattatgcaacatattatg |
| | | | | ccgattcagtgaaagacaggtt |
| | | | | caccatctccagagatgattca |
| | | | | aaaaacactgcctatctacaaa |
| | | | | tgaacaacttgaaaactgagga |
| | | | | cactgccgtgtactactgtgtg |
| | | | | agacatgggaacttcggtaata |
| | | | | gctacgtttcctggtttgctta |
| | | | | ctggggccaagggactctggtc |
| | | | | accgtctcctcaggtggtggtg |
| | | | | gttctggcggcggcggctccgg |
| | | | | tggtggtggttctgagctcgtt |
| | | | | gtgactcaggaatctgcactca |
| | | | | ccacatcacctggtgaaacagt |
| | | | | cacactcacttgtcgctcaagt |
| | | | | actggggctgttacaactagta |
| | | | | actatgccaactgggtccaaga |
| | | | | aaaaccagatcatttattcact |
| | | | | ggtctaataggtggtaccaaca |
| | | | | agcgagcaccaggtgtgcctgc |
| | | | | cagattctcaggctccctgatt |
| | | | | ggagacaaggctgccctcacca |
| | | | | tcacaggggcacagactgagga |
| | | | | tgaggcaatatatttctgtgct |
| | | | | ctatggtacagcaacctctggg |
| | | | | tgttcggtggaggaaccaaact |
| | | | | gactgtcctacatcatcaccat |
| | | | | catcat |
| 126 | FN18 VH | hybri- | NA | caggtccagctgcagcagtctg |
| | | doma | | aagctgaactggcaagacctgg |
| | | | | ggcctcagtgaagatgtcctgc |
| | | | | aaggcttctggctacaccttta |
| | | | | ctgactacacgatacactggtt |
| | | | | aaaacagaggcctggacagggt |
| | | | | ctggactggattggatatttta |
| | | | | atcctagcagtgaatctactga |
| | | | | atacaatcggaaattcaaggac |
| | | | | aggaccatattgactgcagaca |
| | | | | gatcctcaaccacagcctacat |
| | | | | gcaactgagcagcctgacatct |
| | | | | gaggactctgcggtctattact |
| | | | | gttcaaggaaaggggagaaact |
| | | | | acttggtaaccgttactggtac |
| | | | | ttcgatgtctggggcgcaggga |
| | | | | cctcggtcaccgtctcctca |
| 127 | FN18 VH | hybri- | AA | QVQLQQSEAELARPGASVKMSC |
| | | doma | | KASGYTFTDYTIHWLKQRPGQG |
| | | | | LDWIGYFNPSSESTEYNRKFKD |
| | | | | RTILTADRSSTTAYMQLSSLTS |
| | | | | EDSAVYYCSRKGEKLLGNRYWY |
| | | | | FDVWGAGTSVTVSS |
| 128 | FN18 VL | hybri- | NA | gacattgtgatgtcacagtctc |
| | | doma | | catcctccctagctgtgtcagt |
| | | | | tggagagaaggttactatgagc |
| | | | | tgcaagtccagtcagagccttt |
| | | | | tatatagtagcaatcaaaagaa |
| | | | | ctacttggcctggtaccagcag |
| | | | | aagccagggcagtctcctaaat |
| | | | | tgctgattaactgggcatccac |
| | | | | cagggaatctggggtccctgat |
| | | | | cgcttcacaggcagtggatcta |
| | | | | ggacagatttcactctcaccat |
| | | | | cagcagtgtgaaggctgaagac |
| | | | | ctggcagtttatttctgtcagc |
| | | | | aattttatagttatcctccgac |
| | | | | gttcggtggaggcaccaagctg |
| | | | | gaaatcaaa |
| 129 | FN18 VL | hybri- | AA | DIVMSQSPSSLAVSVGEKVTMS |
| | | doma | | CKSSQSLLYSSNQKNYLAWYQQ |
| | | | | KPGQSPKLLINWASTRESGVPD |
| | | | | RFTGSGSRTDFTLTISSVKAED |
| | | | | LAVYFCQQFYSYPPTFGGGTKL |
| | | | | EIK |
| 130 | FN18 VH- | arti- | NA | caggtccagctgcagcagtctg |
| | VL scFv | ficial | | aagctgaactggcaagacctgg |
| | | | | ggcctcagtgaagatgtcctgc |
| | | | | aaggcttctggctacaccttta |
| | | | | ctgactacacgatacactggtt |
| | | | | aaaacagaggcctggacagggt |
| | | | | ctggactggattggatatttta |
| | | | | atcctagcagtgaatctactga |
| | | | | atacaatcggaaattcaaggac |
| | | | | aggaccatattgactgcagaca |
| | | | | gatcctcaaccacagcctacat |
| | | | | gcaactgagcagcctgacatct |
| | | | | gaggactctgcggtctattact |
| | | | | gttcaaggaaaggggagaaact |
| | | | | acttggtaaccgttactggtac |
| | | | | ttcgatgtctggggcgcaggga |
| | | | | cctcggtcaccgtctcctcagg |
| | | | | tggtggtggttctggcggcggc |
| | | | | ggctccggtggtggtggttctg |
| | | | | acattgtgatgtcacagtctcc |
| | | | | atcctccctagctgtgtcagtt |
| | | | | ggagagaaggttactatgagct |
| | | | | gcaagtccagtcagagcctttt |
| | | | | atatagtagcaatcaaaagaac |
| | | | | tacttggcctggtaccagcaga |
| | | | | agccagggcagtctcctaaatt |
| | | | | gctgattaactgggcatccacc |
| | | | | agggaatctggggtccctgatc |
| | | | | gcttcacaggcagtggatctag |
| | | | | gacagatttcactctcaccatc |
| | | | | agcagtgtgaaggctgaagacc |
| | | | | tggcagtttatttctgtcagca |
| | | | | attttatagttatcctccgacg |
| | | | | ttcggtggaggcaccaagctgg |
| | | | | aaatcaaa |
| 131 | FN18 VH- | arti- | AA | QVQLQQSEAELARPGASVKMSC |
| | VL scFv | ficial | | KASGYTFTDYTIHWLKQRPGQG |
| | | | | LDWIGYFNPSSESTEYNRKFKD |
| | | | | RTILTADRSSTTAYMQLSSLTS |
| | | | | EDSAVYYCSRKGEKLLGNRYWY |
| | | | | FDVWGAGTSVTVSSGGGGSGGG |
| | | | | GSGGGGSDIVMSQSPSSLAVSV |
| | | | | GEKVTMSCKSSQSLLYSSNQKN |
| | | | | YLAWYQQKPGQSPKLLINWAST |
| | | | | RESGVPDRFTGSGSRTDFTLTI |
| | | | | SSVKAEDLAVYFCQQFYSYPPT |
| | | | | FGGGTKIEIK |
| 132 | FN18 VL- | arti- | NA | gacattgtgatgtcacagtctc |
| | VH scFv | ficial | | catcctccctagctgtgtcagt |
| | | | | tggagagaaggttactatgagc |
| | | | | tgcaagtccagtcagagccttt |
| | | | | tatatagtagcaatcaaaagaa |
| | | | | ctacttggcctggtaccagcag |
| | | | | aagccagggcagtctcctaaat |
| | | | | tgctgattaactgggcatccac |
| | | | | cagggaatctggggtccctgat |
| | | | | cgcttcacaggcagtggatcta |
| | | | | ggacagatttcactctcaccat |
| | | | | cagcagtgtgaaggctgaagac |
| | | | | ctggcagtttatttctgtcagc |
| | | | | aattttatagttatcctccgac |
| | | | | gttcggtggaggcaccaagctg |
| | | | | gaaatcaaaggtggtggtggtt |
| | | | | ctggcggcggcggctccggtgg |
| | | | | tggtggttctcaggtccagctg |
| | | | | cagcagtctgaagctgaactgg |
| | | | | caagacctggggcctcagtgaa |
| | | | | gatgtcctgcaaggcttctggc |
| | | | | tacacctttactgactacacga |
| | | | | tacactggttaaaacagaggcc |
| | | | | tggacagggtctggactggatt |
| | | | | ggatattttaatcctagcagtg |
| | | | | aatctactgaatacaatcggaa |
| | | | | attcaaggacaggaccatattg |
| | | | | actgcagacagatcctcaacca |
| | | | | cagcctacatgcaactgagcag |
| | | | | cctgacatctgaggactctgcg |
| | | | | gtctattactgttcaaggaaag |
| | | | | gggagaaactacttggtaaccg |
| | | | | ttactggtacttcgatgtctgg |
| | | | | ggcgcagggacctcggtcaccg |
| | | | | tctcctca |
| 133 | FN18 VL- | arti- | AA | DIVMSQSPSSLAVSVGEKVTMS |
| | VH scFv | ficial | | CKSSQSLLYSSNQKNYLAWYQQ |
| | | | | KPGQSPKLLINWASTRESGVPD |
| | | | | RFTGSGSRTDFTLTISSVKAED |
| | | | | LAVYFCQQFYSYPPTFGGGTKL |
| | | | | EIKGGGGSGGGGSGGGGSQVQL |
| | | | | QQSEAELARPGASVKMSCKASG |
| | | | | YTFTDYTIHWLKQRPGQGLDWI |
| | | | | GYFNPSSESTEYNRKFKDRTIL |
| | | | | TADRSSTTAYMQLSSLTSEDSA |
| | | | | VYYCSRKGEKLLGNRYWYFDVW |
| | | | | GAGTSVTVSS |
| 134 | CD3 | human | AA | QDGNEEMGGITQTPYKVSISGT |
| | epsilon | cDNA; | | TVILTCPQYPGSEILWQHNDKN |
| | extra- | NM_ | | IGGDEDDKNIGSDEDHLSLKEF |
| | cellular | 000733 | | SELEQSGYYVCYPRGSKPEDAN |
| | portion | | | FYLYLRARVCENCMEMD |
| 135 | CD3 | Cyno- | AA | QDGNEEMGSITQTPYQVSISGT |
| | epsilon | molgus | | TVILTCSQHLGSEAQWQHNGKN |
| | extra- | cDNA; AB | | KEDSGDRLFLPEFSEMEQSGYY |
| | cellular | 073993 | | VCYPRGSNPEDASHHLYLKARV |
| | portion | | | CENCMEMD |
| | FN18+ | | | |
| 136 | CD3 | Cyno- | AA | QDGNEEMGSITQTPYQVSISGT |
| | epsilon | molgus | | TVILTCSQHLGSEAQWQHNGKN |
| | extra- | cDNA; AB | | KGDSGDQLFLPEFSEMEQSGYY |
| | cellular | 073994 | | VCYPRGSNPEDASHHLYLKARV |
| | portion | | | CENCMEMD |
| | FN18− | | | |
| 137 | EpCAM | human | AA | QEECVCENYKLAVNCFVNNNRQ |
| | extra- | cDNA | | CQCTSVGAQNTVICSKLAAKCL |
| | cellular | | | VMKAEMNGSKLGRRAKPEGALQ |
| | portion | | | NNDGLYDPDCDESGLFKAKQCN |
| | | | | GTSTCWCVNTAGVRRTDKDTEI |
| | | | | TCSERVRTYWIIIELKHKAREK |
| | | | | PYDSKSLRTALQKEITTRYQLD |
| | | | | PKFITSILYENNVITIDLVQNS |
| | | | | SQKTQNDVDIADVAYYFEKDVK |
| | | | | GESLFHSKKMDLTVNGEQLDLD |
| | | | | PGQTLIYYVDEKAPEFSMQGLK |
| 138 | EpCAM | chimp | AA | QEECVCENYKLAVNCFVNNNHQ |
| | extra- | cDNA | | CQCTSIGAQNTVICSKLAAKCL |
| | cellular | | | VMKAEMNGSKLGRRAKPEGALQ |
| | portion | | | NNDGLYDPDCDESGLFKAKQCN |
| | | | | GTSTCWCVNTAGVRRTDKDTEI |
| | | | | TCSERVRTYWIIIELKHKAREK |
| | | | | PYDGKSLRTALQKEITTRYQLD |
| | | | | PKFITNILYENNVITIDLVQNS |
| | | | | SQKTQNDVDIADVAYYFEKDVK |
| | | | | GESLFHSKKMDLTVNGEQLDLD |
| | | | | PGQTLIYYVDEKAPEFSMQGLK |
| 139 | EpCAM | rhesus | AA | IDENTICAL WITH CYNO- |
| | extra- | cDNA | | MOLGUS EPCAM SHOWN IN |
| | cellular | | | FIG.6 AND SEQ ID NO. |
| | portion | | | 48 |
| 142 | human CD3 | human | AA | QSIKGNHLVKVYDYQEDGSVLL |
| | gamma | cDNA; | | TCDAEAKNITWFKDGKMIGFLT |
| | extra- | NM_ | | EDKKKWNLGSNAKDPRGMYQCK |
| | cellular | 000073 | | GSQNKSKPLQVYYRMCQNCIEL |
| | portion | | | N |
| 143 | human CD3 | human | AA | FKIPIEELEDRVFVNCNTSITW |
| | delta | cDNA; | | VEGTVGTLLSDITRLDLGKRIL |
| | extra- | NM_ | | DPRGIYRCNGTDIYKDKESTVQ |
| | cellular | 000732 | | VHYRMCQSCVELDPAT |
| | portion | | | |
| 144 | cyno- | cyno- | AA | QSFEENRKLNVYNQEDGSVLLT |
| | molgus | molgus | | CHVKNTNITWFKEGKMIDILTA |
| | CD3 gamma | cDNA; | | HKNKWNLGSNTKDPRGVYQCKG |
| | extra- | AB073992 | | SKDKSKTLQVYYRMCQNCIELN |
| | cellular | | | |
| | portion | | | |
| 145 | cyno- | cyno- | AA | FKIPVEELEDRVFVKCNTSVTW |
| | molgus | molgus | | VEGTVGTLLTNNTRLDLGKRIL |
| | CD3 | cDNA; | | DPRGIYRCNGTDIYKDKESAVQ |
| | delta | AB073991 | | VHYRMCQNCVELDPAT |
| | extra- | | | |
| | cellular | | | |
| | portion | | | |
| 146 | Human- | arti- | AA | EVQLLESGGGLVQPGGSLKLSC |
| | like VH | ficial | | AASGFTFNTYAMNWVRQAPGKG |
| | (SEQ ID | | | LEWVARIRSKYNNYATYYADSV |
| | NO. 110) | | | KDRFTISRDDSKNTAYLQMNNL |
| | x murine | | | KTEDTAVYYCVRHGNFGNSYVS |
| | VL (SEQ | | | WFAYWGQGTLVTVSSGGGGSGG |
| | ID NO. | | | GGSGGGGSELVVTQESALTTSP |
| | 148) scFv | | | GETVTLTCRSSTGAVTTSNYAN |
| | | | | WVQEKPDHLFTGLIGGTNKRAP |
| | | | | GVPARFSGSLIGDKAALTITGA |
| | | | | QTEDEAIYFCALWYSNLWVFGG |
| | | | | GTKLTVL |
| 147 | Human- | arti- | NA | gaggtgcagctgctcgagtctg |
| | like VH | ficial | | gaggaggattggtgcagcctgg |
| | (SEQ ID | | | agggtcattgaaactctcatgt |
| | NO. 110) | | | gcagcctctggattcaccttca |
| | x murine | | | atacctacgccatgaactgggt |
| | VL (SEQ | | | ccgccaggctccaggaaagggt |
| | ID NO. | | | ttggaatgggttgctcgcataa |
| | 148) scFv | | | gaagtaaatataataattatgc |
| | | | | aacatattatgccgattcagtg |
| | | | | aaagacaggttcaccatctcca |
| | | | | gagatgattcaaaaaacactgc |
| | | | | ctatctacaaatgaacaacttg |
| | | | | aaaactgaggacactgccgtgt |
| | | | | actactgtgtgagacatgggaa |
| | | | | cttcggtaatagctacgtttcc |
| | | | | tggtttgcttactggggccaag |
| | | | | ggactctggtcaccgtctcctc |
| | | | | aggtggtggtggttctggcggc |
| | | | | ggcggctccggtggtggtggtt |
| | | | | ctgagctcgttgtgactcagga |
| | | | | atctgcactcaccacatcacct |
| | | | | ggtgaaacagtcacactcactt |
| | | | | gtcgctcaagtactggggctgt |
| | | | | tacaactagtaactatgccaac |
| | | | | tgggtccaagaaaaaccagatc |
| | | | | atttattcactggtctaatagg |
| | | | | tggtaccaacaagcgagcacca |
| | | | | ggtgtgcctgccagattctcag |
| | | | | gctccctgattggagacaaggc |
| | | | | tgccctcaccatcacaggggca |
| | | | | cagactgaggatgaggcaatat |
| | | | | atttctgtgctctatggtacag |
| | | | | caacctctgggtgttcggtgga |
| | | | | ggaaccaaactgactgtccta |
| 148 | murine | arti- | AA | ELVVTQESALTTSPGETVTLTC |
| | VL | ficial | | RSSTGAVTTSNYANWVQEKPDH |
| | [identical | | | LFTGLIGGTNKRAPGVPARFSG |
| | to VL of | | | SLIGDKAALTITGAQTEDEAIY |
| | SEQ ID | | | FCALWYSNLWVFGGGTKLTVL |
| | NO. 4, | | | |
| | with | | | |
| | exception | | | |
| | of the | | | |
| | first two | | | |
| | amino acid | | | |
| | residues] | | | |
| 149 | murine VL | arti- | NA | Gagctcgttgtgactcaggaat |
| | VL | ficial | | ctgcactcaccacatcacctgg |
| | [identical | | | tgaaacagtcacactcacttgt |
| | to VL of | | | cgctcaagtactggggctgtta |
| | SEQ ID | | | caactagtaactatgccaactg |
| | NO. 4, | | | ggtccaagaaaaaccagatcat |
| | with | | | ttattcactggtctaataggtg |
| | exception | | | gtaccaacaagcgagcaccagg |
| | of | | | tgtgcctgccagattctcaggc |
| | nucleotides | | | tccctgattggagacaaggctg |
| | encoding | | | ccctcaccatcacaggggcaca |
| | the first | | | gactgaggatgaggcaatatat |
| | two amino | | | ttctgtgctctatggtacagca |
| | acid | | | acctctgggtgttcggtggagg |
| | residues] | | | aaccaaactgactgtccta |
| 150 | CAIX LH | arti- | NA | gacattgtgatgacccagtctc |
| | scFv | ficial | | aaagattcatgtccacaacagt |
| | | | | aggagacagggtcagcatcacc |
| | | | | tgcaaggccagtcagaatgtgg |
| | | | | tttctgctgttgcctggtatca |
| | | | | acagaaaccaggacaatctcct |
| | | | | aaactactgatttactcagcat |
| | | | | ccaatcggtacactggagtccc |
| | | | | tgatcgcttcacaggcagtgga |
| | | | | tctgggacagatttcactctca |
| | | | | ccattagcaatatgcagtctga |
| | | | | agacctggctgattttttctgt |
| | | | | caacaatatagcaactatccgt |
| | | | | ggacgttcggtggaggcaccaa |
| | | | | gctggaaatcaaaggtggtggt |
| | | | | ggttctggcggcggcggctccg |
| | | | | gtggtggtggttctgacgtgaa |
| | | | | gctcgtggagtctgggggaggc |
| | | | | ttagtgaagcttggagggtccc |
| | | | | tgaaactctcctgtgcagcctc |
| | | | | tggattcactttcagtaactat |
| | | | | tacatgtcttgggttcgccaga |
| | | | | ctccagagaagaggctggagtt |
| | | | | ggtcgcagccattaatagtgat |
| | | | | ggtggtatcacctactatctag |
| | | | | acactgtgaagggccgattcac |
| | | | | catttcaagagacaatgccaag |
| | | | | aacaccctgtacctgcaaatga |
| | | | | gcagtctgaagtctgaggacac |
| | | | | agccttgttttactgtgcaaga |
| | | | | caccgctcgggctacttttcta |
| | | | | tggactactggggtcaaggaac |
| | | | | ctcagtcaccgtctcctcc |
| 151 | CAIX LH | arti- | AA | DIVMTQSQRFMSTTVGDRVSIT |
| | scFv | ficial | | CKASQNVVSAVAWYQQKPGQSP |
| | | | | KLLIYSASNRYTGVPDRFTGSG |
| | | | | SGTDFTLTISNMQSEDLADFFC |
| | | | | QQYSNYPWTFGGGTKLEIKGGG |
| | | | | GSGGGGSGGGGSDVKLVESGGG |
| | | | | LVKLGGSLKLSCAASGFTFSNY |
| | | | | YMSWVRQTPEKRLELVAAINSD |
| | | | | GGITYYLDTVKGRFTISRDNAK |
| | | | | NTLYLQMSSLKSEDTALFYCAR |
| | | | | HRSGYFSMDYWGQGTSVTVSS |
| 152 | EGFR21 LH | arti- | NA | gacattgtgctgacacagtctc |
| | scFv | ficial | | ctgcttccttacctgtgtctct |
| | | | | ggggcagagggccaccatctca |
| | | | | tgcagggccagccaaagtgtca |
| | | | | gttcatctacttatagttatat |
| | | | | acactggtaccaacagaaacca |
| | | | | ggacagccacccaaactcctca |
| | | | | tcacgtatgcatccaacctaga |
| | | | | atctggggtccctgccaggttc |
| | | | | agtggcagtgggtctgggacag |
| | | | | acttcaccctcgacatccatcc |
| | | | | tgtggaggaggatgattcttca |
| | | | | acatattactgtcagcacagtt |
| | | | | gggagattccatttacgttcgg |
| | | | | ctcggggacaaagttggaaata |
| | | | | aaaggtggtggtggttctggcg |
| | | | | gcggcggctccggtggtggtgg |
| | | | | ttctcaggttcagctgcagcag |
| | | | | tctggacctgatctggtgaagc |
| | | | | ctggggcctcagtgaagatgtc |
| | | | | ctgcaaggcttctggacacact |
| | | | | ttcactgactgtgttataatct |
| | | | | gggtgaaacagagagctggaca |
| | | | | gggccttgagtggattggacag |
| | | | | atttatccagggactggtcgtt |
| | | | | cttactacaatgagattttcaa |
| | | | | gggcaaggccacactgactgca |
| | | | | gacaaatcctccaacacagtcc |
| | | | | acattcaactcagcagcctgac |
| | | | | atctgaggactctgcggtctat |
| | | | | ttctgtgccctatctactctta |
| | | | | ttcacgggacctggttttctta |
| | | | | ttggggccaagggactctggtc |
| | | | | actgtctcttcc |
| 153 | EGFR21 LH | arti- | AA | DIVLTQSPASLPVSLGQRATIS |
| | scFv | ficial | | CRASQSVSSSTYSYIHWYQQKP |
| | | | | GQPPKLLITYASNLESGVPARF |
| | | | | SGSGSGTDFTLDIHPVEEDDSS |
| | | | | TYYCQHSWEIPFTFGSGTKLEI |
| | | | | KGGGGSGGGGSGGGGSQVQLQQ |
| | | | | SGPDLVKPGASVKMSCKASGHT |
| | | | | FTDCVIIWVKQRAGQGLEWIGQ |
| | | | | IYPGTGRSYYNEIFKGKATLTA |
| | | | | DKSSNTVHIQLSSLTSEDSAVY |
| | | | | FCALSTLIHGTWFSYWGQGTLV |
| | | | | TVSS |
| 154 | EGFRvIII- | arti- | NA | gatgttgtgatgacccagactc |
| | LH scFv | ficial | | cactcactttgtcggttaccat |
| | | | | tggacaaccagcctctatctct |
| | | | | tgcaagtcaagtcagagcctct |
| | | | | tatatagtaatggaaaaaccta |
| | | | | tttgaattggttattacagagg |
| | | | | ccaggccagtctccaaagcgcc |
| | | | | taatctatctggtatctaaact |
| | | | | ggactctggagtccctgacagg |
| | | | | ttcactggcagtggatcaggaa |
| | | | | cagattttacactgaaaatcag |
| | | | | cagagtggaggctgaggatttg |
| | | | | ggaatttattactgcgtgcaag |
| | | | | atacacattttcctcagacatt |
| | | | | cggtggaggcaccaagctggaa |
| | | | | atcaaaggtggtggtggttctg |
| | | | | gcggcggcggctccggtggtgg |
| | | | | tggttctgaggtccagctgcaa |
| | | | | cagtctggacctgagctgctga |
| | | | | agcctggggcttcagtgaagat |
| | | | | atcctgcaagacttctggatac |
| | | | | acattcactgaatacaccatac |
| | | | | actgggtgaagcagagccatgg |
| | | | | aaagagccttgagtggattgga |
| | | | | ggtattgatcctaacaatggtg |
| | | | | gtactatgtataaccaaaaatt |
| | | | | caagggcaaggccacattgact |
| | | | | gtagacaagtcttccagcacag |
| | | | | cctacacggacctccgcagcct |
| | | | | gacgtctgaggattctgcagtc |
| | | | | tattactgcacaagagcagagg |
| | | | | ctatggactactggggtcaagg |
| | | | | aacctcagtcaccgtctcctcc |
| 155 | EGFRvIII- | arti- | AA | DVVMTQTPLTLSVTIGQPASIS |
| | LH scFv | ficial | | CKSSQSLLYSNGKTYLNWLLQR |
| | | | | PGQSPKRLIYLVSKLDSGVPDR |
| | | | | FTGSGSGTDFTLKISRVEAEDL |
| | | | | GIYYCVQDTHFPQTFGGGTKLE |
| | | | | IKGGGGSGGGGSGGGGSEVQLQ |
| | | | | QSGPELLKPGASVKISCKTSGY |
| | | | | TFTEYTIHWVKQSHGKSLEWIG |
| | | | | GIDPNNGGTMYNQKFKGKATLT |
| | | | | VDKSSSTAYTDLRSLTSEDSAV |
| | | | | YYCTRAEAMDYWGQGTSVTVSS |
| 156 | CAIX LHx | arti- | NA | gacattgtgatgacccagtctc |
| | SEQ ID | ficial | | aaagattcatgtccacaacagt |
| | NO. 10 | | | aggagacagggtcagcatcacc |
| | | | | tgcaaggccagtcagaatgtgg |
| | | | | tttctgctgttgcctggtatca |
| | | | | acagaaaccaggacaatctcct |
| | | | | aaactactgatttactcagcat |
| | | | | ccaatcggtacactggagtccc |
| | | | | tgatcgcttcacaggcagtgga |
| | | | | tctgggacagatttcactctca |
| | | | | ccattagcaatatgcagtctga |
| | | | | agacctggctgattttttctgt |
| | | | | caacaatatagcaactatccgt |
| | | | | ggacgttcggtggaggcaccaa |
| | | | | gctggaaatcaaaggtggtggt |
| | | | | ggttctggcggcggcggctccg |
| | | | | gtggtggtggttctgacgtgaa |
| | | | | gctcgtggagtctgggggaggc |
| | | | | ttagtgaagcttggagggtccc |
| | | | | tgaaactctcctgtgcagcctc |
| | | | | tggattcactttcagtaactat |
| | | | | tacatgtcttgggttcgccaga |
| | | | | ctccagagaagaggctggagtt |
| | | | | ggtcgcagccattaatagtgat |
| | | | | ggtggtatcacctactatctag |
| | | | | acactgtgaagggccgattcac |
| | | | | catttcaagagacaatgccaag |
| | | | | aacaccctgtacctgcaaatga |
| | | | | gcagtctgaagtctgaggacac |
| | | | | agccttgttttactgtgcaaga |
| | | | | caccgctcgggctacttttcta |
| | | | | tggactactggggtcaaggaac |
| | | | | ctcagtcaccgtctcctccgga |
| | | | | ggtggtggatccgaggtgaagc |
| | | | | ttctcgagtctggaggaggatt |
| | | | | ggtgcagcctaaagggtcattg |
| | | | | aaactctcatgtgcagcctctg |
| | | | | gattcaccttcaatacctacgc |
| | | | | catgaactgggtccgccaggct |
| | | | | ccaggaaagggtttggaatggg |
| | | | | ttgctcgcataagaagtaaata |
| | | | | taataattatgcaacatattat |
| | | | | gccgattcagtgaaagacaggt |
| | | | | tcaccatctccagagatgattc |
| | | | | acaaagcattctctatctacaa |
| | | | | atgaacaacttgaaaactgagg |
| | | | | acacagccatgtactactgtgt |
| | | | | gagacatgggaacttcggtaat |
| | | | | agctacgtttcctggtttgctt |
| | | | | actggggccaagggactctggt |
| | | | | cactgtctctgcaggtggtggt |
| | | | | ggttctggcggcggcggctccg |
| | | | | gtggtggtggttctcaggctgt |
| | | | | tgtgactcaggaatctgcactc |
| | | | | accacatcacctggtgaaacag |
| | | | | tcacactcacttgtcgctcaag |
| | | | | tactggggctgttacaactagt |
| | | | | aactatgccaactgggtccaag |
| | | | | aaaaaccagatcatttattcac |
| | | | | tggtctaataggtggtaccaac |
| | | | | aagcgagctccaggtgtgcctg |
| | | | | ccagattctcaggctccctgat |
| | | | | tggagacaaggctgccctcacc |
| | | | | atcacaggggcacagactgagg |
| | | | | atgaggcaatatatttctgtgc |
| | | | | tctatggtacagcaacctctgg |
| | | | | gtgttcggtggaggaaccaaac |
| | | | | tgactgtccta |
| 157 | CAIX LHx | arti- | AA | DIVMTQSQRFMSTTVGDRVSIT |
| | SEQ ID | ficial | | CKASQNVVSAVAWYQQKPGQSP |
| | NO. 10 | | | KLLIYSASNRYTGVPDRFTGSG |
| | | | | SGTDFTLTISNMQSEDLADFFC |
| | | | | QQYSNYPWTFGGGTKLEIKGGG |
| | | | | GSGGGGSGGGGSDVKLVESGGG |
| | | | | LVKLGGSLKLSCAASGFTFSNY |
| | | | | YMSWVRQTPEKRLELVAAINSD |
| | | | | GGITYYLDTVKGRFTISRDNAK |
| | | | | NTLYLQMSSLKSEDTALFYCAR |
| | | | | HRSGYFSMDYWGQGTSVTVSSG |
| | | | | GGGSEVKLLESGGGLVQPKGSL |
| | | | | KLSCAASGFTFNTYAMNWVRQA |
| | | | | PGKGLEWVARIRSKYNNYATYY |
| | | | | ADSVKDRFTISRDDSQSILYLQ |
| | | | | MNNLKTEDTAMYYCVRHGNFGN |
| | | | | SYVSWFAYWGQGTLVTVSAGGG |
| | | | | GSGGGGSGGGGSQAVVTQESAL |
| | | | | TTSPGETVTLTCRSSTGAVTTS |
| | | | | NYANWVQEKPDHLFTGLIGGTN |
| | | | | KRAPGVPARFSGSLIGDKAALT |
| | | | | ITGAQTEDEAIYFCALWYSNLW |
| | | | | VFGGGTKLTVL |
| 158 | EGFR21 | arti- | NA | gacattgtgctgacacagtctc |
| | LHx | ficial | | ctgcttccttacctgtgtctct |
| | SEQ ID | | | ggggcagagggccaccatctca |
| | NO. 10 | | | tgcagggccagccaaagtgtca |
| | | | | gttcatctacttatagttatat |
| | | | | acactggtaccaacagaaacca |
| | | | | ggacagccacccaaactcctca |
| | | | | tcacgtatgcatccaacctaga |
| | | | | atctggggtccctgccaggttc |
| | | | | agtggcagtgggtctgggacag |
| | | | | acttcaccctcgacatccatcc |
| | | | | tgtggaggaggatgattcttca |
| | | | | acatattactgtcagcacagtt |
| | | | | gggagattccatttacgttcgg |
| | | | | ctcggggacaaagttggaaata |
| | | | | aaaggtggtggtggttctggcg |
| | | | | gcggcggctccggtggtggtgg |
| | | | | ttctcaggttcagctgcagcag |
| | | | | tctggacctgatctggtgaagc |
| | | | | ctggggcctcagtgaagatgtc |
| | | | | ctgcaaggcttctggacacact |
| | | | | ttcactgactgtgttataatct |
| | | | | gggtgaaacagagagctggaca |
| | | | | gggccttgagtggattggacag |
| | | | | atttatccagggactggtcgtt |
| | | | | cttactacaatgagattttcaa |
| | | | | gggcaaggccacactgactgca |
| | | | | gacaaatcctccaacacagtcc |
| | | | | acattcaactcagcagcctgac |
| | | | | atctgaggactctgcggtctat |
| | | | | ttctgtgccctatctactctta |
| | | | | ttcacgggacctggttttctta |
| | | | | ttggggccaagggactctggtc |
| | | | | actgtctcttccggaggtggtg |
| | | | | gatccgaggtgaagcttctcga |
| | | | | gtctggaggaggattggtgcag |
| | | | | cctaaagggtcattgaaactct |
| | | | | catgtgcagcctctggattcac |
| | | | | cttcaatacctacgccatgaac |
| | | | | tgggtccgccaggctccaggaa |
| | | | | agggtttggaatgggttgctcg |
| | | | | cataagaagtaaatataataat |
| | | | | tatgcaacatattatgccgatt |
| | | | | cagtgaaagacaggttcaccat |
| | | | | ctccagagatgattcacaaagc |
| | | | | attctctatctacaaatgaaca |
| | | | | acttgaaaactgaggacacagc |
| | | | | catgtactactgtgtgagacat |
| | | | | gggaacttcggtaatagctacg |
| | | | | tttcctggtttgcttactgggg |
| | | | | ccaagggactctggtcactgtc |
| | | | | tctgcaggtggtggtggttctg |
| | | | | gcggcggcggctccggtggtgg |
| | | | | tggttctcaggctgttgtgact |
| | | | | caggaatctgcactcaccacat |
| | | | | cacctggtgaaacagtcacact |
| | | | | cacttgtcgctcaagtactggg |
| | | | | gctgttacaactagtaactatg |
| | | | | ccaactgggtccaagaaaaacc |
| | | | | agatcatttattcactggtcta |
| | | | | ataggtggtaccaacaagcgag |
| | | | | ctccaggtgtgcctgccagatt |
| | | | | ctcaggctccctgattggagac |
| | | | | aaggctgccctcaccatcacag |
| | | | | gggcacagactgaggatgaggc |
| | | | | aatatatttctgtgctctatgg |
| | | | | tacagcaacctctgggtgttcg |
| | | | | gtggaggaaccaaactgactgt |
| | | | | ccta |
| 159 | EGFR21 | arti- | AA | DIVLTQSPASLPVSLGQRATIS |
| | LHx | ficial | | CRASQSVSSSTYSYIHWYQQKP |
| | SEQ ID | | | GQPPKLLITYASNLESGVPARF |
| | NO. 10 | | | SGSGSGTDFTLDIHPVEEDDSS |
| | | | | TYYCQHSWEIPFTFGSGTKLEI |
| | | | | KGGGGSGGGGSGGGGSQVQLQQ |
| | | | | SGPDLVKPGASVKMSCKASGHT |
| | | | | FTDCVIIWVKQRAGQGLEWIGQ |
| | | | | IYPGTGRSYYNEIFKGKATLTA |
| | | | | DKSSNTVHIQLSSLTSEDSAVY |
| | | | | FCALSTLIHGTWFSYWGQGTLV |
| | | | | TVSSGGGGSEVKLLESGGGLVQ |
| | | | | PKGSLKLSCAASGFTFNTYAMN |
| | | | | WVRQAPGKGLEWVARIRSKYNN |
| | | | | YATYYADSVKDRFTISRDDSQS |
| | | | | ILYLQMNNLKTEDTAMYYCVRH |
| | | | | GNFGNSYVSWFAYWGQGTLVTV |
| | | | | SAGGGGSGGGGSGGGGSQAVVT |
| | | | | QESALTTSPGETVTLTCRSSTG |
| | | | | AVTTSNYANWVQEKPDHLFTGL |
| | | | | IGGTNKRAPGVPARFSGSLIGD |
| | | | | KAALTITGAQTEDEAIYFCALW |
| | | | | YSNLWVFGGGTKLTVL |
| 160 | EGFRvIII- | arti- | NA | gatgttgtgatgacccagactc |
| | LHx | ficial | | cactcactttgtcggttaccat |
| | SEQ ID | | | tggacaaccagcctctatctct |
| | NO. 10 | | | tgcaagtcaagtcagagcctct |
| | | | | tatatagtaatggaaaaaccta |
| | | | | tttgaattggttattacagagg |
| | | | | ccaggccagtctccaaagcgcc |
| | | | | taatctatctggtatctaaact |
| | | | | ggactctggagtccctgacagg |
| | | | | ttcactggcagtggatcaggaa |
| | | | | cagattttacactgaaaatcag |
| | | | | cagagtggaggctgaggatttg |
| | | | | ggaatttattactgcgtgcaag |
| | | | | atacacattttcctcagacatt |
| | | | | cggtggaggcaccaagctggaa |
| | | | | atcaaaggtggtggtggttctg |
| | | | | gcggcggcggctccggtggtgg |
| | | | | tggttctgaggtccagctgcaa |
| | | | | cagtctggacctgagctgctga |
| | | | | agcctggggcttcagtgaagat |
| | | | | atcctgcaagacttctggatac |
| | | | | acattcactgaatacaccatac |
| | | | | actgggtgaagcagagccatgg |
| | | | | aaagagccttgagtggattgga |
| | | | | ggtattgatcctaacaatggtg |
| | | | | gtactatgtataaccaaaaatt |
| | | | | caagggcaaggccacattgact |
| | | | | gtagacaagtcttccagcacag |
| | | | | cctacacggacctccgcagcct |
| | | | | gacgtctgaggattctgcagtc |
| | | | | tattactgcacaagagcagagg |
| | | | | ctatggactactggggtcaagg |
| | | | | aacctcagtcaccgtctcctcc |
| | | | | ggaggtggtggatccgaggtga |
| | | | | agcttctcgagtctggaggagg |
| | | | | attggtgcagcctaaagggtca |
| | | | | ttgaaactctcatgtgcagcct |
| | | | | ctggattcaccttcaataccta |
| | | | | cgccatgaactgggtccgccag |
| | | | | gctccaggaaagggtttggaat |
| | | | | gggttgctcgcataagaagtaa |
| | | | | atataataattatgcaacatat |
| | | | | tatgccgattcagtgaaagaca |
| | | | | ggttcaccatctccagagatga |
| | | | | ttcacaaagcattctctatcta |
| | | | | caaatgaacaacttgaaaactg |
| | | | | aggacacagccatgtactactg |
| | | | | tgtgagacatgggaacttcggt |
| | | | | aatagctacgtttcctggtttg |
| | | | | cttactggggccaagggactct |
| | | | | ggtcactgtctctgcaggtggt |
| | | | | ggtggttctggcggcggcggct |
| | | | | ccggtggtggtggttctcaggc |
| | | | | tgttgtgactcaggaatctgca |
| | | | | ctcaccacatcacctggtgaaa |
| | | | | cagtcacactcacttgtcgctc |
| | | | | aagtactggggctgttacaact |
| | | | | agtaactatgccaactgggtcc |
| | | | | aagaaaaaccagatcatttatt |
| | | | | cactggtctaataggtggtacc |
| | | | | aacaagcgagctccaggtgtgc |
| | | | | ctgccagattctcaggctccct |
| | | | | gattggagacaaggctgccctc |
| | | | | accatcacaggggcacagactg |
| | | | | aggatgaggcaatatatttctg |
| | | | | tgctctatggtacagcaacctc |
| | | | | tgggtgttcggtggaggaacca |
| | | | | aactgactgtccta |
| 161 | EGFRvIII- | arti- | AA | DVVMTQTPLTLSVTIGQPASIS |
| | LHx | ficial | | CKSSQSLLYSNGKTYLNWLLQR |
| | SEQ ID | | | PGQSPKRLIYLVSKLDSGVPDR |
| | NO. 10 | | | FTGSGSGTDFTLKISRVEAEDL |
| | | | | GIYYCVQDTHFPQTFGGGTKLE |
| | | | | IKGGGGSGGGGSGGGGSEVQLQ |
| | | | | QSGPELLKPGASVKISCKTSGY |
| | | | | TFTEYTIHWVKQSHGKSLEWIG |
| | | | | GIDPNNGGTMYNQKFKGKATLT |
| | | | | VDKSSSTAYTDLRSLTSEDSAV |
| | | | | YYCTRAEAMDYWGQGTSVTVSS |
| | | | | GGGGSEVKLLESGGGLVQPKGS |
| | | | | LKLSCAASGFTFNTYAMNWVRQ |
| | | | | APGKGLEWVARIRSKYNNYATY |
| | | | | YADSVKDRFTISRDDSQSILYL |
| | | | | QMNNLKTEDTAMYYCVRHGNFG |
| | | | | NSYVSWFAYWGQGTLVTVSAGG |
| | | | | GGSGGGGSGGGGSQAVVTQESA |
| | | | | LTTSPGETVTLTCRSSTGAVTT |
| | | | | SNYANWVQEKPDHLFTGLIGGT |
| | | | | NKRAPGVPARFSGSLIGDKAAL |
| | | | | TITGAQTEDEAIYFCALWYSNL |
| | | | | WVFGGGTKLTVL |
| 162 | anti CD3 | arti- | AA | DIKLQQSGAELARPGASVKMSC |
| | (as used | ficial | | KTSGYTFTRYTMHWVKQRPGQG |
| | in WO | | | LEWIGYINPSRGYTNYNQKFKD |
| | 99/54440 | | | KATLTTDKSSSTAYMQLSSLTS |
| | | | | EDSAVYYCARYYDDHYCLDYWG |
| | | | | QGTTLTVSSVEGGSGGSGGSGG |
| | | | | SGGVDDIQLTQSPAIMSASPGE |
| | | | | KVTMTCRASSSVSYMNWYQQKS |
| | | | | GTSPKRWIYDTSKVASGVPYRF |
| | | | | SGSGSGTSYSLTISSMEAEDAA |
| | | | | TYYCQQWSSNPLTFGAGTKLEL |
| | | | | K |
| 163 | deimmunised | arti- | AA | DVQLVQSGAEVKKPGASVKVSC |
| | (di)-anti | ficial | | KASGYTFTRYTMHWVRQAPGQG |
| | CD3 | | | LEWIGYINPSRGYTNYADSVKG |
| | | | | RFTITTDKSTSTAYMELSSLRS |
| | | | | EDTATYYCARYYDDHYCLDYWG |
| | | | | QGTTVTVSSGEGTSTGSGGSGG |
| | | | | SGGADDIVLTQSPATLSLSPGE |
| | | | | RATLSCRASQSVSYMNWYQQKP |
| | | | | GKAPKRWIYDTSKVASGVPARY |
| | | | | SGSGSGTDYSLTINSLEAEDAA |
| | | | | TYYCQQWSSNPLTFGGGTKVEI |
| | | | | K |
| 164 | VL: CDR3 | murine | AA | QQWSRNPPT |
| 165 | VL: CDR2 | murine | AA | DSSKLAS |
| 166 | VL: CDR1 | murine | AA | SASSSVSYMN |
| 167 | Human-like | arti- | NA | gagctcgttgtgactcaggaac |
| | VL | ficial | | cttcactcaccgtatcacctgg |
| | | | | tggaacagtcacactcacttgt |
| | | | | cgctcgtcgactggggctgtta |
| | | | | caactagcaactatgccaactg |
| | | | | ggtccaacaaaaaccaggtcag |
| | | | | gcaccccgtggtctaataggtg |
| | | | | gtaccaacaagcgcgcaccagg |
| | | | | tactcctgccagattctcaggc |
| | | | | tccctgcttggaggcaaggctg |
| | | | | ccctcaccctctcaggggtaca |
| | | | | gccagaggatgaggcagaatat |
| | | | | tactgtgctctatggtacagca |
| | | | | acctctgggtgttcggtggagg |
| | | | | aaccaaactgactgtccta |
| 168 | Human-like | arti- | AA | ELVVTQEPSLTVSPGGTVTLTC |
| | VL | ficial | | RSSTGAVTTSNYANWVQQKPGQ |
| | | | | APRGLIGGTNKRAPGTPARFSG |
| | | | | SLLGGKAALTLSGVQPEDEAEY |
| | | | | YCALWYSNLWvFGGGTKLTVL |
| 169 | Human-like | arti- | NA | gaggtgcagctgctcgagtctg |
| | VH (SEQ | ficial | | gaggaggattggtgcagcctgg |
| | ID NO. | | | agggtcattgaaactctcatgt |
| | 110) x | | | gcagcctctggattcaccttca |
| | Human- | | | atacctacgccatgaactgggt |
| | like VL | | | ccgccaggctccaggaaagggt |
| | (SEQ ID | | | ttggaatgggttgctcgcataa |
| | NO. 168) | | | gaagtaaatataataattatgc |
| | scFv | | | aacatattatgccgattcagtg |
| | | | | aaagacaggttcaccatctcca |
| | | | | gagatgattcaaaaaacactgc |
| | | | | ctatctacaaatgaacaacttg |
| | | | | aaaactgaggacactgccgtgt |
| | | | | actactgtgtgagacatgggaa |
| | | | | cttcggtaatagctacgtttcc |
| | | | | tggtttgcttactggggccaag |
| | | | | ggactctggtcaccgtctcctc |
| | | | | aggtggtggtggttctggcggc |
| | | | | ggcggctccggtggtggtggtt |
| | | | | ctgagctcgttgtgactcagga |
| | | | | accttcactcaccgtatcacct |
| | | | | ggtggaacagtcacactcactt |
| | | | | gtcgctcgtcgactggggctgt |
| | | | | tacaactagcaactatgccaac |
| | | | | tgggtccaacaaaaaccaggtc |
| | | | | aggcaccccgtggtctaatagg |
| | | | | tggtaccaacaagcgcgcacca |
| | | | | ggtactcctgccagattctcag |
| | | | | gctccctgcttggaggcaaggc |
| | | | | tgccctcaccctctcaggggta |
| | | | | cagccagaggatgaggcagaat |
| | | | | attactgtgctctatggtacag |
| | | | | caacctctgggtgttcggtgga |
| | | | | ggaaccaaactgactgtccta |
| 170 | Human-like | arti- | AA | EVQLLESGGGLVQPGGSLKLSC |
| | VH (SEQ | ficial | | AASGFTFNTYAMNWVRQAPGKG |
| | ID NO. | | | LEWVARIRSKYNNYATYYADSV |
| | 110) x | | | KDRFTISRDDSKNTAYLQMNNL |
| | Human- | | | KTEDTAVYYCVRHGNFGNSYVS |
| | like VL | | | WFAYWGQGTLVTVSSGGGGSGG |
| | (SEQ ID | | | GGSGGGGSELVVTQEPSLTVSP |
| | NO. 168) | | | GGTVTLTCRSSTGAVTTSNYAN |
| | scFv | | | WVQQKPGQAPRGLIGGTNKRAP |
| | | | | GTPARFSGSLLGGKAALTLSGV |
| | | | | QPEDEAEYYCALWYSNLWVFGG |
| | | | | GTKLTVL |
| 171 | EGFR HL x | arti- | NA | caggtgcagctgcagcagtctg |
| | SEQ ID | ficial | | ggcctgatctggtgaagcctgg |
| | NO. 170 | | | ggcctcagtgaagatgtcctgc |
| | | | | aaggcttctggacacactttca |
| | | | | ctgactgtgttataatctgggt |
| | | | | gaaacagagagctggacagggc |
| | | | | cttgagtggattggacagattt |
| | | | | atccagggactggtcgttctta |
| | | | | ctacaatgagattttcaagggc |
| | | | | aaggccacactgactgcagaca |
| | | | | aatcctccaacacagtccacat |
| | | | | tcaactcagcagcctgacatct |
| | | | | gaggactctgcggtctatttct |
| | | | | gtgccctatctactcttattca |
| | | | | cgggacctggttttcttattgg |
| | | | | ggccaagggactctggtcactg |
| | | | | tctcttccggtggtggtggttc |
| | | | | tggcggcggcggctccggtggt |
| | | | | ggtggttctgacattgtactga |
| | | | | cccagtctccagcttccttacc |
| | | | | tgtgtctctggggcagagggcc |
| | | | | accatctcatgcagggccagcc |
| | | | | aaagtgtcagttcatctactta |
| | | | | tagttatatacactggtaccaa |
| | | | | cagaaaccaggacagccaccca |
| | | | | aactcctcatcacgtatgcatc |
| | | | | caacctagaatctggggtccct |
| | | | | gccaggttcagtggcagtgggt |
| | | | | ctgggacagacttcaccctcga |
| | | | | catccatcctgtggaggaggat |
| | | | | gattcttcaacatattactgtc |
| | | | | agcacagttgggagattccatt |
| | | | | tacgttcggctcggggacaaag |
| | | | | ttggaaataaaatccggaggtg |
| | | | | gtggctccgaggtgcagctggt |
| | | | | ggagtctggaggaggattggtg |
| | | | | cagcctggagggtcattgaaac |
| | | | | tctcatgtgcagcctctggatt |
| | | | | caccttcaatacctacgccatg |
| | | | | aactgggtccgccaggctccag |
| | | | | gaaagggtttggaatgggttgc |
| | | | | tcgcataagaagtaaatataat |
| | | | | aattatgcaacatattatgccg |
| | | | | attcagtgaaagacaggttcac |
| | | | | catctccagagatgattcaaaa |
| | | | | aacactgcctatctacaaatga |
| | | | | acaacttgaaaactgaggacac |
| | | | | tgccgtgtactactgtgtgaga |
| | | | | catgggaacttcggtaatagct |
| | | | | acgtttcctggtttgcttactg |
| | | | | gggccaagggactctggtcacc |
| | | | | gtctcctcaggtggtggtggtt |
| | | | | ctggcggcggcggctccggtgg |
| | | | | tggtggttctcagaccgttgtg |
| | | | | actcaggaaccttcactcaccg |
| | | | | tatcacctggtggaacagtcac |
| | | | | actcacttgtcgctcgtccact |
| | | | | ggggctgttacaactagcaact |
| | | | | atgccaactgggtccaacaaaa |
| | | | | accaggtcaggcaccccgtggt |
| | | | | ctaataggtggtaccaacaagc |
| | | | | gcgcaccaggtactcctgccag |
| | | | | attctcaggctccctgcttgga |
| | | | | ggcaaggctgccctcaccctct |
| | | | | caggggtacagccagaggatga |
| | | | | ggcagaatattactgtgctcta |
| | | | | tggtacagcaacctctgggtgt |
| | | | | tcggtggaggaaccaaactgac |
| | | | | tgtcctacatcatcaccatcat |
| | | | | cattaggtcgac |
| 172 | EGFR HL x | arti- | AA | QVQLQQSGPDLVKPGASVKMSC |
| | SEQ ID | ficial | | KASGHTFTDCVIIWVKQRAGQG |
| | NO. 170 | | | LEWIGQIYPGTGRSYYNEIFKG |
| | | | | KATLTADKSSNTVHIQLSSLTS |
| | | | | EDSAVYFCALSTLIHGTWFSYW |
| | | | | GQGTLVTVSSGGGGSGGGGSGG |
| | | | | GGSDIVLTQSPASLPVSLGQRA |
| | | | | TISCRASQSVSSSTYSYIHWYQ |
| | | | | QKPGQPPKLLITYASNLESGVP |
| | | | | ARFSGSGSGTDFTLDIHPVEED |
| | | | | DSSTYYCQHSWEIPFTFGSGTK |
| | | | | LEIKSGGGGSEVQLVESGGGLV |
| | | | | QPGGSLKLSCAASGFTFNTYAM |
| | | | | NWVRQAPGKGLEWVARIRSKYN |
| | | | | NYATYYADSVKDRFTISRDDSK |
| | | | | NTAYLQMNNLKTEDTAVYYCVR |
| | | | | HGNFGNSYVSWFAYWGQGTLVT |
| | | | | VSSGGGGSGGGGSGGGGSQTVV |
| | | | | TQEPSLTVSPGGTVTLTCRSST |
| | | | | GAVTTSNYANWVQQKPGQAPRG |
| | | | | LIGGTNKRAPGTPARFSGSLLG |
| | | | | GKAALTLSGVQPEDEAEYYCAL |
| | | | | WYSNLWVFGGGTKLTVLHHHHH |
| | | | | H*VD |
| 173 | EGFR LH x | arti- | NA | gacattgtgctgacacagtctc |
| | SEQ ID | ficial | | ctgcttccttacctgtgtctct |
| | NO. 170 | | | ggggcagagggccaccatctca |
| | | | | tgcagggccagccaaagtgtca |
| | | | | gttcatctacttatagttatat |
| | | | | acactggtaccaacagaaacca |
| | | | | ggacagccacccaaactcctca |
| | | | | tcacgtatgcatccaacctaga |
| | | | | atctggggtccctgccaggttc |
| | | | | agtggcagtgggtctgggacag |
| | | | | acttcaccctcgacatccatcc |
| | | | | tgtggaggaggatgattcttca |
| | | | | acatattactgtcagcacagtt |
| | | | | gggagattccatttacgttcgg |
| | | | | ctcggggacaaagttggaaata |
| | | | | aaaggtggtggtggttctggcg |
| | | | | gcggcggctccggtggtggtgg |
| | | | | ttctcaggttcagctgcagcag |
| | | | | tctggacctgatctggtgaagc |
| | | | | ctggggcctcagtgaagatgtc |
| | | | | ctgcaaggcttctggacacact |
| | | | | ttcactgactgtgttataatct |
| | | | | gggtgaaacagagagctggaca |
| | | | | gggccttgagtggattggacag |
| | | | | atttatccagggactggtcgtt |
| | | | | cttactacaatgagattttcaa |
| | | | | gggcaaggccacactgactgca |
| | | | | gacaaatcctccaacacagtcc |
| | | | | acattcaactcagcagcctgac |
| | | | | atctgaggactctgcggtctat |
| | | | | ttctgtgccctatctactctta |
| | | | | ttcacgggacctggttttctta |
| | | | | ttggggccaagggactctggtc |
| | | | | actgtctcttccggaggtggtg |
| | | | | gctccgaggtgcagctggtgga |
| | | | | gtctggaggaggattggtgcag |
| | | | | cctggagggtcattgaaactct |
| | | | | catgtgcagcctctggattcac |
| | | | | cttcaatacctacgccatgaac |
| | | | | tgggtccgccaggctccaggaa |
| | | | | agggtttggaatgggttgctcg |
| | | | | cataagaagtaaatataataat |
| | | | | tatgcaacatattatgccgatt |
| | | | | cagtgaaagacaggttcaccat |
| | | | | ctccagagatgattcaaaaaac |
| | | | | actgcctatctacaaatgaaca |
| | | | | acttgaaaactgaggacactgc |
| | | | | cgtgtactactgtgtgagacat |
| | | | | gggaacttcggtaatagctacg |
| | | | | tttcctggtttgcttactgggg |
| | | | | ccaagggactctggtcaccgtc |
| | | | | tcctcaggtggtggtggttctg |
| | | | | gcggcggcggctccggtggtgg |
| | | | | tggttctcagaccgttgtgact |
| | | | | caggaaccttcactcaccgtat |
| | | | | cacctggtggaacagtcacact |
| | | | | cacttgtcgctcgtccactggg |
| | | | | gctgttacaactagcaactatg |
| | | | | ccaactgggtccaacaaaaacc |
| | | | | aggtcaggcaccccgtggtcta |
| | | | | ataggtggtaccaacaagcgcg |
| | | | | caccaggtactcctgccagatt |
| | | | | ctcaggctccctgcttggaggc |
| | | | | aaggctgccctcaccctctcag |
| | | | | gggtacagccagaggatgaggc |
| | | | | agaatattactgtgctctatgg |
| | | | | tacagcaacctctgggtgttcg |
| | | | | gtggaggaaccaaactgactgt |
| | | | | cctacatcatcaccatcatcat |
| | | | | taggtcgac |
| 174 | EGFR LH x | arti- | AA | DIVLTQSPASLPVSLGQRATIS |
| | SEQ ID | ficial | | CRASQSVSSSTYSYIHWYQQKP |
| | NO. 170 | | | GQPPKLLITYASNLESGVPARF |
| | | | | SGSGSGTDFTLDIHPVEEDDSS |
| | | | | TYYCQHSWEIPFTFGSGTKLEI |
| | | | | KGGGGSGGGGSGGGGSQVQLQQ |
| | | | | SGPDLVKPGASVKMSCKASGHT |
| | | | | FTDCVIIWVKQRAGQGLEWIGQ |
| | | | | IYPGTGRSYYNEIFKGKATLTA |
| | | | | DKSSNTVHIQLSSLTSEDSAVY |
| | | | | FCALSTLIHGTWFSYWGQGTLV |
| | | | | TVSSGGGGSEVQLVESGGGLVQ |
| | | | | PGGSLKLSCAASGFTFNTYAMN |
| | | | | WVRQAPGKGLEWVARIRSKYNN |
| | | | | YATYYADSVKDRFTISRDDSKN |
| | | | | TAYLQMNNLKTEDTAVYYCVRH |
| | | | | GNFGNSYVSWFAYWGQGTLVTV |
| | | | | SSGGGGSGGGGSGGGGSQTVVT |
| | | | | QEPSLTVSPGGTVTLTCRSSTG |
| | | | | AVTTSNYANWVQQKPGQAPRGL |
| | | | | IGGTNKRAPGTPARFSGSLLGG |
| | | | | KAALTLSGVQPEDEAEYYCALW |
| | | | | YSNLWVFGGGTKLTVLHHHHHH |
| | | | | *VD |
| 175 | EGFR HL x | arti- | NA | caggtgcagctgcagcagtctg |
| | SEQ ID | ficial | | ggcctgatctggtgaagcctgg |
| | NO. 194 | | | ggcctcagtgaagatgtcctgc |
| | | | | aaggcttctggacacactttca |
| | | | | ctgactgtgttataatctgggt |
| | | | | gaaacagagagctggacagggc |
| | | | | cttgagtggattggacagattt |
| | | | | atccagggactggtcgttctta |
| | | | | ctacaatgagattttcaagggc |
| | | | | aaggccacactgactgcagaca |
| | | | | aatcctccaacacagtccacat |
| | | | | tcaactcagcagcctgacatct |
| | | | | gaggactctgcggtctatttct |
| | | | | gtgccctatctactcttattca |
| | | | | cgggacctggttttcttattgg |
| | | | | ggccaagggactctggtcactg |
| | | | | tctcttccggtggtggtggttc |
| | | | | tggcggcggcggctccggtggt |
| | | | | ggtggttctgacattgtactga |
| | | | | cccagtctccagcttccttacc |
| | | | | tgtgtctctggggcagagggcc |
| | | | | accatctcatgcagggccagcc |
| | | | | aaagtgtcagttcatctactta |
| | | | | tagttatatacactggtaccaa |
| | | | | cagaaaccaggacagccaccca |
| | | | | aactcctcatcacgtatgcatc |
| | | | | caacctagaatctggggtccct |
| | | | | gccaggttcagtggcagtgggt |
| | | | | ctgggacagacttcaccctcga |
| | | | | catccatcctgtggaggaggat |
| | | | | gattcttcaacatattactgtc |
| | | | | agcacagttgggagattccatt |
| | | | | tacgttcggctcggggacaaag |
| | | | | ttggaaataaaatccggaggtg |
| | | | | gtggctcccagaccgttgtgac |
| | | | | tcaggaaccttcactcaccgta |
| | | | | tcacctggtggaacagtcacac |
| | | | | tcacttgtcgctcgtccactgg |
| | | | | ggctgttacaactagcaactat |
| | | | | gccaactgggtccaacaaaaac |
| | | | | caggtcaggcaccccgtggtct |
| | | | | aataggtggtaccaacaagcgc |
| | | | | gcaccaggtactcctgccagat |
| | | | | tctcaggctccctgcttggagg |
| | | | | caaggctgccctcaccctctca |
| | | | | ggggtacagccagaggatgagg |
| | | | | cagaatattactgtgctctatg |
| | | | | gtacagcaacctctgggtgttc |
| | | | | ggtggaggaaccaaactgactg |
| | | | | tcctaggtggtggtggttctgg |
| | | | | cggcggcggctccggtggtggt |
| | | | | ggttctgaggtgcagctggtgg |
| | | | | agtctggaggaggattggtgca |
| | | | | gcctggagggtcattgaaactc |
| | | | | tcatgtgcagcctctggattca |
| | | | | ccttcaatacctacgccatgaa |
| | | | | ctgggtccgccaggctccagga |
| | | | | aagggtttggaatgggttgctc |
| | | | | gcataagaagtaaatataataa |
| | | | | ttatgcaacatattatgccgat |
| | | | | tcagtgaaagacaggttcacca |
| | | | | tctccagagatgattcaaaaaa |
| | | | | cactgcctatctacaaatgaac |
| | | | | aacttgaaaactgaggacactg |
| | | | | ccgtgtactactgtgtgagaca |
| | | | | tgggaacttcggtaatagctac |
| | | | | gtttcctggtttgcttactggg |
| | | | | gccaagggactctggtcaccgt |
| | | | | ctcctcacatcatcaccatcat |
| | | | | cattaggtcgac |
| 176 | EGFR HL x | arti- | AA | QVQLQQSGPDLVKPGASVKMSC |
| | SEQ ID | ficial | | KASGHTFTDCVIIWVKQRAGQG |
| | NO. 194 | | | LEWIGQIYPGTGRSYYNEIFKG |
| | | | | KATLTADKSSNTVHIQLSSLTS |
| | | | | EDSAVYFCALSTLIHGTWFSYW |
| | | | | GQGTLVTVSSGGGGSGGGGSGG |
| | | | | GGSDIVLTQSPASLPVSLGQRA |
| | | | | TISCRASQSVSSSTYSYIHWYQ |
| | | | | QKPGQPPKLLITYASNLESGVP |
| | | | | ARFSGSGSGTDFTLDIHPVEED |
| | | | | DSSTYYCQHSWEIPFTFGSGTK |
| | | | | LEIKSGGGGSQTVVTQEPSLTV |
| | | | | SPGGTVTLTCRSSTGAVTTSNY |
| | | | | ANWVQQKPGQAPRGLIGGTNKR |
| | | | | APGTPARFSGSLLGGKAALTLS |
| | | | | GVQPEDEAEYYCALWYSNLWVF |
| | | | | GGGTKLTVLGGGGSGGGGSGGG |
| | | | | GSEVQLVESGGGLVQPGGSLKL |
| | | | | SCAASGFTFNTYAMNWVRQAPG |
| | | | | KGLEWVARIRSKYNNYATYYAD |
| | | | | SVKDRFTISRDDSKNTAYLQMN |
| | | | | NLKTEDTAVYYCVRHGNFGNSY |
| | | | | VSWFAYWGQGTLVTVSSHHHHH |
| | | | | H*VD |
| 177 | EGFR LH x | arti- | NA | gacattgtgctgacacagtctc |
| | SEQ ID | ficial | | ctgcttccttacctgtgtctct |
| | NO. 194 | | | ggggcagagggccaccatctca |
| | | | | tgcagggccagccaaagtgtca |
| | | | | gttcatctacttatagttatat |
| | | | | acactggtaccaacagaaacca |
| | | | | ggacagccacccaaactcctca |
| | | | | tcacgtatgcatccaacctaga |
| | | | | atctggggtccctgccaggttc |
| | | | | agtggcagtgggtctgggacag |
| | | | | acttcaccctcgacatccatcc |
| | | | | tgtggaggaggatgattcttca |
| | | | | acatattactgtcagcacagtt |
| | | | | gggagattccatttacgttcgg |
| | | | | ctcggggacaaagttggaaata |
| | | | | aaaggtggtggtggttctggcg |
| | | | | gcggcggctccggtggtggtgg |
| | | | | ttctcaggttcagctgcagcag |
| | | | | tctggacctgatctggtgaagc |
| | | | | ctggggcctcagtgaagatgtc |
| | | | | ctgcaaggcttctggacacact |
| | | | | ttcactgactgtgttataatct |
| | | | | gggtgaaacagagagctggaca |
| | | | | gggccttgagtggattggacag |
| | | | | atttatccagggactggtcgtt |
| | | | | cttactacaatgagattttcaa |
| | | | | gggcaaggccacactgactgca |
| | | | | gacaaatcctccaacacagtcc |
| | | | | acattcaactcagcagcctgac |
| | | | | atctgaggactctgcggtctat |
| | | | | ttctgtgccctatctactctta |
| | | | | ttcacgggacctggttttctta |
| | | | | ttggggccaagggactctggtc |
| | | | | actgtctcttcctccggaggtg |
| | | | | gtggctcccagaccgttgtgac |
| | | | | tcaggaaccttcactcaccgta |
| | | | | tcacctggtggaacagtcacac |
| | | | | tcacttgtcgctcgtccactgg |
| | | | | ggctgttacaactagcaactat |
| | | | | gccaactgggtccaacaaaaac |
| | | | | caggtcaggcaccccgtggtct |
| | | | | aataggtggtaccaacaagcgc |
| | | | | gcaccaggtactcctgccagat |
| | | | | tctcaggctccctgcttggagg |
| | | | | caaggctgccctcaccctctca |
| | | | | ggggtacagccagaggatgagg |
| | | | | cagaatattactgtgctctatg |
| | | | | gtacagcaacctctgggtgttc |
| | | | | ggtggaggaaccaaactgactg |
| | | | | tcctaggtggtggtggttctgg |
| | | | | cggcggcggctccggtggtggt |
| | | | | ggttctgaggtgcagctggtgg |
| | | | | agtctggaggaggattggtgca |
| | | | | gcctggagggtcattgaaactc |
| | | | | tcatgtgcagcctctggattca |
| | | | | ccttcaatacctacgccatgaa |
| | | | | ctgggtccgccaggctccagga |
| | | | | aagggtttggaatgggttgctc |
| | | | | gcataagaagtaaatataataa |
| | | | | ttatgcaacatattatgccgat |
| | | | | tcagtgaaagacaggttcacca |
| | | | | tctccagagatgattcaaaaaa |
| | | | | cactgcctatctacaaatgaac |
| | | | | aacttgaaaactgaggacactg |
| | | | | ccgtgtactactgtgtgagaca |
| | | | | tgggaacttcggtaatagctac |
| | | | | gtttcctggtttgcttactggg |
| | | | | gccaagggactctggtcaccgt |
| | | | | ctcctcacatcatcaccatcat |
| | | | | cattaggtcgac |
| 178 | EGFR LH x | arti- | AA | DIVLTQSPASLPVSLGQRATIS |
| | SEQ ID | ficial | | CRASQSVSSSTYSYIHWYQQKP |
| | NO. 194 | | | GQPPKLLITYASNLESGVPARF |
| | | | | SGSGSGTDFTLDIHPVEEDDSS |
| | | | | TYYCQHSWEIPFTFGSGTKLEI |
| | | | | KGGGGSGGGGSGGGGSQVQLQQ |
| | | | | SGPDLVKPGASVKMSCKASGHT |
| | | | | FTDCVIIWVKQRAGQGLEWIGQ |
| | | | | IYPGTGRSYYNEIFKGKATLTA |
| | | | | DKSSNTVHIQLSSLTSEDSAVY |
| | | | | FCALSTLIHGTWFSYWGQGTLV |
| | | | | TVSSSGGGGSQTVVTQEPSLTV |
| | | | | SPGGTVTLTCRSSTGAVTTSNY |
| | | | | ANWVQQKPGQAPRGLIGGTNKR |
| | | | | APGTPARFSGSLLGGKAALTLS |
| | | | | GVQPEDEAEYYCALWYSNLWVF |
| | | | | GGGTKLTVLGGGGSGGGGSGGG |
| | | | | GSEVQLVESGGGLVQPGGSLKL |
| | | | | SCAASGFTFNTYAMNWVRQAPG |
| | | | | KGLEWVARIRSKYNNYATYYAD |
| | | | | SVKDRFTISRDDSKNTAYLQMN |
| | | | | NLKTEDTAVYYCVRHGNFGNSY |
| | | | | VSWFAYWGQGTLVTVSSHHHHH |
| | | | | H*VD |
| 179 | SEQ ID | arti- | NA | gaggtgcagctggtggagtctg |
| | NO. 170 x | ficial | | gaggaggattggtgcagcctgg |
| | EGFR HL | | | agggtcattgaaactctcatgt |
| | | | | gcagcctctggattcaccttca |
| | | | | atacctacgccatgaactgggt |
| | | | | ccgccaggctccaggaaagggt |
| | | | | ttggaatgggttgctcgcataa |
| | | | | gaagtaaatataataattatgc |
| | | | | aacatattatgccgattcagtg |
| | | | | aaagacaggttcaccatctcca |
| | | | | gagatgattcaaaaaacactgc |
| | | | | ctatctacaaatgaacaacttg |
| | | | | aaaactgaggacactgccgtgt |
| | | | | actactgtgtgagacatgggaa |
| | | | | cttcggtaatagctacgtttcc |
| | | | | tggtttgcttactggggccaag |
| | | | | ggactctggtcaccgtctcctc |
| | | | | aggtggtggtggttctggcggc |
| | | | | ggcggctccggtggtggtggtt |
| | | | | ctcagaccgttgtgactcagga |
| | | | | accttcactcaccgtatcacct |
| | | | | ggtggaacagtcacactcactt |
| | | | | gtcgctcgtccactggggctgt |
| | | | | tacaactagcaactatgccaac |
| | | | | tgggtccaacaaaaaccaggtc |
| | | | | aggcaccccgtggtctaatagg |
| | | | | tggtaccaacaagcgcgcacca |
| | | | | ggtactcctgccagattctcag |
| | | | | gctccctgcttggaggcaaggc |
| | | | | tgccctcaccctctcaggggta |
| | | | | cagccagaggatgaggcagaat |
| | | | | attactgtgctctatggtacag |
| | | | | caacctctgggtgttcggtgga |
| | | | | ggaaccaaactgactgtcctat |
| | | | | ccggaggtggtggctcccaggt |
| | | | | gcagctgcagcagtctgggcct |
| | | | | gatctggtgaagcctggggcct |
| | | | | cagtgaagatgtcctgcaaggc |
| | | | | ttctggacacactttcactgac |
| | | | | tgtgttataatctgggtgaaac |
| | | | | agagagctggacagggccttga |
| | | | | gtggattggacagatttatcca |
| | | | | gggactggtcgttcttactaca |
| | | | | atgagattttcaagggcaaggc |
| | | | | cacactgactgcagacaaatcc |
| | | | | tccaacacagtccacattcaac |
| | | | | tcagcagcctgacatctgagga |
| | | | | ctctgcggtctatttctgtgcc |
| | | | | ctatctactcttattcacggga |
| | | | | cctggttttcttattggggcca |
| | | | | agggactctggtcactgtctct |
| | | | | tccggtggtggtggttctggcg |
| | | | | gcggcggctccggtggtggtgg |
| | | | | ttctgacattgtactgacccag |
| | | | | tctccagcttccttacctgtgt |
| | | | | ctctggggcagagggccaccat |
| | | | | ctcatgcagggccagccaaagt |
| | | | | gtcagttcatctacttatagtt |
| | | | | atatacactggtaccaacagaa |
| | | | | accaggacagccacccaaactc |
| | | | | ctcatcacgtatgcatccaacc |
| | | | | tagaatctggggtccctgccag |
| | | | | gttcagtggcagtgggtctggg |
| | | | | acagacttcaccctcgacatcc |
| | | | | atcctgtggaggaggatgattc |
| | | | | ttcaacatattactgtcagcac |
| | | | | agttgggagattccatttacgt |
| | | | | tcggctcggggacaaagttgga |
| | | | | aataaaacatcatcaccatcat |
| | | | | cattaggtcgac |
| 180 | SEQ ID | arti- | AA | EVQLVESGGGLVQPGGSLKLSC |
| | NO. 170 x | ficial | | AASGFTFNTYAMNWVRQAPGKG |
| | EGFR HL | | | LEWVARIRSKYNNYATYYADSV |
| | | | | KDRFTISRDDSKNTAYLQMNNL |
| | | | | KTEDTAVYYCVRHGNFGNSYVS |
| | | | | WFAYWGQGTLVTVSSGGGGSGG |
| | | | | GGSGGGGSQTVVTQEPSLTVSP |
| | | | | GGTVTLTCRSSTGAVTTSNYAN |
| | | | | WVQQKPGQAPRGLIGGTNKRAP |
| | | | | GTPARFSGSLLGGKAALTLSGV |
| | | | | QPEDEAEYYCALWYSNLWVFGG |
| | | | | GTKLTVLSGGGGSQVQLQQSGP |
| | | | | DLVKPGASVKMSCKASGHTFTD |
| | | | | CVIIWVKQRAGQGLEWIGQIYP |
| | | | | GTGRSYYNEIFKGKATLTADKS |
| | | | | SNTVHIQLSSLTSEDSAVYFCA |
| | | | | LSTLIHGTWFSYWGQGTLVTVS |
| | | | | SGGGGSGGGGSGGGGSDIVLTQ |
| | | | | SPASLPVSLGQRATISCRASQS |
| | | | | VSSSTYSYIHWYQQKPGQPPKL |
| | | | | LITYASNLESGVPARFSGSGSG |
| | | | | TDFTLDIHPVEEDDSSTYYCQH |
| | | | | SWEIPFTFGSGTKLEIKHHHHH |
| | | | | H*VD |
| 181 | SEQ ID | arti- | NA | cagaccgttgtgactcaggaac |
| | NO. 194 x | ficial | | cttcactcaccgtatcacctgg |
| | EGFR HL | | | tggaacagtcacactcacttgt |
| | | | | cgctcgtccactggggctgtta |
| | | | | caactagcaactatgccaactg |
| | | | | ggtccaacaaaaaccaggtcag |
| | | | | gcaccccgtggtctaataggtg |
| | | | | gtaccaacaagcgcgcaccagg |
| | | | | tactcctgccagattctcaggc |
| | | | | tccctgcttggaggcaaggctg |
| | | | | ccctcaccctctcaggggtaca |
| | | | | gccagaggatgaggcagaatat |
| | | | | tactgtgctctatggtacagca |
| | | | | acctctgggtgttcggtggagg |
| | | | | aaccaaactgactgtcctaggt |
| | | | | ggtggtggttctggcggcggcg |
| | | | | gctccggtggtggtggttctga |
| | | | | ggtgcagctggtggagtctgga |
| | | | | ggaggattggtgcagcctggag |
| | | | | ggtcattgaaactctcatgtgc |
| | | | | agcctctggattcaccttcaat |
| | | | | acctacgccatgaactgggtcc |
| | | | | gccaggctccaggaaagggttt |
| | | | | ggaatgggttgctcgcataaga |
| | | | | agtaaatataataattatgcaa |
| | | | | catattatgccgattcagtgaa |
| | | | | agacaggttcaccatctccaga |
| | | | | gatgattcaaaaaacactgcct |
| | | | | atctacaaatgaacaacttgaa |
| | | | | aactgaggacactgccgtgtac |
| | | | | tactgtgtgagacatgggaact |
| | | | | tcggtaatagctacgtttcctg |
| | | | | gtttgcttactggggccaaggg |
| | | | | actctggtcaccgtctcctcat |
| | | | | ccggaggtggtggctcccaggt |
| | | | | gcagctgcagcagtctgggcct |
| | | | | gatctggtgaagcctggggcct |
| | | | | cagtgaagatgtcctgcaaggc |
| | | | | ttctggacacactttcactgac |
| | | | | tgtgttataatctgggtgaaac |
| | | | | agagagctggacagggccttga |
| | | | | gtggattggacagatttatcca |
| | | | | gggactggtcgttcttactaca |
| | | | | atgagattttcaagggcaaggc |
| | | | | cacactgactgcagacaaatcc |
| | | | | tccaacacagtccacattcaac |
| | | | | tcagcagcctgacatctgagga |
| | | | | ctctgcggtctatttctgtgcc |
| | | | | ctatctactcttattcacggga |
| | | | | cctggttttcttattggggcca |
| | | | | agggactctggtcactgtctct |
| | | | | tccggtggtggtggttctggcg |
| | | | | gcggcggctccggtggtggtgg |
| | | | | ttctgacattgtactgacccag |
| | | | | tctccagcttccttacctgtgt |
| | | | | ctctggggcagagggccaccat |
| | | | | ctcatgcagggccagccaaagt |
| | | | | gtcagttcatctacttatagtt |
| | | | | atatacactggtaccaacagaa |
| | | | | accaggacagccacccaaactc |
| | | | | ctcatcacgtatgcatccaacc |
| | | | | tagaatctggggtccctgccag |
| | | | | gttcagtggcagtgggtctggg |
| | | | | acagacttcaccctcgacatcc |
| | | | | atcctgtggaggaggatgattc |
| | | | | ttcaacatattactgtcagcac |
| | | | | agttgggagattccatttacgt |
| | | | | tcggctcggggacaaagttgga |
| | | | | aataaaacatcatcaccatcat |
| | | | | cattaggtcgac |
| 182 | SEQ ID | arti- | AA | QTVVTQEPSLTVSPGGTVTLTC |
| | NO. 194 x | ficial | | RSSTGAVTTSNYANWVQQKPGQ |
| | EGFR HL | | | APRGLIGGTNKRAPGTPARFSG |
| | | | | SLLGGKAALTLSGVQPEDEAEY |
| | | | | YCALWYSNLWVFGGGTKLTVLG |
| | | | | GGGSGGGGSGGGGSEVQLVESG |
| | | | | GGLVQPGGSLKLSCAASGFTFN |
| | | | | TYAMNWVRQAPGKGLEWVARIR |
| | | | | SKYNNYATYYADSVKDRFTISR |
| | | | | DDSKNTAYLQMNNLKTEDTAVY |
| | | | | YCVRHGNFGNSYVSWFAYWGQG |
| | | | | TLVTVSSSGGGGSQVQLQQSGP |
| | | | | DLVKPGASVKMSCKASGHTFTD |
| | | | | CVIIWVKQRAGQGLEWIGQIYP |
| | | | | GTGRSYYNEIFKGKATLTADKS |
| | | | | SNTVHIQLSSLTSEDSAVYFCA |
| | | | | LSTLIHGTWFSYWGQGTLVTVS |
| | | | | SGGGGSGGGGSGGGGSDIVLTQ |
| | | | | SPASLPVSLGQRATISCRASQS |
| | | | | VSSSTYSYIHWYQQKGQPPKLL |
| | | | | ITYASNLESGVPARFSGSGSGT |
| | | | | DFTLDIHPVEEDDSSTYYCQHS |
| | | | | WEIPFTFGSGTKLEIKHHHHHH |
| | | | | *VD |
| 183 | SEQ ID | arti- | NA | gaggtgcagctggtggagtctg |
| | NO. 170 x | ficial | | gaggaggattggtgcagcctgg |
| | EGFR HL | | | agggtcattgaaactctcatgt |
| | | | | gcagcctctggattcaccttca |
| | | | | atacctacgccatgaactgggt |
| | | | | ccgccaggctccaggaaagggt |
| | | | | ttggaatgggttgctcgcataa |
| | | | | gaagtaaatataataattatgc |
| | | | | aacatattatgccgattcagtg |
| | | | | aaagacaggttcaccatctcca |
| | | | | gagatgattcaaaaaacactgc |
| | | | | ctatctacaaatgaacaacttg |
| | | | | aaaactgaggacactgccgtgt |
| | | | | actactgtgtgagacatgggaa |
| | | | | cttcggtaatagctacgtttcc |
| | | | | tggtttgcttactggggccaag |
| | | | | ggactctggtcaccgtctcctc |
| | | | | aggtggtggtggttctggcggc |
| | | | | ggcggctccggtggtggtggtt |
| | | | | ctcagaccgttgtgactcagga |
| | | | | accttcactcaccgtatcacct |
| | | | | ggtggaacagtcacactcactt |
| | | | | gtcgctcgtccactggggctgt |
| | | | | tacaactagcaactatgccaac |
| | | | | tgggtccaacaaaaaccaggtc |
| | | | | aggcaccccgtggtctaatagg |
| | | | | tggtaccaacaagcgcgcacca |
| | | | | ggtactcctgccagattctcag |
| | | | | gctccctgcttggaggcaaggc |
| | | | | tgccctcaccctctcaggggta |
| | | | | cagccagaggatgaggcagaat |
| | | | | attactgtgctctatggtacag |
| | | | | caacctctgggtgttcggtgga |
| | | | | ggaaccaaactgactgtcctat |
| | | | | ccggaggtggtggctccgacat |
| | | | | tgtgctgacacagtctcctgct |
| | | | | tccttacctgtgtctctggggc |
| | | | | agagggccaccatctcatgcag |
| | | | | ggccagccaaagtgtcagttca |
| | | | | tctacttatagttatatacact |
| | | | | ggtaccaacagaaaccaggaca |
| | | | | gccacccaaactcctcatcacg |
| | | | | tatgcatccaacctagaatctg |
| | | | | gggtccctgccaggttcagtgg |
| | | | | cagtgggtctgggacagacttc |
| | | | | accctcgacatccatcctgtgg |
| | | | | aggaggatgattcttcaacata |
| | | | | ttactgtcagcacagttgggag |
| | | | | attccatttacgttcggctcgg |
| | | | | ggacaaagttggaaataaaagg |
| | | | | tggtggtggttctggcggcggc |
| | | | | ggctccggtggtggtggttctc |
| | | | | aggttcagctgcagcagtctgg |
| | | | | acctgatctggtgaagcctggg |
| | | | | gcctcagtgaagatgtcctgca |
| | | | | aggcttctggacacactttcac |
| | | | | tgactgtgttataatctgggtg |
| | | | | aaacagagagctggacagggcc |
| | | | | ttgagtggattggacagattta |
| | | | | tccagggactggtcgttcttac |
| | | | | tacaatgagattttcaagggca |
| | | | | aggccacactgactgcagacaa |
| | | | | atcctccaacacagtccacatt |
| | | | | caactcagcagcctgacatctg |
| | | | | aggactctgcggtctatttctg |
| | | | | tgccctatctactcttattcac |
| | | | | gggacctggttttcttattggg |
| | | | | gccaagggactctggtcactgt |
| | | | | ctcttcccatcatcaccatcat |
| | | | | cattaggtcgac |
| 184 | SEQ ID | arti- | AA | EVQLVESGGGLVQPGGSLKLSC |
| | NO. 170 x | ficial | | AASGFTFNTYAMNWVRQAPGKG |
| | EGFR HL | | | LEWVARIRSKYNNYATYYADSV |
| | | | | KDRFTISRDDSKNTAYLQMNNL |
| | | | | KTEDTAVYYCVRHGNFGNSYVS |
| | | | | WFAYWGQGTLVTVSSGGGGSGG |
| | | | | GGSGGGGSQTVVTQEPSLTVSP |
| | | | | GGTVTLTCRSSTGAVTTSNYAN |
| | | | | WVQQKPGQAPRGLIGGTNKRAP |
| | | | | GTPARFSGSLLGGKAALTLSGV |
| | | | | QPEDEAEYYCALWYSNLWVFGG |
| | | | | GTKLTVLSGGGGSDIVLTQSPA |
| | | | | SLPVSLGQRATISCRASQSVSS |
| | | | | STYSYIHWYQQKPGQPPKLLIT |
| | | | | YASNLESGVPARFSGSGSGTDF |
| | | | | TLDIHPVEEDDSSTYYCQHSWE |
| | | | | IPFTFGSGTKLEIKGGGGSGGG |
| | | | | GSGGGGSQVQLQQSGPDLVKPG |
| | | | | ASVKMSCKASGHTFTDCVIIWV |
| | | | | KQRAGQGLEWIGQIYPGTGRSY |
| | | | | YNEIFKGKATLTADKSSNTVHI |
| | | | | QLSSLTSEDSAVYFCALSTLIH |
| | | | | GTWFSYWGQGTLVTVSSHHHHH |
| | | | | H*VD |
| 185 | SEQ ID | arti- | NA | cagaccgttgtgactcaggaac |
| | NO. 194 x | ficial | | cttcactcaccgtatcacctgg |
| | EGFR HL | | | tggaacagtcacactcacttgt |
| | | | | cgctcgtccactggggctgtta |
| | | | | caactagcaactatgccaactg |
| | | | | ggtccaacaaaaaccaggtcag |
| | | | | gcaccccgtggtctaataggtg |
| | | | | gtaccaacaagcgcgcaccagg |
| | | | | tactcctgccagattctcaggc |
| | | | | tccctgcttggaggcaaggctg |
| | | | | ccctcaccctctcaggggtaca |
| | | | | gccagaggatgaggcagaatat |
| | | | | tactgtgctctatggtacagca |
| | | | | acctctgggtgttcggtggagg |
| | | | | aaccaaactgactgtcctaggt |
| | | | | ggtggtggttctggcggcggcg |
| | | | | gctccggtggtggtggttctga |
| | | | | ggtgcagctggtggagtctgga |
| | | | | ggaggattggtgcagcctggag |
| | | | | ggtcattgaaactctcatgtgc |
| | | | | agcctctggattcaccttcaat |
| | | | | acctacgccatgaactgggtcc |
| | | | | gccaggctccaggaaagggttt |
| | | | | ggaatgggttgctcgcataaga |
| | | | | agtaaatataataattatgcaa |
| | | | | catattatgccgattcagtgaa |
| | | | | agacaggttcaccatctccaga |
| | | | | gatgattcaaaaaacactgcct |
| | | | | atctacaaatgaacaacttgaa |
| | | | | aactgaggacactgccgtgtac |
| | | | | tactgtgtgagacatgggaact |
| | | | | tcggtaatagctacgtttcctg |
| | | | | gtttgcttactggggccaaggg |
| | | | | actctggtcaccgtctcctcat |
| | | | | ccggaggtggtggctccgacat |
| | | | | tgtgctgacacagtctcctgct |
| | | | | tccttacctgtgtctctggggc |
| | | | | agagggccaccatctcatgcag |
| | | | | ggccagccaaagtgtcagttca |
| | | | | tctacttatagttatatacact |
| | | | | ggtaccaacagaaaccaggaca |
| | | | | gccacccaaactcctcatcacg |
| | | | | tatgcatccaacctagaatctg |
| | | | | gggtccctgccaggttcagtgg |
| | | | | cagtgggtctgggacagacttc |
| | | | | accctcgacatccatcctgtgg |
| | | | | aggaggatgattcttcaacata |
| | | | | ttactgtcagcacagttgggag |
| | | | | attccatttacgttcggctcgg |
| | | | | ggacaaagttggaaataaaagg |
| | | | | tggtggtggttctggcggcggc |
| | | | | ggctccggtggtggtggttctc |
| | | | | aggttcagctgcagcagtctgg |
| | | | | acctgatctggtgaagcctggg |
| | | | | gcctcagtgaagatgtcctgca |
| | | | | aggcttctggacacactttcac |
| | | | | tgactgtgttataatctgggtg |
| | | | | aaacagagagctggacagggcc |
| | | | | ttgagtggattggacagattta |
| | | | | tccagggactggtcgttcttac |
| | | | | tacaatgagattttcaagggca |
| | | | | aggccacactgactgcagacaa |
| | | | | atcctccaacacagtccacatt |
| | | | | caactcagcagcctgacatctg |
| | | | | aggactctgcggtctatttctg |
| | | | | tgccctatctactcttattcac |
| | | | | gggacctggttttcttattggg |
| | | | | gccaagggactctggtcactgt |
| | | | | ctcttcccatcatcaccatcat |
| | | | | cattaggtcgac |
| 186 | SEQ ID | arti- | AA | QTVVTQEPSLTVSPGGTVTLTC |
| | NO. 194 x | ficial | | RSSTGAVTTSNYANWVQQKPGQ |
| | EGFR HL | | | APRGLIGGTNKRAPGTPARFSG |
| | | | | SLLGGKAALTLSGVQPEDEAEY |
| | | | | YCALWYSNLWVFGGGTKLTVLG |
| | | | | GGGSGGGGSGGGGSEVQLVESG |
| | | | | GGLVQPGGSLKLSCAASGFTFN |
| | | | | TYAMNWVRQAPGKGLEWVARIR |
| | | | | SKYNNYATYYADSVKDRFTISR |
| | | | | DDSKNTAYLQMNNLKTEDTAVY |
| | | | | YCVRHGNFGNSYVSWFAYWGQG |
| | | | | TLVTVSSSGGGGSDIVLTQSPA |
| | | | | SLPVSLGQRATISCRASQSVSS |
| | | | | STYSYIHWYQQKPGQPPKLLIT |
| | | | | YASNLESGVPARFSGSGSGTDF |
| | | | | TLDIHPVEEDDSSTYYCQHSWE |
| | | | | IPFTFGSGTKLEIKGGGGSGGG |
| | | | | GSGGGGSQVQLQQSGPDLVKPG |
| | | | | ASVKMSCKASGHTFTDCVIIWV |
| | | | | KQRAGQGLEWIGQIYPGTGRSY |
| | | | | YNEIFKGKATLTADKSSNTVHI |
| | | | | QLSSLTSEDSAVYFCALSTLIH |
| | | | | GTWFSYWGQGTLVTVSSHHHHH |
| | | | | H*VD |
| 187 | CAIX HL x | arti- | NA | gacgtgaagctcgtggagtctg |
| | SEQ ID | ficial | | ggggaggcttagtgaagcttgg |
| | NO. 194 | | | agggtccctgaaactctcctgt |
| | | | | gcagcctctggattcactttca |
| | | | | gtaactattacatgtcttgggt |
| | | | | tcgccagactccagagaagagg |
| | | | | ctggagttggtcgcagccatta |
| | | | | atagtgatggtggtatcaccta |
| | | | | ctatctagacactgtgaagggc |
| | | | | cgattcaccatttcaagagaca |
| | | | | atgccaagaacaccctgtacct |
| | | | | gcaaatgagcagtctgaagtct |
| | | | | gaggacacagccttgttttact |
| | | | | gtgcaagacaccgctcgggcta |
| | | | | cttttctatggactactggggt |
| | | | | caaggaacctcagtcaccgtct |
| | | | | cctcaggtggtggtggttctgg |
| | | | | cggcggcggctccggtggtggt |
| | | | | ggttctgacattgtgatgaccc |
| | | | | agtctcaaagattcatgtccac |
| | | | | aacagtaggagacagggtcagc |
| | | | | atcacctgcaaggccagtcaga |
| | | | | atgtggtttctgctgttgcctg |
| | | | | gtatcaacagaaaccaggacaa |
| | | | | tctcctaaactactgatttact |
| | | | | cagcatccaatcggtacactgg |
| | | | | agtccctgatcgcttcacaggc |
| | | | | agtggatctgggacagatttca |
| | | | | ctctcaccattagcaatatgca |
| | | | | gtctgaagacctggctgatttt |
| | | | | ttctgtcaacaatatagcaact |
| | | | | atccgtggacgttcggtggagg |
| | | | | caccaagctggaaatcaaatcc |
| | | | | ggaggtggtggctcccagaccg |
| | | | | ttgtgactcaggaaccttcact |
| | | | | caccgtatcacctggtggaaca |
| | | | | gtcacactcacttgtcgctcgt |
| | | | | ccactggggctgttacaactag |
| | | | | caactatgccaactgggtccaa |
| | | | | caaaaaccaggtcaggcacccc |
| | | | | gtggtctaataggtggtaccaa |
| | | | | caagcgcgcaccaggtactcct |
| | | | | gccagattctcaggctccctgc |
| | | | | ttggaggcaaggctgccctcac |
| | | | | cctctcaggggtacagccagag |
| | | | | gatgaggcagaatattactgtg |
| | | | | ctctatggtacagcaacctctg |
| | | | | ggtgttcggtggaggaaccaaa |
| | | | | ctgactgtcctaggtggtggtg |
| | | | | gttctggcggcggcggctccgg |
| | | | | tggtggtggttctgaggtgcag |
| | | | | ctggtggagtctggaggaggat |
| | | | | tggtgcagcctggagggtcatt |
| | | | | gaaactctcatgtgcagcctct |
| | | | | ggattcaccttcaatacctacg |
| | | | | ccatgaactgggtccgccaggc |
| | | | | tccaggaaagggtttggaatgg |
| | | | | gttgctcgcataagaagtaaat |
| | | | | ataataattatgcaacatatta |
| | | | | tgccgattcagtgaaagacagg |
| | | | | ttcaccatctccagagatgatt |
| | | | | caaaaaacactgcctatctaca |
| | | | | aatgaacaacttgaaaactgag |
| | | | | gacactgccgtgtactactgtg |
| | | | | tgagacatgggaacttcggtaa |
| | | | | tagctacgtttcctggtttgct |
| | | | | tactggggccaagggactctgg |
| | | | | tcaccgtctcctcacatcatca |
| | | | | ccatcatcattaggtcgac |
| 188 | CAIX HL x | arti- | AA | DVKLVESGGGLVKLGGSLKLSC |
| | SEQ ID | ficial | | AASGFTFSNYYMSWVRQTPEKR |
| | NO. 194 | | | LELVAAINSDGGITYYLDTVKG |
| | | | | RFTISRDNAKNTLYLQMSSLKS |
| | | | | EDTALFYCARHRSGYFSMDYWG |
| | | | | QGTSVTVSSGGGGSGGGGSGGG |
| | | | | GSDIVMTQSQRFMSTTVGDRVS |
| | | | | ITCKASQNVVSAVAWYQQKPGQ |
| | | | | SPKLLIYSASNRYTGVPDRFTG |
| | | | | SGSGTDFTLTISNMQSEDLADF |
| | | | | FCQQYSNYPWTFGGGTKLEIKS |
| | | | | GGGGSQTVVTQEPSLTVSPGGT |
| | | | | VTLTCRSSTGAVTTSNYANWVQ |
| | | | | QKPGQAPRGLIGGTNKRAPGTP |
| | | | | ARFSGSLLGGKAALTLSGVQPE |
| | | | | DEAEYYCALWYSNLWVFGGGTK |
| | | | | LTVLGGGGSGGGGSGGGGSEVQ |
| | | | | LVESGGGLVQPGGSLKLSCAAS |
| | | | | GFTFNTYAMNWVRQAPGKGLEW |
| | | | | VARIRSKYNNYATYYADSVKDR |
| | | | | FTISRDDSKNTAYLQMNNLKTE |
| | | | | DTAVYYCVRHGNFGNSYVSWFA |
| | | | | YWGQGTLVTVSSHHHHHH*VD |
| 189 | CAIX HL x | arti- | NA | gacgtgaagctcgtggagtctg |
| | SEQ ID | ficial | | ggggaggcttagtgaagcttgg |
| | NO. 170 | | | agggtccctgaaactctcctgt |
| | | | | gcagcctctggattcactttca |
| | | | | gtaactattacatgtcttgggt |
| | | | | tcgccagactccagagaagagg |
| | | | | ctggagttggtcgcagccatta |
| | | | | atagtgatggtggtatcaccta |
| | | | | ctatctagacactgtgaagggc |
| | | | | cgattcaccatttcaagagaca |
| | | | | atgccaagaacaccctgtacct |
| | | | | gcaaatgagcagtctgaagtct |
| | | | | gaggacacagccttgttttact |
| | | | | gtgcaagacaccgctcgggcta |
| | | | | cttttctatggactactggggt |
| | | | | caaggaacctcagtcaccgtct |
| | | | | cctcaggtggtggtggttctgg |
| | | | | cggcggcggctccggtggtggt |
| | | | | ggttctgacattgtgatgaccc |
| | | | | agtctcaaagattcatgtccac |
| | | | | aacagtaggagacagggtcagc |
| | | | | atcacctgcaaggccagtcaga |
| | | | | atgtggtttctgctgttgcctg |
| | | | | gtatcaacagaaaccaggacaa |
| | | | | tctcctaaactactgatttact |
| | | | | cagcatccaatcggtacactgg |
| | | | | agtccctgatcgcttcacaggc |
| | | | | agtggatctgggacagatttca |
| | | | | ctctcaccattagcaatatgca |
| | | | | gtctgaagacctggctgatttt |
| | | | | ttctgtcaacaatatagcaact |
| | | | | atccgtggacgttcggtggagg |
| | | | | caccaagctggaaatcaaatcc |
| | | | | ggaggtggtggctccgaggtgc |
| | | | | agctggtggagtctggaggagg |
| | | | | attggtgcagcctggagggtca |
| | | | | ttgaaactctcatgtgcagcct |
| | | | | ctggattcaccttcaataccta |
| | | | | cgccatgaactgggtccgccag |
| | | | | gctccaggaaagggtttggaat |
| | | | | gggttgctcgcataagaagtaa |
| | | | | atataataattatgcaacatat |
| | | | | tatgccgattcagtgaaagaca |
| | | | | ggttcaccatctccagagatga |
| | | | | ttcaaaaaacactgcctatcta |
| | | | | caaatgaacaacttgaaaactg |
| | | | | aggacactgccgtgtactactg |
| | | | | tgtgagacatgggaacttcggt |
| | | | | aatagctacgtttcctggtttg |
| | | | | cttactggggccaagggactct |
| | | | | ggtcaccgtctcctcaggtggt |
| | | | | ggtggttctggcggcggcggct |
| | | | | ccggtggtggtggttctcagac |
| | | | | cgttgtgactcaggaaccttca |
| | | | | ctcaccgtatcacctggtggaa |
| | | | | cagtcacactcacttgtcgctc |
| | | | | gtccactggggctgttacaact |
| | | | | agcaactatgccaactgggtcc |
| | | | | aacaaaaaccaggtcaggcacc |
| | | | | ccgtggtctaataggtggtacc |
| | | | | aacaagcgcgcaccaggtactc |
| | | | | ctgccagattctcaggctccct |
| | | | | gcttggaggcaaggctgccctc |
| | | | | accctctcaggggtacagccag |
| | | | | aggatgaggcagaatattactg |
| | | | | tgctctatggtacagcaacctc |
| | | | | tgggtgttcggtggaggaacca |
| | | | | aactgactgtcctacatcatca |
| | | | | ccatcatcattaggtcgac |
| 190 | CAIX HL x | arti- | AA | DVKLVESGGGLVKLGGSLKLSC |
| | SEQ ID | ficial | | AASGFTFSNYYMSWVRQTPEKR |
| | NO. 170 | | | LELVAAINSDGGITYYLDTVKG |
| | | | | RFTISRDNAKNTLYLQMSSLKS |
| | | | | EDTALFYCARHRSGYFSMDYWG |
| | | | | QGTSVTVSSGGGGSGGGGSGGG |
| | | | | GSDIVMTQSQRFMSTTVGDRVS |
| | | | | ITCKASQNVVSAVAWYQQKPGQ |
| | | | | SPKLLIYSASNRYTGVPDRFTG |
| | | | | SGSGTDFTLTISNMQSEDLADF |
| | | | | FCQQYSNYPWTFGGGTKLEIKS |
| | | | | GGGGSEVQLVESGGGLVQPGGS |
| | | | | LKLSCAASGFTFNTYAMNWVRQ |
| | | | | APGKGLEWVARIRSKYNNYATY |
| | | | | YADSVKDRFTISRDDSKNTAYL |
| | | | | QMNNLKTEDTAVYYCVRHGNFG |
| | | | | NSYVSWFAYWGQGTLVTVSSGG |
| | | | | GGSGGGGSGGGGSQTVVTQEPS |
| | | | | LTVSPGGTVTLTCRSSTGAVTT |
| | | | | SNYANWVQQKPGQAPRGLIGGT |
| | | | | NKRAPGTPARFSGSLLGGKAAL |
| | | | | TLSGVQPEDEAEYYCALWYSNL |
| | | | | WVFGGGTKLTVLHHHHHH*VD |
| 191 | CAIX LH x | arti- | NA | gacattgtgatgacccagtctc |
| | SEQ ID | ficial | | aaagattcatgtccacaacagt |
| | NO. 170 | | | aggagacagggtcagcatcacc |
| | | | | tgcaaggccagtcagaatgtgg |
| | | | | tttctgctgttgcctggtatca |
| | | | | acagaaaccaggacaatctcct |
| | | | | aaactactgatttactcagcat |
| | | | | ccaatcggtacactggagtccc |
| | | | | tgatcgcttcacaggcagtgga |
| | | | | tctgggacagatttcactctca |
| | | | | ccattagcaatatgcagtctga |
| | | | | agacctggctgattttttctgt |
| | | | | caacaatatagcaactatccgt |
| | | | | ggacgttcggtggaggcaccaa |
| | | | | gctggaaatcaaaggtggtggt |
| | | | | ggttctggcggcggcggctccg |
| | | | | gtggtggtggttctgacgtgaa |
| | | | | gctcgtggagtctgggggaggc |
| | | | | ttagtgaagcttggagggtccc |
| | | | | tgaaactctcctgtgcagcctc |
| | | | | tggattcactttcagtaactat |
| | | | | tacatgtcttgggttcgccaga |
| | | | | ctccagagaagaggctggagtt |
| | | | | ggtcgcagccattaatagtgat |
| | | | | ggtggtatcacctactatctag |
| | | | | acactgtgaagggccgattcac |
| | | | | catttcaagagacaatgccaag |
| | | | | aacaccctgtacctgcaaatga |
| | | | | gcagtctgaagtctgaggacac |
| | | | | agccttgttttactgtgcaaga |
| | | | | caccgctcgggctacttttcta |
| | | | | tggactactggggtcaaggaac |
| | | | | ctcagtcaccgtctcctcctcc |
| | | | | ggaggtggtggctccgaggtgc |
| | | | | agctggtggagtctggaggagg |
| | | | | attggtgcagcctggagggtca |
| | | | | ttgaaactctcatgtgcagcct |
| | | | | ctggattcaccttcaataccta |
| | | | | cgccatgaactgggtccgccag |
| | | | | gctccaggaaagggtttggaat |
| | | | | gggttgctcgcataagaagtaa |
| | | | | atataataattatgcaacatat |
| | | | | tatgccgattcagtgaaagaca |
| | | | | ggttcaccatctccagagatga |
| | | | | ttcaaaaaacactgcctatcta |
| | | | | caaatgaacaacttgaaaactg |
| | | | | aggacactgccgtgtactactg |
| | | | | tgtgagacatgggaacttcggt |
| | | | | aatagctacgtttcctggtttg |
| | | | | cttactggggccaagggactct |
| | | | | ggtcaccgtctcctcaggtggt |
| | | | | ggtggttctggcggcggcggct |
| | | | | ccggtggtggtggttctcagac |
| | | | | cgttgtgactcaggaaccttca |
| | | | | ctcaccgtatcacctggtggaa |
| | | | | cagtcacactcacttgtcgctc |
| | | | | gtccactggggctgttacaact |
| | | | | agcaactatgccaactgggtcc |
| | | | | aacaaaaaccaggtcaggcacc |
| | | | | ccgtggtctaataggtggtacc |
| | | | | aacaagcgcgcaccaggtactc |
| | | | | ctgccagattctcaggctccct |
| | | | | gcttggaggcaaggctgccctc |
| | | | | accctctcaggggtacagccag |
| | | | | aggatgaggcagaatattactg |
| | | | | tgctctatggtacagcaacctc |
| | | | | tgggtgttcggtggaggaacca |
| | | | | aactgactgtcctacatcatca |
| | | | | ccatcatcattaggtcgac |
| 192 | CAIX LH x | arti- | AA | DIVMTQSQRFMSTTVGDRVSIT |
| | SEQ ID | ficial | | CKASQNVVSAVAWYQQKPGQSP |
| | NO. 170 | | | KLLIYSASNRYTGVPDRFTGSG |
| | | | | SGTDFTLTISNMQSEDLADFFC |
| | | | | QQYSNYPWTFGGGTKLEIKGGG |
| | | | | GSGGGGSGGGGSDVKLVESGGG |
| | | | | LVKLGGSLKLSCAASGFTFSNY |
| | | | | YMSWVRQTPEKRLELVAAINSD |
| | | | | GGITYYLDTVKGRFTISRDNAK |
| | | | | NTLYLQMSSLKSEDTALFYCAR |
| | | | | HRSGYFSMDYWGQGTSVTVSSS |
| | | | | GGGGSEVQLVESGGGLVQPGGS |
| | | | | LKLSCAASGFTFNTYAMNWVRQ |
| | | | | APGKGLEWVARIRSKYNNYATY |
| | | | | YADSVKDRFTISRDDSKNTAYL |
| | | | | QMNNLKTEDTAVYYCVRHGNFG |
| | | | | NSYVSWFAYWGQGTLVTVSSGG |
| | | | | GGSGGGGSGGGGSQTVVTQEPS |
| | | | | LTVSPGGTVTLTCRSSTGAVTT |
| | | | | SNYANWVQQKPGQAPRGLIGGT |
| | | | | NKRAPGTPARFSGSLLGGKAAL |
| | | | | TLSGVQPEDEAEYYCALWYSNL |
| | | | | WVFGGGTKLTVLHHHHHH*VD |
| 193 | Human-like | arti- | NA | cagaccgttgtgactcaggaac |
| | VL (SEQ | ficial | | cttcactcaccgtatcacctgg |
| | ID NO. | | | tggaacagtcacactcacttgt |
| | 168) x | | | cgctcgtccactggggctgtta |
| | Human- | | | caactagcaactatgccaactg |
| | like VH | | | ggtccaacaaaaaccaggtcag |
| | (SEQ ID | | | gcaccccgtggtctaataggtg |
| | NO. 110) | | | gtaccaacaagcgcgcaccagg |
| | scFv | | | tactcctgccagattctcaggc |
| | | | | tccctgcttggaggcaaggctg |
| | | | | ccctcaccctctcaggggtaca |
| | | | | gccagaggatgaggcagaatat |
| | | | | tactgtgctctatggtacagca |
| | | | | acctctgggtgttcggtggagg |
| | | | | aaccaaactgactgtcctaggt |
| | | | | ggtggtggttctggcggcggcg |
| | | | | gctccggtggtggtggttctga |
| | | | | ggtgcagctggtggagtctgga |
| | | | | ggaggattggtgcagcctggag |
| | | | | ggtcattgaaactctcatgtgc |
| | | | | agcctctggattcaccttcaat |
| | | | | acctacgccatgaactgggtcc |
| | | | | gccaggctccaggaaagggttt |
| | | | | ggaatgggttgctcgcataaga |
| | | | | agtaaatataataattatgcaa |
| | | | | catattatgccgattcagtgaa |
| | | | | agacaggttcaccatctccaga |
| | | | | gatgattcaaaaaacactgcct |
| | | | | atctacaaatgaacaacttgaa |
| | | | | aactgaggacactgccgtgtac |
| | | | | tactgtgtgagacatgggaact |
| | | | | tcggtaatagctacgtttcctg |
| | | | | gtttgcttactggggccaaggg |
| | | | | actctggtcaccgtctcctca |
| 194 | Human-like | arti- | AA | QTVVTQEPSLTVSPGGTVTLTC |
| | VL (SEQ | ficial | | RSSTGAVTTSNYANWVQQKPGQ |
| | ID NO. | | | APRGLIGGTNKRAPGTPARFSG |
| | 168) x | | | SLLGGKAALTLSGVQPEDEAEY |
| | Human- | | | YCALWYSNLWVFGGGTKLTVLG |
| | like VH | | | GGGSGGGGSGGGGSEVQLVESG |
| | (SEQ ID | | | GGLVQPGGSLKLSCAASGFTFN |
| | NO. 110) | | | TYAMNWVRQAPGKGLEWVARIR |
| | scFv | | | SKYNNYATYYADSVKDRFTISR |
| | | | | DDSKNTAYLQMNNLKTEDTAVY |
| | | | | YCVRHGNFGNSYVSWFAYWGQG |
| | | | | TLVTVSS |
| 195 | epitope | arti- | AA | EFSELEQSGYYVC |
| | | ficial | | |
| 196 | epitope | arti- | AA | EFSELEQSGYYVK |
| | | ficial | | |
| 197 | 5′ EGFR | arti- | NA | GGTCTAGAGCATGCGACCCTCC |
| | XbaI | ficial | | GGGACGGCCGGG |
| 198 | 3′ EGFR | arti- | NA | TTTTAAGTCGACTCATGCTCCA |
| | SalI | ficial | | ATAAATTCACTGCT |
| 199 | epitope | arti- | AA | QDGNEEMGSITQT |
| | | ficial | | |
| 200 | epitope | arti- | AA | YYVSYPRGSNPED |
| | | ficial | | |
| 201 | epitope | arti- | AA | EFSEMEQSGYYVC |
| | | ficial | | |
| 202 | epitope | arti- | AA | FSEXE; X as in SEQ ID |
| | | ficial | | NO: 204 |
| 203 | epitope | arti- | AA | QYPGSEILWQHND |
| | | ficial | | |
| 204 | epitope | arti- | AA | FSEXE, wherein X |
| | | ficial | | represents L (Leucine) |
| | | | | or M (Methionine) |
| 205 | epitope | arti- | AA | FSELE |
| | | ficial | | |
| 206 | epitope | arti- | AA | FSEME |
| | | ficial | | |
| 207 | epitope | arti- | AA | EFSEXEQSGYYVC, wherein |
| | | ficial | | X represents L |
| | | | | (Leucine) or M |
| | | | | (Methionine) |

Abbreviations:
scFv = single chain Fv
AA = amino acid sequence
NA = nuclei acid sequence
L = VL region
H = VH region
Single letter code as used in the sequence listing:
B = C or G or T
D = A or G or T
H = A or C or T
K = G or T
M = A or C
N = A or C or G or T
R = A or G
S = C or G
V = A or C or G
W = A or T
Y = C or T

1-43. (canceled)

44. A nucleic acid comprising a nucleotide sequence encoding a bispecific single chain antibody, the antibody comprising:
(a) a first binding domain which binds to CD3, wherein the first binding domain comprises the amino acid sequence of CDR-L1 (SEQ ID NO: 118), CDR-L2 (SEQ ID NO: 117), and CDR-L3 (SEQ ID NO: 116), and CDR-H1 (SEQ ID NO: 115), CDR-H2 (SEQ ID NO: 114) and CDR-H3 (SEQ ID NO: 112 or 113); and
(b) a second binding domain which binds to a cell surface antigen.

45. The nucleic acid of claim 44, wherein the nucleotide sequence encodes a first binding domain comprising a VH region comprising at least 90% sequence identity to the amino acid sequence of SEQ ID NO: 110 and/or a VL region comprising at least 90% sequence identity to the amino acid sequence of SEQ ID NO: 168.

46. The nucleic acid of claim 44, wherein the nucleotide sequence encodes a first binding domain comprising a VH region comprising at least 95% sequence identity to the amino acid sequence of SEQ ID NO: 110 and/or a VL region comprising at least 95% sequence identity to the amino acid sequence of SEQ ID NO: 168.

47. The nucleic acid of claim 44, wherein the nucleotide sequence encodes a first binding domain comprising a VH region comprising the amino acid sequence of SEQ ID NO: 110 and/or a VL region comprising the amino acid sequence of SEQ ID NO: 168.

48. The nucleic acid of claim 44, wherein the nucleotide sequence encodes a first binding domain comprising an scFv comprising at least 90% sequence identity to the amino acid sequence of SEQ ID NO: 170 or 194.

49. The nucleic acid of claim 44, wherein the nucleotide sequence encodes a first binding domain comprising an scFv comprising at least 95% sequence identity to the amino acid sequence of SEQ ID NO: 170 or 194.

50. The nucleic acid of claim 44, wherein the nucleotide sequence encodes a first binding domain comprising an scFv comprising the amino acid sequence of SEQ ID NO: 170 or 194.

51. The nucleic acid of claim 44, wherein the nucleotide sequence encodes a first binding domain comprising an scFv comprising the amino acid sequence of SEQ ID NO: 170.

52. The nucleic acid of claim 44, wherein the nucleotide sequence encodes a first binding domain comprising an scFv comprising the amino acid sequence of SEQ ID NO: 194.

53. The nucleic acid of claim 44, wherein the nucleic acid encoding the first binding domain comprises a nucleotide sequence comprising at least 90% sequence identity to the nucleotide sequence of SEQ ID NO: 169 or 193.

54. The nucleic acid of claim 44, wherein the nucleic acid encoding the first binding domain comprises a nucleotide sequence comprising at least 95% sequence identity to the nucleotide sequence of SEQ ID NO: 169 or 193.

55. The nucleic acid of claim 44, wherein the nucleic acid encoding the first binding domain comprises the nucleotide sequence of SEQ ID NO: 169 or 193.

56. The nucleic acid of claim 44, wherein the nucleic acid encoding the first binding domain comprises the nucleotide sequence of SEQ ID NO: 169.

57. The nucleic acid of claim 44, wherein the nucleic acid encoding the first binding domain comprises the nucleotide sequence of SEQ ID NO: 193.

58. The nucleic acid of claim 44, wherein the cell surface antigen is a tumor antigen.

59. The nucleic acid of claim 44, wherein the cell surface antigen is EpCAM, EGFR, EGFRvIII or Carboanhydrase IX (MN/CA IX).

60. A composition comprising the nucleic acid of claim 44 and a carrier, stabilizer, and/or excipient.


SEQUENCE LISTING (207 sequences)

SEQ ID NO: 1 - DNA, 375 bp, Mus musculus
  misc_feature 1..375: note = VH region
  source 1..375: mol_type = genomic DNA
gaggtgaagcttctcgagtctggaggaggattggtgcagcctaaagggtcattgaaactctcatgtgcagcctctggattcaccttcaatacctacgccatgaactgggtccgccaggctccaggaaagggtttggaatgggttgctcgcataagaagtaaatataataattatgcaacatattatgccgattcagtgaaagacaggttcaccatctccagagatgattcacaaagcattctctatctacaaatgaacaacttgaaaactgaggacacagccatgtactactgtgtgagacatgggaacttcggtaatagctacgtttcctggtttgcttactggggccaagggactctggtcactgtctctgca

SEQ ID NO: 2 - protein, 125 aa, Mus musculus
  REGION 1..125: note = misc_feature - VH region
EVKLLESGGGLVQPKGSLKLSCAASGFTFNTYAMNWVRQAPGKGLEWVARIRSKYNNYATYYADSVKDRFTISRDDSQSILYLQMNNLKTEDTAMYYCVRHGNFGNSYVSWFAYWGQGTLVTVSA

SEQ ID NO: 3 - DNA, 327 bp, Mus musculus
  misc_feature 1..327: note = VL region
  source 1..327: mol_type = genomic DNA
caggctgttgtgactcaggaatctgcactcaccacatcacctggtgaaacagtcacactcacttgtcgctcaagtactggggctgttacaactagtaactatgccaactgggtccaagaaaaaccagatcatttattcactggtctaataggtggtaccaacaagcgagctccaggtgtgcctgccagattctcaggctccctgattggagacaaggctgccctcaccatcacaggggcacagactgaggatgaggcaatatatttctgtgctctatggtacagcaacctctgggtgttcggtggaggaaccaaactgactgtccta

SEQ ID NO: 4 - protein, 109 aa, Mus musculus
  REGION 1..109: note = misc_feature - VL region
QAVVTQESALTTSPGETVTLTCRSSTGAVTTSNYANWVQEKPDHLFTGLIGGTNKRAPGVPARFSGSLIGDKAALTITGAQTEDEAIYFCALWYSNLWVFGGGTKLTVL

SEQ ID NO: 5 - DNA, 363 bp, Mus musculus
  misc_feature 1..363: note = VH region
  source 1..363: mol_type = genomic DNA
caggtccagctgcagcagtctggggctgaactggcaagacctggggcctcagtgaagatgtcctgcaaggcttctggctacacctttactagatctacgatgcactgggtaaaacagaggcctggacagggtctggaatggattggatacattaatcctagcagtgcttatactaattacaatcagaaattcaaggacaaggccacattgactgcagacaaatcctccagtacagcctacatgcaactgagtagcctgacatctgaggactctgcagtctattactgtgcaagtccgcaagtccactatgattacaacgggtttccttactggggccaagggactctggtcactgtctctgca

SEQ ID NO: 6 - protein, 121 aa, Mus musculus
  REGION 1..121: note = misc_feature - VH region
QVQLQQSGAELARPGASVKMSCKASGYTFTRSTMHWVKQRPGQGLEWIGYINPSSAYTNYNQKFKDKATLTADKSSSTAYMQLSSLTSEDSAVYYCASPQVHYDYNGFPYWGQGTLVTVSA

SEQ ID NO: 7 - DNA, 318 bp, Mus musculus
  misc_feature 1..318: note = VL region
  source 1..318: mol_type = genomic DNA
caagttgttctcacccagtctccagcaatcatgtctgcatttccaggggagaaggtcaccatgacctgcagtgccagctcaagtgtaagttacatgaactggtaccagcagaagtcaggcacctcccccaaaagatggatttatgactcatccaaactggcttctggagtccctgctcgcttcagtggcagtgggtctgggacctcttattctctcacaatcagcagcatggagactgaagatgctgccacttattactgccagcagtggagtcgtaacccacccacgttcggaggggggaccaagctacaaattaca

SEQ ID NO: 8 - protein, 106 aa, Mus musculus
  REGION 1..106: note = misc_feature - VL region
QVVLTQSPAIMSAFPGEKVTMTCSASSSVSYMNWYQQKSGTSPKRWIYDSSKLASGVPARFSGSGSGTSYSLTISSMETEDAATYYCQQWSRNPPTFGGGTKLQIT

SEQ ID NO: 9 - DNA, 747 bp, synthetic construct
  misc_feature 1..747: note = VH-VL scFV single chain Fv
  source 1..747: mol_type = other DNA
gaggtgaagcttctcgagtctggaggaggattggtgcagcctaaagggtcattgaaactctcatgtgcagcctctggattcaccttcaatacctacgccatgaactgggtccgccaggctccaggaaagggtttggaatgggttgctcgcataagaagtaaatataataattatgcaacatattatgccgattcagtgaaagacaggttcaccatctccagagatgattcacaaagcattctctatctacaaatgaacaacttgaaaactgaggacacagccatgtactactgtgtgagacatgggaacttcggtaatagctacgtttcctggtttgcttactggggccaagggactctggtcactgtctctgcaggtggtggtggttctggcggcggcggctccggtggtggtggttctcaggctgttgtgactcaggaatctgcactcaccacatcacctggtgaaacagtcacactcacttgtcgctcaagtactggggctgttacaactagtaactatgccaactgggtccaagaaaaaccagatcatttattcactggtctaataggtggtaccaacaagcgagctccaggtgtgcctgccagattctcaggctccctgattggagacaaggctgccctcaccatcacaggggcacagactgaggatgaggcaatatatttctgtgctctatggtacagcaacctctgggtgttcggtggaggaaccaaactgactgtccta

SEQ ID NO: 10 - protein, 249 aa, synthetic construct
  REGION 1..249: note = VH-VL scFv single chain Fv
EVKLLESGGGLVQPKGSLKLSCAASGFTFNTYAMNWVRQAPGKGLEWVARIRSKYNNYATYYADSVKDRFTISRDDSQSILYLQMNNLKTEDTAMYYCVRHGNFGNSYVSWFAYWGQGTLVTVSAGGGGSGGGGSGGGGSQAVVTQESALTTSPGETVTLTCRSSTGAVTTSNYANWVQEKPDHLFTGLIGGTNKRAPGVPARFSGSLIGDKAALTITGAQTEDEAIYFCALWYSNLWVFGGGTKLTVL

SEQ ID NO: 11 - DNA, 747 bp, synthetic construct
  misc_feature 1..747: note = VL-VH scFv single chain Fv
  source 1..747: mol_type = other DNA
caggctgttgtgactcaggaatctgcactcaccacatcacctggtgaaacagtcacactcacttgtcgctcaagtactggggctgttacaactagtaactatgccaactgggtccaagaaaaaccagatcatttattcactggtctaataggtggtaccaacaagcgagctccaggtgtgcctgccagattctcaggctccctgattggagacaaggctgccctcaccatcacaggggcacagactgaggatgaggcaatatatttctgtgctctatggtacagcaacctctgggtgttcggtggaggaaccaaactgactgtcctaggtggtggtggttctggcggcggcggctccggtggtggtggttctgaggtgaagcttctcgagtctggaggaggattggtgcagcctaaagggtcattgaaactctcatgtgcagcctctggattcaccttcaatacctacgccatgaactgggtccgccaggctccaggaaagggtttggaatgggttgctcgcataagaagtaaatataataattatgcaacatattatgccgattcagtgaaagacaggttcaccatctccagagatgattcacaaagcattctctatctacaaatgaacaacttgaaaactgaggacacagccatgtactactgtgtgagacatgggaacttcggtaatagctacgtttcctggtttgcttactggggccaagggactctggtcactgtctctgca

SEQ ID NO: 12 - protein, 249 aa, synthetic construct
  REGION 1..249: note = VL-VH scFv single chain Fv
QAVVTQESALTTSPGETVTLTCRSSTGAVTTSNYANWVQEKPDHLFTGLIGGTNKRAPGVPARFSGSLIGDKAALTITGAQTEDEAIYFCALWYSNLWVFGGGTKLTVLGGGGSGGGGSGGGGSEVKLLESGGGLVQPKGSLKLSCAASGFTFNTYAMNWVRQAPGKGLEWVARIRSKYNNYATYYADSVKDRFTISRDDSQSILYLQMNNLKTEDTAMYYCVRHGNFGNSYVSWFAYWGQGTLVTVSA

SEQ ID NO: 13 - DNA, 726 bp, synthetic construct
  misc_feature 1..726: note = VH-VL scFv single chain Fv
  source 1..726: mol_type = other DNA
caggtccagctgcagcagtctggggctgaactggcaagacctggggcctcagtgaagatgtcctgcaaggcttctggctacacctttactagatctacgatgcactgggtaaaacagaggcctggacagggtctggaatggattggatacattaatcctagcagtgcttatactaattacaatcagaaattcaaggacaaggccacattgactgcagacaaatcctccagtacagcctacatgcaactgagtagcctgacatctgaggactctgcagtctattactgtgcaagtccgcaagtccactatgattacaacgggtttccttactggggccaagggactctggtcactgtctctgcaggtggtggtggttctggcggcggcggctccggtggtggtggttctcaagttgttctcacccagtctccagcaatcatgtctgcatttccaggggagaaggtcaccatgacctgcagtgccagctcaagtgtaagttacatgaactggtaccagcagaagtcaggcacctcccccaaaagatggatttatgactcatccaaactggcttctggagtccctgctcgcttcagtggcagtgggtctgggacctcttattctctcacaatcagcagcatggagactgaagatgctgccacttattactgccagcagtggagtcgtaacccacccacgttcggaggggggaccaagctacaaattaca

SEQ ID NO: 14 - protein, 242 aa, synthetic construct
  REGION 1..242: note = VH-VL scFv single chain Fv
QVQLQQSGAELARPGASVKMSCKASGYTFTRSTMHWVKQRPGQGLEWIGYINPSSAYTNYNQKFKDKATLTADKSSSTAYMQLSSLTSEDSAVYYCASPQVHYDYNGFPYWGQGTLVTVSAGGGGSGGGGSGGGGSQVVLTQSPAIMSAFPGEKVTMTCSASSSVSYMNWYQQKSGTSPKRWIYDSSKLASGVPARFSGSGSGTSYSLTISSMETEDAATYYCQQWSRNPPTFGGGTKLQIT

SEQ ID NO: 15 - DNA, 726 bp, synthetic construct
  misc_feature 1..726: note = VL-VH scFv single chain Fv
  source 1..726: mol_type = other DNA
caagttgttctcacccagtctccagcaatcatgtctgcatttccaggggagaaggtcaccatgacctgcagtgccagctcaagtgtaagttacatgaactggtaccagcagaagtcaggcacctcccccaaaagatggatttatgactcatccaaactggcttctggagtccctgctcgcttcagtggcagtgggtctgggacctcttattctctcacaatcagcagcatggagactgaagatgctgccacttattactgccagcagtggagtcgtaacccacccacgttcggaggggggaccaagctacaaattacaggtggtggtggttctggcggcggcggctccggtggtggtggttctcaggtccagctgcagcagtctggggctgaactggcaagacctggggcctcagtgaagatgtcctgcaaggcttctggctacacctttactagatctacgatgcactgggtaaaacagaggcctggacagggtctggaatggattggatacattaatcctagcagtgcttatactaattacaatcagaaattcaaggacaaggccacattgactgcagacaaatcctccagtacagcctacatgcaactgagtagcctgacatctgaggactctgcagtctattactgtgcaagtccgcaagtccactatgattacaacgggtttccttactggggccaagggactctggtcactgtctctgca

SEQ ID NO: 16 - protein, 242 aa, synthetic construct
  REGION 1..242: note = VL-VH scFv single chain Fv
QVVLTQSPAIMSAFPGEKVTMTCSASSSVSYMNWYQQKSGTSPKRWIYDSSKLASGVPARFSGSGSGTSYSLTISSMETEDAATYYCQQWSRNPPTFGGGTKLQITGGGGSGGGGSGGGGSQVQLQQSGAELARPGASVKMSCKASGYTFTRSTMHWVKQRPGQGLEWIGYINPSSAYTNYNQKFKDKATLTADKSSSTAYMQLSSLTSEDSAVYYCASPQVHYDYNGFPYWGQGTLVTVSA

SEQ ID NO: 17 - DNA, 47 bp, synthetic construct
  misc_feature 1..47: note = 5' LH oligonucleotide
  source 1..47: mol_type = other DNA
acatccggaggtggtggatcccaggctgttgtgactcaggaatctgc

SEQ ID NO: 18 - DNA, 51 bp, synthetic construct
  misc_feature 1..51: note = 3' VL Linker oligonucleotide
  source 1..51: mol_type = other DNA
ggagccgccgccgccagaaccaccaccacctaggacagtcagtttggttcc

SEQ ID NO: 19 - DNA, 70 bp, synthetic construct
  misc_feature 1..70: note = 5' VH Linker oligonucleotide
  source 1..70: mol_type = other DNA
tctggcggcggcggctccggtggtggtggttctgaggtgaagcttctcgagtctggaggaggattggtgc

SEQ ID NO: 20 - DNA, 56 bp, synthetic construct
  misc_feature 1..56: note = 3' LH oligonucleotide
  source 1..56: mol_type = other DNA
agtgggtcgacctaatgatgatggtgatgatgtgcagagacagtgaccagagtccc

SEQ ID NO: 21 - DNA, 58 bp, synthetic construct
  misc_feature 1..58: note = 5' HL oligonucleotide
  source 1..58: mol_type = other DNA
acatccggaggtggtggatccgaggtgaagcttctcgagtctggaggaggattggtgc

SEQ ID NO: 22 - DNA, 54 bp, synthetic construct
  misc_feature 1..54: note = 3' VH Linker oligonucleotide
  source 1..54: mol_type = other DNA
ggagccgccgccgccagaaccaccaccacctgcagagacagtgaccagagtccc

SEQ ID NO: 23 - DNA, 59 bp, synthetic construct
  misc_feature 1..59: note = 5' VL Linker oligonucleotide
  source 1..59: mol_type = other DNA
tctggcggcggcggctccggtggtggtggttctcaggctgttgtgactcaggaatctgc

SEQ ID NO: 24 - DNA, 56 bp, synthetic construct
  misc_feature 1..56: note = 3' HL oligonucleotide
  source 1..56: mol_type = other DNA
agtgggtcgacctaatgatgatggtgatgatgtaggacagtcagtttggttcctcc

SEQ ID NO: 25 - DNA, 44 bp, synthetic construct
  misc_feature 1..44: note = 5' LH oligonucleotide
  source 1..44: mol_type = other DNA
acatccggaggtggtggatcccaagttgttctcacccagtctcc

SEQ ID NO: 26 - DNA, 53 bp, synthetic construct
  misc_feature 1..53: note = 3' VL Linker oligonucleotide
  source 1..53: mol_type = other DNA
ggagccgccgccgccagaaccaccaccacctgtaatttgtagcttggtccccc

SEQ ID NO: 27 - DNA, 56 bp, synthetic construct
  misc_feature 1..56: note = 5' VH Linker oligonucleotide
  source 1..56: mol_type = other DNA
tctggcggcggcggctccggtggtggtggttctcaggtccagctgcagcagtctgg

SEQ ID NO: 28 - DNA, 55 bp, synthetic construct
  misc_feature 1..55: note = 3' LH oligonucleotide
  source 1..55: mol_type = other DNA
agtgggtcgacctaatgatgatggtgatgatgtgcagagacagtgaccagagtcc

SEQ ID NO: 29 - DNA, 44 bp, synthetic construct
  misc_feature 1..44: note = 5' HL oligonucleotide
  source 1..44: mol_type = other DNA
acatccggaggtggtggatcccaggtccagctgcagcagtctgg

SEQ ID NO: 30 - DNA, 53 bp, synthetic construct
  misc_feature 1..53: note = 3' VH Linker oligonucleotide
  source 1..53: mol_type = other DNA
ggagccgccgccgccagaaccaccaccacctgcagagacagtgaccagagtcc

SEQ ID NO: 31 - DNA, 56 bp, synthetic construct
  misc_feature 1..56: note = 5' VL Linker oligonucleotide
  source 1..56: mol_type = other DNA
tctggcggcggcggctccggtggtggtggttctcaagttgttctcacccagtctcc

SEQ ID NO: 32 - DNA, 55 bp, synthetic construct
  misc_feature 1..55: note = 3' HL oligonucleotide
  source 1..55: mol_type = other DNA
agtgggtcgacctaatgatgatggtgatgatgtgtaatttgtagcttggtccccc

SEQ ID NO: 33 - DNA, 744 bp, synthetic construct
  misc_feature 1..744: note = 5-10 LH scFv single chain Fv
  source 1..744: mol_type = other DNA
gagctcgtgatgacacagtctccatcctccctgactgtgacagcaggagagaaggtcactatgagctgcaagtccagtcagagtctgttaaacagtggaaatcaaaagaactacttgacctggtaccagcagaaaccagggcagcctcctaaactgttgatctactgggcatccactagggaatctggggtccctgatcgcttcacaggcagtggatctggaacagatttcactctcaccatcagcagtgtgcaggctgaagacctggcagtttattactgtcagaatgattatagttatccgctcacgttcggtgctgggaccaagcttgagatcaaaggtggtggtggttctggcggcggcggctccggtggtggtggttctgaggtgcagctgctcgagcagtctggagctgagctggtaaggcctgggacttcagtgaagatatcctgcaaggcttctggatacgccttcactaactactggctaggttgggtaaagcagaggcctggacatggacttgagtggattggagatattttccctggaagtggtaatatccactacaatgagaagttcaagggcaaagccacactgactgcagacaaatcttcgagcacagcctatatgcagctcagtagcctgacatttgaggactctgctgtctatttctgtgcaagactgaggaactgggacgagcctatggactactggggccaagggaccacggtcaccgtctcctcc

SEQ ID NO: 34 - protein, 248 aa, synthetic construct
  REGION 1..248: note = 5-10 LH scFv single chain Fv
ELVMTQSPSSLTVTAGEKVTMSCKSSQSLLNSGNQKNYLTWYQQKPGQPPKLLIYWASTRESGVPDRFTGSGSGTDFTLTISSVQAEDLAVYYCQNDYSYPLTFGAGTKLEIKGGGGSGGGGSGGGGSEVQLLEQSGAELVRPGTSVKISCKASGYAFTNYWLGWVKQRPGHGLEWIGDIFPGSGNIHYNEKFKGKATLTADKSSSTAYMQLSSLTFEDSAVYFCARLRNWDEPMDYWGQGTTVTVSS

SEQ ID NO: 35 - DNA, 57 bp, Mus musculus
  misc_feature 1..57: note = leader peptide
  source 1..57: mol_type = genomic DNA
atgggatggagctgtatcatcctcttcttggtagcaacagctacaggtgtacactcc

SEQ ID NO: 36 - protein, 19 aa, Mus musculus
  REGION 1..19: note = misc_feature - leader peptide
MGWSCIILFLVATATGVHS

SEQ ID NO: 37 - DNA, 1506 bp, synthetic construct
  misc_feature 1..1506: note = 5-10 LHxSEQ ID NO: 12 ; Bispecific single chain antibody
  source 1..1506: mol_type = other DNA
gagctcgtgatgacacagtctccatcctccctgactgtgacagcaggagagaaggtcactatgagctgcaagtccagtcagagtctgttaaacagtggaaatcaaaagaactacttgacctggtaccagcagaaaccagggcagcctcctaaactgttgatctactgggcatccactagggaatctggggtccctgatcgcttcacaggcagtggatctggaacagatttcactctcaccatcagcagtgtgcaggctgaagacctggcagtttattactgtcagaatgattatagttatccgctcacgttcggtgctgggaccaagcttgagatcaaaggtggtggtggttctggcggcggcggctccggtggtggtggttctgaggtgcagctgctcgagcagtctggagctgagctggtaaggcctgggacttcagtgaagatatcctgcaaggcttctggatacgccttcactaactactggctaggttgggtaaagcagaggcctggacatggacttgagtggattggagatattttccctggaagtggtaatatccactacaatgagaagttcaagggcaaagccacactgactgcagacaaatcttcgagcacagcctatatgcagctcagtagcctgacatttgaggactctgctgtctatttctgtgcaagactgaggaactgggacgagcctatggactactggggccaagggaccacggtcaccgtctcctccggaggtggtggatcccaggctgttgtgactcaggaatctgcactcaccacatcacctggtgaaacagtcacactcacttgtcgctcaagtactggggctgttacaactagtaactatgccaactgggtccaagaaaaaccagatcatttattcactggtctaataggtggtaccaacaagcgagctccaggtgtgcctgccagattctcaggctccctgattggagacaaggctgccctcaccatcacaggggcacagactgaggatgaggcaatatatttctgtgctctatggtacagcaacctctgggtgttcggtggaggaaccaaactgactgtcctaggtggtggtggttctggcggcggcggctccggtggtggtggttctgaggtgaagcttctcgagtctggaggaggattggtgcagcctaaagggtcattgaaactctcatgtgcagcctctggattcaccttcaatacctacgccatgaactgggtccgccaggctccaggaaagggtttggaatgggttgctcgcataagaagtaaatataataattatgcaacatattatgccgattcagtgaaagacaggttcaccatctccagagatgattcacaaagcattctctatctacaaatgaacaacttgaaaactgaggacacagccatgtactactgtgtgagacatgggaacttcggtaatagctacgtttcctggtttgcttactggggccaagggactctggtcactgtctctgca

SEQ ID NO: 38 - protein, 502 aa, synthetic construct
  REGION 1..502: note = 5-10 LHxSEQ ID NO: 12 ; Bispecific single chain antibody
ELVMTQSPSSLTVTAGEKVTMSCKSSQSLLNSGNQKNYLTWYQQKPGQPPKLLIYWASTRESGVPDRFTGSGSGTDFTLTISSVQAEDLAVYYCQNDYSYPLTFGAGTKLEIKGGGGSGGGGSGGGGSEVQLLEQSGAELVRPGTSVKISCKASGYAFTNYWLGWVKQRPGHGLEWIGDIFPGSGNIHYNEKFKGKATLTADKSSSTAYMQLSSLTFEDSAVYFCARLRNWDEPMDYWGQGTTVTVSSGGGGSQAVVTQESALTTSPGETVTLTCRSSTGAVTTSNYANWVQEKPDHLFTGLIGGTNKRAPGVPARFSGSLIGDKAALTITGAQTEDEAIYFCALWYSNLWVFGGGTKLTVLGGGGSGGGGSGGGGSEVKLLESGGGLVQPKGSLKLSCAASGFTFNTYAMNWVRQAPGKGLEWVARIRSKYNNYATYYADSVKDRFTISRDDSQSILYLQMNNLKTEDTAMYYCVRHGNFGNSYVSWFAYWGQGTLVTVSA

SEQ ID NO: 39 - DNA, 1506 bp, synthetic construct
  misc_feature 1..1506: note = 5-10 LHx SEQ ID NO: 10 ; bispecific single chain antibdoy
  source 1..1506: mol_type = other DNA
gagctcgtgatgacacagtctccatcctccctgactgtgacagcaggagagaaggtcactatgagctgcaagtccagtcagagtctgttaaacagtggaaatcaaaagaactacttgacctggtaccagcagaaaccagggcagcctcctaaactgttgatctactgggcatccactagggaatctggggtccctgatcgcttcacaggcagtggatctggaacagatttcactctcaccatcagcagtgtgcaggctgaagacctggcagtttattactgtcagaatgattatagttatccgctcacgttcggtgctgggaccaagcttgagatcaaaggtggtggtggttctggcggcggcggctccggtggtggtggttctgaggtgcagctgctcgagcagtctggagctgagctggtaaggcctgggacttcagtgaagatatcctgcaaggcttctggatacgccttcactaactactggctaggttgggtaaagcagaggcctggacatggacttgagtggattggagatattttccctggaagtggtaatatccactacaatgagaagttcaagggcaaagccacactgactgcagacaaatcttcgagcacagcctatatgcagctcagtagcctgacatttgaggactctgctgtctatttctgtgcaagactgaggaactgggacgagcctatggactactggggccaagggaccacggtcaccgtctcctccggaggtggtggatccgaggtgaagcttctcgagtctggaggaggattggtgcagcctaaagggtcattgaaactctcatgtgcagcctctggattcaccttcaatacctacgccatgaactgggtccgccaggctccaggaaagggtttggaatgggttgctcgcataagaagtaaatataataattatgcaacatattatgccgattcagtgaaagacaggttcaccatctccagagatgattcacaaagcattctctatctacaaatgaacaacttgaaaactgaggacacagccatgtactactgtgtgagacatgggaacttcggtaatagctacgtttcctggtttgcttactggggccaagggactctggtcactgtctctgcaggtggtggtggttctggcggcggcggctccggtggtggtggttctcaggctgttgtgactcaggaatctgcactcaccacatcacctggtgaaacagtcacactcacttgtcgctcaagtactggggctgttacaactagtaactatgccaactgggtccaagaaaaaccagatcatttattcactggtctaataggtggtaccaacaagcgagctccaggtgtgcctgccagattctcaggctccctgattggagacaaggctgccctcaccatcacaggggcacagactgaggatgaggcaatatatttctgtgctctatggtacagcaacctctgggtgttcggtggaggaaccaaactgactgtccta

SEQ ID NO: 40 - protein, 502 aa, synthetic construct
  REGION 1..502: note = 5-10 LHx SEQ ID NO: 10 ; bispecific single chain antibody
ELVMTQSPSSLTVTAGEKVTMSCKSSQSLLNSGNQKNYLTWYQQKPGQPPKLLIYWASTRESGVPDRFTGSGSGTDFTLTISSVQAEDLAVYYCQNDYSYPLTFGAGTKLEIKGGGGSGGGGSGGGGSEVQLLEQSGAELVRPGTSVKISCKASGYAFTNYWLGWVKQRPGHGLEWIGDIFPGSGNIHYNEKFKGKATLTADKSSSTAYMQLSSLTFEDSAVYFCARLRNWDEPMDYWGQGTTVTVSSGGGGSEVKLLESGGGLVQPKGSLKLSCAASGFTFNTYAMNWVRQAPGKGLEWVARIRSKYNNYATYYADSVKDRFTISRDDSQSILYLQMNNLKTEDTAMYYCVRHGNFGNSYVSWFAYWGQGTLVTVSAGGGGSGGGGSGGGGSQAVVTQESALTTSPGETVTLTCRSSTGAVTTSNYANWVQEKPDHLFTGLIGGTNKRAPGVPARFSGSLIGDKAALTITGAQTEDEAIYFCALWYSNLWVFGGGTKLTVL

SEQ ID NO: 41 - DNA, 1485 bp, synthetic construct
  misc_feature 1..1485: note = 5-10 LHx SEQ ID NO: 16 ; bispecific single chain antibody
  source 1..1485: mol_type = other DNA
gagctcgtgatgacacagtctccatcctccctgactgtgacagcaggagagaaggtcactatgagctgcaagtccagtcagagtctgttaaacagtggaaatcaaaagaactacttgacctggtaccagcagaaaccagggcagcctcctaaactgttgatctactgggcatccactagggaatctggggtccctgatcgcttcacaggcagtggatctggaacagatttcactctcaccatcagcagtgtgcaggctgaagacctggcagtttattactgtcagaatgattatagttatccgctcacgttcggtgctgggaccaagcttgagatcaaaggtggtggtggttctggcggcggcggctccggtggtggtggttctgaggtgcagctgctcgagcagtctggagctgagctggtaaggcctgggacttcagtgaagatatcctgcaaggcttctggatacgccttcactaactactggctaggttgggtaaagcagaggcctggacatggacttgagtggattggagatattttccctggaagtggtaatatccactacaatgagaagttcaagggcaaagccacactgactgcagacaaatcttcgagcacagcctatatgcagctcagtagcctgacatttgaggactctgctgtctatttctgtgcaagactgaggaactgggacgagcctatggactactggggccaagggaccacggtcaccgtctcctccggaggtggtggatcccaagttgttctcacccagtctccagcaatcatgtctgcatttccaggggagaaggtcaccatgacctgcagtgccagctcaagtgtaagttacatgaactggtaccagcagaagtcaggcacctcccccaaaagatggatttatgactcatccaaactggcttctggagtccctgctcgcttcagtggcagtgggtctgggacctcttattctctcacaatcagcagcatggagactgaagatgctgccacttattactgccagcagtggagtcgtaacccacccacgttcggaggggggaccaagctacaaattacaggtggtggtggttctggcggcggcggctccggtggtggtggttctcaggtccagctgcagcagtctggggctgaactggcaagacctggggcctcagtgaagatgtcctgcaaggcttctggctacacctttactagatctacgatgcactgggtaaaacagaggcctggacagggtctggaatggattggatacattaatcctagcagtgcttatactaattacaatcagaaattcaaggacaaggccacattgactgcagacaaatcctccagtacagcctacatgcaactgagtagcctgacatctgaggactctgcagtctattactgtgcaagtccgcaagtccactatgattacaacgggtttccttactggggccaagggactctggtcactgtctctgca

SEQ ID NO: 42 - protein, 495 aa, synthetic construct
  REGION 1..495: note = 5-10 LHxSEQ ID NO: 16 ; bispecific single chain antibody
ELVMTQSPSSLTVTAGEKVTMSCKSSQSLLNSGNQKNYLTWYQQKPGQPPKLLIYWASTRESGVPDRFTGSGSGTDFTLTISSVQAEDLAVYYCQNDYSYPLTFGAGTKLEIKGGGGSGGGGSGGGGSEVQLLEQSGAELVRPGTSVKISCKASGYAFTNYWLGWVKQRPGHGLEWIGDIFPGSGNIHYNEKFKGKATLTADKSSSTAYMQLSSLTFEDSAVYFCARLRNWDEPMDYWGQGTTVTVSSGGGGSQVVLTQSPAIMSAFPGEKVTMTCSASSSVSYMNWYQQKSGTSPKRWIYDSSKLASGVPARFSGSGSGTSYSLTISSMETEDAATYYCQQWSRNPPTFGGGTKLQITGGGGSGGGGSGGGGSQVQLQQSGAELARPGASVKMSCKASGYTFTRSTMHWVKQRPGQGLEWIGYINPSSAYTNYNQKFKDKATLTADKSSSTAYMQLSSLTSEDSAVYYCASPQVHYDYNGFPYWGQGTLVTVSA

SEQ ID NO: 43 - DNA, 1485 bp, synthetic construct
  misc_feature 1..1485: note = 5-10 LHxSEQ ID NO: 14 ; bispecific single chain antibody
  source 1..1485: mol_type = other DNA
gagctcgtgatgacacagtctccatcctccctgactgtgacagcaggagagaaggtcactatgagctgcaagtccagtcagagtctgttaaacagtggaaatcaaaagaactacttgacctggtaccagcagaaaccagggcagcctcctaaactgttgatctactgggcatccactagggaatctggggtccctgatcgcttcacaggcagtggatctggaacagatttcactctcaccatcagcagtgtgcaggctgaagacctggcagtttattactgtcagaatgattatagttatccgctcacgttcggtgctgggaccaagcttgagatcaaaggtggtggtggttctggcggcggcggctccggtggtggtggttctgaggtgcagctgctcgagcagtctggagctgagctggtaaggcctgggacttcagtgaagatatcctgcaaggcttctggatacgccttcactaactactggctaggttgggtaaagcagaggcctggacatggacttgagtggattggagatattttccctggaagtggtaatatccactacaatgagaagttcaagggcaaagccacactgactgcagacaaatcttcgagcacagcctatatgcagctcagtagcctgacatttgaggactctgctgtctatttctgtgcaagactgaggaactgggacgagcctatggactactggggccaagggaccacggtcaccgtctcctccggaggtggtggatcccaggtccagctgcagcagtctggggctgaactggcaagacctggggcctcagtgaagatgtcctgcaaggcttctggctacacctttactagatctacgatgcactgggtaaaacagaggcctggacagggtctggaatggattggatacattaatcctagcagtgcttatactaattacaatcagaaattcaaggacaaggccacattgactgcagacaaatcctccagtacagcctacatgcaactgagtagcctgacatctgaggactctgcagtctattactgtgcaagtccgcaagtccactatgattacaacgggtttccttactggggccaagggactctggtcactgtctctgcaggtggtggtggttctggcggcggcggctccggtggtggtggttctcaagttgttctcacccagtctccagcaatcatgtctgcatttccaggggagaaggtcaccatgacctgcagtgccagctcaagtgtaagttacatgaactggtaccagcagaagtcaggcacctcccccaaaagatggatttatgactcatccaaactggcttctggagtccctgctcgcttcagtggcagtgggtctgggacctcttattctctcacaatcagcagcatggagactgaagatgctgccacttattactgccagcagtggagtcgtaacccacccacgttcggaggggggaccaagctacaaattaca

SEQ ID NO: 44 - protein, 495 aa, synthetic construct
  REGION 1..495: note = 5-10 LHxSEQ ID NO: 14 ; bispecific single chain antibody
ELVMTQSPSSLTVTAGEKVTMSCKSSQSLLNSGNQKNYLTWYQQKPGQPPKLLIYWASTRESGVPDRFTGSGSGTDFTLTISSVQAEDLAVYYCQNDYSYPLTFGAGTKLEIKGGGGSGGGGSGGGGSEVQLLEQSGAELVRPGTSVKISCKASGYAFTNYWLGWVKQRPGHGLEWIGDIFPGSGNIHYNEKFKGKATLTADKSSSTAYMQLSSLTFEDSAVYFCARLRNWDEPMDYWGQGTTVTVSSGGGGSQVQLQQSGAELARPGASVKMSCKASGYTFTRSTMHWVKQRPGQGLEWIGYINPSSAYTNYNQKFKDKATLTADKSSSTAYMQLSSLTSEDSAVYYCASPQVHYDYNGFPYWGQGTLVTVSAGGGGSGGGGSGGGGSQVVLTQSPAIMSAFPGEKVTMTCSASSSVSYMNWYQQKSGTSPKRWIYDSSKLASGVPARFSGSGSGTSYSLTISSMETEDAATYYCQQWSRNPPTFGGGTKLQIT

SEQ ID NO: 45 - DNA, 43 bp, synthetic construct
  misc_feature 1..43: note = 5' EpCAM oligonucleotide
  source 1..43: mol_type = other DNA
ggttctagaccaccatggcgcccccgcaggtcctcgcgttcgg

SEQ ID NO: 46 - DNA, 42 bp, synthetic construct
  misc_feature 1..42: note = 3' EpCAM oligonucleotide
  source 1..42: mol_type = other DNA
agtgggtcgacttatgcattgagttccctatgcatctcaccc

SEQ ID NO: 47 - DNA, 726 bp, Macaca fascicularis
  misc_feature 1..726: note = cynomolgus EpCAM extracellular portion
  source 1..726: mol_type = genomic DNA
cagaaagaatgtgtctgtgaaaactacaagctggccgtaaactgctttttgaatgacaatggtcaatgccagtgtacttcgattggtgcacaaaatactgtcctttgctcaaagctggctgccaaatgtttggtgatgaaggcagaaatgaacggctcaaaacttgggagaagagcgaaacctgaaggggctctccagaacaatgatggcctttacgatcctgactgcgatgagagcgggctctttaaggccaagcagtgcaacggcacctccacgtgctggtgtgtgaacactgctggggtcagaagaactgacaaggacactgaaataacctgctctgagcgagtgagaacctactggatcatcattgaattaaaacacaaagcaagagaaaaaccttatgatgttcaaagtttgcggactgcacttgaggaggcgatcaaaacgcgttatcaactggatccaaaatttatcacaaatattttgtatgaggataatgttatcactattgatctggttcaaaattcttctcagaaaactcagaatgatgtggacatagctgatgtggcttattattttgaaaaagatgttaaaggtgaatccttgtttcattctaagaaaatggacctgagagtaaatggggaacaactggatctggatcctggtcaaactttaatttattatgtcgatgaaaaagcacctgaattctcaatgcagggtctaaaa

SEQ ID NO: 48 - protein, 242 aa, Macaca fascicularis
  REGION 1..242: note = misc_feature - cynomolgus EpCAM extracellular portion
QKECVCENYKLAVNCFLNDNGQCQCTSIGAQNTVLCSKLAAKCLVMKAEMNGSKLGRRAKPEGALQNNDGLYDPDCDESGLFKAKQCNGTSTCWCVNTAGVRRTDKDTEITCSERVRTYWIIIELKHKAREKPYDVQSLRTALEEAIKTRYQLDPKFITNILYEDNVITIDLVQNSSQKTQNDVDIADVAYYFEKDVKGESLFHSKKMDLRVNGEQLDLDPGQTLIYYVDEKAPEFSMQGLK

SEQ ID NO: 49 - DNA, 366 bp, synthetic construct
  misc_feature 1..366: note = Hybridoma
  misc_feature 1..366: note = 2G8 VH VH region of monoclonal antibody 2G8
  source 1..366: mol_type = other DNA
gaggttcagctgcagcagtctggggcagagcttgtgaggtcaggggcctcagtcaagttgtcctgcacagcttctggcttcaacattaaagactactatttgcactgggtgaagcagaggcctgaacagggcctggagtggattgcctggattgatcttgagaatggtgatattaaatatgccccgaagtttcagggcaaggccactataactgcagacacatcctccaacacagcctacctgcagctcagcagcctgacatctgaggacactgccgtctattactgtaatccctattactacggtagtaactacgactatgctatggactactggggtcaaggaacctcagtcaccgtctcctca

SEQ ID NO: 50 - protein, 122 aa, synthetic construct
  REGION 1..122: note = Hybridoma
  REGION 1..122: note = misc_feature - 2G8 VH VH region of monoclonal antibody 2G8
EVQLQQSGAELVRSGASVKLSCTASGFNIKDYYLHWVKQRPEQGLEWIAWIDLENGDIKYAPKFQGKATITADTSSNTAYLQLSSLTSEDTAVYYCNPYYYGSNYDYAMDYWGQGTSVTVSS

SEQ ID NO: 51 - DNA, 336 bp, synthetic construct
  misc_feature 1..336: note = Hybridoma
  misc_feature 1..336: note = 2G8 VL VL region of monoclonal antibody 2G8
  source 1..336: mol_type = other DNA
gatgttgtgatgacccagactccactcactttgtcggttaccattggacaaccagcctctatctcttgcaagtcaagtcagagcctcttatatagtaatggaaaaacctatttgaactggatattacagaggccaggccagtctccaaagcgcctaatctatctggtgtctaaactggactctggagtccctgacaggttcactggcagtggatcaggaacagattttacgctgaaaatcagcagagtggaggctgaggatttgggagtttattactgcgtgcaaggtacacattttcctctcacgttcggtgctgggaccaagctggagctgaaa

SEQ ID NO: 52 - protein, 112 aa, synthetic construct
  REGION 1..112: note = Hybridoma
  REGION 1..112: note = misc_feature - 2G8 VL VL region of monoclonal antibody 2G8
DVVMTQTPLTLSVTIGQPASISCKSSQSLLYSNGKTYLNWILQRPGQSPKRLIYLVSKLDSGVPDRFTGSGSGTDFTLKISRVEAEDLGVYYCVQGTHFPLTFGAGTKLELK

SEQ ID NO: 53 - DNA, 747 bp, synthetic construct
  misc_feature 1..747: note = 2G8VH-VL scFv single chain Fv
  source 1..747: mol_type = other DNA
gaggttcagctgcagcagtctggggcagagcttgtgaggtcaggggcctcagtcaagttgtcctgcacagcttctggcttcaacattaaagactactatttgcactgggtgaagcagaggcctgaacagggcctggagtggattgcctggattgatcttgagaatggtgatattaaatatgccccgaagtttcagggcaaggccactataactgcagacacatcctccaacacagcctacctgcagctcagcagcctgacatctgaggacactgccgtctattactgtaatccctattactacggtagtaactacgactatgctatggactactggggtcaaggaacctcagtcaccgtctcctcaggtggtggtggttctggcggcggcggctccggtggtggtggttctgatgttgtgatgacccagactccactcactttgtcggttaccattggacaaccagcctctatctcttgcaagtcaagtcagagcctcttatatagtaatggaaaaacctatttgaactggatattacagaggccaggccagtctccaaagcgcctaatctatctggtgtctaaactggactctggagtccctgacaggttcactggcagtggatcaggaacagattttacgctgaaaatcagcagagtggaggctgaggatttgggagtttattactgcgtgcaaggtacacattttcctctcacgttcggtgctgggaccaagctggagctgaaa

SEQ ID NO: 54 - protein, 249 aa, synthetic construct
  REGION 1..249: note = 2G8VH-VL scFv single chain Fv
EVQLQQSGAELVRSGASVKLSCTASGFNIKDYYLHWVKQRPEQGLEWIAWIDLENGDIKYAPKFQGKATITADTSSNTAYLQLSSLTSEDTAVYYCNPYYYGSNYDYAMDYWGQGTSVTVSSGGGGSGGGGSGGGGSDVVMTQTPLTLSVTIGQPASISCKSSQSLLYSNGKTYLNWILQRPGQSPKRLIYLVSKLDSGVPDRFTGSGSGTDFTLKISRVEAEDLGVYYCVQGTHFPLTFGAGTKLELK

SEQ ID NO: 55 - DNA, 747 bp, synthetic construct
  misc_feature 1..747: note = 2G8VL-VH scFv single chain Fv
  source 1..747: mol_type = other DNA
gatgttgtgatgacccagactccactcactttgtcggttaccattggacaaccagcctctatctcttgcaagtcaagtcagagcctcttatatagtaatggaaaaacctatttgaactggatattacagaggccaggccagtctccaaagcgcctaatctatctggtgtctaaactggactctggagtccctgacaggttcactggcagtggatcaggaacagattttacgctgaaaatcagcagagtggaggctgaggatttgggagtttattactgcgtgcaaggtacacattttcctctcacgttcggtgctgggaccaagctggagctgaaaggtggtggtggttctggcggcggcggctccggtggtggtggttctgaggttcagctgcagcagtctggggcagagcttgtgaggtcaggggcctcagtcaagttgtcctgcacagcttctggcttcaacattaaagactactatttgcactgggtgaagcagaggcctgaacagggcctggagtggattgcctggattgatcttgagaatggtgatattaaatatgccccgaagtttcagggcaaggccactataactgcagacacatcctccaacacagcctacctgcagctcagcagcctgacatctgaggacactgccgtctattactgtaatccctattactacggtagtaactacgactatgctatggactactggggtcaaggaacctcagtcaccgtctcctcc

SEQ ID NO: 56 - protein, 249 aa, synthetic construct
  REGION 1..249: note = 2G8VL-VH scFv single chain Fv
DVVMTQTPLTLSVTIGQPASISCKSSQSLLYSNGKTYLNWILQRPGQSPKRLIYLVSKLDSGVPDRFTGSGSGTDFTLKISRVEAEDLGVYYCVQGTHFPLTFGAGTKLELKGGGGSGGGGSGGGGSEVQLQQSGAELVRSGASVKLSCTASGFNIKDYYLHWVKQRPEQGLEWIAWIDLENGDIKYAPKFQGKATITADTSSNTAYLQLSSLTSEDTAVYYCNPYYYGSNYDYAMDYWGQGTSVTVSS

SEQ ID NO: 57 - DNA, 49 bp, synthetic construct
  misc_feature 1..49: note = 5'2G8 LH oligonucleotide
  source 1..49: mol_type = other DNA
aggtgtacactccgatgttgtgatgacccagactccactcactttgtcg

SEQ ID NO: 58 - DNA, 54 bp, synthetic construct
  misc_feature 1..54: note = 3'2G8 VL Linker oligonucleotide
  source 1..54: mol_type = other DNA
ggagccgccgccgccagaaccaccaccacctttcagctccagcttggtcccagc

SEQ ID NO: 59 - DNA, 56 bp, synthetic construct
  misc_feature 1..56: note = 5'2G8 VH Linker oligonucleotide
  source 1..56: mol_type = other DNA
tctggcggcggcggctccggtggtggtggttctgaggttcagctgcagcagtctgg

SEQ ID NO: 60 - DNA, 30 bp, synthetic construct
  misc_feature 1..30: note = 3'2G8 LH oligonucleotide
  source 1..30: mol_type = other DNA
acatccggaggagacggtgactgaggttcc

SEQ ID NO: 61 - DNA, 37 bp, synthetic construct
  misc_feature 1..37: note = 5' 2G8 HL oligonucleotide
  source 1..37: mol_type = other DNA
aggtgtacactccgaggttcagctgcagcagtctggg

SEQ ID NO: 62 - DNA, 54 bp, synthetic construct
  misc_feature 1..54: note = 3' 2G8 VH Linker oligonucleotide
  source 1..54: mol_type = other DNA
ggagccgccgccgccagaaccaccaccacctgaggagacggtgactgaggttcc

SEQ ID NO: 63 - DNA, 69 bp, synthetic construct
  misc_feature 1..69: note = 5' 2G8 VL Linker oligonucleotide
  source 1..69: mol_type = other DNA
tctggcggcggcggctccggtggtggtggttctgatgttgtgatgacccagactccactcactttgtcg

SEQ ID NO: 64 - DNA, 33 bp, synthetic construct
  misc_feature 1..33: note = 3' 2G8 HL oligonucleotide
  source 1..33: mol_type = other DNA
acatccggatttcagctccagcttggtcccagc

SEQ ID NO: 65 - DNA, 1509 bp, synthetic construct
  misc_feature 1..1509: note = 2G8LHxSEQ ID NO: 12 ; bispecific single chain antibody
  source 1..1509: mol_type = other DNA
gatgttgtgatgacccagactccactcactttgtcggttaccattggacaaccagcctctatctcttgcaagtcaagtcagagcctcttatatagtaatggaaaaacctatttgaactggatattacagaggccaggccagtctccaaagcgcctaatctatctggtgtctaaactggactctggagtccctgacaggttcactggcagtggatcaggaacagattttacgctgaaaatcagcagagtggaggctgaggatttgggagtttattactgcgtgcaaggtacacattttcctctcacgttcggtgctgggaccaagctggagctgaaaggtggtggtggttctggcggcggcggctccggtggtggtggttctgaggttcagctgcagcagtctggggcagagcttgtgaggtcaggggcctcagtcaagttgtcctgcacagcttctggcttcaacattaaagactactatttgcactgggtgaagcagaggcctgaacagggcctggagtggattgcctggattgatcttgagaatggtgatattaaatatgccccgaagtttcagggcaaggccactataactgcagacacatcctccaacacagcctacctgcagctcagcagcctgacatctgaggacactgccgtctattactgtaatccctattactacggtagtaactacgactatgctatggactactggggtcaaggaacctcagtcaccgtctcctccggaggtggtggatcccaggctgttgtgactcaggaatctgcactcaccacatcacctggtgaaacagtcacactcacttgtcgctcaagtactggggctgttacaactagtaactatgccaactgggtccaagaaaaaccagatcatttattcactggtctaataggtggtaccaacaagcgagctccaggtgtgcctgccagattctcaggctccctgattggagacaaggctgccctcaccatcacaggggcacagactgaggatgaggcaatatatttctgtgctctatggtacagcaacctctgggtgttcggtggaggaaccaaactgactgtcctaggtggtggtggttctggcggcggcggctccggtggtggtggttctgaggtgaagcttctcgagtctggaggaggattggtgcagcctaaagggtcattgaaactctcatgtgcagcctctggattcaccttcaatacctacgccatgaactgggtccgccaggctccaggaaagggtttggaatgggttgctcgcataagaagtaaatataataattatgcaacatattatgccgattcagtgaaagacaggttcaccatctccagagatgattcacaaagcattctctatctacaaatgaacaacttgaaaactgaggacacagccatgtactactgtgtgagacatgggaacttcggtaatagctacgtttcctggtttgcttactggggccaagggactctggtcactgtctctgca

SEQ ID NO: 66 - protein, 503 aa, synthetic construct
  REGION 1..503: note = 2G8LHxSEQ ID NO: 12 ; bispecific single chain antibody
DVVMTQTPLTLSVTIGQPASISCKSSQSLLYSNGKTYLNWILQRPGQSPKRLIYLVSKLDSGVPDRFTGSGSGTDFTLKISRVEAEDLGVYYCVQGTHFPLTFGAGTKLELKGGGGSGGGGSGGGGSEVQLQQSGAELVRSGASVKLSCTASGFNIKDYYLHWVKQRPEQGLEWIAWIDLENGDIKYAPKFQGKATITADTSSNTAYLQLSSLTSEDTAVYYCNPYYYGSNYDYAMDYWGQGTSVTVSSGGGGSQAVVTQESALTTSPGETVTLTCRSSTGAVTTSNYANWVQEKPDHLFTGLIGGTNKRAPGVPARFSGSLIGDKAALTITGAQTEDEAIYFCALWYSNLWVFGGGTKLTVLGGGGSGGGGSGGGGSEVKLLESGGGLVQPKGSLKLSCAASGFTFNTYAMNWVRQAPGKGLEWVARIRSKYNNYATYYADSVKDRFTISRDDSQSILYLQMNNLKTEDTAMYYCVRHGNFGNSYVSWFAYWGQGTLVTVSA

SEQ ID NO: 67 - DNA, 1509 bp, synthetic construct
  misc_feature 1..1509: note = 2G8LHx SEQ ID NO: 10 ; bispecific single chain antibody
  source 1..1509: mol_type = other DNA
gatgttgtgatgacccagactccactcactttgtcggttaccattggacaaccagcctctatctcttgcaagtcaagtcagagcctcttatatagtaatggaaaaacctatttgaactggatattacagaggccaggccagtctccaaagcgcctaatctatctggtgtctaaactggactctggagtccctgacaggttcactggcagtggatcaggaacagattttacgctgaaaatcagcagagtggaggctgaggatttgggagtttattactgcgtgcaaggtacacattttcctctcacgttcggtgctgggaccaagctggagctgaaaggtggtggtggttctggcggcggcggctccggtggtggtggttctgaggttcagctgcagcagtctggggcagagcttgtgaggtcaggggcctcagtcaagttgtcctgcacagcttctggcttcaacattaaagactactatttgcactgggtgaagcagaggcctgaacagggcctggagtggattgcctggattgatcttgagaatggtgatattaaatatgccccgaagtttcagggcaaggccactataactgcagacacatcctccaacacagcctacctgcagctcagcagcctgacatctgaggacactgccgtctattactgtaatccctattactacggtagtaactacgactatgctatggactactggggtcaaggaacctcagtcaccgtctcctccggaggtggtggatccgaggtgaagcttctcgagtctggaggaggattggtgcagcctaaagggtcattgaaactctcatgtgcagcctctggattcaccttcaatacctacgccatgaactgggtccgccaggctccaggaaagggtttggaatgggttgctcgcataagaagtaaatataataattatgcaacatattatgccgattcagtgaaagacaggttcaccatctccagagatgattcacaaagcattctctatctacaaatgaacaacttgaaaactgaggacacagccatgtactactgtgtgagacatgggaacttcggtaatagctacgtttcctggtttgcttactggggccaagggactctggtcactgtctctgcaggtggtggtggttctggcggcggcggctccggtggtggtggttctcaggctgttgtgactcaggaatctgcactcaccacatcacctggtgaaacagtcacactcacttgtcgctcaagtactggggctgttacaactagtaactatgccaactgggtccaagaaaaaccagatcatttattcactggtctaataggtggtaccaacaagcgagctccaggtgtgcctgccagattctcaggctccctgattggagacaaggctgccctcaccatcacaggggcacagactgaggatgaggcaatatatttctgtgctctatggtacagcaacctctgggtgttcggtggaggaaccaaactgactgtccta

SEQ ID NO: 68 - protein, 503 aa, synthetic construct
  REGION 1..503: note = 2G8LHxSEQ ID NO: 10 ; bispecific single chain antibody
DVVMTQTPLTLSVTIGQPASISCKSSQSLLYSNGKTYLNWILQRPGQSPKRLIYLVSKLDSGVPDRFTGSGSGTDFTLKISRVEAEDLGVYYCVQGTHFPLTFGAGTKLELKGGGGSGGGGSGGGGSEVQLQQSGAELVRSGASVKLSCTASGFNIKDYYLHWVKQRPEQGLEWIAWIDLENGDIKYAPKFQGKATITADTSSNTAYLQLSSLTSEDTAVYYCNPYYYGSNYDYAMDYWGQGTSVTVSSGGGGSEVKLLESGGGLVQPKGSLKLSCAASGFTFNTYAMNWVRQAPGKGLEWVARIRSKYNNYATYYADSVKDRFTISRDDSQSILYLQMNNLKTEDTAMYYCVRHGNFGNSYVSWFAYWGQGTLVTVSAGGGGSGGGGSGGGGSQAVVTQESALTTSPGETVTLTCRSSTGAVTTSNYANWVQEKPDHLFTGLIGGTNKRAPGVPARFSGSLIGDKAALTITGAQTEDEAIYFCALWYSNLWVFGGGTKLTVL

SEQ ID NO: 69 - DNA, 1488 bp, synthetic construct
  misc_feature 1..1488: note = 2G8LHxSEQ ID NO: 16 ; bispecific single chain antibody
  source 1..1488: mol_type = other DNA
gatgttgtgatgacccagactccactcactttgtcggttaccattggacaaccagcctctatctcttgcaagtcaagtcagagcctcttatatagtaatggaaaaacctatttgaactggatattacagaggccaggccagtctccaaagcgcctaatctatctggtgtctaaactggactctggagtccctgacaggttcactggcagtggatcaggaacagattttacgctgaaaatcagcagagtggaggctgaggatttgggagtttattactgcgtgcaaggtacacattttcctctcacgttcggtgctgggaccaagctggagctgaaaggtggtggtggttctggcggcggcggctccggtggtggtggttctgaggttcagctgcagcagtctggggcagagcttgtgaggtcaggggcctcagtcaagttgtcctgcacagcttctggcttcaacattaaagactactatttgcactgggtgaagcagaggcctgaacagggcctggagtggattgcctggattgatcttgagaatggtgatattaaatatgccccgaagtttcagggcaaggccactataactgcagacacatcctccaacacagcctacctgcagctcagcagcctgacatctgaggacactgccgtctattactgtaatccctattactacggtagtaactacgactatgctatggactactggggtcaaggaacctcagtcaccgtctcctccggaggtggtggatcccaagttgttctcacccagtctccagcaatcatgtctgcatttccaggggagaaggtcaccatgacctgcagtgccagctcaagtgtaagttacatgaactggtaccagcagaagtcaggcacctcccccaaaagatggatttatgactcatccaaactggcttctggagtccctgctcgcttcagtggcagtgggtctgggacctcttattctctcacaatcagcagcatggagactgaagatgctgccacttattactgccagcagtggagtcgtaacccacccacgttcggaggggggaccaagctacaaattacaggtggtggtggttctggcggcggcggctccggtggtggtggttctcaggtccagctgcagcagtctggggctgaactggcaagacctggggcctcagtgaagatgtcctgcaaggcttctggctacacctttactagatctacgatgcactgggtaaaacagaggcctggacagggtctggaatggattggatacattaatcctagcagtgcttatactaattacaatcagaaattcaaggacaaggccacattgactgcagacaaatcctccagtacagcctacatgcaactgagtagcctgacatctgaggactctgcagtctattactgtgcaagtccgcaagtccactatgattacaacgggtttccttactggggccaagggactctggtcactgtctctgca

SEQ ID NO: 70 - protein, 496 aa, synthetic construct
  REGION 1..496: note = 2G8LHxSEQ ID NO: 16 ; bispecific single chain antibody
DVVMTQTPLTLSVTIGQPASISCKSSQSLLYSNGKTYLNWILQRPGQSPKRLIYLVSKLDSGVPDRFTGSGSGTDFTLKISRVEAEDLGVYYCVQGTHFPLTFGAGTKLELKGGGGSGGGGSGGGGSEVQLQQSGAELVRSGASVKLSCTASGFNIKDYYLHWVKQRPEQGLEWIAWIDLENGDIKYAPKFQGKATITADTSSNTAYLQLSSLTSEDTAVYYCNPYYYGSNYDYAMDYWGQGTSVTVSSGGGGSQVVLTQSPAIMSAFPGEKVTMTCSASSSVSYMNWYQQKSGTSPKRWIYDSSKLASGVPARFSGSGSGTSYSLTISSMETEDAATYYCQQWSRNPPTFGGGTKLQITGGGGSGGGGSGGGGSQVQLQQSGAELARPGASVKMSCKASGYTFTRSTMHWVKQRPGQGLEWIGYINPSSAYTNYNQKFKDKATLTADKSSSTAYMQLSSLTSEDSAVYYCASPQVHYDYNGFPYWGQGTLVTVSA

SEQ ID NO: 71 - DNA, 1488 bp, synthetic construct
  misc_feature 1..1488: note = 2G8LHxSEQ ID NO: 14 ; bispecific single chain antibody
  source 1..1488: mol_type = other DNA
gatgttgtgatgacccagactccactcactttgtcggttaccattggacaaccagcctctatctcttgcaagtcaagtcagagcctcttatatagtaatggaaaaacctatttgaactggatattacagaggccaggccagtctccaaagcgcctaatctatctggtgtctaaactggactctggagtccctgacaggttcactggcagtggatcaggaacagattttacgctgaaaatcagcagagtggaggctgaggatttgggagtttattactgcgtgcaaggtacacattttcctctcacgttcggtgctgggaccaagctggagctgaaaggtggtggtggttctggcggcggcggctccggtggtggtggttctgaggttcagctgcagcagtctggggcagagcttgtgaggtcaggggcctcagtcaagttgtcctgcacagcttctggcttcaacattaaagactactatttgcactgggtgaagcagaggcctgaacagggcctggagtggattgcctggattgatcttgagaatggtgatattaaatatgccccgaagtttcagggcaaggccactataactgcagacacatcctccaacacagcctacctgcagctcagcagcctgacatctgaggacactgccgtctattactgtaatccctattactacggtagtaactacgactatgctatggactactggggtcaaggaacctcagtcaccgtctcctccggaggtggtggatcccaggtccagctgcagcagtctggggctgaactggcaagacctggggcctcagtgaagatgtcctgcaaggcttctggctacacctttactagatctacgatgcactgggtaaaacagaggcctggacagggtctggaatggattggatacattaatcctagcagtgcttatactaattacaatcagaaattcaaggacaaggccacattgactgcagacaaatcctccagtacagcctacatgcaactgagtagcctgacatctgaggactctgcagtctattactgtgcaagtccgcaagtccactatgattacaacgggtttccttactggggccaagggactctggtcactgtctctgcaggtggtggtggttctggcggcggcggctccggtggtggtggttctcaagttgttctcacccagtctccagcaatcatgtctgcatttccaggggagaaggtcaccatgacctgcagtgccagctcaagtgtaagttacatgaactggtaccagcagaagtcaggcacctcccccaaaagatggatttatgactcatccaaactggcttctggagtccctgctcgcttcagtggcagtgggtctgggacctcttattctctcacaatcagcagcatggagactgaagatgctgccacttattactgccagcagtggagtcgtaacccacccacgttcggaggggggaccaagctacaaattaca

SEQ ID NO: 72 - protein, 496 aa, synthetic construct
  REGION 1..496: note = 2G8LHxSEQ ID NO: 14 ; bispecific single chain antibody
DVVMTQTPLTLSVTIGQPASISCKSSQSLLYSNGKTYLNWILQRPGQSPKRLIYLVSKLDSGVPDRFTGSGSGTDFTLKISRVEAEDLGVYYCVQGTHFPLTFGAGTKLELKGGGGSGGGGSGGGGSEVQLQQSGAELVRSGASVKLSCTASGFNIKDYYLHWVKQRPEQGLEWIAWIDLENGDIKYAPKFQGKATITADTSSNTAYLQLSSLTSEDTAVYYCNPYYYGSNYDYAMDYWGQGTSVTVSSGGGGSQVQLQQSGAELARPGASVKMSCKASGYTFTRSTMHWVKQRPGQGLEWIGYINPSSAYTNYNQKFKDKATLTADKSSSTAYMQLSSLTSEDSAVYYCASPQVHYDYNGFPYWGQGTLVTVSAGGGGSGGGGSGGGGSQVVLTQSPAIMSAFPGEKVTMTCSASSSVSYMNWYQQKSGTSPKRWIYDSSKLASGVPARFSGSGSGTSYSLTISSMETEDAATYYCQQWSRNPPTFGGGTKLQIT

SEQ ID NO: 73 - DNA, 1512 bp, synthetic construct
  misc_feature 1..1512: note = 2G8HLxSEQ ID NO: 12 ; bispecific single chain antibody
  source 1..1512: mol_type = other DNA
gaggttcagctgcagcagtctggggcagagcttgtgaggtcaggggcctcagtcaagttgtcctgcacagcttctggcttcaacattaaagactactatttgcactgggtgaagcagaggcctgaacagggcctggagtggattgcctggattgatcttgagaatggtgatattaaatatgccccgaagtttcagggcaaggccactataactgcagacacatcctccaacacagcctacctgcagctcagcagcctgacatctgaggacactgccgtctattactgtaatccctattactacggtagtaactacgactatgctatggactactggggtcaaggaacctcagtcaccgtctcctcaggtggtggtggttctggcggcggcggctccggtggtggtggttctgatgttgtgatgacccagactccactcactttgtcggttaccattggacaaccagcctctatctcttgcaagtcaagtcagagcctcttatatagtaatggaaaaacctatttgaactggatattacagaggccaggccagtctccaaagcgcctaatctatctggtgtctaaactggactctggagtccctgacaggttcactggcagtggatcaggaacagattttacgctgaaaatcagcagagtggaggctgaggatttgggagtttattactgcgtgcaaggtacacattttcctctcacgttcggtgctgggaccaagctggagctgaaatccggaggtggtggatcccaggctgttgtgactcaggaatctgcactcaccacatcacctggtgaaacagtcacactcacttgtcgctcaagtactggggctgttacaactagtaactatgccaactgggtccaagaaaaaccagatcatttattcactggtctaataggtggtaccaacaagcgagctccaggtgtgcctgccagattctcaggctccctgattggagacaaggctgccctcaccatcacaggggcacagactgaggatgaggcaatatatttctgtgctctatggtacagcaacctctgggtgttcggtggaggaaccaaactgactgtcctaggtggtggtggttctggcggcggcggctccggtggtggtggttctgaggtgaagcttctcgagtctggaggaggattggtgcagcctaaagggtcattgaaactctcatgtgcagcctctggattcaccttcaatacctacgccatgaactgggtccgccaggctccaggaaagggtttggaatgggttgctcgcataagaagtaaatataataattatgcaacatattatgccgattcagtgaaagacaggttcaccatctccagagatgattcacaaagcattctctatctacaaatgaacaacttgaaaactgaggacacagccatgtactactgtgtgagacatgggaacttcggtaatagctacgtttcctggtttgcttactggggccaagggactctggtcactgtctctgca

SEQ ID NO: 74 - protein, 504 aa, synthetic construct
  REGION 1..504: note = 2G8HLxSEQ ID NO: 12 ; bispecific single chain antibody
EVQLQQSGAELVRSGASVKLSCTASGFNIKDYYLHWVKQRPEQGLEWIAWIDLENGDIKYAPKFQGKATITADTSSNTAYLQLSSLTSEDTAVYYCNPYYYGSNYDYAMDYWGQGTSVTVSSGGGGSGGGGSGGGGSDVVMTQTPLTLSVTIGQPASISCKSSQSLLYSNGKTYLNWILQRPGQSPKRLIYLVSKLDSGVPDRFTGSGSGTDFTLKISRVEAEDLGVYYCVQGTHFPLTFGAGTKLELKSGGGGSQAVVTQESALTTSPGETVTLTCRSSTGAVTTSNYANWVQEKPDHLFTGLIGGTNKRAPGVPARFSGSLIGDKAALTITGAQTEDEAIYFCALWYSNLWVFGGGTKLTVLGGGGSGGGGSGGGGSEVKLLESGGGLVQPKGSLKLSCAASGFTFNTYAMNWVRQAPGKGLEWVARIRSKYNNYATYYADSVKDRFTISRDDSQSILYLQMNNLKTEDTAMYYCVRHGNFGNSYVSWFAYWGQGTLVTVSA

SEQ ID NO: 75 - DNA, 1512 bp, synthetic construct
  misc_feature 1..1512: note = 2G8HLxSEQ ID NO: 10 ; bispecific single chain antibody
  source 1..1512: mol_type = other DNA
gaggttcagctgcagcagtctggggcagagcttgtgaggtcaggggcctcagtcaagttgtcctgcacagcttctggcttcaacattaaagactactatttgcactgggtgaagcagaggcctgaacagggcctggagtggattgcctggattgatcttgagaatggtgatattaaatatgccccgaagtttcagggcaaggccactataactgcagacacatcctccaacacagcctacctgcagctcagcagcctgacatctgaggacactgccgtctattactgtaatccctattactacggtagtaactacgactatgctatggactactggggtcaaggaacctcagtcaccgtctcctcaggtggtggtggttctggcggcggcggctccggtggtggtggttctgatgttgtgatgacccagactccactcactttgtcggttaccattggacaaccagcctctatctcttgcaagtcaagtcagagcctcttatatagtaatggaaaaacctatttgaactggatattacagaggccaggccagtctccaaagcgcctaatctatctggtgtctaaactggactctggagtccctgacaggttcactggcagtggatcaggaacagattttacgctgaaaatcagcagagtggaggctgaggatttgggagtttattactgcgtgcaaggtacacattttcctctcacgttcggtgctgggaccaagctggagctgaaatccggaggtggtggatccgaggtgaagcttctcgagtctggaggaggattggtgcagcctaaagggtcattgaaactctcatgtgcagcctctggattcaccttcaatacctacgccatgaactgggtccgccaggctccaggaaagggtttggaatgggttgctcgcataagaagtaaatataataattatgcaacatattatgccgattcagtgaaagacaggttcaccatctccagagatgattcacaaagcattctctatctacaaatgaacaacttgaaaactgaggacacagccatgtactactgtgtgagacatgggaacttcggtaatagctacgtttcctggtttgcttactggggccaagggactctggtcactgtctctgcaggtggtggtggttctggcggcggcggctccggtggtggtggttctcaggctgttgtgactcaggaatctgcactcaccacatcacctggtgaaacagtcacactcacttgtcgctcaagtactggggctgttacaactagtaactatgccaactgggtccaagaaaaaccagatcatttattcactggtctaataggtggtaccaacaagcgagctccaggtgtgcctgccagattctcaggctccctgattggagacaaggctgccctcaccatcacaggggcacagactgaggatgaggcaatatatttctgtgctctatggtacagcaacctctgggtgttcggtggaggaaccaaactgactgtccta

SEQ ID NO: 76 - protein, 504 aa, synthetic construct
  REGION 1..504: note = 2G8HLxSEQ ID NO: 10 ; bispecific single chain antibody
EVQLQQSGAELVRSGASVKLSCTASGFNIKDYYLHWVKQRPEQGLEWIAWIDLENGDIKYAPKFQGKATITADTSSNTAYLQLSSLTSEDTAVYYCNPYYYGSNYDYAMDYWGQGTSVTVSSGGGGSGGGGSGGGGSDVVMTQTPLTLSVTIGQPASISCKSSQSLLYSNGKTYLNWILQRPGQSPKRLIYLVSKLDSGVPDRFTGSGSGTDFTLKISRVEAEDLGVYYCVQGTHFPLTFGAGTKLELKSGGGGSEVKLLESGGGLVQPKGSLKLSCAASGFTFNTYAMNWVRQAPGKGLEWVARIRSKYNNYATYYADSVKDRFTISRDDSQSILYLQMNNLKTEDTAMYYCVRHGNFGNSYVSWFAYWGQGTLVTVSAGGGGSGGGGSGGGGSQAVVTQESALTTSPGETVTLTCRSSTGAVTTSNYANWVQEKPDHLFTGLIGGTNKRAPGVPARFSGSLIGDKAALTITGAQTEDEAIYFCALWYSNLWVFGGGTKLTVL

SEQ ID NO: 77 - DNA, 1491 bp, synthetic construct
  misc_feature 1..1491: note = 2G8HLxSEQ ID NO: 16 ; bispecific single chain antibody
  source 1..1491: mol_type = other DNA
gaggttcagctgcagcagtctggggcagagcttgtgaggtcaggggcctcagtcaagttgtcctgcacagcttctggcttcaacattaaagactactatttgcactgggtgaagcagaggcctgaacagggcctggagtggattgcctggattgatcttgagaatggtgatattaaatatgccccgaagtttcagggcaaggccactataactgcagacacatcctccaacacagcctacctgcagctcagcagcctgacatctgaggacactgccgtctattactgtaatccctattactacggtagtaactacgactatgctatggactactggggtcaaggaacctcagtcaccgtctcctcaggtggtggtggttctggcggcggcggctccggtggtggtggttctgatgttgtgatgacccagactccactcactttgtcggttaccattggacaaccagcctctatctcttgcaagtcaagtcagagcctcttatatagtaatggaaaaacctatttgaactggatattacagaggccaggccagtctccaaagcgcctaatctatctggtgtctaaactggactctggagtccctgacaggttcactggcagtggatcaggaacagattttacgctgaaaatcagcagagtggaggctgaggatttgggagtttattactgcgtgcaaggtacacattttcctctcacgttcggtgctgggaccaagctggagctgaaatccggaggtggtggatcccaagttgttctcacccagtctccagcaatcatgtctgcatttccaggggagaaggtcaccatgacctgcagtgccagctcaagtgtaagttacatgaactggtaccagcagaagtcaggcacctcccccaaaagatggatttatgactcatccaaactggcttctggagtccctgctcgcttcagtggcagtgggtctgggacctcttattctctcacaatcagcagcatggagactgaagatgctgccacttattactgccagcagtggagtcgtaacccacccacgttcggaggggggaccaagctacaaattacaggtggtggtggttctggcggcggcggctccggtggtggtggttctcaggtccagctgcagcagtctggggctgaactggcaagacctggggcctcagtgaagatgtcctgcaaggcttctggctacacctttactagatctacgatgcactgggtaaaacagaggcctggacagggtctggaatggattggatacattaatcctagcagtgcttatactaattacaatcagaaattcaaggacaaggccacattgactgcagacaaatcctccagtacagcctacatgcaactgagtagcctgacatctgaggactctgcagtctattactgtgcaagtccgcaagtccactatgattacaacgggtttccttactggggccaagggactctggtcactgtctctgca

SEQ ID NO: 78 - protein, 497 aa, synthetic construct
  REGION 1..497: note = 2G8HLxSEQ ID NO: 16 ; bispecific single chain antibody
EVQLQQSGAELVRSGASVKLSCTASGFNIKDYYLHWVKQRPEQGLEWIAWIDLENGDIKYAPKFQGKATITADTSSNTAYLQLSSLTSEDTAVYYCNPYYYGSNYDYAMDYWGQGTSVTVSSGGGGSGGGGSGGGGSDVVMTQTPLTLSVTIGQPASISCKSSQSLLYSNGKTYLNWILQRPGQSPKRLIYLVSKLDSGVPDRFTGSGSGTDFTLKISRVEAEDLGVYYCVQGTHFPLTFGAGTKLELKSGGGGSQVVLTQSPAIMSAFPGEKVTMTCSASSSVSYMNWYQQKSGTSPKRWIYDSSKLASGVPARFSGSGSGTSYSLTISSMETEDAATYYCQQWSRNPPTFGGGTKLQITGGGGSGGGGSGGGGSQVQLQQSGAELARPGASVKMSCKASGYTFTRSTMHWVKQRPGQGLEWIGYINPSSAYTNYNQKFKDKATLTADKSSSTAYMQLSSLTSEDSAVYYCASPQVHYDYNGFPYWGQGTLVTVSA

SEQ ID NO: 79 - DNA, 1491 bp, synthetic construct
  misc_feature 1..1491: note = 2G8HLxSEQ ID NO: 14 ; bispecific single chain antibody
  source 1..1491: mol_type = other DNA
gaggttcagctgcagcagtctggggcagagcttgtgaggtcaggggcctcagtcaagttgtcctgcacagcttctggcttcaacattaaagactactatttgcactgggtgaagcagaggcctgaacagggcctggagtggattgcctggattgatcttgagaatggtgatattaaatatgccccgaagtttcagggcaaggccactataactgcagacacatcctccaacacagcctacctgcagctcagcagcctgacatctgaggacactgccgtctattactgtaatccctattactacggtagtaactacgactatgctatggactactggggtcaaggaacctcagtcaccgtctcctcaggtggtggtggttctggcggcggcggctccggtggtggtggttctgatgttgtgatgacccagactccactcactttgtcggttaccattggacaaccagcctctatctcttgcaagtcaagtcagagcctcttatatagtaatggaaaaacctatttgaactggatattacagaggccaggccagtctccaaagcgcctaatctatctggtgtctaaactggactctggagtccctgacaggttcactggcagtggatcaggaacagattttacgctgaaaatcagcagagtggaggctgaggatttgggagtttattactgcgtgcaaggtacacattttcctctcacgttcggtgctgggaccaagctggagctgaaatccggaggtggtggatcccaggtccagctgcagcagtctggggctgaactggcaagacctggggcctcagtgaagatgtcctgcaaggcttctggctacacctttactagatctacgatgcactgggtaaaacagaggcctggacagggtctggaatggattggatacattaatcctagcagtgcttatactaattacaatcagaaattcaaggacaaggccacattgactgcagacaaatcctccagtacagcctacatgcaactgagtagcctgacatctgaggactctgcagtctattactgtgcaagtccgcaagtccactatgattacaacgggtttccttactggggccaagggactctggtcactgtctctgcaggtggtggtggttctggcggcggcggctccggtggtggtggttctcaagttgttctcacccagtctccagcaatcatgtctgcatttccaggggagaaggtcaccatgacctgcagtgccagctcaagtgtaagttacatgaactggtaccagcagaagtcaggcacctcccccaaaagatggatttatgactcatccaaactggcttctggagtccctgctcgcttcagtggcagtgggtctgggacctcttattctctcacaatcagcagcatggagactgaagatgctgccacttattactgccagcagtggagtcgtaacccacccacgttcggaggggggaccaagctacaaattaca

SEQ ID NO: 80 - protein, 497 aa, synthetic construct
  REGION 1..497: note = 2G8HLxSEQ ID NO: 14 ; bispecific single chain antibody
EVQLQQSGAELVRSGASVKLSCTASGFNIKDYYLHWVKQRPEQGLEWIAWIDLENGDIKYAPKFQGKATITADTSSNTAYLQLSSLTSEDTAVYYCNPYYYGSNYDYAMDYWGQGTSVTVSSGGGGSGGGGSGGGGSDVVMTQTPLTLSVTIGQPASISCKSSQSLLYSNGKTYLNWILQRPGQSPKRLIYLVSKLDSGVPDRFTGSGSGTDFTLKISRVEAEDLGVYYCVQGTHFPLTFGAGTKLELKSGGGGSQVQLQQSGAELARPGASVKMSCKASGYTFTRSTMHWVKQRPGQGLEWIGYINPSSAYTNYNQKFKDKATLTADKSSSTAYMQLSSLTSEDSAVYYCASPQVHYDYNGFPYWGQGTLVTVSAGGGGSGGGGSGGGGSQVVLTQSPAIMSAFPGEKVTMTCSASSSVSYMNWYQQKSGTSPKRWIYDSSKLASGVPARFSGSGSGTSYSLTISSMETEDAATYYCQQWSRNPPTFGGGTKLQIT

SEQ ID NO: 81 - DNA, 27 bp, synthetic construct
  misc_feature 1..27: note = 5' primer VH ; oligonucleotide
  source 1..27: mol_type = other DNA
saggtgcagctcgaggagtcaggacct

SEQ ID NO: 82 - DNA, 27 bp, synthetic construct
  misc_feature 1..27: note = 5' primer VH ; oligonucleotide
  source 1..27: mol_type = other DNA
gaggtccagctcgagcagtctggacct

SEQ ID NO: 83 - DNA, 27 bp, synthetic construct
  misc_feature 1..27: note = 5' primer VH ; oligonucleotide
  source 1..27: mol_type = other DNA
caggtccaactcgagcagcctggggct

SEQ ID NO: 84 - DNA, 27 bp, synthetic construct
  misc_feature 1..27: note = 5' primer VH ; oligonucleotide
  source 1..27: mol_type = other DNA
gaggttcagctcgagcagtctggggca

SEQ ID NO: 85 - DNA, 27 bp, synthetic construct
  misc_feature 1..27: note = 5' primer VH ; oligonucleotide
  source 1..27: mol_type = other DNA
gargtgaagctcgaggagtctggagga

SEQ ID NO: 86 - DNA, 27 bp, synthetic construct
  misc_feature 1..27: note = 5' primer VH ; oligonucleotide
  source 1..27: mol_type = other DNA
gaggtgaagcttctcgagtctggaggt

SEQ ID NO: 87 - DNA, 27 bp, synthetic construct
  misc_feature 1..27: note = 5' primer VH ; oligonucleotide
  source 1..27: mol_type = other DNA
gaagtgaagctcgaggagtctggggga

SEQ ID NO: 88 - DNA, 27 bp, synthetic construct
  misc_feature 1..27: note = 5' primer VH ; oligonucleotide
  source 1..27: mol_type = other DNA
gaggttcagctcgagcagtctggagct

SEQ ID NO: 89 - DNA, 39 bp, synthetic construct
  misc_feature 1..39: note = 5' primer VH ; oligonucleotide
  source 1..39: mol_type = other DNA
gggctcgagcaccatggratgsagctgkgtmatsctctt

SEQ ID NO: 90 - DNA, 39 bp, synthetic construct
  misc_feature 1..39: note = 5' primer VH ; oligonucleotide
  source 1..39: mol_type = other DNA
gggctcgagcaccatgracttcgggytgagctkggtttt

SEQ ID NO: 91 - DNA, 38 bp, synthetic construct
  misc_feature 1..38: note = 5' primer VH ; oligonucleotide
  source 1..38: mol_type = other DNA
gggctcgagcaccatggctgtcttggggctgctcttct

SEQ ID NO: 92 - DNA, 34 bp, synthetic construct
  misc_feature 1..34: note = 3' primer VH ; oligonucleotide
  source 1..34: mol_type = other DNA
gaggaattcgaactggacagggatccagagttcc

SEQ ID NO: 93 - DNA, 34 bp, synthetic construct
  misc_feature 1..34: note = 3' primer VH ; oligonucleotide
  source 1..34: mol_type = other DNA
cggaattcgaatgacatggacatctgggtcatcc

SEQ ID NO: 94 - DNA, 32 bp, synthetic construct
  misc_feature 1..32: note = 5' primer VL ; oligonucleotide
  source 1..32: mol_type = other DNA
ccagttccgagctcgttgtgactcaggaatct

SEQ ID NO: 95 - DNA, 32 bp, synthetic construct
  misc_feature 1..32: note = 5' primer VL ; oligonucleotide
  source 1..32: mol_type = other DNA
ccagttccgagctcgtgttgacgcagccgccc

SEQ ID NO: 96 - DNA, 32 bp, synthetic construct
  misc_feature 1..32: note = 5' primer VL ; oligonucleotide
  source 1..32: mol_type = other DNA
ccagttccgagctcgtgctcacccagtctcca

SEQ ID NO: 97 - DNA, 32 bp, synthetic construct
  misc_feature 1..32: note = 5' primer VL ; oligonucleotide
  source 1..32: mol_type = other DNA
ccagttccgagctccagatgacccagtctcca

SEQ ID NO: 98 - DNA, 32 bp, synthetic construct
  misc_feature 1..32: note = 5' primer VL ; oligonucleotide
  source 1..32: mol_type = other DNA
ccagatgtgagctcgtgatgacccagactcca

SEQ ID NO: 99 - DNA, 32 bp, synthetic construct
  misc_feature 1..32: note = 5' primer VL ; oligonucleotide
  source 1..32: mol_type = other DNA
ccagatgtgagctcgtcatgacccagtctcca

SEQ ID NO: 100 - DNA, 32 bp, synthetic construct
  misc_feature 1..32: note = 5' primer VL ; oligonucleotide
  source 1..32: mol_type = other DNA
ccagttccgagctcgtgatgacacagtctcca

SEQ ID NO: 101 - DNA, 38 bp, synthetic construct
  misc_feature 1..38: note = 5' primer VL ; oligonucleotide
  source 1..38: mol_type = other DNA
ggggagctccaccatggagacagacacactcctgctat

SEQ ID NO: 102 - DNA, 39 bp, synthetic construct
  misc_feature 1..39: note = 5' primer VL ; oligonucleotide
  source 1..39: mol_type = other DNA
ggggagctccaccatggattttcaagtgcagattttcag

SEQ ID NO: 103 - DNA, 40 bp, synthetic construct
  misc_feature 1..40: note = 5' primer VL ; oligonucleotide
  source 1..40: mol_type = other DNA
ggggagctccaccatggagwcacakwctcaggtctttrta

SEQ ID NO: 104 - DNA, 36 bp, synthetic construct
  misc_feature 1..36: note = 5' primer VL ; oligonucleotide
  source 1..36: mol_type = other DNA
ggggagctccaccatgkccccwrctcagytyctkgt

SEQ ID NO: 105 - DNA, 31 bp, synthetic construct
  misc_feature 1..31: note = 3' primer VL ; oligonucleotide
  source 1..31: mol_type = other DNA
gaggaattcgaactgctcactggatggtggg

SEQ ID NO: 106 - DNA, 35 bp, synthetic construct
  misc_feature 1..35: note = 3' primer VL ; oligonucleotide
  source 1..35: mol_type = other DNA
cggaattcgaacaaactcttctccacagtgtgacc

SEQ ID NO: 107 - DNA, 30 bp, synthetic construct
  misc_feature 1..30: note = 3' primer VH ; oligonucleotide
  source 1..30: mol_type = other DNA
tatgcaactagtacaaccacaatccctggg

SEQ ID NO: 108 - DNA, 34 bp, synthetic construct
  misc_feature 1..34: note = 3' primer VL ; oligonucleotide
  source 1..34: mol_type = other DNA
gcgccgtctagaattaacactcattcctgttgaa

SEQ ID NO: 109 - protein, 496 aa, synthetic construct
  REGION 1..496: note = 5-10 LH x deimmunised (di) anti-CD3 ; bispecific single chain antibody
ELVMTQSPSSLTVTAGEKVTMSCKSSQSLLNSGNQKNYLTWYQQKPGQPPKLLIYWASTRESGVPDRFTGSGSGTDFTLTISSVQAEDLAVYYCQNDYSYPLTFGAGTKLEIKGGGGSGGGGSGGGGSEVQLLEQSGAELVRPGTSVKISCKASGYAFTNYWLGWVKQRPGHGLEWIGDIFPGSGNIHYNEKFKGKATLTADKSSSTAYMQLSSLTFEDSAVYFCARLRNWDEPMDYWGQGTTVTVSSGGGGSDVQLVQSGAEVKKPGASVKVSCKASGYTFTRYTMHWVRQAPGQGLEWIGYINPSRGYTNYADSVKGRFTITTDKSTSTAYMELSSLRSEDTATYYCARYYDDHYCLDYWGQGTTVTVSSGEGTSTGSGGSGGSGGADDIVLTQSPATLSLSPGERATLSCRASQSVSYMNWYQQKPGKAPKRWIYDTSKVASGVPARFSGSGSGTDYSLTINSLEAEDAATYYCQQWSSNPLTFGGGTKVEIK

SEQ ID NO: 110 - protein, 125 aa, synthetic construct
  REGION 1..125: note = Human-like VH VH region
EVQLLESGGGLVQPGGSLKLSCAASGFTFNTYAMNWVRQAPGKGLEWVARIRSKYNNYATYYADSVKDRFTISRDDSKNTAYLQMNNLKTEDTAVYYCVRHGNFGNSYVSWFAYWGQGTLVTVSS

SEQ ID NO: 111 - DNA, 375 bp, synthetic construct
  misc_feature 1..375: note = Human-like VH VH region
  source 1..375: mol_type = other DNA
gaggtgcagctgctcgagtctggaggaggattggtgcagcctggagggtcattgaaactctcatgtgcagcctctggattcaccttcaatacctacgccatgaactgggtccgccaggctccaggaaagggtttggaatgggttgctcgcataagaagtaaatataataattatgcaacatattatgccgattcagtgaaagacaggttcaccatctccagagatgattcaaaaaacactgcctatctacaaatgaacaacttgaaaactgaggacactgccgtgtactactgtgtgagacatgggaacttcggtaatagctacgtttcctggtttgcttactggggccaagggactctggtcaccgtctcctca

SEQ ID NO: 112 - protein, 14 aa, Mus musculus
  SITE 1: note = misc_feature - CDR3 of VH
HGNFGNSYVSWFAY

SEQ ID NO: 113 - protein, 6 aa, Mus musculus
  REGION 1..6: note = misc_feature - VH: short CDR3 short CDR3 of SEQ ID NO: 112
VSWFAY

SEQ ID NO: 114 - protein, 19 aa, Mus musculus
  REGION 1..19: note = misc_feature - CDR2 of VH
RIRSKYNNYATYYADSVKD

SEQ ID NO: 115 - protein, 5 aa, Mus musculus
  REGION 1..5: note = misc_feature - CDR1 of VH
TYAMN

SEQ ID NO: 116 - protein, 9 aa, Mus musculus
  REGION 1..9: note = misc_feature - CDR3 of VL
ALWYSNLWV

SEQ ID NO: 117 - protein, 7 aa, Mus musculus
  REGION 1..7: note = misc_feature - CDR2 of VL
GTNKRAP

SEQ ID NO: 118 - protein, 14 aa, Mus musculus
  REGION 1..14: note = misc_feature - CDR1 of VL
RSSTGAVTTSNYAN

SEQ ID NO: 119 - protein, 12 aa, Mus musculus
  REGION 1..12: note = misc_feature - CDR3 of VH
PQVHYDYNGFPY

SEQ ID NO: 120 - protein, 17 aa, Mus musculus
  REGION 1..17: note = misc_feature - CDR2 of VH
YINPSSAYTNYNQKFKD

SEQ ID NO: 121 - protein, 10 aa, Mus musculus
  REGION 1..10: note = misc_feature - CDR1 of VH
GYTFTRSTMH

SEQ ID NO: 122 - protein, 509 aa, synthetic construct
  REGION 1..509: note = 2G8 LHx SEQ ID NO: 146 ; bispecific single chain antibody
DVVMTQTPLTLSVTIGQPASISCKSSQSLLYSNGKTYLNWILQRPGQSPKRLIYLVSKLDSGVPDRFTGSGSGTDFTLKISRVEAEDLGVYYCVQGTHFPLTFGAGTKLELKGGGGSGGGGSGGGGSEVQLQQSGAELVRSGASVKLSCTASGFNIKDYYLHWVKQRPEQGLEWIAWIDLENGDIKYAPKFQGKATITADTSSNTAYLQLSSLTSEDTAVYYCNPYYYGSNYDYAMDYWGQGTSVTVSSGGGGSEVKLLESGGGLVQPGGSLKLSCAASGFTFNTYAMNWVRQAPGKGLEWVARIRSKYNNYATYYADSVKDRFTISRDDSKNTAYLQMNNLKTEDTAVYYCVRHGNFGNSYVSWFAYWGQGTLVTVSSGGGGSGGGGSGGGGSELVVTQESALTTSPGETVTLTCRSSTGAVTTSNYANWVQEKPDHLFTGLIGGTNKRAPGVPARFSGSLIGDKAALTITGAQTEDEAIYFCALWYSNLWVFGGGTKLTVLHHHHHH

SEQ ID NO: 123 - DNA, 1527 bp, synthetic construct
  misc_feature 1..1527: note = 2G8 LHxSEQ ID NO: 146 ; bispecific single chain antibody
  source 1..1527: mol_type = other DNA
gatgttgtgatgacccagactccactcactttgtcggttaccattggacaaccagcctctatctcttgcaagtcaagtcagagcctcttatatagtaatggaaaaacctatttgaactggatattacagaggccaggccagtctccaaagcgcctaatctatctggtgtctaaactggactctggagtccctgacaggttcactggcagtggatcaggaacagattttacgctgaaaatcagcagagtggaggctgaggatttgggagtttattactgcgtgcaaggtacacattttcctctcacgttcggtgctgggaccaagctggagctgaaaggtggtggtggttctggcggcggcggctccggtggtggtggttctgaggttcagctgcagcagtctggggcagagcttgtgaggtcaggggcctcagtcaagttgtcctgcacagcttctggcttcaacattaaagactactatttgcactgggtgaagcagaggcctgaacagggcctggagtggattgcctggattgatcttgagaatggtgatattaaatatgccccgaagtttcagggcaaggccactataactgcagacacatcctccaacacagcctacctgcagctcagcagcctgacatctgaggacactgccgtctattactgtaatccctattactacggtagtaactacgactatgctatggactactggggtcaaggaacctcagtcaccgtctcctccggaggtggtggatccgaggtgaagcttctcgagtctggaggaggattggtgcagcctggagggtcattgaaactctcatgtgcagcctctggattcaccttcaatacctacgccatgaactgggtccgccaggctccaggaaagggtttggaatgggttgctcgcataagaagtaaatataataattatgcaacatattatgccgattcagtgaaagacaggttcaccatctccagagatgattcaaaaaacactgcctatctacaaatgaacaacttgaaaactgaggacactgccgtgtactactgtgtgagacatgggaacttcggtaatagctacgtttcctggtttgcttactggggccaagggactctggtcaccgtctcctcaggtggtggtggttctggcggcggcggctccggtggtggtggttctgagctcgttgtgactcaggaatctgcactcaccacatcacctggtgaaacagtcacactcacttgtcgctcaagtactggggctgttacaactagtaactatgccaactgggtccaagaaaaaccagatcatttattcactggtctaataggtggtaccaacaagcgagcaccaggtgtgcctgccagattctcaggctccctgattggagacaaggctgccctcaccatcacaggggcacagactgaggatgaggcaatatatttctgtgctctatggtacagcaacctctgggtgttcggtggaggaaccaaactgactgtcctacatcatcaccatcatcat

SEQ ID NO: 124 - protein, 508 aa, synthetic construct
  REGION 1..508: note = 5-10 LHxSEQ ID NO: 146 ; bispecific single chain antibody
ELVMTQSPSSLTVTAGEKVTMSCKSSQSLLNSGNQKNYLTWYQQKPGQPPKLLIYWASTRESGVPDRFTGSGSGTDFTLTISSVQAEDLAVYYCQNDYSYPLTFGAGTKLEIKGGGGSGGGGSGGGGSEVQLLEQSGAELVRPGTSVKISCKASGYAFTNYWLGWVKQRPGHGLEWIGDIFPGSGNIHYNEKFKGKATLTADKSSSTAYMQLSSLTFEDSAVYFCARLRNWDEPMDYWGQGTTVTVSSGGGGSEVKLLESGGGLVQPGGSLKLSCAASGFTFNTYAMNWVRQAPGKGLEWVARIRSKYNNYATYYADSVKDRFTISRDDSKNTAYLQMNNLKTEDTAVYYCVRHGNFGNSYVSWFAYWGQGTLVTVSSGGGGSGGGGSGGGGSELVVTQESALTTSPGETVTLTCRSSTGAVTTSNYANWVQEKPDHLFTGLIGGTNKRAPGVPARFSGSLIGDKAALTITGAQTEDEAIYFCALWYSNLWVFGGGTKLTVLHHHHHH

SEQ ID NO: 125 - DNA, 1524 bp, synthetic construct
  misc_feature 1..1524: note = 5-10 LHxSEQ ID NO: 146 ; bispecific single chain antibody
  source 1..1524: mol_type = other DNA
gagctcgtgatgacacagtctccatcctccctgactgtgacagcaggagagaaggtcactatgagctgcaagtccagtcagagtctgttaaacagtggaaatcaaaagaactacttgacctggtaccagcagaaaccagggcagcctcctaaactgttgatctactgggcatccactagggaatctggggtccctgatcgcttcacaggcagtggatctggaacagatttcactctcaccatcagcagtgtgcaggctgaagacctggcagtttattactgtcagaatgattatagttatccgctcacgttcggtgctgggaccaagcttgagatcaaaggtggtggtggttctggcggcggcggctccggtggtggtggttctgaggtgcagctgctcgagcagtctggagctgagctggtaaggcctgggacttcagtgaagatatcctgcaaggcttctggatacgccttcactaactactggctaggttgggtaaagcagaggcctggacatggacttgagtggattggagatattttccctggaagtggtaatatccactacaatgagaagttcaagggcaaagccacactgactgcagacaaatcttcgagcacagcctatatgcagctcagtagcctgacatttgaggactctgctgtctatttctgtgcaagactgaggaactgggacgagcctatggactactggggccaagggaccacggtcaccgtctcctccggaggtggtggatccgaggtgaagcttctcgagtctggaggaggattggtgcagcctggagggtcattgaaactctcatgtgcagcctctggattcaccttcaatacctacgccatgaactgggtccgccaggctccaggaaagggtttggaatgggttgctcgcataagaagtaaatataataattatgcaacatattatgccgattcagtgaaagacaggttcaccatctccagagatgattcaaaaaacactgcctatctacaaatgaacaacttgaaaactgaggacactgccgtgtactactgtgtgagacatgggaacttcggtaatagctacgtttcctggtttgcttactggggccaagggactctggtcaccgtctcctcaggtggtggtggttctggcggcggcggctccggtggtggtggttctgagctcgttgtgactcaggaatctgcactcaccacatcacctggtgaaacagtcacactcacttgtcgctcaagtactggggctgttacaactagtaactatgccaactgggtccaagaaaaaccagatcatttattcactggtctaataggtggtaccaacaagcgagcaccaggtgtgcctgccagattctcaggctccctgattggagacaaggctgccctcaccatcacaggggcacagactgaggatgaggcaatatatttctgtgctctatggtacagcaacctctgggtgttcggtggaggaaccaaactgactgtcctacatcatcaccatcatcat

SEQ ID NO: 126 - DNA, 372 bp, synthetic construct
  misc_feature 1..372: note = Hybridoma
  misc_feature 1..372: note = FN18 VH VH region of monoclonal antibody FN18
  source 1..372: mol_type = other DNA
caggtccagctgcagcagtctgaagctgaactggcaagacctggggcctcagtgaagatgtcctgcaaggcttctggctacacctttactgactacacgatacactggttaaaacagaggcctggacagggtctggactggattggatattttaatcctagcagtgaatctactgaatacaatcggaaattcaaggacaggaccatattgactgcagacagatcctcaaccacagcctacatgcaactgagcagcctgacatctgaggactctgcggtctattactgttcaaggaaaggggagaaactacttggtaaccgttactggtacttcgatgtctggggcgcagggacctcggtcaccgtctcctca

SEQ ID NO: 127 - protein, 124 aa, synthetic construct
  REGION 1..124: note = Hybridoma
  REGION 1..124: note = misc_feature - FN18 VH VH region of monoclonal antibody FN18
QVQLQQSEAELARPGASVKMSCKASGYTFTDYTIHWLKQRPGQGLDWIGYFNPSSESTEYNRKFKDRTILTADRSSTTAYMQLSSLTSEDSAVYYCSRKGEKLLGNRYWYFDVWGAGTSVTVSS

SEQ ID NO: 128 - DNA, 339 bp, synthetic construct
  misc_feature 1..339: note = Hybridoma
  misc_feature 1..339: note = FN18 VL VL region of monoclonal antibody FN18
  source 1..339: mol_type = other DNA
gacattgtgatgtcacagtctccatcctccctagctgtgtcagttggagagaaggttactatgagctgcaagtccagtcagagccttttatatagtagcaatcaaaagaactacttggcctggtaccagcagaagccagggcagtctcctaaattgctgattaactgggcatccaccagggaatctggggtccctgatcgcttcacaggcagtggatctaggacagatttcactctcaccatcagcagtgtgaaggctgaagacctggcagtttatttctgtcagcaattttatagttatcctccgacgttcggtggaggcaccaagctggaaatcaaa

SEQ ID NO: 129 - protein, 113 aa, synthetic construct
  REGION 1..113: note = Hybridoma
  REGION 1..113: note = misc_feature - FN18 VL VL region of monoclonal antibody FN18
DIVMSQSPSSLAVSVGEKVTMSCKSSQSLLYSSNQKNYLAWYQQKPGQSPKLLINWASTRESGVPDRFTGSGSRTDFTLTISSVKAEDLAVYFCQQFYSYPPTFGGGTKLEIK

SEQ ID NO: 130 - DNA, 756 bp, synthetic construct
  misc_feature 1..756: note = FN18 VH-VL scFv ; single chain Fv
  source 1..756: mol_type = other DNA
caggtccagctgcagcagtctgaagctgaactggcaagacctggggcctcagtgaagatgtcctgcaaggcttctggctacacctttactgactacacgatacactggttaaaacagaggcctggacagggtctggactggattggatattttaatcctagcagtgaatctactgaatacaatcggaaattcaaggacaggaccatattgactgcagacagatcctcaaccacagcctacatgcaactgagcagcctgacatctgaggactctgcggtctattactgttcaaggaaaggggagaaactacttggtaaccgttactggtacttcgatgtctggggcgcagggacctcggtcaccgtctcctcaggtggtggtggttctggcggcggcggctccggtggtggtggttctgacattgtgatgtcacagtctccatcctccctagctgtgtcagttggagagaaggttactatgagctgcaagtccagtcagagccttttatatagtagcaatcaaaagaactacttggcctggtaccagcagaagccagggcagtctcctaaattgctgattaactgggcatccaccagggaatctggggtccctgatcgcttcacaggcagtggatctaggacagatttcactctcaccatcagcagtgtgaaggctgaagacctggcagtttatttctgtcagcaattttatagttatcctccgacgttcggtggaggcaccaagctggaaatcaaa

SEQ ID NO: 131 - protein, 252 aa, synthetic construct
  REGION 1..252: note = FN18 VH-VL scFv ; single chain Fv
QVQLQQSEAELARPGASVKMSCKASGYTFTDYTIHWLKQRPGQGLDWIGYFNPSSESTEYNRKFKDRTILTADRSSTTAYMQLSSLTSEDSAVYYCSRKGEKLLGNRYWYFDVWGAGTSVTVSSGGGGSGGGGSGGGGSDIVMSQSPSSLAVSVGEKVTMSCKSSQSLLYSSNQKNYLAWYQQKPGQSPKLLINWASTRESGVPDRFTGSGSRTDFTLTISSVKAEDLAVYFCQQFYSYPPTFGGGTKLEIK

SEQ ID NO: 132 - DNA, 756 bp, synthetic construct
  misc_feature 1..756: note = FN18 VL-VH scFv ; single chain Fv
  source 1..756: mol_type = other DNA
gacattgtgatgtcacagtctccatcctccctagctgtgtcagttggagagaaggttactatgagctgcaagtccagtcagagccttttatatagtagcaatcaaaagaactacttggcctggtaccagcagaagccagggcagtctcctaaattgctgattaactgggcatccaccagggaatctggggtccctgatcgcttcacaggcagtggatctaggacagatttcactctcaccatcagcagtgtgaaggctgaagacctggcagtttatttctgtcagcaattttatagttatcctccgacgttcggtggaggcaccaagctggaaatcaaaggtggtggtggttctggcggcggcggctccggtggtggtggttctcaggtccagctgcagcagtctgaagctgaactggcaagacctggggcctcagtgaagatgtcctgcaaggcttctggctacacctttactgactacacgatacactggttaaaacagaggcctggacagggtctggactggattggatattttaatcctagcagtgaatctactgaatacaatcggaaattcaaggacaggaccatattgactgcagacagatcctcaaccacagcctacatgcaactgagcagcctgacatctgaggactctgcggtctattactgttcaaggaaaggggagaaactacttggtaaccgttactggtacttcgatgtctggggcgcagggacctcggtcaccgtctcctca

SEQ ID NO: 133 - protein, 252 aa, synthetic construct
  REGION 1..252: note = FN18 VL-VH scFv ; single chain Fv
DIVMSQSPSSLAVSVGEKVTMSCKSSQSLLYSSNQKNYLAWYQQKPGQSPKLLINWASTRESGVPDRFTGSGSRTDFTLTISSVKAEDLAVYFCQQFYSYPPTFGGGTKLEIKGGGGSGGGGSGGGGSQVQLQQSEAELARPGASVKMSCKASGYTFTDYTIHWLKQRPGQGLDWIGYFNPSSESTEYNRKFKDRTILTADRSSTTAYMQLSSLTSEDSAVYYCSRKGEKLLGNRYWYFDVWGAGTSVTVSS

SEQ ID NO: 134 - protein, 105 aa, Homo sapiens
  REGION 1..105: note = misc_feature - CD3 epsilon extracellular portion
QDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMD

SEQ ID NO: 135 - protein, 96 aa, Macaca fascicularis
  REGION 1..96: note = misc_feature - CD3 epsilon extracellular portion FN18+
QDGNEEMGSITQTPYQVSISGTTVILTCSQHLGSEAQWQHNGKNKEDSGDRLFLPEFSEMEQSGYYVCYPRGSNPEDASHHLYLKARVCENCMEMD

SEQ ID NO: 136 - protein, 96 aa, Macaca fascicularis
  REGION 1..96: note = misc_feature - CD3 epsilon extracellular portion FN18-
QDGNEEMGSITQTPYQVSISGTTVILTCSQHLGSEAQWQHNGKNKGDSGDQLFLPEFSEMEQSGYYVCYPRGSNPEDASHHLYLKARVCENCMEMD

SEQ ID NO: 137 - protein, 242 aa, Homo sapiens
  REGION 1..242: note = misc_feature - EpCAM extracellular portion
QEECVCENYKLAVNCFVNNNRQCQCTSVGAQNTVICSKLAAKCLVMKAEMNGSKLGRRAKPEGALQNNDGLYDPDCDESGLFKAKQCNGTSTCWCVNTAGVRRTDKDTEITCSERVRTYWIIIELKHKAREKPYDSKSLRTALQKEITTRYQLDPKFITSILYENNVITIDLVQNSSQKTQNDVDIADVAYYFEKDVKGESLFHSKKMDLTVNGEQLDLDPGQTLIYYVDEKAPEFSMQGLK

SEQ ID NO: 138 - protein, 242 aa, Pan troglodytes
  REGION 1..242: note = misc_feature - EpCAM extracellular portion
QEECVCENYKLAVNCFVNNNHQCQCTSIGAQNTVICSKLAAKCLVMKAEMNGSKLGRRAKPEGALQNNDGLYDPDCDESGLFKAKQCNGTSTCWCVNTAGVRRTDKDTEITCSERVRTYWIIIELKHKAREKPYDGKSLRTALQKEITTRYQLDPKFITNILYENNVITIDLVQNSSQKTQNDVDIADVAYYFEKDVKGESLFHSKKMDLTVNGEQLDLDPGQTLIYYVDEKAPEFSMQGLK

SEQ ID NO: 139 - protein, 242 aa, Macaca mulatta; identical to SEQ ID NO: 48
  REGION 1..242: note = misc_feature - EpCAM extracellular portion

SEQ ID NO: 140 - protein, 242 aa, Pan troglodytes; identical to SEQ ID NO: 138
  REGION 1..242: note = misc_feature - EpCAM extracellular portion

SEQ ID NO: 141 - protein, 242 aa, Macaca mulatta; identical to SEQ ID NO: 48
  REGION 1..242: note = misc_feat - EpCAM extracellular portion

SEQ ID NO: 142 - protein, 89 aa, Homo sapiens
  REGION 1..89: note = misc_feature - human CD3 gamma extracellular portion
QSIKGNHLVKVYDYQEDGSVLLTCDAEAKNITWFKDGKMIGFLTEDKKKWNLGSNAKDPRGMYQCKGSQNKSKPLQVYYRMCQNCIELN

SEQ ID NO: 143 - protein, 82 aa, Homo sapiens
  REGION 1..82: note = misc_feature - human CD3 delta extracellular portion
FKIPIEELEDRVFVNCNTSITWVEGTVGTLLSDITRLDLGKRILDPRGIYRCNGTDIYKDKESTVQVHYRMCQSCVELDPAT

SEQ ID NO: 144 - protein, 88 aa, Macaca fascicularis
  REGION 1..88: note = misc_feature - cynomolgus CD3 gamma extracellular portion
QSFEENRKLNVYNQEDGSVLLTCHVKNTNITWFKEGKMIDILTAHKNKWNLGSNTKDPRGVYQCKGSKDKSKTLQVYYRMCQNCIELN

SEQ ID NO: 145 - protein, 82 aa, Macaca fascicularis
  REGION 1..82: note = misc_feature - cynomolgus CD3 delta extracellular portion
FKIPVEELEDRVFVKCNTSVTWVEGTVGTLLTNNTRLDLGKRILDPRGIYRCNGTDIYKDKESAVQVHYRMCQNCVELDPAT

SEQ ID NO: 146 - protein, 249 aa, synthetic construct
  REGION 1..249: note = Human-like VH (SEQ ID NO: 110) x murine VL (SEQ ID NO: 148) scFv ; single chain Fv
EVQLLESGGGLVQPGGSLKLSCAASGFTFNTYAMNWVRQAPGKGLEWVARIRSKYNNYATYYADSVKDRFTISRDDSKNTAYLQMNNLKTEDTAVYYCVRHGNFGNSYVSWFAYWGQGTLVTVSSGGGGSGGGGSGGGGSELVVTQESALTTSPGETVTLTCRSSTGAVTTSNYANWVQEKPDHLFTGLIGGTNKRAPGVPARFSGSLIGDKAALTITGAQTEDEAIYFCALWYSNLWVFGGGTKLTVL

SEQ ID NO: 147 - DNA, 747 bp, synthetic construct
  misc_feature 1..747: note = Human-like VH (SEQ ID NO: 110) x murine VL (SEQ ID NO: 148) scFv ; single chain Fv
  source 1..747: mol_type = other DNA
gaggtgcagctgctcgagtctggaggaggattggtgcagcctggagggtcattgaaactctcatgtgcagcctctggattcaccttcaatacctacgccatgaactgggtccgccaggctccaggaaagggtttggaatgggttgctcgcataagaagtaaatataataattatgcaacatattatgccgattcagtgaaagacaggttcaccatctccagagatgattcaaaaaacactgcctatctacaaatgaacaacttgaaaactgaggacactgccgtgtactactgtgtgagacatgggaacttcggtaatagctacgtttcctggtttgcttactggggccaagggactctggtcaccgtctcctcaggtggtggtggttctggcggcggcggctccggtggtggtggttctgagctcgttgtgactcaggaatctgcactcaccacatcacctggtgaaacagtcacactcacttgtcgctcaagtactggggctgttacaactagtaactatgccaactgggtccaagaaaaaccagatcatttattcactggtctaataggtggtaccaacaagcgagcaccaggtgtgcctgccagattctcaggctccctgattggagacaaggctgccctcaccatcacaggggcacagactgaggatgaggcaatatatttctgtgctctatggtacagcaacctctgggtgttcggtggaggaaccaaactgactgtccta

SEQ ID NO: 148 - protein, 109 aa, synthetic construct
  REGION 1..109: note = murine VL
ELVVTQESALTTSPGETVTLTCRSSTGAVTTSNYANWVQEKPDHLFTGLIGGTNKRAPGVPARFSGSLIGDKAALTITGAQTEDEAIYFCALWYSNLWVFGGGTKLTVL

SEQ ID NO: 149 - DNA, 327 bp, synthetic construct
  misc_feature 1..327: note = murine VL
  source 1..327: mol_type = other DNA
gagctcgttgtgactcaggaatctgcactcaccacatcacctggtgaaacagtcacactcacttgtcgctcaagtactggggctgttacaactagtaactatgccaactgggtccaagaaaaaccagatcatttattcactggtctaataggtggtaccaacaagcgagcaccaggtgtgcctgccagattctcaggctccctgattggagacaaggctgccctcaccatcacaggggcacagactgaggatgaggcaatatatttctgtgctctatggtacagcaacctctgggtgttcggtggaggaaccaaactgactgtccta

SEQ ID NO: 150 - DNA, 723 bp, synthetic construct
  misc_feature 1..723: note = CAIX LH scFv , single chain Fv
  source 1..723: mol_type = other DNA
gacattgtgatgacccagtctcaaagattcatgtccacaacagtaggagacagggtcagcatcacctgcaaggccagtcagaatgtggtttctgctgttgcctggtatcaacagaaaccaggacaatctcctaaactactgatttactcagcatccaatcggtacactggagtccctgatcgcttcacaggcagtggatctgggacagatttcactctcaccattagcaatatgcagtctgaagacctggctgattttttctgtcaacaatatagcaactatccgtggacgttcggtggaggcaccaagctggaaatcaaaggtggtggtggttctggcggcggcggctccggtggtggtggttctgacgtgaagctcgtggagtctgggggaggcttagtgaagcttggagggtccctgaaactctcctgtgcagcctctggattcactttcagtaactattacatgtcttgggttcgccagactccagagaagaggctggagttggtcgcagccattaatagtgatggtggtatcacctactatctagacactgtgaagggccgattcaccatttcaagagacaatgccaagaacaccctgtacctgcaaatgagcagtctgaagtctgaggacacagccttgttttactgtgcaagacaccgctcgggctacttttctatggactactggggtcaaggaacctcagtcaccgtctcctcc

SEQ ID NO: 151 - protein, 241 aa, synthetic construct
  REGION 1..241: note = CAIX LH scFv ; single chain Fv
DIVMTQSQRFMSTTVGDRVSITCKASQNVVSAVAWYQQKPGQSPKLLIYSASNRYTGVPDRFTGSGSGTDFTLTISNMQSEDLADFFCQQYSNYPWTFGGGTKLEIKGGGGSGGGGSGGGGSDVKLVESGGGLVKLGGSLKLSCAASGFTFSNYYMSWVRQTPEKRLELVAAINSDGGITYYLDTVKGRFTISRDNAKNTLYLQMSSLKSEDTALFYCARHRSGYFSMDYWGQGTSVTVSS

SEQ ID NO: 152 - DNA, 738 bp, synthetic construct
  misc_feature 1..738: note = EGFR21 LH scFv ; single chain Fv
  source 1..738: mol_type = other DNA
gacattgtgctgacacagtctcctgcttccttacctgtgtctctggggcagagggccaccatctcatgcagggccagccaaagtgtcagttcatctacttatagttatatacactggtaccaacagaaaccaggacagccacccaaactcctcatcacgtatgcatccaacctagaatctggggtccctgccaggttcagtggcagtgggtctgggacagacttcaccctcgacatccatcctgtggaggaggatgattcttcaacatattactgtcagcacagttgggagattccatttacgttcggctcggggacaaagttggaaataaaaggtggtggtggttctggcggcggcggctccggtggtggtggttctcaggttcagctgcagcagtctggacctgatctggtgaagcctggggcctcagtgaagatgtcctgcaaggcttctggacacactttcactgactgtgttataatctgggtgaaacagagagctggacagggccttgagtggattggacagatttatccagggactggtcgttcttactacaatgagattttcaagggcaaggccacactgactgcagacaaatcctccaacacagtccacattcaactcagcagcctgacatctgaggactctgcggtctatttctgtgccctatctactcttattcacgggacctggttttcttattggggccaagggactctggtcactgtctcttcc

SEQ ID NO: 153 - protein, 246 aa, synthetic construct
  REGION 1..246: note = EGFR21 LH scFv ; single chain Fv
DIVLTQSPASLPVSLGQRATISCRASQSVSSSTYSYIHWYQQKPGQPPKLLITYASNLESGVPARFSGSGSGTDFTLDIHPVEEDDSSTYYCQHSWEIPFTFGSGTKLEIKGGGGSGGGGSGGGGSQVQLQQSGPDLVKPGASVKMSCKASGHTFTDCVIIWVKQRAGQGLEWIGQIYPGTGRSYYNEIFKGKATLTADKSSNTVHIQLSSLTSEDSAVYFCALSTLIHGTWFSYWGQGTLVTVSS

SEQ ID NO: 154 - DNA, 726 bp, synthetic construct
  misc_feature 1..726: note = EGFRvIII-LH scFv ; single chain Fv
  source 1..726: mol_type = other DNA
gatgttgtgatgacccagactccactcactttgtcggttaccattggacaaccagcctctatctcttgcaagtcaagtcagagcctcttatatagtaatggaaaaacctatttgaattggttattacagaggccaggccagtctccaaagcgcctaatctatctggtatctaaactggactctggagtccctgacaggttcactggcagtggatcaggaacagattttacactgaaaatcagcagagtggaggctgaggatttgggaatttattactgcgtgcaagatacacattttcctcagacattcggtggaggcaccaagctggaaatcaaaggtggtggtggttctggcggcggcggctccggtggtggtggttctgaggtccagctgcaacagtctggacctgagctgctgaagcctggggcttcagtgaagatatcctgcaagacttctggatacacattcactgaatacaccatacactgggtgaagcagagccatggaaagagccttgagtggattggaggtattgatcctaacaatggtggtactatgtataaccaaaaattcaagggcaaggccacattgactgtagacaagtcttccagcacagcctacacggacctccgcagcctgacgtctgaggattctgcagtctattactgcacaagagcagaggctatggactactggggtcaaggaacctcagtcaccgtctcctcc

SEQ ID NO: 155 - protein, 242 aa, synthetic construct
  REGION 1..242: note = EGFRvIII-LH scFv ; single chain Fv
DVVMTQTPLTLSVTIGQPASISCKSSQSLLYSNGKTYLNWLLQRPGQSPKRLIYLVSKLDSGVPDRFTGSGSGTDFTLKISRVEAEDLGIYYCVQDTHFPQTFGGGTKLEIKGGGGSGGGGSGGGGSEVQLQQSGPELLKPGASVKISCKTSGYTFTEYTIHWVKQSHGKSLEWIGGIDPNNGGTMYNQKFKGKATLTVDKSSSTAYTDLRSLTSEDSAVYYCTRAEAMDYWGQGTSVTVSS

SEQ ID NO: 156 - DNA, 1485 bp, synthetic construct
  misc_feature 1..1485: note = CAIX LHxSEQ ID NO: 10 ; bispecific single chain antibody
  source 1..1485: mol_type = other DNA
gacattgtgatgacccagtctcaaagattcatgtccacaacagtaggagacagggtcagcatcacctgcaaggccagtcagaatgtggtttctgctgttgcctggtatcaacagaaaccaggacaatctcctaaactactgatttactcagcatccaatcggtacactggagtccctgatcgcttcacaggcagtggatctgggacagatttcactctcaccattagcaatatgcagtctgaagacctggctgattttttctgtcaacaatatagcaactatccgtggacgttcggtggaggcaccaagctggaaatcaaaggtggtggtggttctggcggcggcggctccggtggtggtggttctgacgtgaagctcgtggagtctgggggaggcttagtgaagcttggagggtccctgaaactctcctgtgcagcctctggattcactttcagtaactattacatgtcttgggttcgccagactccagagaagaggctggagttggtcgcagccattaatagtgatggtggtatcacctactatctagacactgtgaagggccgattcaccatttcaagagacaatgccaagaacaccctgtacctgcaaatgagcagtctgaagtctgaggacacagccttgttttactgtgcaagacaccgctcgggctacttttctatggactactggggtcaaggaacctcagtcaccgtctcctccggaggtggtggatccgaggtgaagcttctcgagtctggaggaggattggtgcagcctaaagggtcattgaaactctcatgtgcagcctctggattcaccttcaatacctacgccatgaactgggtccgccaggctccaggaaagggtttggaatgggttgctcgcataagaagtaaatataataattatgcaacatattatgccgattcagtgaaagacaggttcaccatctccagagatgattcacaaagcattctctatctacaaatgaacaacttgaaaactgaggacacagccatgtactactgtgtgagacatgggaacttcggtaatagctacgtttcctggtttgcttactggggccaagggactctggtcactgtctctgcaggtggtggtggttctggcggcggcggctccggtggtggtggttctcaggctgttgtgactcaggaatctgcactcaccacatcacctggtgaaacagtcacactcacttgtcgctcaagtactggggctgttacaactagtaactatgccaactgggtccaagaaaaaccagatcatttattcactggtctaataggtggtaccaacaagcgagctccaggtgtgcctgccagattctcaggctccctgattggagacaaggctgccctcaccatcacaggggcacagactgaggatgaggcaatatatttctgtgctctatggtacagcaacctctgggtgttcggtggaggaaccaaactgactgtccta

SEQ ID NO: 157 - protein, 495 aa, synthetic construct
  REGION 1..495: note = CAIX LHxSEQ ID NO: 10 ; bispecific single chain antibody
DIVMTQSQRFMSTTVGDRVSITCKASQNVVSAVAWYQQKPGQSPKLLIYSASNRYTGVPDRFTGSGSGTDFTLTISNMQSEDLADFFCQQYSNYPWTFGGGTKLEIKGGGGSGGGGSGGGGSDVKLVESGGGLVKLGGSLKLSCAASGFTFSNYYMSWVRQTPEKRLELVAAINSDGGITYYLDTVKGRFTISRDNAKNTLYLQMSSLKSEDTALFYCARHRSGYFSMDYWGQGTSVTVSSGGGGSEVKLLESGGGLVQPKGSLKLSCAASGFTFNTYAMNWVRQAPGKGLEWVARIRSKYNNYATYYADSVKDRFTISRDDSQSILYLQMNNLKTEDTAMYYCVRHGNFGNSYVSWFAYWGQGTLVTVSAGGGGSGGGGSGGGGSQAVVTQESALTTSPGETVTLTCRSSTGAVTTSNYANWVQEKPDHLFTGLIGGTNKRAPGVPARFSGSLIGDKAALTITGAQTEDEAIYFCALWYSNLWVFGGGTKLTVL

SEQ ID NO: 158 - DNA, 1500 bp, synthetic construct
  misc_feature 1..1500: note = EGFR21 LHxSEQ ID NO: 10 ; bispecific single chain antibody
  source 1..1500: mol_type = other DNA
gacattgtgctgacacagtctcctgcttccttacctgtgtctctggggcagagggccaccatctcatgcagggccagccaaagtgtcagttcatctacttatagttatatacactggtaccaacagaaaccaggacagccacccaaactcctcatcacgtatgcatccaacctagaatctggggtccctgccaggttcagtggcagtgggtctgggacagacttcaccctcgacatccatcctgtggaggaggatgattcttcaacatattactgtcagcacagttgggagattccatttacgttcggctcggggacaaagttggaaataaaaggtggtggtggttctggcggcggcggctccggtggtggtggttctcaggttcagctgcagcagtctggacctgatctggtgaagcctggggcctcagtgaagatgtcctgcaaggcttctggacacactttcactgactgtgttataatctgggtgaaacagagagctggacagggccttgagtggattggacagatttatccagggactggtcgttcttactacaatgagattttcaagggcaaggccacactgactgcagacaaatcctccaacacagtccacattcaactcagcagcctgacatctgaggactctgcggtctatttctgtgccctatctactcttattcacgggacctggttttcttattggggccaagggactctggtcactgtctcttccggaggtggtggatccgaggtgaagcttctcgagtctggaggaggattggtgcagcctaaagggtcattgaaactctcatgtgcagcctctggattcaccttcaatacctacgccatgaactgggtccgccaggctccaggaaagggtttggaatgggttgctcgcataagaagtaaatataataattatgcaacatattatgccgattcagtgaaagacaggttcaccatctccagagatgattcacaaagcattctctatctacaaatgaacaacttgaaaactgaggacacagccatgtactactgtgtgagacatgggaacttcggtaatagctacgtttcctggtttgcttactggggccaagggactctggtcactgtctctgcaggtggtggtggttctggcggcggcggctccggtggtggtggttctcaggctgttgtgactcaggaatctgcactcaccacatcacctggtgaaacagtcacactcacttgtcgctcaagtactggggctgttacaactagtaactatgccaactgggtccaagaaaaaccagatcatttattcactggtctaataggtggtaccaacaagcgagctccaggtgtgcctgccagattctcaggctccctgattggagacaaggctgccctcaccatcacaggggcacagactgaggatgaggcaatatatttctgtgctctatggtacagcaacctctgggtgttcggtggaggaaccaaactgactgtccta

SEQ ID NO: 159 - protein, 500 aa, synthetic construct
  REGION 1..500: note = EGFR21 LHxSEQ ID NO: 10 ; bispecific single chain antibody
DIVLTQSPASLPVSLGQRATISCRASQSVSSSTYSYIHWYQQKPGQPPKLLITYASNLESGVPARFSGSGSGTDFTLDIHPVEEDDSSTYYCQHSWEIPFTFGSGTKLEIKGGGGSGGGGSGGGGSQVQLQQSGPDLVKPGASVKMSCKASGHTFTDCVIIWVKQRAGQGLEWIGQIYPGTGRSYYNEIFKGKATLTADKSSNTVHIQLSSLTSEDSAVYFCALSTLIHGTWFSYWGQGTLVTVSSGGGGSEVKLLESGGGLVQPKGSLKLSCAASGFTFNTYAMNWVRQAPGKGLEWVARIRSKYNNYATYYADSVKDRFTISRDDSQSILYLQMNNLKTEDTAMYYCVRHGNFGNSYVSWFAYWGQGTLVTVSAGGGGSGGGGSGGGGSQAVVTQESALTTSPGETVTLTCRSSTGAVTTSNYANWVQEKPDHLFTGLIGGTNKRAPGVPARFSGSLIGDKAALTITGAQTEDEAIYFCALWYSNLWVFGGGTKLTVL

SEQ ID NO: 160 - DNA, 1488 bp, synthetic construct
  misc_feature 1..1488: note = EGFRvIII-LHxSEQ ID NO: 10 ; bispecific single chain antibody
  source 1..1488: mol_type = other DNA
gatgttgtgatgacccagactccactcactttgtcggttaccattggacaaccagcctctatctcttgcaagtcaagtcagagcctcttatatagtaatggaaaaacctatttgaattggttattacagaggccaggccagtctccaaagcgcctaatctatctggtatctaaactggactctggagtccctgacaggttcactggcagtggatcaggaacagattttacactgaaaatcagcagagtggaggctgaggatttgggaatttattactgcgtgcaagatacacattttcctcagacattcggtggaggcaccaagctggaaatcaaaggtggtggtggttctggcggcggcggctccggtggtggtggttctgaggtccagctgcaacagtctggacctgagctgctgaagcctggggcttcagtgaagatatcctgcaagacttctggatacacattcactgaatacaccatacactgggtgaagcagagccatggaaagagccttgagtggattggaggtattgatcctaacaatggtggtactatgtataaccaaaaattcaagggcaaggccacattgactgtagacaagtcttccagcacagcctacacggacctccgcagcctgacgtctgaggattctgcagtctattactgcacaagagcagaggctatggactactggggtcaaggaacctcagtcaccgtctcctccggaggtggtggatccgaggtgaagcttctcgagtctggaggaggattggtgcagcctaaagggtcattgaaactctcatgtgcagcctctggattcaccttcaatacctacgccatgaactgggtccgccaggctccaggaaagggtttggaatgggttgctcgcataagaagtaaatataataattatgcaacatattatgccgattcagtgaaagacaggttcaccatctccagagatgattcacaaagcattctctatctacaaatgaacaacttgaaaactgaggacacagccatgtactactgtgtgagacatgggaacttcggtaatagctacgtttcctggtttgcttactggggccaagggactctggtcactgtctctgcaggtggtggtggttctggcggcggcggctccggtggtggtggttctcaggctgttgtgactcaggaatctgcactcaccacatcacctggtgaaacagtcacactcacttgtcgctcaagtactggggctgttacaactagtaactatgccaactgggtccaagaaaaaccagatcatttattcactggtctaataggtggtaccaacaagcgagctccaggtgtgcctgccagattctcaggctccctgattggagacaaggctgccctcaccatcacaggggcacagactgaggatgaggcaatatatttctgtgctctatggtacagcaacctctgggtgttcggtggaggaaccaaactgactgtccta

SEQ ID NO: 161 - protein, 496 aa, synthetic construct
  REGION 1..496: note = EGFRvIII-LHxSEQ ID NO: 10 ; bispecific single chain antibody
DVVMTQTPLTLSVTIGQPASISCKSSQSLLYSNGKTYLNWLLQRPGQSPKRLIYLVSKLDSGVPDRFTGSGSGTDFTLKISRVEAEDLGIYYCVQDTHFPQTFGGGTKLEIKGGGGSGGGGSGGGGSEVQLQQSGPELLKPGASVKISCKTSGYTFTEYTIHWVKQSHGKSLEWIGGIDPNNGGTMYNQKFKGKATLTVDKSSSTAYTDLRSLTSEDSAVYYCTRAEAMDYWGQGTSVTVSSGGGGSEVKLLESGGGLVQPKGSLKLSCAASGFTFNTYAMNWVRQAPGKGLEWVARIRSKYNNYATYYADSVKDRFTISRDDSQSILYLQMNNLKTEDTAMYYCVRHGNFGNSYVSWFAYWGQGTLVTVSAGGGGSGGGGSGGGGSQAVVTQESALTTSPGETVTLTCRSSTGAVTTSNYANWVQEKPDHLFTGLIGGTNKRAPGVPARFSGSLIGDKAALTITGAQTEDEAIYFCALWYSNLWVFGGGTKLTVL

SEQ ID NO: 162 - protein, 243 aa, synthetic construct
  REGION 1..243: note = anti CD3
DIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 163 - protein, 243 aa, synthetic construct
  REGION 1..243: note = deimmunised(di)-anti CD3 deimmunised single chain Fv
DVQLVQSGAEVKKPGASVKVSCKASGYTFTRYTMHWVRQAPGQGLEWIGYINPSRGYTNYADSVKGRFTITTDKSTSTAYMELSSLRSEDTATYYCARYYDDHYCLDYWGQGTTVTVSSGEGTSTGSGGSGGSGGADDIVLTQSPATLSLSPGERATLSCRASQSVSYMNWYQQKPGKAPKRWIYDTSKVASGVPARFSGSGSGTDYSLTINSLEAEDAATYYCQQWSSNPLTFGGGTKVEIK

SEQ ID NO: 164 - protein, 9 aa, Mus musculus
QQWSRNPPT

SEQ ID NO: 165 - protein, 7 aa, Mus musculus
DSSKLAS

SEQ ID NO: 166 - protein, 10 aa, Mus musculus
SASSSVSYMN

SEQ ID NO: 167 - DNA, 327 bp, synthetic construct
  misc_feature 1..327: note = Human-like VL VL region
  source 1..327: mol_type = other DNA
gagctcgttgtgactcaggaaccttcactcaccgtatcacctggtggaacagtcacactcacttgtcgctcgtcgactggggctgttacaactagcaactatgccaactgggtccaacaaaaaccaggtcaggcaccccgtggtctaataggtggtaccaacaagcgcgcaccaggtactcctgccagattctcaggctccctgcttggaggcaaggctgccctcaccctctcaggggtacagccagaggatgaggcagaatattactgtgctctatggtacagcaacctctgggtgttcggtggaggaaccaaactgactgtccta

SEQ ID NO: 168 - protein, 109 aa, synthetic construct
  REGION 1..109: note = Human-like VL VL region
ELVVTQEPSLTVSPGGTVTLTCRSSTGAVTTSNYANWVQQKPGQAPRGLIGGTNKRAPGTPARFSGSLLGGKAALTLSGVQPEDEAEYYCALWYSNLWVFGGGTKLTVL

SEQ ID NO: 169 - DNA, 747 bp, synthetic construct
  misc_feature 1..747: note = Human-like VH (SEQ ID NO: 110) x Human-like VL (SEQ ID NO: 168) scFv ; single chain Fv
  source 1..747: mol_type = other DNA
gaggtgcagctgctcgagtctggaggaggattggtgcagcctggagggtcattgaaactctcatgtgcagcctctggattcaccttcaatacctacgccatgaactgggtccgccaggctccaggaaagggtttggaatgggttgctcgcataagaagtaaatataataattatgcaacatattatgccgattcagtgaaagacaggttcaccatctccagagatgattcaaaaaacactgcctatctacaaatgaacaacttgaaaactgaggacactgccgtgtactactgtgtgagacatgggaacttcggtaatagctacgtttcctggtttgcttactggggccaagggactctggtcaccgtctcctcaggtggtggtggttctggcggcggcggctccggtggtggtggttctgagctcgttgtgactcaggaaccttcactcaccgtatcacctggtggaacagtcacactcacttgtcgctcgtcgactggggctgttacaactagcaactatgccaactgggtccaacaaaaaccaggtcaggcaccccgtggtctaataggtggtaccaacaagcgcgcaccaggtactcctgccagattctcaggctccctgcttggaggcaaggctgccctcaccctctcaggggtacagccagaggatgaggcagaatattactgtgctctatggtacagcaacctctgggtgttcggtggaggaaccaaactgactgtccta

SEQ ID NO: 170 - protein, 249 aa, synthetic construct
  REGION 1..249: note = Human-like VH (SEQ ID NO: 110) x Human-like VL (SEQ ID NO: 168) scFv ; single chain Fv
EVQLLESGGGLVQPGGSLKLSCAASGFTFNTYAMNWVRQAPGKGLEWVARIRSKYNNYATYYADSVKDRFTISRDDSKNTAYLQMNNLKTEDTAVYYCVRHGNFGNSYVSWFAYWGQGTLVTVSSGGGGSGGGGSGGGGSELVVTQEPSLTVSPGGTVTLTCRSSTGAVTTSNYANWVQQKPGQAPRGLIGGTNKRAPGTPARFSGSLLGGKAALTLSGVQPEDEAEYYCALWYSNLWVFGGGTKLTVL

SEQ ID NO: 171 - DNA, 1530 bp, synthetic construct
  misc_feature 1..1530: note = EGFR HL x SEQ ID NO: 170 ; bispecific single chain antibody
  source 1..1530: mol_type = other DNA
caggtgcagctgcagcagtctgggcctgatctggtgaagcctggggcctcagtgaagatgtcctgcaaggcttctggacacactttcactgactgtgttataatctgggtgaaacagagagctggacagggccttgagtggattggacagatttatccagggactggtcgttcttactacaatgagattttcaagggcaaggccacactgactgcagacaaatcctccaacacagtccacattcaactcagcagcctgacatctgaggactctgcggtctatttctgtgccctatctactcttattcacgggacctggttttcttattggggccaagggactctggtcactgtctcttccggtggtggtggttctggcggcggcggctccggtggtggtggttctgacattgtactgacccagtctccagcttccttacctgtgtctctggggcagagggccaccatctcatgcagggccagccaaagtgtcagttcatctacttatagttatatacactggtaccaacagaaaccaggacagccacccaaactcctcatcacgtatgcatccaacctagaatctggggtccctgccaggttcagtggcagtgggtctgggacagacttcaccctcgacatccatcctgtggaggaggatgattcttcaacatattactgtcagcacagttgggagattccatttacgttcggctcggggacaaagttggaaataaaatccggaggtggtggctccgaggtgcagctggtggagtctggaggaggattggtgcagcctggagggtcattgaaactctcatgtgcagcctctggattcaccttcaatacctacgccatgaactgggtccgccaggctccaggaaagggtttggaatgggttgctcgcataagaagtaaatataataattatgcaacatattatgccgattcagtgaaagacaggttcaccatctccagagatgattcaaaaaacactgcctatctacaaatgaacaacttgaaaactgaggacactgccgtgtactactgtgtgagacatgggaacttcggtaatagctacgtttcctggtttgcttactggggccaagggactctggtcaccgtctcctcaggtggtggtggttctggcggcggcggctccggtggtggtggttctcagaccgttgtgactcaggaaccttcactcaccgtatcacctggtggaacagtcacactcacttgtcgctcgtccactggggctgttacaactagcaactatgccaactgggtccaacaaaaaccaggtcaggcaccccgtggtctaataggtggtaccaacaagcgcgcaccaggtactcctgccagattctcaggctccctgcttggaggcaaggctgccctcaccctctcaggggtacagccagaggatgaggcagaatattactgtgctctatggtacagcaacctctgggtgttcggtggaggaaccaaactgactgtcctacatcatcaccatcatcattaggtcgac

SEQ ID NO: 172 - protein, 507 aa, synthetic construct
  REGION 1..507: note = EGFR HL x SEQ ID NO: 170 ; bispecific single chain antibody
QVQLQQSGPDLVKPGASVKMSCKASGHTFTDCVIIWVKQRAGQGLEWIGQIYPGTGRSYYNEIFKGKATLTADKSSNTVHIQLSSLTSEDSAVYFCALSTLIHGTWFSYWGQGTLVTVSSGGGGSGGGGSGGGGSDIVLTQSPASLPVSLGQRATISCRASQSVSSSTYSYIHWYQQKPGQPPKLLITYASNLESGVPARFSGSGSGTDFTLDIHPVEEDDSSTYYCQHSWEIPFTFGSGTKLEIKSGGGGSEVQLVESGGGLVQPGGSLKLSCAASGFTFNTYAMNWVRQAPGKGLEWVARIRSKYNNYATYYADSVKDRFTISRDDSKNTAYLQMNNLKTEDTAVYYCVRHGNFGNSYVSWFAYWGQGTLVTVSSGGGGSGGGGSGGGGSQTVVTQEPSLTVSPGGTVTLTCRSSTGAVTTSNYANWVQQKPGQAPRGLIGGTNKRAPGTPARFSGSLLGGKAALTLSGVQPEDEAEYYCALWYSNLWVFGGGTKLTVLHHHHHH

SEQ ID NO: 173 - DNA, 1527 bp, synthetic construct
  misc_feature 1..1527: note = EGFR LH x SEQ ID NO: 170 ; bispecific single chain antibody
  source 1..1527: mol_type = other DNA
gacattgtgctgacacagtctcctgcttccttacctgtgtctctggggcagagggccaccatctcatgcagggccagccaaagtgtcagttcatctacttatagttatatacactggtaccaacagaaaccaggacagccacccaaactcctcatcacgtatgcatccaacctagaatctggggtccctgccaggttcagtggcagtgggtctgggacagacttcaccctcgacatccatcctgtggaggaggatgattcttcaacatattactgtcagcacagttgggagattccatttacgttcggctcggggacaaagttggaaataaaaggtggtggtggttctggcggcggcggctccggtggtggtggttctcaggttcagctgcagcagtctggacctgatctggtgaagcctggggcctcagtgaagatgtcctgcaaggcttctggacacactttcactgactgtgttataatctgggtgaaacagagagctggacagggccttgagtggattggacagatttatccagggactggtcgttcttactacaatgagattttcaagggcaaggccacactgactgcagacaaatcctccaacacagtccacattcaactcagcagcctgacatctgaggactctgcggtctatttctgtgccctatctactcttattcacgggacctggttttcttattggggccaagggactctggtcactgtctcttccggaggtggtggctccgaggtgcagctggtggagtctggaggaggattggtgcagcctggagggtcattgaaactctcatgtgcagcctctggattcaccttcaatacctacgccatgaactgggtccgccaggctccaggaaagggtttggaatgggttgctcgcataagaagtaaatataataattatgcaacatattatgccgattcagtgaaagacaggttcaccatctccagagatgattcaaaaaacactgcctatctacaaatgaacaacttgaaaactgaggacactgccgtgtactactgtgtgagacatgggaacttcggtaatagctacgtttcctggtttgcttactggggccaagggactctggtcaccgtctcctcaggtggtggtggttctggcggcggcggctccggtggtggtggttctcagaccgttgtgactcaggaaccttcactcaccgtatcacctggtggaacagtcacactcacttgtcgctcgtccactggggctgttacaactagcaactatgccaactgggtccaacaaaaaccaggtcaggcaccccgtggtctaataggtggtaccaacaagcgcgcaccaggtactcctgccagattctcaggctccctgcttggaggcaaggctgccctcaccctctcaggggtacagccagaggatgaggcagaatattactgtgctctatggtacagcaacctctgggtgttcggtggaggaaccaaactgactgtcctacatcatcaccatcatcattaggtcgac

SEQ ID NO: 174 - protein, 506 aa, synthetic construct
  REGION 1..506: note = EGFR LH x SEQ ID NO: 170 ; bispecific single chain antibody
DIVLTQSPASLPVSLGQRATISCRASQSVSSSTYSYIHWYQQKPGQPPKLLITYASNLESGVPARFSGSGSGTDFTLDIHPVEEDDSSTYYCQHSWEIPFTFGSGTKLEIKGGGGSGGGGSGGGGSQVQLQQSGPDLVKPGASVKMSCKASGHTFTDCVIIWVKQRAGQGLEWIGQIYPGTGRSYYNEIFKGKATLTADKSSNTVHIQLSSLTSEDSAVYFCALSTLIHGTWFSYWGQGTLVTVSSGGGGSEVQLVESGGGLVQPGGSLKLSCAASGFTFNTYAMNWVRQAPGKGLEWVARIRSKYNNYATYYADSVKDRFTISRDDSKNTAYLQMNNLKTEDTAVYYCVRHGNFGNSYVSWFAYWGQGTLVTVSSGGGGSGGGGSGGGGSQTVVTQEPSLTVSPGGTVTLTCRSSTGAVTTSNYANWVQQKPGQAPRGLIGGTNKRAPGTPARFSGSLLGGKAALTLSGVQPEDEAEYYCALWYSNLWVFGGGTKLTVLHHHHHH

SEQ ID NO: 175 - DNA, 1530 bp, synthetic construct
  misc_feature 1..1530: note = EGFR HL x SEQ ID NO: 194 ; bispecific single chain antibody
  source 1..1530: mol_type = other DNA
caggtgcagctgcagcagtctgggcctgatctggtgaagcctggggcctcagtgaagatgtcctgcaaggcttctggacacactttcactgactgtgttataatctgggtgaaacagagagctggacagggccttgagtggattggacagatttatccagggactggtcgttcttactacaatgagattttcaagggcaaggccacactgactgcagacaaatcctccaacacagtccacattcaactcagcagcctgacatctgaggactctgcggtctatttctgtgccctatctactcttattcacgggacctggttttcttattggggccaagggactctggtcactgtctcttccggtggtggtggttctggcggcggcggctccggtggtggtggttctgacattgtactgacccagtctccagcttccttacctgtgtctctggggcagagggccaccatctcatgcagggccagccaaagtgtcagttcatctacttatagttatatacactggtaccaacagaaaccaggacagccacccaaactcctcatcacgtatgcatccaacctagaatctggggtccctgccaggttcagtggcagtgggtctgggacagacttcaccctcgacatccatcctgtggaggaggatgattcttcaacatattactgtcagcacagttgggagattccatttacgttcggctcggggacaaagttggaaataaaatccggaggtggtggctcccagaccgttgtgactcaggaaccttcactcaccgtatcacctggtggaacagtcacactcacttgtcgctcgtccactggggctgttacaactagcaactatgccaactgggtccaacaaaaaccaggtcaggcaccccgtggtctaataggtggtaccaacaagcgcgcaccaggtactcctgccagattctcaggctccctgcttggaggcaaggctgccctcaccctctcaggggtacagccagaggatgaggcagaatattactgtgctctatggtacagcaacctctgggtgttcggtggaggaaccaaactgactgtcctaggtggtggtggttctggcggcggcggctccggtggtggtggttctgaggtgcagctggtggagtctggaggaggattggtgcagcctggagggtcattgaaactctcatgtgcagcctctggattcaccttcaatacctacgccatgaactgggtccgccaggctccaggaaagggtttggaatgggttgctcgcataagaagtaaatataataattatgcaacatattatgccgattcagtgaaagacaggttcaccatctccagagatgattcaaaaaacactgcctatctacaaatgaacaacttgaaaactgaggacactgccgtgtactactgtgtgagacatgggaacttcggtaatagctacgtttcctggtttgcttactggggccaagggactctggtcaccgtctcctcacatcatcaccatcatcattaggtcgac

SEQ ID NO: 176 - protein, 507 aa, synthetic construct
  REGION 1..507: note = EGFR HL x SEQ ID NO: 194 ; bispecific single chain antibody
QVQLQQSGPDLVKPGASVKMSCKASGHTFTDCVIIWVKQRAGQGLEWIGQIYPGTGRSYYNEIFKGKATLTADKSSNTVHIQLSSLTSEDSAVYFCALSTLIHGTWFSYWGQGTLVTVSSGGGGSGGGGSGGGGSDIVLTQSPASLPVSLGQRATISCRASQSVSSSTYSYIHWYQQKPGQPPKLLITYASNLESGVPARFSGSGSGTDFTLDIHPVEEDDSSTYYCQHSWEIPFTFGSGTKLEIKSGGGGSQTVVTQEPSLTVSPGGTVTLTCRSSTGAVTTSNYANWVQQKPGQAPRGLIGGTNKRAPGTPARFSGSLLGGKAALTLSGVQPEDEAEYYCALWYSNLWVFGGGTKLTVLGGGGSGGGGSGGGGSEVQLVESGGGLVQPGGSLKLSCAASGFTFNTYAMNWVRQAPGKGLEWVARIRSKYNNYATYYADSVKDRFTISRDDSKNTAYLQMNNLKTEDTAVYYCVRHGNFGNSYVSWFAYWGQGTLVTVSSHHHHHH

SEQ ID NO: 177 - DNA, 1530 bp, synthetic construct
  misc_feature 1..1530: note = EGFR LH x SEQ ID NO: 194 ; bispecific single chain antibody
  source 1..1530: mol_type = other DNA
gacattgtgctgacacagtctcctgcttccttacctgtgtctctggggcagagggccaccatctcatgcagggccagccaaagtgtcagttcatctacttatagttatatacactggtaccaacagaaaccaggacagccacccaaactcctcatcacgtatgcatccaacctagaatctggggtccctgccaggttcagtggcagtgggtctgggacagacttcaccctcgacatccatcctgtggaggaggatgattcttcaacatattactgtcagcacagttgggagattccatttacgttcggctcggggacaaagttggaaataaaaggtggtggtggttctggcggcggcggctccggtggtggtggttctcaggttcagctgcagcagtctggacctgatctggtgaagcctggggcctcagtgaagatgtcctgcaaggcttctggacacactttcactgactgtgttataatctgggtgaaacagagagctggacagggccttgagtggattggacagatttatccagggactggtcgttcttactacaatgagattttcaagggcaaggccacactgactgcagacaaatcctccaacacagtccacattcaactcagcagcctgacatctgaggactctgcggtctatttctgtgccctatctactcttattcacgggacctggttttcttattggggccaagggactctggtcactgtctcttcctccggaggtggtggctcccagaccgttgtgactcaggaaccttcactcaccgtatcacctggtggaacagtcacactcacttgtcgctcgtccactggggctgttacaactagcaactatgccaactgggtccaacaaaaaccaggtcaggcaccccgtggtctaataggtggtaccaacaagcgcgcaccaggtactcctgccagattctcaggctccctgcttggaggcaaggctgccctcaccctctcaggggtacagccagaggatgaggcagaatattactgtgctctatggtacagcaacctctgggtgttcggtggaggaaccaaactgactgtcctaggtggtggtggttctggcggcggcggctccggtggtggtggttctgaggtgcagctggtggagtctggaggaggattggtgcagcctggagggtcattgaaactctcatgtgcagcctctggattcaccttcaatacctacgccatgaactgggtccgccaggctccaggaaagggtttggaatgggttgctcgcataagaagtaaatataataattatgcaacatattatgccgattcagtgaaagacaggttcaccatctccagagatgattcaaaaaacactgcctatctacaaatgaacaacttgaaaactgaggacactgccgtgtactactgtgtgagacatgggaacttcggtaatagctacgtttcctggtttgcttactggggccaagggactctggtcaccgtctcctcacatcatcaccatcatcattaggtcgac

SEQ ID NO: 178 - protein, 507 aa, synthetic construct
  REGION 1..507: note = EGFR LH x SEQ ID NO: 194 ; bispecific single chain antibody
DIVLTQSPASLPVSLGQRATISCRASQSVSSSTYSYIHWYQQKPGQPPKLLITYASNLESGVPARFSGSGSGTDFTLDIHPVEEDDSSTYYCQHSWEIPFTFGSGTKLEIKGGGGSGGGGSGGGGSQVQLQQSGPDLVKPGASVKMSCKASGHTFTDCVIIWVKQRAGQGLEWIGQIYPGTGRSYYNEIFKGKATLTADKSSNTVHIQLSSLTSEDSAVYFCALSTLIHGTWFSYWGQGTLVTVSSSGGGGSQTVVTQEPSLTVSPGGTVTLTCRSSTGAVTTSNYANWVQQKPGQAPRGLIGGTNKRAPGTPARFSGSLLGGKAALTLSGVQPEDEAEYYCALWYSNLWVFGGGTKLTVLGGGGSGGGGSGGGGSEVQLVESGGGLVQPGGSLKLSCAASGFTFNTYAMNWVRQAPGKGLEWVARIRSKYNNYATYYADSVKDRFTISRDDSKNTAYLQMNNLKTEDTAVYYCVRHGNFGNSYVSWFAYWGQGTLVTVSSHHHHHH

SEQ ID NO: 179 - DNA, 1530 bp, synthetic construct
  misc_feature 1..1530: note = SEQ ID NO: 170 x EGFR HL ; bispecific single chain antibody
  source 1..1530: mol_type = other DNA
gaggtgcagctggtggagtctggaggaggattggtgcagcctggagggtcattgaaactctcatgtgcagcctctggattcaccttcaatacctacgccatgaactgggtccgccaggctccaggaaagggtttggaatgggttgctcgcataagaagtaaatataataattatgcaacatattatgccgattcagtgaaagacaggttcaccatctccagagatgattcaaaaaacactgcctatctacaaatgaacaacttgaaaactgaggacactgccgtgtactactgtgtgagacatgggaacttcggtaatagctacgtttcctggtttgcttactggggccaagggactctggtcaccgtctcctcaggtggtggtggttctggcggcggcggctccggtggtggtggttctcagaccgttgtgactcaggaaccttcactcaccgtatcacctggtggaacagtcacactcacttgtcgctcgtccactggggctgttacaactagcaactatgccaactgggtccaacaaaaaccaggtcaggcaccccgtggtctaataggtggtaccaacaagcgcgcaccaggtactcctgccagattctcaggctccctgcttggaggcaaggctgccctcaccctctcaggggtacagccagaggatgaggcagaatattactgtgctctatggtacagcaacctctgggtgttcggtggaggaaccaaactgactgtcctatccggaggtggtggctcccaggtgcagctgcagcagtctgggcctgatctggtgaagcctggggcctcagtgaagatgtcctgcaaggcttctggacacactttcactgactgtgttataatctgggtgaaacagagagctggacagggccttgagtggattggacagatttatccagggactggtcgttcttactacaatgagattttcaagggcaaggccacactgactgcagacaaatcctccaacacagtccacattcaactcagcagcctgacatctgaggactctgcggtctatttctgtgccctatctactcttattcacgggacctggttttcttattggggccaagggactctggtcactgtctcttccggtggtggtggttctggcggcggcggctccggtggtggtggttctgacattgtactgacccagtctccagcttccttacctgtgtctctggggcagagggccaccatctcatgcagggccagccaaagtgtcagttcatctacttatagttatatacactggtaccaacagaaaccaggacagccacccaaactcctcatcacgtatgcatccaacctagaatctggggtccctgccaggttcagtggcagtgggtctgggacagacttcaccctcgacatccatcctgtggaggaggatgattcttcaacatattactgtcagcacagttgggagattccatttacgttcggctcggggacaaagttggaaataaaacatcatcaccatcatcattaggtcgac

SEQ ID NO: 180 - protein, 507 aa, synthetic construct
  REGION 1..507: note = SEQ ID NO: 170 x EGFR HL , bispecific single chain antibody
EVQLVESGGGLVQPGGSLKLSCAASGFTFNTYAMNWVRQAPGKGLEWVARIRSKYNNYATYYADSVKDRFTISRDDSKNTAYLQMNNLKTEDTAVYYCVRHGNFGNSYVSWFAYWGQGTLVTVSSGGGGSGGGGSGGGGSQTVVTQEPSLTVSPGGTVTLTCRSSTGAVTTSNYANWVQQKPGQAPRGLIGGTNKRAPGTPARFSGSLLGGKAALTLSGVQPEDEAEYYCALWYSNLWVFGGGTKLTVLSGGGGSQVQLQQSGPDLVKPGASVKMSCKASGHTFTDCVIIWVKQRAGQGLEWIGQIYPGTGRSYYNEIFKGKATLTADKSSNTVHIQLSSLTSEDSAVYFCALSTLIHGTWFSYWGQGTLVTVSSGGGGSGGGGSGGGGSDIVLTQSPASLPVSLGQRATISCRASQSVSSSTYSYIHWYQQKPGQPPKLLITYASNLESGVPARFSGSGSGTDFTLDIHPVEEDDSSTYYCQHSWEIPFTFGSGTKLEIKHHHHHH

SEQ ID NO: 181 - DNA, 1530 bp, synthetic construct
  misc_feature 1..1530: note = SEQ ID NO: 194 x EGFR HL ; bispecific single chain antibody
  source 1..1530: mol_type = other DNA
cagaccgttgtgactcaggaaccttcactcaccgtatcacctggtggaacagtcacactcacttgtcgctcgtccactggggctgttacaactagcaactatgccaactgggtccaacaaaaaccaggtcaggcaccccgtggtctaataggtggtaccaacaagcgcgcaccaggtactcctgccagattctcaggctccctgcttggaggcaaggctgccctcaccctctcaggggtacagccagaggatgaggcagaatattactgtgctctatggtacagcaacctctgggtgttcggtggaggaaccaaactgactgtcctaggtggtggtggttctggcggcggcggctccggtggtggtggttctgaggtgcagctggtggagtctggaggaggattggtgcagcctggagggtcattgaaactctcatgtgcagcctctggattcaccttcaatacctacgccatgaactgggtccgccaggctccaggaaagggtttggaatgggttgctcgcataagaagtaaatataataattatgcaacatattatgccgattcagtgaaagacaggttcaccatctccagagatgattcaaaaaacactgcctatctacaaatgaacaacttgaaaactgaggacactgccgtgtactactgtgtgagacatgggaacttcggtaatagctacgtttcctggtttgcttactggggccaagggactctggtcaccgtctcctcatccggaggtggtggctcccaggtgcagctgcagcagtctgggcctgatctggtgaagcctggggcctcagtgaagatgtcctgcaaggcttctggacacactttcactgactgtgttataatctgggtgaaacagagagctggacagggccttgagtggattggacagatttatccagggactggtcgttcttactacaatgagattttcaagggcaaggccacactgactgcagacaaatcctccaacacagtccacattcaactcagcagcctgacatctgaggactctgcggtctatttctgtgccctatctactcttattcacgggacctggttttcttattggggccaagggactctggtcactgtctcttccggtggtggtggttctggcggcggcggctccggtggtggtggttctgacattgtactgacccagtctccagcttccttacctgtgtctctggggcagagggccaccatctcatgcagggccagccaaagtgtcagttcatctacttatagttatatacactggtaccaacagaaaccaggacagccacccaaactcctcatcacgtatgcatccaacctagaatctggggtccctgccaggttcagtggcagtgggtctgggacagacttcaccctcgacatccatcctgtggaggaggatgattcttcaacatattactgtcagcacagttgggagattccatttacgttcggctcggggacaaagttggaaataaaacatcatcaccatcatcattaggtcgac

SEQ ID NO: 182 - protein, 507 aa, synthetic construct
  REGION 1..507: note = SEQ ID NO: 194 x EGFR HL ; bispecific single chain antibody
QTVVTQEPSLTVSPGGTVTLTCRSSTGAVTTSNYANWVQQKPGQAPRGLIGGTNKRAPGTPARFSGSLLGGKAALTLSGVQPEDEAEYYCALWYSNLWVFGGGTKLTVLGGGGSGGGGSGGGGSEVQLVESGGGLVQPGGSLKLSCAASGFTFNTYAMNWVRQAPGKGLEWVARIRSKYNNYATYYADSVKDRFTISRDDSKNTAYLQMNNLKTEDTAVYYCVRHGNFGNSYVSWFAYWGQGTLVTVSSSGGGGSQVQLQQSGPDLVKPGASVKMSCKASGHTFTDCVIIWVKQRAGQGLEWIGQIYPGTGRSYYNEIFKGKATLTADKSSNTVHIQLSSLTSEDSAVYFCALSTLIHGTWFSYWGQGTLVTVSSGGGGSGGGGSGGGGSDIVLTQSPASLPVSLGQRATISCRASQSVSSSTYSYIHWYQQKPGQPPKLLITYASNLESGVPARFSGSGSGTDFTLDIHPVEEDDSSTYYCQHSWEIPFTFGSGTKLEIKHHHHHH

SEQ ID NO: 183 - DNA, 1530 bp, synthetic construct
  misc_feature 1..1530: note = SEQ ID NO: 170 x EGFR LH ; bispecific single chain antibody
  source 1..1530: mol_type = other DNA
gaggtgcagctggtggagtctggaggaggattggtgcagcctggagggtcattgaaactctcatgtgcagcctctggattcaccttcaatacctacgccatgaactgggtccgccaggctccaggaaagggtttggaatgggttgctcgcataagaagtaaatataataattatgcaacatattatgccgattcagtgaaagacaggttcaccatctccagagatgattcaaaaaacactgcctatctacaaatgaacaacttgaaaactgaggacactgccgtgtactactgtgtgagacatgggaacttcggtaatagctacgtttcctggtttgcttactggggccaagggactctggtcaccgtctcctcaggtggtggtggttctggcggcggcggctccggtggtggtggttctcagaccgttgtgactcaggaaccttcactcaccgtatcacctggtggaacagtcacactcacttgtcgctcgtccactggggctgttacaactagcaactatgccaactgggtccaacaaaaaccaggtcaggcaccccgtggtctaataggtggtaccaacaagcgcgcaccaggtactcctgccagattctcaggctccctgcttggaggcaaggctgccctcaccctctcaggggtacagccagaggatgaggcagaatattactgtgctctatggtacagcaacctctgggtgttcggtggaggaaccaaactgactgtcctatccggaggtggtggctccgacattgtgctgacacagtctcctgcttccttacctgtgtctctggggcagagggccaccatctcatgcagggccagccaaagtgtcagttcatctacttatagttatatacactggtaccaacagaaaccaggacagccacccaaactcctcatcacgtatgcatccaacctagaatctggggtccctgccaggttcagtggcagtgggtctgggacagacttcaccctcgacatccatcctgtggaggaggatgattcttcaacatattactgtcagcacagttgggagattccatttacgttcggctcggggacaaagttggaaataaaaggtggtggtggttctggcggcggcggctccggtggtggtggttctcaggttcagctgcagcagtctggacctgatctggtgaagcctggggcctcagtgaagatgtcctgcaaggcttctggacacactttcactgactgtgttataatctgggtgaaacagagagctggacagggccttgagtggattggacagatttatccagggactggtcgttcttactacaatgagattttcaagggcaaggccacactgactgcagacaaatcctccaacacagtccacattcaactcagcagcctgacatctgaggactctgcggtctatttctgtgccctatctactcttattcacgggacctggttttcttattggggccaagggactctggtcactgtctcttcccatcatcaccatcatcattaggtcgac

SEQ ID NO: 184 - protein, 507 aa, synthetic construct
  REGION 1..507: note = SEQ ID NO: 170 x EGFR LH ; bispecific single chain antibody
EVQLVESGGGLVQPGGSLKLSCAASGFTFNTYAMNWVRQAPGKGLEWVARIRSKYNNYATYYADSVKDRFTISRDDSKNTAYLQMNNLKTEDTAVYYCVRHGNFGNSYVSWFAYWGQGTLVTVSSGGGGSGGGGSGGGGSQTVVTQEPSLTVSPGGTVTLTCRSSTGAVTTSNYANWVQQKPGQAPRGLIGGTNKRAPGTPARFSGSLLGGKAALTLSGVQPEDEAEYYCALWYSNLWVFGGGTKLTVLSGGGGSDIVLTQSPASLPVSLGQRATISCRASQSVSSSTYSYIHWYQQKPGQPPKLLITYASNLESGVPARFSGSGSGTDFTLDIHPVEEDDSSTYYCQHSWEIPFTFGSGTKLEIKGGGGSGGGGSGGGGSQVQLQQSGPDLVKPGASVKMSCKASGHTFTDCVIIWVKQRAGQGLEWIGQIYPGTGRSYYNEIFKGKATLTADKSSNTVHIQLSSLTSEDSAVYFCALSTLIHGTWFSYWGQGTLVTVSSHHHHHH

SEQ ID NO: 185 - DNA, 1530 bp, synthetic construct
  misc_feature 1..1530: note = SEQ ID NO: 194 x EGFR LH ; bispecific single chain antibody
  source 1..1530: mol_type = other DNA
cagaccgttgtgactcaggaaccttcactcaccgtatcacctggtggaacagtcacactcacttgtcgctcgtccactggggctgttacaactagcaactatgccaactgggtccaacaaaaaccaggtcaggcaccccgtggtctaataggtggtaccaacaagcgcgcaccaggtactcctgccagattctcaggctccctgcttggaggcaaggctgccctcaccctctcaggggtacagccagaggatgaggcagaatattactgtgctctatggtacagcaacctctgggtgttcggtggaggaaccaaactgactgtcctaggtggtggtggttctggcggcggcggctccggtggtggtggttctgaggtgcagctggtggagtctggaggaggattggtgcagcctggagggtcattgaaactctcatgtgcagcctctggattcaccttcaatacctacgccatgaactgggtccgccaggctccaggaaagggtttggaatgggttgctcgcataagaagtaaatataataattatgcaacatattatgccgattcagtgaaagacaggttcaccatctccagagatgattcaaaaaacactgcctatctacaaatgaacaacttgaaaactgaggacactgccgtgtactactgtgtgagacatgggaacttcggtaatagctacgtttcctggtttgcttactggggccaagggactctggtcaccgtctcctcatccggaggtggtggctccgacattgtgctgacacagtctcctgcttccttacctgtgtctctggggcagagggccaccatctcatgcagggccagccaaagtgtcagttcatctacttatagttatatacactggtaccaacagaaaccaggacagccacccaaactcctcatcacgtatgcatccaacctagaatctggggtccctgccaggttcagtggcagtgggtctgggacagacttcaccctcgacatccatcctgtggaggaggatgattcttcaacatattactgtcagcacagttgggagattccatttacgttcggctcggggacaaagttggaaataaaaggtggtggtggttctggcggcggcggctccggtggtggtggttctcaggttcagctgcagcagtctggacctgatctggtgaagcctggggcctcagtgaagatgtcctgcaaggcttctggacacactttcactgactgtgttataatctgggtgaaacagagagctggacagggccttgagtggattggacagatttatccagggactggtcgttcttactacaatgagattttcaagggcaaggccacactgactgcagacaaatcctccaacacagtccacattcaactcagcagcctgacatctgaggactctgcggtctatttctgtgccctatctactcttattcacgggacctggttttcttattggggccaagggactctggtcactgtctcttcccatcatcaccatcatcattaggtcgac

SEQ ID NO: 186 - protein, 507 aa, synthetic construct
  REGION 1..507: note = SEQ ID NO: 194 x EGFR LH ; bispecific single chain antibody
QTVVTQEPSLTVSPGGTVTLTCRSSTGAVTTSNYANWVQQKPGQAPRGLIGGTNKRAPGTPARFSGSLLGGKAALTLSGVQPEDEAEYYCALWYSNLWVFGGGTKLTVLGGGGSGGGGSGGGGSEVQLVESGGGLVQPGGSLKLSCAASGFTFNTYAMNWVRQAPGKGLEWVARIRSKYNNYATYYADSVKDRFTISRDDSKNTAYLQMNNLKTEDTAVYYCVRHGNFGNSYVSWFAYWGQGTLVTVSSSGGGGSDIVLTQSPASLPVSLGQRATISCRASQSVSSSTYSYIHWYQQKPGQPPKLLITYASNLESGVPARFSGSGSGTDFTLDIHPVEEDDSSTYYCQHSWEIPFTFGSGTKLEIKGGGGSGGGGSGGGGSQVQLQQSGPDLVKPGASVKMSCKASGHTFTDCVIIWVKQRAGQGLEWIGQIYPGTGRSYYNEIFKGKATLTADKSSNTVHIQLSSLTSEDSAVYFCALSTLIHGTWFSYWGQGTLVTVSSHHHHHH

SEQ ID NO: 187 - DNA, 1515 bp, synthetic construct
  misc_feature 1..1515: note = CAIX HL x SEQ ID NO: 194 ; bispecific single chain antibody
  source 1..1515: mol_type = other DNA
gacgtgaagctcgtggagtctgggggaggcttagtgaagcttggagggtccctgaaactctcctgtgcagcctctggattcactttcagtaactattacatgtcttgggttcgccagactccagagaagaggctggagttggtcgcagccattaatagtgatggtggtatcacctactatctagacactgtgaagggccgattcaccatttcaagagacaatgccaagaacaccctgtacctgcaaatgagcagtctgaagtctgaggacacagccttgttttactgtgcaagacaccgctcgggctacttttctatggactactggggtcaaggaacctcagtcaccgtctcctcaggtggtggtggttctggcggcggcggctccggtggtggtggttctgacattgtgatgacccagtctcaaagattcatgtccacaacagtaggagacagggtcagcatcacctgcaaggccagtcagaatgtggtttctgctgttgcctggtatcaacagaaaccaggacaatctcctaaactactgatttactcagcatccaatcggtacactggagtccctgatcgcttcacaggcagtggatctgggacagatttcactctcaccattagcaatatgcagtctgaagacctggctgattttttctgtcaacaatatagcaactatccgtggacgttcggtggaggcaccaagctggaaatcaaatccggaggtggtggctcccagaccgttgtgactcaggaaccttcactcaccgtatcacctggtggaacagtcacactcacttgtcgctcgtccactggggctgttacaactagcaactatgccaactgggtccaacaaaaaccaggtcaggcaccccgtggtctaataggtggtaccaacaagcgcgcaccaggtactcctgccagattctcaggctccctgcttggaggcaaggctgccctcaccctctcaggggtacagccagaggatgaggcagaatattactgtgctctatggtacagcaacctctgggtgttcggtggaggaaccaaactgactgtcctaggtggtggtggttctggcggcggcggctccggtggtggtggttctgaggtgcagctggtggagtctggaggaggattggtgcagcctggagggtcattgaaactctcatgtgcagcctctggattcaccttcaatacctacgccatgaactgggtccgccaggctccaggaaagggtttggaatgggttgctcgcataagaagtaaatataataattatgcaacatattatgccgattcagtgaaagacaggttcaccatctccagagatgattcaaaaaacactgcctatctacaaatgaacaacttgaaaactgaggacactgccgtgtactactgtgtgagacatgggaacttcggtaatagctacgtttcctggtttgcttactggggccaagggactctggtcaccgtctcctcacatcatcaccatcatcattaggtcgac

SEQ ID NO: 188 - protein, 502 aa, synthetic construct
  REGION 1..502: note = CAIX HL x SEQ ID NO: 194 ; bispecific single chain antibody
DVKLVESGGGLVKLGGSLKLSCAASGFTFSNYYMSWVRQTPEKRLELVAAINSDGGITYYLDTVKGRFTISRDNAKNTLYLQMSSLKSEDTALFYCARHRSGYFSMDYWGQGTSVTVSSGGGGSGGGGSGGGGSDIVMTQSQRFMSTTVGDRVSITCKASQNVVSAVAWYQQKPGQSPKLLIYSASNRYTGVPDRFTGSGSGTDFTLTISNMQSEDLADFFCQQYSNYPWTFGGGTKLEIKSGGGGSQTVVTQEPSLTVSPGGTVTLTCRSSTGAVTTSNYANWVQQKPGQAPRGLIGGTNKRAPGTPARFSGSLLGGKAALTLSGVQPEDEAEYYCALWYSNLWVFGGGTKLTVLGGGGSGGGGSGGGGSEVQLVESGGGLVQPGGSLKLSCAASGFTFNTYAMNWVRQAPGKGLEWVARIRSKYNNYATYYADSVKDRFTISRDDSKNTAYLQMNNLKTEDTAVYYCVRHGNFGNSYVSWFAYWGQGTLVTVSSHHHHHH

SEQ ID NO: 189 - DNA, 1515 bp, synthetic construct
  misc_feature 1..1515: note = CAIX HL x SEQ ID NO: 170 ; bispecific single chain antibody
  source 1..1515: mol_type = other DNA
gacgtgaagctcgtggagtctgggggaggcttagtgaagcttggagggtccctgaaactctcctgtgcagcctctggattcactttcagtaactattacatgtcttgggttcgccagactccagagaagaggctggagttggtcgcagccattaatagtgatggtggtatcacctactatctagacactgtgaagggccgattcaccatttcaagagacaatgccaagaacaccctgtacctgcaaatgagcagtctgaagtctgaggacacagccttgttttactgtgcaagacaccgctcgggctacttttctatggactactggggtcaaggaacctcagtcaccgtctcctcaggtggtggtggttctggcggcggcggctccggtggtggtggttctgacattgtgatgacccagtctcaaagattcatgtccacaacagtaggagacagggtcagcatcacctgcaaggccagtcagaatgtggtttctgctgttgcctggtatcaacagaaaccaggacaatctcctaaactactgatttactcagcatccaatcggtacactggagtccctgatcgcttcacaggcagtggatctgggacagatttcactctcaccattagcaatatgcagtctgaagacctggctgattttttctgtcaacaatatagcaactatccgtggacgttcggtggaggcaccaagctggaaatcaaatccggaggtggtggctccgaggtgcagctggtggagtctggaggaggattggtgcagcctggagggtcattgaaactctcatgtgcagcctctggattcaccttcaatacctacgccatgaactgggtccgccaggctccaggaaagggtttggaatgggttgctcgcataagaagtaaatataataattatgcaacatattatgccgattcagtgaaagacaggttcaccatctccagagatgattcaaaaaacactgcctatctacaaatgaacaacttgaaaactgaggacactgccgtgtactactgtgtgagacatgggaacttcggtaatagctacgtttcctggtttgcttactggggccaagggactctggtcaccgtctcctcaggtggtggtggttctggcggcggcggctccggtggtggtggttctcagaccgttgtgactcaggaaccttcactcaccgtatcacctggtggaacagtcacactcacttgtcgctcgtccactggggctgttacaactagcaactatgccaactgggtccaacaaaaaccaggtcaggcaccccgtggtctaataggtggtaccaacaagcgcgcaccaggtactcctgccagattctcaggctccctgcttggaggcaaggctgccctcaccctctcaggggtacagccagaggatgaggcagaatattactgtgctctatggtacagcaacctctgggtgttcggtggaggaaccaaactgactgtcctacatcatcaccatcatcattaggtcgac

SEQ ID NO: 190 - protein, 502 aa, synthetic construct
  REGION 1..502: note = CAIX HL x SEQ ID NO: 170 ; bispecific single chain antibody
DVKLVESGGGLVKLGGSLKLSCAASGFTFSNYYMSWVRQTPEKRLELVAAINSDGGITYYLDTVKGRFTISRDNAKNTLYLQMSSLKSEDTALFYCARHRSGYFSMDYWGQGTSVTVSSGGGGSGGGGSGGGGSDIVMTQSQRFMSTTVGDRVSITCKASQNVVSAVAWYQQKPGQSPKLLIYSASNRYTGVPDRFTGSGSGTDFTLTISNMQSEDLADFFCQQYSNYPWTFGGGTKLEIKSGGGGSEVQLVESGGGLVQPGGSLKLSCAASGFTFNTYAMNWVRQAPGKGLEWVARIRSKYNNYATYYADSVKDRFTISRDDSKNTAYLQMNNLKTEDTAVYYCVRHGNFGNSYVSWFAYWGQGTLVTVSSGGGGSGGGGSGGGGSQTVVTQEPSLTVSPGGTVTLTCRSSTGAVTTSNYANWVQQKPGQAPRGLIGGTNKRAPGTPARFSGSLLGGKAALTLSGVQPEDEAEYYCALWYSNLWVFGGGTKLTVLHHHHHH

SEQ ID NO: 191 - DNA, 1515 bp, synthetic construct
  misc_feature 1..1515: note = CAIX LH x SEQ ID NO: 170 ; bispecific single chain antibody
  source 1..1515: mol_type = other DNA
gacattgtgatgacccagtctcaaagattcatgtccacaacagtaggagacagggtcagcatcacctgcaaggccagtcagaatgtggtttctgctgttgcctggtatcaacagaaaccaggacaatctcctaaactactgatttactcagcatccaatcggtacactggagtccctgatcgcttcacaggcagtggatctgggacagatttcactctcaccattagcaatatgcagtctgaagacctggctgattttttctgtcaacaatatagcaactatccgtggacgttcggtggaggcaccaagctggaaatcaaaggtggtggtggttctggcggcggcggctccggtggtggtggttctgacgtgaagctcgtggagtctgggggaggcttagtgaagcttggagggtccctgaaactctcctgtgcagcctctggattcactttcagtaactattacatgtcttgggttcgccagactccagagaagaggctggagttggtcgcagccattaatagtgatggtggtatcacctactatctagacactgtgaagggccgattcaccatttcaagagacaatgccaagaacaccctgtacctgcaaatgagcagtctgaagtctgaggacacagccttgttttactgtgcaagacaccgctcgggctacttttctatggactactggggtcaaggaacctcagtcaccgtctcctcctccggaggtggtggctccgaggtgcagctggtggagtctggaggaggattggtgcagcctggagggtcattgaaactctcatgtgcagcctctggattcaccttcaatacctacgccatgaactgggtccgccaggctccaggaaagggtttggaatgggttgctcgcataagaagtaaatataataattatgcaacatattatgccgattcagtgaaagacaggttcaccatctccagagatgattcaaaaaacactgcctatctacaaatgaacaacttgaaaactgaggacactgccgtgtactactgtgtgagacatgggaacttcggtaatagctacgtttcctggtttgcttactggggccaagggactctggtcaccgtctcctcaggtggtggtggttctggcggcggcggctccggtggtggtggttctcagaccgttgtgactcaggaaccttcactcaccgtatcacctggtggaacagtcacactcacttgtcgctcgtccactggggctgttacaactagcaactatgccaactgggtccaacaaaaaccaggtcaggcaccccgtggtctaataggtggtaccaacaagcgcgcaccaggtactcctgccagattctcaggctccctgcttggaggcaaggctgccctcaccctctcaggggtacagccagaggatgaggcagaatattactgtgctctatggtacagcaacctctgggtgttcggtggaggaaccaaactgactgtcctacatcatcaccatcatcattaggtcgac

SEQ ID NO: 192 - protein, 502 aa, synthetic construct
  REGION 1..502: note = CAIX LH x SEQ ID NO: 170 ; bispecific single chain antibody
DIVMTQSQRFMSTTVGDRVSITCKASQNVVSAVAWYQQKPGQSPKLLIYSASNRYTGVPDRFTGSGSGTDFTLTISNMQSEDLADFFCQQYSNYPWTFGGGTKLEIKGGGGSGGGGSGGGGSDVKLVESGGGLVKLGGSLKLSCAASGFTFSNYYMSWVRQTPEKRLELVAAINSDGGITYYLDTVKGRFTISRDNAKNTLYLQMSSLKSEDTALFYCARHRSGYFSMDYWGQGTSVTVSSSGGGGSEVQLVESGGGLVQPGGSLKLSCAASGFTFNTYAMNWVRQAPGKGLEWVARIRSKYNNYATYYADSVKDRFTISRDDSKNTAYLQMNNLKTEDTAVYYCVRHGNFGNSYVSWFAYWGQGTLVTVSSGGGGSGGGGSGGGGSQTVVTQEPSLTVSPGGTVTLTCRSSTGAVTTSNYANWVQQKPGQAPRGLIGGTNKRAPGTPARFSGSLLGGKAALTLSGVQPEDEAEYYCALWYSNLWVFGGGTKLTVLHHHHHH

SEQ ID NO: 193 - DNA, 747 bp, synthetic construct
  misc_feature 1..747: note = Human-like VL (SEQ ID NO: 168) x Human-like VH (SEQ ID NO: 110) scFv ; single chain Fv
  source 1..747: mol_type = other DNA
cagaccgttgtgactcaggaaccttcactcaccgtatcacctggtggaacagtcacactcacttgtcgctcgtccactggggctgttacaactagcaactatgccaactgggtccaacaaaaaccaggtcaggcaccccgtggtctaataggtggtaccaacaagcgcgcaccaggtactcctgccagattctcaggctccctgcttggaggcaaggctgccctcaccctctcaggggtacagccagaggatgaggcagaatattactgtgctctatggtacagcaacctctgggtgttcggtggaggaaccaaactgactgtcctaggtggtggtggttctggcggcggcggctccggtggtggtggttctgaggtgcagctggtggagtctggaggaggattggtgcagcctggagggtcattgaaactctcatgtgcagcctctggattcaccttcaatacctacgccatgaactgggtccgccaggctccaggaaagggtttggaatgggttgctcgcataagaagtaaatataataattatgcaacatattatgccgattcagtgaaagacaggttcaccatctccagagatgattcaaaaaacactgcctatctacaaatgaacaacttgaaaactgaggacactgccgtgtactactgtgtgagacatgggaacttcggtaatagctacgtttcctggtttgcttactggggccaagggactctggtcaccgtctcctca

SEQ ID NO: 194 - protein, 249 aa, synthetic construct
  REGION 1..249: note = Human-like VL (SEQ ID NO: 168) x Human-like VH (SEQ ID NO: 110) scFv ; single chain Fv
QTVVTQEPSLTVSPGGTVTLTCRSSTGAVTTSNYANWVQQKPGQAPRGLIGGTNKRAPGTPARFSGSLLGGKAALTLSGVQPEDEAEYYCALWYSNLWVFGGGTKLTVLGGGGSGGGGSGGGGSEVQLVESGGGLVQPGGSLKLSCAASGFTFNTYAMNWVRQAPGKGLEWVARIRSKYNNYATYYADSVKDRFTISRDDSKNTAYLQMNNLKTEDTAVYYCVRHGNFGNSYVSWFAYWGQGTLVTVSS

SEQ ID NO: 195 - protein, 13 aa, synthetic construct
  REGION 1..13: note = epitope
EFSELEQSGYYVC

SEQ ID NO: 196 - protein, 13 aa, synthetic construct
  REGION 1..13: note = epitope
EFSELEQSGYYVK

SEQ ID NO: 197 - DNA, 34 bp, synthetic construct
  misc_feature 1..34: note = 5' EGFR XbaI ; oligonucleotide
  source 1..34: mol_type = other DNA
ggtctagagcatgcgaccctccgggacggccggg

SEQ ID NO: 198 - DNA, 36 bp, synthetic construct
  misc_feature 1..36: note = 3' EGFR SalI ; oligonucleotide
  source 1..36: mol_type = other DNA
ttttaagtcgactcatgctccaataaattcactgct

SEQ ID NO: 199 - protein, 13 aa, synthetic construct
  REGION 1..13: note = epitope
QDGNEEMGSITQT

SEQ ID NO: 200 - protein, 13 aa, synthetic construct
  REGION 1..13: note = epitope
YYVSYPRGSNPED

SEQ ID NO: 201 - protein, 13 aa, synthetic construct
  REGION 1..13: note = epitope
EFSEMEQSGYYVC

SEQ ID NO: 202 - protein, 5 aa, synthetic construct
  REGION 1..5: note = epitope
  SITE 4: note = variation - /replace= "Met"
FSELE

SEQ ID NO: 203 - protein, 13 aa, synthetic construct
  REGION 1..13: note = epitope
QYPGSEILWQHND

SEQ ID NO: 204 - protein, 5 aa, synthetic construct
  REGION 1..5: note = epitope
  SITE 4: note = variation - /replace="Met"
FSELE

SEQ ID NO: 205 - protein, 5 aa, synthetic construct
  REGION 1..5: note = epitope
FSELE

SEQ ID NO: 206 - protein, 5 aa, synthetic construct
  REGION 1..5: note = epitope
FSEME

SEQ ID NO: 207 - protein, 13 aa, synthetic construct
  REGION 1..13: note = epitope
  SITE 5: note = variation - /replace="Met"
EFSELEQSGYYVC